SEQUENCE LISTING (1229 sequences)

SEQ ID NO: 1 - RNA, 45 nt, Unknown
  Description of Unknown GluR2 domain sequence
guggaauaguauaacaauaugcuaaauguuguuauaguaucccac

SEQ ID NO: 2 - RNA, 100 nt, Artificial Sequence
  Description of Artificial Sequence Synthetic polynucleotide
uccugcggcccaacgauuacgucgacgcgugagaauccaucugaauuaacuccuggcggggcucagccaugcugguucaccgauaaccugagauucgaca

SEQ ID NO: 3 - RNA, 100 nt, Artificial Sequence
  Description of Artificial Sequence Synthetic polynucleotide
uccugcggcccaacgauuacgucgacgcgugagaauccaucugaauuaacuccuggcggggcucagccaugcugguucaccgauaaccugauagucgaca

SEQ ID NO: 4 - RNA, 100 nt, Artificial Sequence
  Description of Artificial Sequence Synthetic polynucleotide
uccuagaccccaagagguacguaagagcgugcuauuccauguaacagaacuccuggcggggcucagccaugcugguucaccccgaaccugaaauucgaca

SEQ ID NO: 5 - RNA, 100 nt, Artificial Sequence
  Description of Artificial Sequence Synthetic polynucleotide
uccuagaccccaagagguacguaagagcgugcuauuccauguaacagaacuccuggcggggcucagccaugcugguucaccccgaaccugauagucgaca

SEQ ID NO: 6 - RNA, 100 nt, Artificial Sequence
  Description of Artificial Sequence Synthetic polynucleotide
uccauacccccaugacguacgguacagcguccacuuccaaguguccgaacuccuggcggggcucagccaugcugguucauucuggaccugacucucgaca

SEQ ID NO: 7 - RNA, 100 nt, Artificial Sequence
  Description of Artificial Sequence Synthetic polynucleotide
uccauacccccaugacguacgguacagcguccacuuccaaguguccgaacuccuggcggggcucagccaugcugguucauucuggaccugauagucgaca

SEQ ID NO: 8 - RNA, 100 nt, Artificial Sequence
  Description of Artificial Sequence Synthetic polynucleotide
ucggcucccccuaaccguaccagccagcgaauucuuccccugucucgaacuccuggcggggcucagccaugcugguucacgcgcgaccugacacucgaca

SEQ ID NO: 9 - RNA, 100 nt, Artificial Sequence
  Description of Artificial Sequence Synthetic polynucleotide
ucggcucccccuaaccguaccagccagcgaauucuuccccugucucgaacuccuggcggggcucagccaugcugguucacgcgcgaccugauagucgaca

SEQ ID NO: 10 - RNA, 100 nt, Artificial Sequence
  Description of Artificial Sequence Synthetic polynucleotide
uccugucccaacacccgucauucccagacagaucuuaugcuucuucgaacuccuggcggggcucagccaugcugguucaccgaagaccugagcaucgaca

SEQ ID NO: 11 - RNA, 100 nt, Artificial Sequence
  Description of Artificial Sequence Synthetic polynucleotide
uccugucccaacacccgucauucccagacagaucuuaugcuucuucgaacuccuggcggggcucagccaugcugguucaccgaagaccugauagucgaca

SEQ ID NO: 12 - RNA, 100 nt, Artificial Sequence
  Description of Artificial Sequence Synthetic polynucleotide
uccuguccagaaacccgcuagucccaagaugaucugagacgacuucgaacuccuggcggggcucagccaugcugguucaugucuaaccugagauucgaca

SEQ ID NO: 13 - RNA, 100 nt, Artificial Sequence
  Description of Artificial Sequence Synthetic polynucleotide
uccuguccagaaacccgcuagucccaagaugaucugagacgacuucgaacuccuggcggggcucagccaugcugguucaugucuaaccugauagucgaca

SEQ ID NO: 14 - RNA, 100 nt, Artificial Sequence
  Description of Artificial Sequence Synthetic polynucleotide
uccugucggacaaccccgccgucccucagugaucgaagugcacuucgaacuccuggcggggcucagccaugcugguucaucauagaccugaauaucgaca

SEQ ID NO: 15 - RNA, 100 nt, Artificial Sequence
  Description of Artificial Sequence Synthetic polynucleotide
uccugucggacaaccccgccgucccucagugaucgaagugcacuucgaacuccuggcggggcucagccaugcugguucaucauagaccugauagucgaca

SEQ ID NO: 16 - RNA, 100 nt, Artificial Sequence
  Description of Artificial Sequence Synthetic polynucleotide
uccuguaggccaaccaggacguccgcccgugaugaagauucacuucgaacuccuggcggggcucagccaugcugguucaccuuggaccugaauuucgaca

SEQ ID NO: 17 - RNA, 100 nt, Artificial Sequence
  Description of Artificial Sequence Synthetic polynucleotide
uccuguaggccaaccaggacguccgcccgugaugaagauucacuucgaacuccuggcggggcucagccaugcugguucaccuuggaccugauagucgaca

SEQ ID NO: 18 - RNA, 100 nt, Artificial Sequence
  Description of Artificial Sequence Synthetic polynucleotide
uccugcaacccaacagauacgucggugcgugagggcagaucacuucgaacuccuggcggggcucagccaugcugguucacaacgaaccugacucucgaca

SEQ ID NO: 19 - RNA, 100 nt, Artificial Sequence
  Description of Artificial Sequence Synthetic polynucleotide
uccugcaacccaacagauacgucggugcgugagggcagaucacuucgaacuccuggcggggcucagccaugcugguucacaacgaaccugauagucgaca

SEQ ID NO: 20 - RNA, 100 nt, Artificial Sequence
  Description of Artificial Sequence Synthetic polynucleotide
uccuacaccccaagagguacgugagagcgugcgggagcaucacuucgaacuccuggcggggcucagccaugcugguucacuauaaaccugacaaucgaca

SEQ ID NO: 21 - RNA, 100 nt, Artificial Sequence
  Description of Artificial Sequence Synthetic polynucleotide
uccuacaccccaagagguacgugagagcgugcgggagcaucacuucgaacuccuggcggggcucagccaugcugguucacuauaaaccugauagucgaca

SEQ ID NO: 22 - RNA, 100 nt, Artificial Sequence
  Description of Artificial Sequence Synthetic polynucleotide
uccacacccccacaacguacggcacagcguaccgcaccaucacuucgaacuccuggcggggcucagccaugcugguucaugccagaccugacauucgaca

SEQ ID NO: 23 - RNA, 100 nt, Artificial Sequence
  Description of Artificial Sequence Synthetic polynucleotide
uccacacccccacaacguacggcacagcguaccgcaccaucacuucgaacuccuggcggggcucagccaugcugguucaugccagaccugauagucgaca

SEQ ID NO: 24 - RNA, 100 nt, Artificial Sequence
  Description of Artificial Sequence Synthetic polynucleotide
uccugucccgacacccguuucucccagagcuuacuuccaucacuucgaacuccuggcggggcucagccaugcugguucaccauaaaccugagucucgaca

SEQ ID NO: 25 - RNA, 100 nt, Artificial Sequence
  Description of Artificial Sequence Synthetic polynucleotide
uccugucccgacacccguuucucccagagcuuacuuccaucacuucgaacuccuggcggggcucagccaugcugguucaccauaaaccugauagucgaca

SEQ ID NO: 26 - RNA, 100 nt, Artificial Sequence
  Description of Artificial Sequence Synthetic polynucleotide
uccuguccagaaacccgcaagucccaaauacuucuuccaucacuucgaacuccuggcggggcucagccaugcugguucacacacaaccugaaccucgaca

SEQ ID NO: 27 - RNA, 100 nt, Artificial Sequence
  Description of Artificial Sequence Synthetic polynucleotide
uccuguccagaaacccgcaagucccaaauacuucuuccaucacuucgaacuccuggcggggcucagccaugcugguucacacacaaccugauagucgaca

SEQ ID NO: 28 - RNA, 100 nt, Artificial Sequence
  Description of Artificial Sequence Synthetic polynucleotide
uccugucaggcaacccacccguccccaccauaucuuccaucacuucgaacuccuggcggggcucagccaugcugguucaugauaaaccugacccucgaca

SEQ ID NO: 29 - RNA, 100 nt, Artificial Sequence
  Description of Artificial Sequence Synthetic polynucleotide
uccugucaggcaacccacccguccccaccauaucuuccaucacuucgaacuccuggcggggcucagccaugcugguucaugauaaaccugauagucgaca

SEQ ID NO: 30 - RNA, 100 nt, Artificial Sequence
  Description of Artificial Sequence Synthetic polynucleotide
uccugucaaacaacccagccgucccagcgugaucuuccaucacuucgaacuccuggcggggcucagccaugcugguucauaaaggaccugaaaaucgaca

SEQ ID NO: 31 - RNA, 100 nt, Artificial Sequence
  Description of Artificial Sequence Synthetic polynucleotide
uccugucaaacaacccagccgucccagcgugaucuuccaucacuucgaacuccuggcggggcucagccaugcugguucauaaaggaccugauagucgaca

SEQ ID NO: 32 - RNA, 100 nt, Artificial Sequence
  Description of Artificial Sequence Synthetic polynucleotide
uccugucgaacaacccauccgucccuuagugauccggcaucacggauuucuccuggcggggcucagccaugcugguucaacgacuuccugauagucgaca

SEQ ID NO: 33 - RNA, 100 nt, Artificial Sequence
  Description of Artificial Sequence Synthetic polynucleotide
uccuguagaccaaccaccacguccacccgugaugacccaucagcguauacuccuggcggggcucagccaugcugguucaacucgauccugauagucgaca

SEQ ID NO: 34 - RNA, 100 nt, Artificial Sequence
  Description of Artificial Sequence Synthetic polynucleotide
uccugacacccaacagcuacgucgaugcgugaagcuccaucucaguaaacuccuggcggggcucagccaugcugguucaaccuguuccugauagucgaca

SEQ ID NO: 35 - RNA, 100 nt, Artificial Sequence
  Description of Artificial Sequence Synthetic polynucleotide
uccucaaccccaagggguacguaagagcguguuauuccauauaagagaacuccuggcggggcucagccaugcugguucaaccucuuccugauuugcgaca

SEQ ID NO: 36 - RNA, 100 nt, Artificial Sequence
  Description of Artificial Sequence Synthetic polynucleotide
uccucaaccccaagggguacguaagagcguguuauuccauauaagagaacuccuggcggggcucagccaugcugguucaaccucuuccugauagucgaca

SEQ ID NO: 37 - RNA, 100 nt, Artificial Sequence
  Description of Artificial Sequence Synthetic polynucleotide
uccgggcccccaucacguacggggcagcguuuacuuccagggaggcgaacuccuggcggggcucagccaugcugguucaauaucuuccugauccacgaca

SEQ ID NO: 38 - RNA, 100 nt, Artificial Sequence
  Description of Artificial Sequence Synthetic polynucleotide
uccgggcccccaucacguacggggcagcguuuacuuccagggaggcgaacuccuggcggggcucagccaugcugguucaauaucuuccugauagucgaca

SEQ ID NO: 39 - RNA, 100 nt, Artificial Sequence
  Description of Artificial Sequence Synthetic polynucleotide
ucagaucccccuaaccguaccgaccagcgaccucuuccugccgaucgaacuccuggcggggcucagccaugcugguucaaugagguccugaucaccgaca

SEQ ID NO: 40 - RNA, 100 nt, Artificial Sequence
  Description of Artificial Sequence Synthetic polynucleotide
ucagaucccccuaaccguaccgaccagcgaccucuuccugccgaucgaacuccuggcggggcucagccaugcugguucaaugagguccugauagucgaca

SEQ ID NO: 41 - RNA, 100 nt, Artificial Sequence
  Description of Artificial Sequence Synthetic polynucleotide
uccugucccaguacccgucucucccagaucgaucuugacugucuucgaacuccuggcggggcucagccaugcugguucaacccgguccugauuaacgaca

SEQ ID NO: 42 - RNA, 100 nt, Artificial Sequence
  Description of Artificial Sequence Synthetic polynucleotide
uccuguaggccaaccgaaacguccgcccgugaugcgaguucacuucgaacuccuggcggggcucagccaugcugguucaaccccauccugauuuccgaca

SEQ ID NO: 43 - RNA, 100 nt, Artificial Sequence
  Description of Artificial Sequence Synthetic polynucleotide
uccuguaggccaaccgaaacguccgcccgugaugcgaguucacuucgaacuccuggcggggcucagccaugcugguucaaccccauccugauagucgaca

SEQ ID NO: 44 - RNA, 100 nt, Artificial Sequence
  Description of Artificial Sequence Synthetic polynucleotide
uccuggcacccaacgacuacgucgacgcgugaagacggaucacuucgaacuccuggcggggcucagccaugcugguucaauuccauccugaucaacgaca

SEQ ID NO: 45 - RNA, 100 nt, Artificial Sequence
  Description of Artificial Sequence Synthetic polynucleotide
uccuggcacccaacgacuacgucgacgcgugaagacggaucacuucgaacuccuggcggggcucagccaugcugguucaauuccauccugauagucgaca

SEQ ID NO: 46 - RNA, 100 nt, Artificial Sequence
  Description of Artificial Sequence Synthetic polynucleotide
uccuuggccccaaggaguacgugggagcgugcgguagcaucacuucgaacuccuggcggggcucagccaugcugguucaauuggauccugaucuacgaca

SEQ ID NO: 47 - RNA, 100 nt, Artificial Sequence
  Description of Artificial Sequence Synthetic polynucleotide
uccuuggccccaaggaguacgugggagcgugcgguagcaucacuucgaacuccuggcggggcucagccaugcugguucaauuggauccugauagucgaca

SEQ ID NO: 48 - RNA, 100 nt, Artificial Sequence
  Description of Artificial Sequence Synthetic polynucleotide
uccaaacccccaugacguacgcagcagcguugcgagccaucacuucgaacuccuggcggggcucagccaugcugguucaacuuguuccugauuaccgaca

SEQ ID NO: 49 - RNA, 100 nt, Artificial Sequence
  Description of Artificial Sequence Synthetic polynucleotide
uccaaacccccaugacguacgcagcagcguugcgagccaucacuucgaacuccuggcggggcucagccaugcugguucaacuuguuccugauagucgaca

SEQ ID NO: 50 - RNA, 100 nt, Artificial Sequence
  Description of Artificial Sequence Synthetic polynucleotide
uccugucccaguacccguuaaucccagaucuuccuuccaucacuucgaacuccuggcggggcucagccaugcugguucaacgcuguccugauuuacgaca

SEQ ID NO: 51 - RNA, 100 nt, Artificial Sequence
  Description of Artificial Sequence Synthetic polynucleotide
uccuguccggaaacccgcuggucccauuaucuucuuccaucacuucgaacuccuggcggggcucagccaugcugguucaaucaaguccugauuuccgaca

SEQ ID NO: 52 - RNA, 100 nt, Artificial Sequence
  Description of Artificial Sequence Synthetic polynucleotide
uccuguccggaaacccgcuggucccauuaucuucuuccaucacuucgaacuccuggcggggcucagccaugcugguucaaucaaguccugauagucgaca

SEQ ID NO: 53 - RNA, 100 nt, Artificial Sequence
  Description of Artificial Sequence Synthetic polynucleotide
uccugucaagcaacccuaucgucccagcgugaucuuccaucacuucgaacuccuggcggggcucagccaugcugguucaaucgaauccugaucaccgaca

SEQ ID NO: 54 - RNA, 100 nt, Artificial Sequence
  Description of Artificial Sequence Synthetic polynucleotide
uccugucaagcaacccuaucgucccagcgugaucuuccaucacuucgaacuccuggcggggcucagccaugcugguucaaucgaauccugauagucgaca

SEQ ID NO: 55 - RNA, 100 nt, Artificial Sequence
  Description of Artificial Sequence Synthetic polynucleotide
uccuaagccccaaggaguacgugggagcgugugauuccauagaacagaacuccuggcggggcucagccaugcugguucaaaacaaugcugauauaggaca

SEQ ID NO: 56 - RNA, 100 nt, Artificial Sequence
  Description of Artificial Sequence Synthetic polynucleotide
uccuaagccccaaggaguacgugggagcgugugauuccauagaacagaacuccuggcggggcucagccaugcugguucaaaacaaugcugauagucgaca

SEQ ID NO: 57 - RNA, 100 nt, Artificial Sequence
  Description of Artificial Sequence Synthetic polynucleotide
uccgaccccccacagcguacggagcagcguaaccuuccagacaaacgaacuccuggcggggcucagccaugcugguucaaaauaaugcugauauaagaca

SEQ ID NO: 58 - RNA, 100 nt, Artificial Sequence
  Description of Artificial Sequence Synthetic polynucleotide
uccgaccccccacagcguacggagcagcguaaccuuccagacaaacgaacuccuggcggggcucagccaugcugguucaaaauaaugcugauagucgaca

SEQ ID NO: 59 - RNA, 100 nt, Artificial Sequence
  Description of Artificial Sequence Synthetic polynucleotide
ucagauccccccaaccguacuaaccagcgaauucuucccggggaucgaacuccuggcggggcucagccaugcugguucaaacgagugcugauaacggaca

SEQ ID NO: 60 - RNA, 100 nt, Artificial Sequence
  Description of Artificial Sequence Synthetic polynucleotide
ucagauccccccaaccguacuaaccagcgaauucuucccggggaucgaacuccuggcggggcucagccaugcugguucaaacgagugcugauagucgaca

SEQ ID NO: 61 - RNA, 100 nt, Artificial Sequence
  Description of Artificial Sequence Synthetic polynucleotide
uccuguccgggaacccgccggucccauaaugaucucaugaaacuucgaacuccuggcggggcucagccaugcugguucaaacaugaacugauaugagaca

SEQ ID NO: 62 - RNA, 100 nt, Artificial Sequence
  Description of Artificial Sequence Synthetic polynucleotide
uccuguccgggaacccgccggucccauaaugaucucaugaaacuucgaacuccuggcggggcucagccaugcugguucaaacaugaacugauagucgaca

SEQ ID NO: 63 - RNA, 100 nt, Artificial Sequence
  Description of Artificial Sequence Synthetic polynucleotide
uccugucgagcaaccccaucgucccccggugaucacaaaccacuucgaacuccuggcggggcucagccaugcugguucaaaucuauacugauacgagaca

SEQ ID NO: 64 - RNA, 100 nt, Artificial Sequence
  Description of Artificial Sequence Synthetic polynucleotide
uccugucgagcaaccccaucgucccccggugaucacaaaccacuucgaacuccuggcggggcucagccaugcugguucaaaucuauacugauagucgaca

SEQ ID NO: 65 - RNA, 100 nt, Artificial Sequence
  Description of Artificial Sequence Synthetic polynucleotide
uccugugggccaaccgugacguccauccgugaugacggcucacuucgaacuccuggcggggcucagccaugcugguucaaacuggugcugauaaaagaca

SEQ ID NO: 66 - RNA, 100 nt, Artificial Sequence
  Description of Artificial Sequence Synthetic polynucleotide
uccugugggccaaccgugacguccauccgugaugacggcucacuucgaacuccuggcggggcucagccaugcugguucaaacuggugcugauagucgaca

SEQ ID NO: 67 - RNA, 100 nt, Artificial Sequence
  Description of Artificial Sequence Synthetic polynucleotide
uccuggaacccaacagcuacgucagcgcgugagaccagaucacuucgaacuccuggcggggcucagccaugcugguucaaacugaugcugauaauagaca

SEQ ID NO: 68 - RNA, 100 nt, Artificial Sequence
  Description of Artificial Sequence Synthetic polynucleotide
uccuggaacccaacagcuacgucagcgcgugagaccagaucacuucgaacuccuggcggggcucagccaugcugguucaaacugaugcugauagucgaca

SEQ ID NO: 69 - RNA, 100 nt, Artificial Sequence
  Description of Artificial Sequence Synthetic polynucleotide
uccuacaccccaagaaguacgugggagcgugccaacacaucacuucgaacuccuggcggggcucagccaugcugguucaaaauacugcugauaugagaca

SEQ ID NO: 70 - RNA, 100 nt, Artificial Sequence
  Description of Artificial Sequence Synthetic polynucleotide
uccuacaccccaagaaguacgugggagcgugccaacacaucacuucgaacuccuggcggggcucagccaugcugguucaaaauacugcugauagucgaca

SEQ ID NO: 71 - RNA, 100 nt, Artificial Sequence
  Description of Artificial Sequence Synthetic polynucleotide
ucccaacccccacggcguacggagcagcguccguagccaucacuucgaacuccuggcggggcucagccaugcugguucaaaaacgugcugauacaggaca

SEQ ID NO: 72 - RNA, 100 nt, Artificial Sequence
  Description of Artificial Sequence Synthetic polynucleotide
ucccaacccccacggcguacggagcagcguccguagccaucacuucgaacuccuggcggggcucagccaugcugguucaaaaacgugcugauagucgaca

SEQ ID NO: 73 - RNA, 100 nt, Artificial Sequence
  Description of Artificial Sequence Synthetic polynucleotide
uccugucccgguacccguuauucccaggccuuccuuccaucacuucgaacuccuggcggggcucagccaugcugguucaaacaggugcugauaugagaca

SEQ ID NO: 74 - RNA, 100 nt, Artificial Sequence
  Description of Artificial Sequence Synthetic polynucleotide
uccuguccggaaacccgauagucccacuaaguucuuccaucacuucgaacuccuggcggggcucagccaugcugguucaaacccuuacugauaaaggaca

SEQ ID NO: 75 - RNA, 100 nt, Artificial Sequence
  Description of Artificial Sequence Synthetic polynucleotide
uccuguccggaaacccgauagucccacuaaguucuuccaucacuucgaacuccuggcggggcucagccaugcugguucaaacccuuacugauagucgaca

SEQ ID NO: 76 - RNA, 100 nt, Artificial Sequence
  Description of Artificial Sequence Synthetic polynucleotide
ucagcucccccuugccguacccaccagcgcauucuuccuaccagucgaacuccuggcggggcucagccaugcugguucaaagaacuagugauagucgaca

SEQ ID NO: 77 - RNA, 100 nt, Artificial Sequence
  Description of Artificial Sequence Synthetic polynucleotide
uccugucgaacaacccaaccgucccuuggugauccgugagcacuucgaacuccuggcggggcucagccaugcugguucaaagguaguaugauagggaaca

SEQ ID NO: 78 - RNA, 100 nt, Artificial Sequence
  Description of Artificial Sequence Synthetic polynucleotide
uccugugggccaaccgacacguccgcccgugauaaaguuucacuucgaacuccuggcggggcucagccaugcugguucaaagacguagugauaggguaca

SEQ ID NO: 79 - RNA, 100 nt, Artificial Sequence
  Description of Artificial Sequence Synthetic polynucleotide
uccugugggccaaccgacacguccgcccgugauaaaguuucacuucgaacuccuggcggggcucagccaugcugguucaaagacguagugauagucgaca

SEQ ID NO: 80 - RNA, 100 nt, Artificial Sequence
  Description of Artificial Sequence Synthetic polynucleotide
uccugacacccaacaauuacgucggugcgugagaaaggaucacuucgaacuccuggcggggcucagccaugcugguucaaagcagguaugauagggaaca

SEQ ID NO: 81 - RNA, 100 nt, Artificial Sequence
  Description of Artificial Sequence Synthetic polynucleotide
uccuuagccccaagagguacgugagagcgugcggaagcaucacuucgaacuccuggcggggcucagccaugcugguucaaaggagaaaugauaggguaca

SEQ ID NO: 82 - RNA, 100 nt, Artificial Sequence
  Description of Artificial Sequence Synthetic polynucleotide
uccuuagccccaagagguacgugagagcgugcggaagcaucacuucgaacuccuggcggggcucagccaugcugguucaaaggagaaaugauagucgaca

SEQ ID NO: 83 - RNA, 100 nt, Artificial Sequence
  Description of Artificial Sequence Synthetic polynucleotide
uccuguccgggaacccgcuagucccaccccccucuuccaucacuucgaacuccuggcggggcucagccaugcugguucaaagcaaaaaugauaggguaca

SEQ ID NO: 84 - RNA, 100 nt, Artificial Sequence
  Description of Artificial Sequence Synthetic polynucleotide
uccuguccgggaacccgcuagucccaccccccucuuccaucacuucgaacuccuggcggggcucagccaugcugguucaaagcaaaaaugauagucgaca

SEQ ID NO: 85 - RNA, 100 nt, Artificial Sequence
  Description of Artificial Sequence Synthetic polynucleotide
uccugucagacaacccacucgucccccucuuaucuuccaucacuucgaacuccuggcggggcucagccaugcugguucaaagagugggugauaggguaca

SEQ ID NO: 86 - RNA, 100 nt, Artificial Sequence
  Description of Artificial Sequence Synthetic polynucleotide
uccugucagacaacccacucgucccccucuuaucuuccaucacuucgaacuccuggcggggcucagccaugcugguucaaagagugggugauagucgaca

SEQ ID NO: 87 - RNA, 100 nt, Artificial Sequence
  Description of Artificial Sequence Synthetic polynucleotide
uccugucggacaacccccucgucccagcgugaucuuccaucacuucgaacuccuggcggggcucagccaugcugguucaaagccccagugauagucgaca

SEQ ID NO: 88 - RNA, 100 nt, Artificial Sequence
  Description of Artificial Sequence Synthetic polynucleotide
uccauacccccauagcguacgaaacagcguuuacuuccacguagacgaacuccuggcggggcucagccaugcuauuaguaaguucuaaugauagggaaca

SEQ ID NO: 89 - RNA, 100 nt, Artificial Sequence
  Description of Artificial Sequence Synthetic polynucleotide
uccugaagcccaacgguuacgucagcgcgugacaaaagaucacuucgaacuccuggcggggcucagccaugcuaaccguaaguucuggugauaggguaca

SEQ ID NO: 90 - RNA, 100 nt, Artificial Sequence
  Description of Artificial Sequence Synthetic polynucleotide
uccuguccaggaacccgauggucccaagacucucuuccaucacuucgaacuccuggcggggcucagccaugcuacguauaaguucuaaugauaggguaca

SEQ ID NO: 91 - RNA, 100 nt, Artificial Sequence
  Description of Artificial Sequence Synthetic polynucleotide
uccuguaggccaaccaugacguccaaacgugaugagccaucaagguauacuccuggcggggcucagccaugcugugagauaguucaccugauagucgaca

SEQ ID NO: 92 - RNA, 100 nt, Artificial Sequence
  Description of Artificial Sequence Synthetic polynucleotide
uccugaggcccaacagauacgucgaugcgugagcguccaucuguccaaacuccuggcggggcucagccaugcugcagucuaguucagacgauagucgaca

SEQ ID NO: 93 - RNA, 100 nt, Artificial Sequence
  Description of Artificial Sequence Synthetic polynucleotide
uccugaggcccaacagauacgucgaugcgugagcguccaucuguccaaacuccuggcggggcucagccaugcugcagucuaguucaccugauagucgaca

SEQ ID NO: 94 - RNA, 100 nt, Artificial Sequence
  Description of Artificial Sequence Synthetic polynucleotide
uccucagccccaagggguacguaggagcguguaauuccauauucgggaacuccuggcggggcucagccaugcugcacgcuaguucaggggauagucgaca

SEQ ID NO: 95 - RNA, 100 nt, Artificial Sequence
  Description of Artificial Sequence Synthetic polynucleotide
uccucagccccaagggguacguaggagcguguaauuccauauucgggaacuccuggcggggcucagccaugcugcacgcuaguucaccugauagucgaca

SEQ ID NO: 96 - RNA, 100 nt, Artificial Sequence
  Description of Artificial Sequence Synthetic polynucleotide
uccggacccccauggcguacgagacagcguccccuuccagcguaccgaacuccuggcggggcucagccaugcugauagucaguucaggggauagucgaca

SEQ ID NO: 97 - RNA, 100 nt, Artificial Sequence
  Description of Artificial Sequence Synthetic polynucleotide
uccggacccccauggcguacgagacagcguccccuuccagcguaccgaacuccuggcggggcucagccaugcugauagucaguucaccugauagucgaca

SEQ ID NO: 98 - RNA, 100 nt, Artificial Sequence
  Description of Artificial Sequence Synthetic polynucleotide
ucggaucccccuugccguacuuaccagcggguucuucccgauucucgaacuccuggcggggcucagccaugcuguaggauaguucaacagauagucgaca

SEQ ID NO: 99 - RNA, 100 nt, Artificial Sequence
  Description of Artificial Sequence Synthetic polynucleotide
ucggaucccccuugccguacuuaccagcggguucuucccgauucucgaacuccuggcggggcucagccaugcuguaggauaguucaccugauagucgaca

SEQ ID NO: 100 - RNA, 100 nt, Artificial Sequence
  Description of Artificial Sequence Synthetic polynucleotide
uccugucccgguacccguuucucccaggacgaucuuaauggucuucgaacuccuggcggggcucagccaugcuguaggcuaguucaagggauagucgaca

SEQ ID NO: 101 - RNA, 100 nt, Artificial Sequence
  Description of Artificial Sequence Synthetic polynucleotide
uccuguccggaaacccgauggucccauacugaucucaagagacuucgaacuccuggcggggcucagccaugcugcgaccuaguucaacggauagucgaca

SEQ ID NO: 102 - RNA, 100 nt, Artificial Sequence
  Description of Artificial Sequence Synthetic polynucleotide
uccuguccggaaacccgauggucccauacugaucucaagagacuucgaacuccuggcggggcucagccaugcugcgaccuaguucaccugauagucgaca

SEQ ID NO: 103 - RNA, 100 nt, Artificial Sequence
  Description of Artificial Sequence Synthetic polynucleotide
uccugucgagcaacccuuccgucccuuggugauccuagggcacuucgaacuccuggcggggcucagccaugcuguauguuaguucaaaggauagucgaca

SEQ ID NO: 104 - RNA, 100 nt, Artificial Sequence
  Description of Artificial Sequence Synthetic polynucleotide
uccugucgagcaacccuuccgucccuuggugauccuagggcacuucgaacuccuggcggggcucagccaugcuguauguuaguucaccugauagucgaca

SEQ ID NO: 105 - RNA, 100 nt, Artificial Sequence
  Description of Artificial Sequence Synthetic polynucleotide
uccuguacaccaaccaagacguccguacgugauaaaaguucacuucgaacuccuggcggggcucagccaugcugcaaacuaguucaagggauagucgaca

SEQ ID NO: 106 - RNA, 100 nt, Artificial Sequence
  Description of Artificial Sequence Synthetic polynucleotide
uccuguacaccaaccaagacguccguacgugauaaaaguucacuucgaacuccuggcggggcucagccaugcugcaaacuaguucaccugauagucgaca

SEQ ID NO: 107 - RNA, 100 nt, Artificial Sequence
  Description of Artificial Sequence Synthetic polynucleotide
uccuggaacccaacagcuacgucgaugcgugaacaacaaucacuucgaacuccuggcggggcucagccaugcugcaaugcaguucaggagauagucgaca

SEQ ID NO: 108 - RNA, 100 nt, Artificial Sequence
  Description of Artificial Sequence Synthetic polynucleotide
uccuggaacccaacagcuacgucgaugcgugaacaacaaucacuucgaacuccuggcggggcucagccaugcugcaaugcaguucaccugauagucgaca

SEQ ID NO: 109 - RNA, 100 nt, Artificial Sequence
  Description of Artificial Sequence Synthetic polynucleotide
uccuccgccccaagaaguacguagaagcguguuuaggcaucacuucgaacuccuggcggggcucagccaugcugaugguuaguucaagggauagucgaca

SEQ ID NO: 110 - RNA, 100 nt, Artificial Sequence
  Description of Artificial Sequence Synthetic polynucleotide
uccgggcccccacgacguacgggacagcgucaguugccaucacuucgaacuccuggcggggcucagccaugcugaacacuaguucaaaagauagucgaca

SEQ ID NO: 111 - RNA, 100 nt, Artificial Sequence
  Description of Artificial Sequence Synthetic polynucleotide
uccgggcccccacgacguacgggacagcgucaguugccaucacuucgaacuccuggcggggcucagccaugcugaacacuaguucaccugauagucgaca

SEQ ID NO: 112 - RNA, 100 nt, Artificial Sequence
  Description of Artificial Sequence Synthetic polynucleotide
ucaacucccccucaccguaccgaccagcgccacaguccaucacuucgaacuccuggcggggcucagccaugcuguaguguaguucagaggauagucgaca

SEQ ID NO: 113 - RNA, 100 nt, Artificial Sequence
  Description of Artificial Sequence Synthetic polynucleotide
ucaacucccccucaccguaccgaccagcgccacaguccaucacuucgaacuccuggcggggcucagccaugcuguaguguaguucaccugauagucgaca

SEQ ID NO: 114 - RNA, 100 nt, Artificial Sequence
  Description of Artificial Sequence Synthetic polynucleotide
uccugucccgguacccgucuuucccaggagucacuuccaucacuucgaacuccuggcggggcucagccaugcugauauuuaguucaggcgauagucgaca

SEQ ID NO: 115 - RNA, 100 nt, Artificial Sequence
  Description of Artificial Sequence Synthetic polynucleotide
uccuguccaagaacccgcuagucccauucuauucuuccaucacuucgaacuccuggcggggcucagccaugcugaaaauuaguucaacggauagucgaca

SEQ ID NO: 116 - RNA, 100 nt, Artificial Sequence
  Description of Artificial Sequence Synthetic polynucleotide
uccuguggaccaaccaccacguccaaucgugaugcaccaucaagaguuacuccuggcggggcucagccaugcuggcgggccguucaccugauagucgaca

SEQ ID NO: 117 - RNA, 100 nt, Artificial Sequence
  Description of Artificial Sequence Synthetic polynucleotide
uccugaagcccaacgacuacgucggcgcgugacacuccaucuaagacaacuccuggcggggcucagccaugcuggagacuuguucaccugauagucgaca

SEQ ID NO: 118 - RNA, 100 nt, Artificial Sequence
  Description of Artificial Sequence Synthetic polynucleotide
uccuacaccccaagggguacguggaagcgugcuauuccaugugccagaacuccuggcggggcucagccaugcugggggaccguucacauaauagucgaca

SEQ ID NO: 119 - RNA, 100 nt, Artificial Sequence
  Description of Artificial Sequence Synthetic polynucleotide
uccuacaccccaagggguacguggaagcgugcuauuccaugugccagaacuccuggcggggcucagccaugcugggggaccguucaccugauagucgaca

SEQ ID NO: 120 - RNA, 100 nt, Artificial Sequence
  Description of Artificial Sequence Synthetic polynucleotide
uccgaacccccauaacguacggagcagcguacccuuccaaaaaaacgaacuccuggcggggcucagccaugcuggggggccguucacgcaauagucgaca

SEQ ID NO: 121 - RNA, 100 nt, Artificial Sequence
  Description of Artificial Sequence Synthetic polynucleotide
uccgaacccccauaacguacggagcagcguacccuuccaaaaaaacgaacuccuggcggggcucagccaugcuggggggccguucaccugauagucgaca

SEQ ID NO: 122 - RNA, 100 nt, Artificial Sequence
  Description of Artificial Sequence Synthetic polynucleotide
ucgcaucccccuuaccguacuagccagcgcucucuucccggcagucgaacuccuggcggggcucagccaugcuggcaaucuguucacgccauagucgaca

SEQ ID NO: 123 - RNA, 100 nt, Artificial Sequence
  Description of Artificial Sequence Synthetic polynucleotide
uccugucccagcacccgucauucccaggaggaucuugaggaccuucgaacuccuggcggggcucagccaugcugggaauccguucacggcauagucgaca

SEQ ID NO: 124 - RNA, 100 nt, Artificial Sequence
  Description of Artificial Sequence Synthetic polynucleotide
uccugucccagcacccgucauucccaggaggaucuugaggaccuucgaacuccuggcggggcucagccaugcugggaauccguucaccugauagucgaca

SEQ ID NO: 125 - RNA, 100 nt, Artificial Sequence
  Description of Artificial Sequence Synthetic polynucleotide
uccuguccggaaacccgccggucccauauugaucuauaaaaacuucgaacuccuggcggggcucagccaugcuggcaacucguucacauuauagucgaca

SEQ ID NO: 126 - RNA, 100 nt, Artificial Sequence
  Description of Artificial Sequence Synthetic polynucleotide
uccuguccggaaacccgccggucccauauugaucuauaaaaacuucgaacuccuggcggggcucagccaugcuggcaacucguucaccugauagucgaca

SEQ ID NO: 127 - RNA, 100 nt, Artificial Sequence
  Description of Artificial Sequence Synthetic polynucleotide
uccugucgggcaacccaaucgucccucggugaucgauaaacacuucgaacuccuggcggggcucagccaugcuggaaggccguucacggcauagucgaca

SEQ ID NO: 128 - RNA, 100 nt, Artificial Sequence
  Description of Artificial Sequence Synthetic polynucleotide
uccugucgggcaacccaaucgucccucggugaucgauaaacacuucgaacuccuggcggggcucagccaugcuggaaggccguucaccugauagucgaca

SEQ ID NO: 129 - RNA, 100 nt, Artificial Sequence
  Description of Artificial Sequence Synthetic polynucleotide
uccugugaaccaaccgagacguccauccgugauagauguucacuucgaacuccuggcggggcucagccaugcuggcaugccguucacggcauagucgaca

SEQ ID NO: 130 - RNA, 100 nt, Artificial Sequence
  Description of Artificial Sequence Synthetic polynucleotide
uccugugaaccaaccgagacguccauccgugauagauguucacuucgaacuccuggcggggcucagccaugcuggcaugccguucaccugauagucgaca

SEQ ID NO: 131 - RNA, 100 nt, Artificial Sequence
  Description of Artificial Sequence Synthetic polynucleotide
uccugaaacccaacaauuacgucggcgcgugagaagcaaucacuucgaacuccuggcggggcucagccaugcugggguccuguucacgauauagucgaca

SEQ ID NO: 132 - RNA, 100 nt, Artificial Sequence
  Description of Artificial Sequence Synthetic polynucleotide
uccugaaacccaacaauuacgucggcgcgugagaagcaaucacuucgaacuccuggcggggcucagccaugcugggguccuguucaccugauagucgaca

SEQ ID NO: 133 - RNA, 100 nt, Artificial Sequence
  Description of Artificial Sequence Synthetic polynucleotide
uccuuuaccccaagaaguacguaagagcgugccguggcaucacuucgaacuccuggcggggcucagccaugcuggcgugcuguucacgaaauagucgaca

SEQ ID NO: 134 - RNA, 100 nt, Artificial Sequence
  Description of Artificial Sequence Synthetic polynucleotide
uccuuuaccccaagaaguacguaagagcgugccguggcaucacuucgaacuccuggcggggcucagccaugcuggcgugcuguucaccugauagucgaca

SEQ ID NO: 135 - RNA, 100 nt, Artificial Sequence
  Description of Artificial Sequence Synthetic polynucleotide
uccauccccccauaacguacgcaacagcguuuggcgccaucacuucgaacuccuggcggggcucagccaugcuggcuguacguucacauaauagucgaca

SEQ ID NO: 136 - RNA, 100 nt, Artificial Sequence
  Description of Artificial Sequence Synthetic polynucleotide
uccauccccccauaacguacgcaacagcguuuggcgccaucacuucgaacuccuggcggggcucagccaugcuggcuguacguucaccugauagucgaca

SEQ ID NO: 137 - RNA, 100 nt, Artificial Sequence
  Description of Artificial Sequence Synthetic polynucleotide
ucauaucccccuuaccguacugaccagcgcaacgcuccaucacuucgaacuccuggcggggcucagccaugcugggcucucguucacagcauagucgaca

SEQ ID NO: 138 - RNA, 100 nt, Artificial Sequence
  Description of Artificial Sequence Synthetic polynucleotide
ucauaucccccuuaccguacugaccagcgcaacgcuccaucacuucgaacuccuggcggggcucagccaugcugggcucucguucaccugauagucgaca

SEQ ID NO: 139 - RNA, 100 nt, Artificial Sequence
  Description of Artificial Sequence Synthetic polynucleotide
uccugucccgacacccgucauucccaggauucacuuccaucacuucgaacuccuggcggggcucagccaugcugggguuccguucacacaauagucgaca

SEQ ID NO: 140 - RNA, 100 nt, Artificial Sequence
  Description of Artificial Sequence Synthetic polynucleotide
uccugucccgacacccgucauucccaggauucacuuccaucacuucgaacuccuggcggggcucagccaugcugggguuccguucaccugauagucgaca

SEQ ID NO: 141 - RNA, 100 nt, Artificial Sequence
  Description of Artificial Sequence Synthetic polynucleotide
uccuguccgagaacccgguagucccacaacacucuuccaucacuucgaacuccuggcggggcucagccaugcuggcgacgcguucacacaauagucgaca

SEQ ID NO: 142 - RNA, 100 nt, Artificial Sequence
  Description of Artificial Sequence Synthetic polynucleotide
uccuguccgagaacccgguagucccacaacacucuuccaucacuucgaacuccuggcggggcucagccaugcuggcgacgcguucaccugauagucgaca

SEQ ID NO: 143 - RNA, 100 nt, Artificial Sequence
  Description of Artificial Sequence Synthetic polynucleotide
uccugucaggcaacccauccgucccagcgugaucuuccaucacuucgaacuccuggcggggcucagccaugcuggcgaguuguucacgccauagucgaca

SEQ ID NO: 144 - RNA, 100 nt, Artificial Sequence
  Description of Artificial Sequence Synthetic polynucleotide
uccugucaggcaacccauccgucccagcgugaucuuccaucacuucgaacuccuggcggggcucagccaugcuggcgaguuguucaccugauagucgaca

SEQ ID NO: 145 - RNA, 100 nt, Artificial Sequence
  Description of Artificial Sequence Synthetic polynucleotide
uccuguccaagaacccgccggucccauacugaucuaagaucacuaccuaauccuggcggggcucagccaugcugguggagcuuucaccauuuagucgaca

SEQ ID NO: 146 - RNA, 100 nt, Artificial Sequence
  Description of Artificial Sequence Synthetic polynucleotide
uccuguccaagaacccgccggucccauacugaucuaagaucacuaccuaauccuggcggggcucagccaugcugguggagcuuucaccugauagucgaca

SEQ ID NO: 147 - RNA, 100 nt, Artificial Sequence
  Description of Artificial Sequence Synthetic polynucleotide
uccugucaaacaacccuaucgucccuuggugaucauacaucacccuaaucuccuggcggggcucagccaugcuggugcuucuuucaccugauagucgaca

SEQ ID NO: 148 - RNA, 100 nt, Artificial Sequence
  Description of Artificial Sequence Synthetic polynucleotide
uccuguggaccaaccgccacguccguccgugaugccccaucaggagucacuccuggcggggcucagccaugcuggucuguauuucaccaacuagucgaca

SEQ ID NO: 149 - RNA, 100 nt, Artificial Sequence
  Description of Artificial Sequence Synthetic polynucleotide
uccuguggaccaaccgccacguccguccgugaugccccaucaggagucacuccuggcggggcucagccaugcuggucuguauuucaccugauagucgaca

SEQ ID NO: 150 - RNA, 100 nt, Artificial Sequence
  Description of Artificial Sequence Synthetic polynucleotide
uccugaagcccaacgauuacgucagugcgugacgauccaucuuuguaaacuccuggcggggcucagccaugcugguaccacuuucaccaucuagucgaca

SEQ ID NO: 151 - RNA, 100 nt, Artificial Sequence
  Description of Artificial Sequence Synthetic polynucleotide
uccugaagcccaacgauuacgucagugcgugacgauccaucuuuguaaacuccuggcggggcucagccaugcugguaccacuuucaccugauagucgaca

SEQ ID NO: 152 - RNA, 100 nt, Artificial Sequence
  Description of Artificial Sequence Synthetic polynucleotide
ucccaacccccacgacguacgcggcagcguuaccuuccagacaaccgaacuccuggcggggcucagccaugcugguauggcauucaccauuuagucgaca

SEQ ID NO: 153 - RNA, 100 nt, Artificial Sequence
  Description of Artificial Sequence Synthetic polynucleotide
ucccaacccccacgacguacgcggcagcguuaccuuccagacaaccgaacuccuggcggggcucagccaugcugguauggcauucaccugauagucgaca

SEQ ID NO: 154 - RNA, 100 nt, Artificial Sequence
  Description of Artificial Sequence Synthetic polynucleotide
ucgauucccccucgccguacacgccagcgcuuucuuccccguagucgaacuccuggcggggcucagccaugcuggucuggccuucaccacuuagucgaca

SEQ ID NO: 155 - RNA, 100 nt, Artificial Sequence
  Description of Artificial Sequence Synthetic polynucleotide
ucgauucccccucgccguacacgccagcgcuuucuuccccguagucgaacuccuggcggggcucagccaugcuggucuggccuucaccugauagucgaca

SEQ ID NO: 156 - RNA, 100 nt, Artificial Sequence
  Description of Artificial Sequence Synthetic polynucleotide
uccugucccggcacccgucaaucccagaucgaucuugauggucuucgaacuccuggcggggcucagccaugcuggucccugauucaccgauuagucgaca

SEQ ID NO: 157 - RNA, 100 nt, Artificial Sequence
  Description of Artificial Sequence Synthetic polynucleotide
uccugucccggcacccgucaaucccagaucgaucuugauggucuucgaacuccuggcggggcucagccaugcuggucccugauucaccugauagucgaca

SEQ ID NO: 158 - RNA, 100 nt, Artificial Sequence
  Description of Artificial Sequence Synthetic polynucleotide
uccuguccagaaacccggcagucccauauugaucuauaauaacuucgaacuccuggcggggcucagccaugcugguauuuguuucaccccuuagucgaca

SEQ ID NO: 159 - RNA, 100 nt, Artificial Sequence
  Description of Artificial Sequence Synthetic polynucleotide
uccuguccagaaacccggcagucccauauugaucuauaauaacuucgaacuccuggcggggcucagccaugcugguauuuguuucaccugauagucgaca

SEQ ID NO: 160 - RNA, 100 nt, Artificial Sequence
  Description of Artificial Sequence Synthetic polynucleotide
uccugucaaacaacccccccgucccuuagugaucacguuccacuucgaacuccuggcggggcucagccaugcugguggucguuucaccgccuagucgaca

SEQ ID NO: 161 - RNA, 100 nt, Artificial Sequence
  Description of Artificial Sequence Synthetic polynucleotide
uccugucaaacaacccccccgucccuuagugaucacguuccacuucgaacuccuggcggggcucagccaugcugguggucguuucaccugauagucgaca

SEQ ID NO: 162 - RNA, 100 nt, Artificial Sequence
  Description of Artificial Sequence Synthetic polynucleotide
uccuguggaccaaccgagacguccguacgugaugaugauucacuucgaacuccuggcggggcucagccaugcuggucuuuuuuucaccacuuagucgaca

SEQ ID NO: 163 - RNA, 100 nt, Artificial Sequence
  Description of Artificial Sequence Synthetic polynucleotide
uccuguggaccaaccgagacguccguacgugaugaugauucacuucgaacuccuggcggggcucagccaugcuggucuuuuuuucaccugauagucgaca

SEQ ID NO: 164 - RNA, 100 nt, Artificial Sequence
  Description of Artificial Sequence Synthetic polynucleotide
uccuggcacccaacgauuacgucaacgcgugaagcaggaucacuucgaacuccuggcggggcucagccaugcuggugugcauuucacccuuuagucgaca

SEQ ID NO: 165 - RNA, 100 nt, Artificial Sequence
  Description of Artificial Sequence Synthetic polynucleotide
uccuggcacccaacgauuacgucaacgcgugaagcaggaucacuucgaacuccuggcggggcucagccaugcuggugugcauuucaccugauagucgaca

SEQ ID NO: 166 - RNA, 100 nt, Artificial Sequence
  Description of Artificial Sequence Synthetic polynucleotide
uccggacccccauagcguacgacacagcguagcgaaccaucacuucgaacuccuggcggggcucagccaugcugguguauacuucaccaucuagucgaca

SEQ ID NO: 167 - RNA, 100 nt, Artificial Sequence
  Description of Artificial Sequence Synthetic polynucleotide
uccggacccccauagcguacgacacagcguagcgaaccaucacuucgaacuccuggcggggcucagccaugcugguguauacuucaccugauagucgaca

SEQ ID NO: 168 - RNA, 100 nt, Artificial Sequence
  Description of Artificial Sequence Synthetic polynucleotide
ucggaucccccuugccguacaagccagcgaacuaauccaucacuucgaacuccuggcggggcucagccaugcugguguugcuuucaccauuuagucgaca

SEQ ID NO: 169 - RNA, 100 nt, Artificial Sequence
  Description of Artificial Sequence Synthetic polynucleotide
ucggaucccccuugccguacaagccagcgaacuaauccaucacuucgaacuccuggcggggcucagccaugcugguguugcuuucaccugauagucgaca

SEQ ID NO: 170 - RNA, 100 nt, Artificial Sequence
  Description of Artificial Sequence Synthetic polynucleotide
uccugucccgauacccgucucucccaggccuugcuuccaucacuucgaacuccuggcggggcucagccaugcuggugaguuauucacccuuuagucgaca

SEQ ID NO: 171 - RNA, 100 nt, Artificial Sequence
  Description of Artificial Sequence Synthetic polynucleotide
uccugucccgauacccgucucucccaggccuugcuuccaucacuucgaacuccuggcggggcucagccaugcuggugaguuauucaccugauagucgaca

SEQ ID NO: 172 - RNA, 100 nt, Artificial Sequence
  Description of Artificial Sequence Synthetic polynucleotide
uccuguccaggaacccggcggucccaaaagauucuuccaucacuucgaacuccuggcggggcucagccaugcuggucuacgcuucaccguuuagucgaca

SEQ ID NO: 173 - RNA, 100 nt, Artificial Sequence
  Description of Artificial Sequence Synthetic polynucleotide
uccuguccaggaacccggcggucccaaaagauucuuccaucacuucgaacuccuggcggggcucagccaugcuggucuacgcuucaccugauagucgaca

SEQ ID NO: 174 - RNA, 100 nt, Artificial Sequence
  Description of Artificial Sequence Synthetic polynucleotide
uccugucacacaacccagccgucccccaauaaucuuccaucacuucgaacuccuggcggggcucagccaugcuggugggcgcuucaccccuuagucgaca

SEQ ID NO: 175 - RNA, 100 nt, Artificial Sequence
  Description of Artificial Sequence Synthetic polynucleotide
uccugucacacaacccagccgucccccaauaaucuuccaucacuucgaacuccuggcggggcucagccaugcuggugggcgcuucaccugauagucgaca

SEQ ID NO: 176 - RNA, 100 nt, Artificial Sequence
  Description of Artificial Sequence Synthetic polynucleotide
uccugucacacaacccuuucgucccagcgugaucuuccaucacuucgaacuccuggcggggcucagccaugcugguaguauuuucacccuuuagucgaca

SEQ ID NO: 177 - RNA, 100 nt, Artificial Sequence
  Description of Artificial Sequence Synthetic polynucleotide
uccugucacacaacccuuucgucccagcgugaucuuccaucacuucgaacuccuggcggggcucagccaugcugguaguauuuucaccugauagucgaca

SEQ ID NO: 178 - RNA, 100 nt, Artificial Sequence
  Description of Artificial Sequence Synthetic polynucleotide
uccugcagcccaacggauacgucaacgcgugaggauccaucuuguauaacuccuggcggggcucagccaugcugguuaugucaucaccugauagucgaca

SEQ ID NO: 179 - RNA, 100 nt, Artificial Sequence
  Description of Artificial Sequence Synthetic polynucleotide
uccuacaccccaaaggguacguagaagcguguaguuccauguagcggaacuccuggcggggcucagccaugcugguuagcacgucaccuuuaagucgaca

SEQ ID NO: 180 - RNA, 100 nt, Artificial Sequence
  Description of Artificial Sequence Synthetic polynucleotide
uccuacaccccaaaggguacguagaagcguguaguuccauguagcggaacuccuggcggggcucagccaugcugguuagcacgucaccugauagucgaca

SEQ ID NO: 181 - RNA, 100 nt, Artificial Sequence
  Description of Artificial Sequence Synthetic polynucleotide
uccaaacccccauggcguacgacacagcguuuacuuccaaacaggcgaacuccuggcggggcucagccaugcugguuacccccucaccuaacagucgaca

SEQ ID NO: 182 - RNA, 100 nt, Artificial Sequence
  Description of Artificial Sequence Synthetic polynucleotide
uccaaacccccauggcguacgacacagcguuuacuuccaaacaggcgaacuccuggcggggcucagccaugcugguuacccccucaccugauagucgaca

SEQ ID NO: 183 - RNA, 100 nt, Artificial Sequence
  Description of Artificial Sequence Synthetic polynucleotide
ucgcaucccccucgccguacaggccagcgcccucuuccccuuggucgaacuccuggcggggcucagccaugcugguuagacccucaccucacagucgaca

SEQ ID NO: 184 - RNA, 100 nt, Artificial Sequence
  Description of Artificial Sequence Synthetic polynucleotide
uccugucccaacacccgucauucccaggaagaucuugggggucuucgaacuccuggcggggcucagccaugcugguuauauacucaccuaagagucgaca

SEQ ID NO: 185 - RNA, 100 nt, Artificial Sequence
  Description of Artificial Sequence Synthetic polynucleotide
uccugucccaacacccgucauucccaggaagaucuugggggucuucgaacuccuggcggggcucagccaugcugguuauauacucaccugauagucgaca

SEQ ID NO: 186 - RNA, 100 nt, Artificial Sequence
  Description of Artificial Sequence Synthetic polynucleotide
uccuguccacaaacccgauggucccaauaugaucuaaguaaacuucgaacuccuggcggggcucagccaugcugguugcucccucaccuuacagucgaca

SEQ ID NO: 187 - RNA, 100 nt, Artificial Sequence
  Description of Artificial Sequence Synthetic polynucleotide
uccuguccacaaacccgauggucccaauaugaucuaaguaaacuucgaacuccuggcggggcucagccaugcugguugcucccucaccugauagucgaca

SEQ ID NO: 188 - RNA, 100 nt, Artificial Sequence
  Description of Artificial Sequence Synthetic polynucleotide
uccugucgagcaacccauucgucccuuagugaucggaggccacuucgaacuccuggcggggcucagccaugcugguuaacacaucaccuuacagucgaca

SEQ ID NO: 189 - RNA, 100 nt, Artificial Sequence
  Description of Artificial Sequence Synthetic polynucleotide
uccugucgagcaacccauucgucccuuagugaucggaggccacuucgaacuccuggcggggcucagccaugcugguuaacacaucaccugauagucgaca

SEQ ID NO: 190 - RNA, 100 nt, Artificial Sequence
  Description of Artificial Sequence Synthetic polynucleotide
uccugugagccaaccaacacguccguacgugaugcuaguucacuucgaacuccuggcggggcucagccaugcugguuaggucgucaccuuccagucgaca

SEQ ID NO: 191 - RNA, 100 nt, Artificial Sequence
  Description of Artificial Sequence Synthetic polynucleotide
uccugcgacccaacgauuacgucggugcgugaaugcggaucacuucgaacuccuggcggggcucagccaugcugguuaaaaugucaccuccgagucgaca

SEQ ID NO: 192 - RNA, 100 nt, Artificial Sequence
  Description of Artificial Sequence Synthetic polynucleotide
uccugcgacccaacgauuacgucggugcgugaaugcggaucacuucgaacuccuggcggggcucagccaugcugguuaaaaugucaccugauagucgaca

SEQ ID NO: 193 - RNA, 100 nt, Artificial Sequence
  Description of Artificial Sequence Synthetic polynucleotide
uccuacgccccaagggguacguaaaagcgugccaaagcaucacuucgaacuccuggcggggcucagccaugcugguuaaaucgucaccuccaagucgaca

SEQ ID NO: 194 - RNA, 100 nt, Artificial Sequence
  Description of Artificial Sequence Synthetic polynucleotide
uccuacgccccaagggguacguaaaagcgugccaaagcaucacuucgaacuccuggcggggcucagccaugcugguuaaaucgucaccugauagucgaca

SEQ ID NO: 195 - RNA, 100 nt, Artificial Sequence
  Description of Artificial Sequence Synthetic polynucleotide
uccauacccccauagcguacggggcagcguuaauacccaucacuucgaacuccuggcggggcucagccaugcugguuacuuaaucaccucucagucgaca

SEQ ID NO: 196 - RNA, 100 nt, Artificial Sequence
  Description of Artificial Sequence Synthetic polynucleotide
uccauacccccauagcguacggggcagcguuaauacccaucacuucgaacuccuggcggggcucagccaugcugguuacuuaaucaccugauagucgaca

SEQ ID NO: 197 - RNA, 100 nt, Artificial Sequence
  Description of Artificial Sequence Synthetic polynucleotide
ucgguucccccuaaccguacauaccagcgaccccauccaucacuucgaacuccuggcggggcucagccaugcugguuguuucaucaccucugagucgaca

SEQ ID NO: 198 - RNA, 100 nt, Artificial Sequence
  Description of Artificial Sequence Synthetic polynucleotide
ucgguucccccuaaccguacauaccagcgaccccauccaucacuucgaacuccuggcggggcucagccaugcugguuguuucaucaccugauagucgaca

SEQ ID NO: 199 - RNA, 100 nt, Artificial Sequence
  Description of Artificial Sequence Synthetic polynucleotide
uccugucccaacacccgucuuucccagauaauacuuccaucacuucgaacuccuggcggggcucagccaugcugguuaaaucgucaccuuccagucgaca

SEQ ID NO: 200 - RNA, 100 nt, Artificial Sequence
  Description of Artificial Sequence Synthetic polynucleotide
uccugucccaacacccgucuuucccagauaauacuuccaucacuucgaacuccuggcggggcucagccaugcugguuaaaucgucaccugauagucgaca

SEQ ID NO: 201 - RNA, 100 nt, Artificial Sequence
  Description of Artificial Sequence Synthetic polynucleotide
uccuguccggaaacccgcaagucccaaauuccucuuccaucacuucgaacuccuggcggggcucagccaugcugguugucuuaucaccuuaaagucgaca

SEQ ID NO: 202 - RNA, 100 nt, Artificial Sequence
  Description of Artificial Sequence Synthetic polynucleotide
uccuguccggaaacccgcaagucccaaauuccucuuccaucacuucgaacuccuggcggggcucagccaugcugguugucuuaucaccugauagucgaca

SEQ ID NO: 203 - RNA, 100 nt, Artificial Sequence
  Description of Artificial Sequence Synthetic polynucleotide
uccugucgaacaacccuuucguccccuaacaaucuuccaucacuucgaacuccuggcggggcucagccaugcugguuguaaccucaccuaugagucgaca

SEQ ID NO: 204 - RNA, 100 nt, Artificial Sequence
  Description of Artificial Sequence Synthetic polynucleotide
uccugucgaacaacccuuucguccccuaacaaucuuccaucacuucgaacuccuggcggggcucagccaugcugguuguaaccucaccugauagucgaca

SEQ ID NO: 205 - RNA, 100 nt, Artificial Sequence
  Description of Artificial Sequence Synthetic polynucleotide
uccugucgaacaacccucucgucccagcgugaucuuccaucacuucgaacuccuggcggggcucagccaugcugguugcaccgucaccucaaagucgaca

SEQ ID NO: 206 - RNA, 100 nt, Artificial Sequence
  Description of Artificial Sequence Synthetic polynucleotide
uccugucgaacaacccucucgucccagcgugaucuuccaucacuucgaacuccuggcggggcucagccaugcugguugcaccgucaccugauagucgaca

SEQ ID NO: 207 - RNA, 100 nt, Artificial Sequence
  Description of Artificial Sequence Synthetic polynucleotide
uccuguccgagaacccgaaagucccauauugaucuaggaucacugccuaauccuggcggggcucagccaugcugguucuaccgacaccugauagucgaca

SEQ ID NO: 208 - RNA, 100 nt, Artificial Sequence
  Description of Artificial Sequence Synthetic polynucleotide
uccugugggccaaccauaacguccgcucgugauacgccaucaacggcuacuccuggcggggcucagccaugcugguuccuuugccaccugcaugucgaca

SEQ ID NO: 209 - RNA, 100 nt, Artificial Sequence
  Description of Artificial Sequence Synthetic polynucleotide
uccugugggccaaccauaacguccgcucgugauacgccaucaacggcuacuccuggcggggcucagccaugcugguuccuuugccaccugauagucgaca

SEQ ID NO: 210 - RNA, 100 nt, Artificial Sequence
  Description of Artificial Sequence Synthetic polynucleotide
uccugacacccaacaacuacgucggugcgugagacuccaucuagguaaacuccuggcggggcucagccaugcugguucuucugacaccugcaugucgaca

SEQ ID NO: 211 - RNA, 100 nt, Artificial Sequence
  Description of Artificial Sequence Synthetic polynucleotide
uccugacacccaacaacuacgucggugcgugagacuccaucuagguaaacuccuggcggggcucagccaugcugguucuucugacaccugauagucgaca

SEQ ID NO: 212 - RNA, 100 nt, Artificial Sequence
  Description of Artificial Sequence Synthetic polynucleotide
uccuaaaccccaaagaguacgugggagcgugcuauuccaugugggagaacuccuggcggggcucagccaugcugguuccuaucgcaccuguuugucgaca

SEQ ID NO: 213 - RNA, 100 nt, Artificial Sequence
  Description of Artificial Sequence Synthetic polynucleotide
uccuaaaccccaaagaguacgugggagcgugcuauuccaugugggagaacuccuggcggggcucagccaugcugguuccuaucgcaccugauagucgaca

SEQ ID NO: 214 - RNA, 100 nt, Artificial Sequence
  Description of Artificial Sequence Synthetic polynucleotide
uccaagcccccaucacguacgcggcagcgucuacuuccacgugagcgaacuccuggcggggcucagccaugcugguucuauacacaccugcgcgucgaca

SEQ ID NO: 215 - RNA, 100 nt, Artificial Sequence
  Description of Artificial Sequence Synthetic polynucleotide
uccaagcccccaucacguacgcggcagcgucuacuuccacgugagcgaacuccuggcggggcucagccaugcugguucuauacacaccugauagucgaca

SEQ ID NO: 216 - RNA, 100 nt, Artificial Sequence
  Description of Artificial Sequence Synthetic polynucleotide
uccugucccaauacccguuaaucccagaaggaucuuauuagucuucgaacuccuggcggggcucagccaugcugguuccuuacacaccugcgcgucgaca

SEQ ID NO: 217 - RNA, 100 nt, Artificial Sequence
  Description of Artificial Sequence Synthetic polynucleotide
uccugucccaauacccguuaaucccagaaggaucuuauuagucuucgaacuccuggcggggcucagccaugcugguuccuuacacaccugauagucgaca

SEQ ID NO: 218 - RNA, 100 nt, Artificial Sequence
  Description of Artificial Sequence Synthetic polynucleotide
uccuguccaggaacccggaagucccauaaugaucuaaauuaacuucgaacuccuggcggggcucagccaugcugguuccccaggcaccugcacgucgaca

SEQ ID NO: 219 - RNA, 100 nt, Artificial Sequence
  Description of Artificial Sequence Synthetic polynucleotide
uccuguccaggaacccggaagucccauaaugaucuaaauuaacuucgaacuccuggcggggcucagccaugcugguuccccaggcaccugauagucgaca

SEQ ID NO: 220 - RNA, 100 nt, Artificial Sequence
  Description of Artificial Sequence Synthetic polynucleotide
uccugucaaacaacccaaucguccccuggugauccaguuacacuucgaacuccuggcggggcucagccaugcugguuccccgcacaccuguaugucgaca

SEQ ID NO: 221 - RNA, 100 nt, Artificial Sequence
  Description of Artificial Sequence Synthetic polynucleotide
uccugucaaacaacccaaucguccccuggugauccaguuacacuucgaacuccuggcggggcucagccaugcugguuccccgcacaccugauagucgaca

SEQ ID NO: 222 - RNA, 100 nt, Artificial Sequence
  Description of Artificial Sequence Synthetic polynucleotide
uccugugggccaaccgcaacguccaaacgugaugacgguucacuucgaacuccuggcggggcucagccaugcugguuccccauacaccugcuugucgaca

SEQ ID NO: 223 - RNA, 100 nt, Artificial Sequence
  Description of Artificial Sequence Synthetic polynucleotide
uccugugggccaaccgcaacguccaaacgugaugacgguucacuucgaacuccuggcggggcucagccaugcugguuccccauacaccugauagucgaca

SEQ ID NO: 224 - RNA, 100 nt, Artificial Sequence
  Description of Artificial Sequence Synthetic polynucleotide
uccugaaacccaacgaauacgucggcgcgugacggacaaucacuucgaacuccuggcggggcucagccaugcugguucuuucaacaccuguacgucgaca

SEQ ID NO: 225 - RNA, 100 nt, Artificial Sequence
  Description of Artificial Sequence Synthetic polynucleotide
uccugaaacccaacgaauacgucggcgcgugacggacaaucacuucgaacuccuggcggggcucagccaugcugguucuuucaacaccugauagucgaca

SEQ ID NO: 226 - RNA, 100 nt, Artificial Sequence
  Description of Artificial Sequence Synthetic polynucleotide
uccuuagccccaaaaaguacgugaaagcguguagaagcaucacuucgaacuccuggcggggcucagccaugcugguucuuucaacaccugauagucgaca

SEQ ID NO: 227 - RNA, 100 nt, Artificial Sequence
  Description of Artificial Sequence Synthetic polynucleotide
uccaggcccccauaacguacgaagcagcguccggggccaucacuucgaacuccuggcggggcucagccaugcugguuccgcugacaccuguccgucgaca

SEQ ID NO: 228 - RNA, 100 nt, Artificial Sequence
  Description of Artificial Sequence Synthetic polynucleotide
uccaggcccccauaacguacgaagcagcguccggggccaucacuucgaacuccuggcggggcucagccaugcugguuccgcugacaccugauagucgaca

SEQ ID NO: 229 - RNA, 100 nt, Artificial Sequence
  Description of Artificial Sequence Synthetic polynucleotide
uccugucccaguacccguuuuucccagaauuaccuuccaucacuucgaacuccuggcggggcucagccaugcugguuccguaaccaccugcgugucgaca

SEQ ID NO: 230 - RNA, 100 nt, Artificial Sequence
  Description of Artificial Sequence Synthetic polynucleotide
uccugucccaguacccguuuuucccagaauuaccuuccaucacuucgaacuccuggcggggcucagccaugcugguuccguaaccaccugauagucgaca

SEQ ID NO: 231 - RNA, 100 nt, Artificial Sequence
  Description of Artificial Sequence Synthetic polynucleotide
uccuguccggaaacccgguggucccaaaucucucuuccaucacuucgaacuccuggcggggcucagccaugcugguuccgccgccaccugcucgucgaca

SEQ ID NO: 232 - RNA, 100 nt, Artificial Sequence
  Description of Artificial Sequence Synthetic polynucleotide
uccuguccggaaacccgguggucccaaaucucucuuccaucacuucgaacuccuggcggggcucagccaugcugguuccgccgccaccugauagucgaca

SEQ ID NO: 233 - RNA, 100 nt, Artificial Sequence
  Description of Artificial Sequence Synthetic polynucleotide
uccugucgagcaaccccaucguccccaacaaaucuuccaucacuucgaacuccuggcggggcucagccaugcugguucucauaacaccugccugucgaca

SEQ ID NO: 234 - RNA, 100 nt, Artificial Sequence
  Description of Artificial Sequence Synthetic polynucleotide
uccugucgagcaaccccaucguccccaacaaaucuuccaucacuucgaacuccuggcggggcucagccaugcugguucucauaacaccugauagucgaca

SEQ ID NO: 235 - RNA, 100 nt, Artificial Sequence
  Description of Artificial Sequence Synthetic polynucleotide
uccugucacacaacccucccgucccagcgugaucuuccaucacuucgaacuccuggcggggcucagccaugcugguuccuccgacaccugccugucgaca

SEQ ID NO: 236 - RNA, 100 nt, Artificial Sequence
  Description of Artificial Sequence Synthetic polynucleotide
uccugucacacaacccucccgucccagcgugaucuuccaucacuucgaacuccuggcggggcucagccaugcugguuccuccgacaccugauagucgaca

SEQ ID NO: 237 - RNA, 100 nt, Artificial Sequence
  Description of Artificial Sequence Synthetic polynucleotide
uccugucacacaacccccccgucccuaagugauccuacaucacggauuucuccuggcggggcucagccaugcugguucaaaguucaccugauagucgaca

SEQ ID NO: 238 - RNA, 100 nt, Artificial Sequence
  Description of Artificial Sequence Synthetic polynucleotide
uccuguggaccaaccgcaacguccauucgugaugaaccaucagagaacacuccuggcggggcucagccaugcugguucaaaguucaccugauagucgaca

SEQ ID NO: 239 - RNA, 100 nt, Artificial Sequence
  Description of Artificial Sequence Synthetic polynucleotide
uccugcaacccaacaaauacgucggcgcgugaagguccaucugagacaacuccuggcggggcucagccaugcugguucaaaguucaccugauagucgaca

SEQ ID NO: 240 - RNA, 100 nt, Artificial Sequence
  Description of Artificial Sequence Synthetic polynucleotide
uccuuggccccaaggaguacguaggagcguguaguuccauguggaggaacuccuggcggggcucagccaugcuggaggaaaguugugcugauaagggaca

SEQ ID NO: 241 - RNA, 100 nt, Artificial Sequence
  Description of Artificial Sequence Synthetic polynucleotide
uccuuggccccaaggaguacguaggagcguguaguuccauguggaggaacuccuggcggggcucagccaugcugguucaaaguucaccugauagucgaca

SEQ ID NO: 242 - RNA, 100 nt, Artificial Sequence
  Description of Artificial Sequence Synthetic polynucleotide
uccaaacccccauagcguacgaagcagcguucccuuccagauagacgaacuccuggcggggcucagccaugcuggguaaaaguuguacugauaccggaca

SEQ ID NO: 243 - RNA, 100 nt, Artificial Sequence
  Description of Artificial Sequence Synthetic polynucleotide
uccaaacccccauagcguacgaagcagcguucccuuccagauagacgaacuccuggcggggcucagccaugcugguucaaaguucaccugauagucgaca

SEQ ID NO: 244 - RNA, 100 nt, Artificial Sequence
  Description of Artificial Sequence Synthetic polynucleotide
ucaacuccccccugccguacuagccagcgacuucuuccuagugaucgaacuccuggcggggcucagccaugcuggccgaaaguugcgcugauaugggaca

SEQ ID NO: 245 - RNA, 100 nt, Artificial Sequence
  Description of Artificial Sequence Synthetic polynucleotide
ucaacuccccccugccguacuagccagcgacuucuuccuagugaucgaacuccuggcggggcucagccaugcugguucaaaguucaccugauagucgaca

SEQ ID NO: 246 - RNA, 100 nt, Artificial Sequence
  Description of Artificial Sequence Synthetic polynucleotide
uccugucccggcacccgucucucccaggaggaucuuauuggucuucgaacuccuggcggggcucagccaugcuggaaaaaaguugcgcugauaagggaca

SEQ ID NO: 247 - RNA, 100 nt, Artificial Sequence
  Description of Artificial Sequence Synthetic polynucleotide
uccugucccggcacccgucucucccaggaggaucuuauuggucuucgaacuccuggcggggcucagccaugcugguucaaaguucaccugauagucgaca

SEQ ID NO: 248 - RNA, 100 nt, Artificial Sequence
  Description of Artificial Sequence Synthetic polynucleotide
uccugucacacaacccuaucgucccuaagugaucacgaaccacuucgaacuccuggcggggcucagccaugcuggacgaaaguuaugcugauauaggaca

SEQ ID NO: 249 - RNA, 100 nt, Artificial Sequence
  Description of Artificial Sequence Synthetic polynucleotide
uccugucacacaacccuaucgucccuaagugaucacgaaccacuucgaacuccuggcggggcucagccaugcugguucaaaguucaccugauagucgaca

SEQ ID NO: 250 - RNA, 100 nt, Artificial Sequence
  Description of Artificial Sequence Synthetic polynucleotide
uccugugaaccaaccaaaacguccaaucgugauagcguuucacuucgaacuccuggcggggcucagccaugcuggccgaaaguuguacugauaugggaca

SEQ ID NO: 251 - RNA, 100 nt, Artificial Sequence
  Description of Artificial Sequence Synthetic polynucleotide
uccugugaaccaaccaaaacguccaaucgugauagcguuucacuucgaacuccuggcggggcucagccaugcugguucaaaguucaccugauagucgaca

SEQ ID NO: 252 - RNA, 100 nt, Artificial Sequence
  Description of Artificial Sequence Synthetic polynucleotide
uccugaaacccaacagcuacgucggugcgugaguagaaaucacuucgaacuccuggcggggcucagccaugcugggagaaaguugcgcugauaaaggaca

SEQ ID NO: 253 - RNA, 100 nt, Artificial Sequence
  Description of Artificial Sequence Synthetic polynucleotide
uccugaaacccaacagcuacgucggugcgugaguagaaaucacuucgaacuccuggcggggcucagccaugcugguucaaaguucaccugauagucgaca

SEQ ID NO: 254 - RNA, 100 nt, Artificial Sequence
  Description of Artificial Sequence Synthetic polynucleotide
uccuaggccccaaaagguacgugagagcguguguagacaucacuucgaacuccuggcggggcucagccaugcuggacgaaaguugugcugauacaagaca

SEQ ID NO: 255 - RNA, 100 nt, Artificial Sequence
  Description of Artificial Sequence Synthetic polynucleotide
uccuaggccccaaaagguacgugagagcguguguagacaucacuucgaacuccuggcggggcucagccaugcugguucaaaguucaccugauagucgaca

SEQ ID NO: 256 - RNA, 100 nt, Artificial Sequence
  Description of Artificial Sequence Synthetic polynucleotide
uccacccccccacggcguacgacacagcguuagaugccaucacuucgaacuccuggcggggcucagccaugcuggcagaaaguugugcugauacaggaca

SEQ ID NO: 257 - RNA, 100 nt, Artificial Sequence
  Description of Artificial Sequence Synthetic polynucleotide
uccacccccccacggcguacgacacagcguuagaugccaucacuucgaacuccuggcggggcucagccaugcugguucaaaguucaccugauagucgaca

SEQ ID NO: 258 - RNA, 100 nt, Artificial Sequence
  Description of Artificial Sequence Synthetic polynucleotide
ucaccucccccuaaccguacuuaccagcgcacacauccaucacuucgaacuccuggcggggcucagccaugcugguucaaaguucaccugauagucgaca

SEQ ID NO: 259 - RNA, 100 nt, Artificial Sequence
  Description of Artificial Sequence Synthetic polynucleotide
uccuguccgggaacccgcuagucccaaaagcuucuuccaucacuucgaacuccuggcggggcucagccaugcuggaaaaaaguuauacugauaaaagaca

SEQ ID NO: 260 - RNA, 100 nt, Artificial Sequence
  Description of Artificial Sequence Synthetic polynucleotide
uccuguccgggaacccgcuagucccaaaagcuucuuccaucacuucgaacuccuggcggggcucagccaugcugguucaaaguucaccugauagucgaca

SEQ ID NO: 261 - RNA, 100 nt, Artificial Sequence
  Description of Artificial Sequence Synthetic polynucleotide
uccugucacacaacccccucgucccuuaaacaucuuccaucacuucgaacuccuggcggggcucagccaugcuggaggaaaguugcacugauaugggaca

SEQ ID NO: 262 - RNA, 100 nt, Artificial Sequence
  Description of Artificial Sequence Synthetic polynucleotide
uccugucacacaacccccucgucccuuaaacaucuuccaucacuucgaacuccuggcggggcucagccaugcugguucaaaguucaccugauagucgaca

SEQ ID NO: 263 - RNA, 100 nt, Artificial Sequence
  Description of Artificial Sequence Synthetic polynucleotide
uccuguccgagaacccggcagucccaaacugaucucagaucacucauccguccuggcggggcucagccacgcugguucauuacagaccugauagucgaca

SEQ ID NO: 264 - RNA, 100 nt, Artificial Sequence
  Description of Artificial Sequence Synthetic polynucleotide
uccugucaagcaaccccuucgucccuaagugaucauacaucacguauuccuccuggcggggcucagccacgcugguucaucacgaaccugacuaucgaca

SEQ ID NO: 265 - RNA, 100 nt, Artificial Sequence
  Description of Artificial Sequence Synthetic polynucleotide
uccugucaagcaaccccuucgucccuaagugaucauacaucacguauuccuccuggcggggcucagccacgcugguucaucacgaaccugauagucgaca

SEQ ID NO: 266 - RNA, 100 nt, Artificial Sequence
  Description of Artificial Sequence Synthetic polynucleotide
uccuguagaccaaccaucacguccgcccgugauagaccaucagacuuuacuccuggcggggcucagccacgcugguucacgcuaaaccugacacucgaca

SEQ ID NO: 267 - RNA, 100 nt, Artificial Sequence
  Description of Artificial Sequence Synthetic polynucleotide
uccuguagaccaaccaucacguccgcccgugauagaccaucagacuuuacuccuggcggggcucagccacgcugguucacgcuaaaccugauagucgaca

SEQ ID NO: 268 - RNA, 100 nt, Artificial Sequence
  Description of Artificial Sequence Synthetic polynucleotide
uccugaaacccaacgguuacgucggugcgugagacuccaucugucccaacuccuggcggggcucagccacgcugguucacuccagaccugaaccucgaca

SEQ ID NO: 269 - RNA, 100 nt, Artificial Sequence
  Description of Artificial Sequence Synthetic polynucleotide
uccugaaacccaacgguuacgucggugcgugagacuccaucugucccaacuccuggcggggcucagccacgcugguucacuccagaccugauagucgaca

SEQ ID NO: 270 - RNA, 100 nt, Artificial Sequence
  Description of Artificial Sequence Synthetic polynucleotide
uccuccgccccaaaagguacguaaaagcgugugguuccaugugacggaacuccuggcggggcucagccacgcugguucacuuaagaccugaaaaucgaca

SEQ ID NO: 271 - RNA, 100 nt, Artificial Sequence
  Description of Artificial Sequence Synthetic polynucleotide
uccuccgccccaaaagguacguaaaagcgugugguuccaugugacggaacuccuggcggggcucagccacgcugguucacuuaagaccugauagucgaca

SEQ ID NO: 272 - RNA, 100 nt, Artificial Sequence
  Description of Artificial Sequence Synthetic polynucleotide
uccgaacccccacggcguacgcggcagcguaaccuuccaggcaagcgaacuccuggcggggcucagccacgcugguucauccaggaccugagccucgaca

SEQ ID NO: 273 - RNA, 100 nt, Artificial Sequence
  Description of Artificial Sequence Synthetic polynucleotide
uccgaacccccacggcguacgcggcagcguaaccuuccaggcaagcgaacuccuggcggggcucagccacgcugguucauccaggaccugauagucgaca

SEQ ID NO: 274 - RNA, 100 nt, Artificial Sequence
  Description of Artificial Sequence Synthetic polynucleotide
ucgguucccccuugccguacccgccagcgcucucuuccuuaaggucgaacuccuggcggggcucagccacgcugguucacuccggaccugaacuucgaca

SEQ ID NO: 275 - RNA, 100 nt, Artificial Sequence
  Description of Artificial Sequence Synthetic polynucleotide
ucgguucccccuugccguacccgccagcgcucucuuccuuaaggucgaacuccuggcggggcucagccacgcugguucacuccggaccugauagucgaca

SEQ ID NO: 276 - RNA, 100 nt, Artificial Sequence
  Description of Artificial Sequence Synthetic polynucleotide
uccugucccgacacccgucaaucccagacagaucuugagacucuucgaacuccuggcggggcucagccacgcugguucacuucaaaccugagucucgaca

SEQ ID NO: 277 - RNA, 100 nt, Artificial Sequence
  Description of Artificial Sequence Synthetic polynucleotide
uccugucccgacacccgucaaucccagacagaucuugagacucuucgaacuccuggcggggcucagccacgcugguucacuucaaaccugauagucgaca

SEQ ID NO: 278 - RNA, 100 nt, Artificial Sequence
  Description of Artificial Sequence Synthetic polynucleotide
uccuguccaggaacccgccggucccacacugaucuggagggacuucgaacuccuggcggggcucagccacgcugguucaucuacgaccugaacaucgaca

SEQ ID NO: 279 - RNA, 100 nt, Artificial Sequence
  Description of Artificial Sequence Synthetic polynucleotide
uccuguccaggaacccgccggucccacacugaucuggagggacuucgaacuccuggcggggcucagccacgcugguucaucuacgaccugauagucgaca

SEQ ID NO: 280 - RNA, 100 nt, Artificial Sequence
  Description of Artificial Sequence Synthetic polynucleotide
uccugucacacaaccccuucguccccuagugauccgguuacacuucgaacuccuggcggggcucagccacgcugguucauuagagaccugaaaaucgaca

SEQ ID NO: 281 - RNA, 100 nt, Artificial Sequence
  Description of Artificial Sequence Synthetic polynucleotide
uccugucacacaaccccuucguccccuagugauccgguuacacuucgaacuccuggcggggcucagccacgcugguucauuagagaccugauagucgaca

SEQ ID NO: 282 - RNA, 100 nt, Artificial Sequence
  Description of Artificial Sequence Synthetic polynucleotide
uccuguacaccaaccgugacguccguucgugauacaggcucacuucgaacuccuggcggggcucagccacgcugguucauuagaaaccugacuuucgaca

SEQ ID NO: 283 - RNA, 100 nt, Artificial Sequence
  Description of Artificial Sequence Synthetic polynucleotide
uccuguacaccaaccgugacguccguucgugauacaggcucacuucgaacuccuggcggggcucagccacgcugguucauuagaaaccugauagucgaca

SEQ ID NO: 284 - RNA, 100 nt, Artificial Sequence
  Description of Artificial Sequence Synthetic polynucleotide
uccugaaacccaacaaauacgucgaugcgugaauaggaaucacuucgaacuccuggcggggcucagccacgcugguucauuugcaaccugacauucgaca

SEQ ID NO: 285 - RNA, 100 nt, Artificial Sequence
  Description of Artificial Sequence Synthetic polynucleotide
uccugaaacccaacaaauacgucgaugcgugaauaggaaucacuucgaacuccuggcggggcucagccacgcugguucauuugcaaccugauagucgaca

SEQ ID NO: 286 - RNA, 100 nt, Artificial Sequence
  Description of Artificial Sequence Synthetic polynucleotide
uccuuggccccaaaagguacguagaagcgugugacggcaucacuucgaacuccuggcggggcucagccacgcugguucaccugggaccugagcuucgaca

SEQ ID NO: 287 - RNA, 100 nt, Artificial Sequence
  Description of Artificial Sequence Synthetic polynucleotide
uccuuggccccaaaagguacguagaagcgugugacggcaucacuucgaacuccuggcggggcucagccacgcugguucaccugggaccugauagucgaca

SEQ ID NO: 288 - RNA, 100 nt, Artificial Sequence
  Description of Artificial Sequence Synthetic polynucleotide
ucccaacccccauggcguacgacacagcguugccagccaucacuucgaacuccuggcggggcucagccacgcugguucauuagcaaccugagccucgaca

SEQ ID NO: 289 - RNA, 100 nt, Artificial Sequence
  Description of Artificial Sequence Synthetic polynucleotide
ucccaacccccauggcguacgacacagcguugccagccaucacuucgaacuccuggcggggcucagccacgcugguucauuagcaaccugauagucgaca

SEQ ID NO: 290 - RNA, 100 nt, Artificial Sequence
  Description of Artificial Sequence Synthetic polynucleotide
uccugucccaauacccgucauucccaggacacccuuccaucacuucgaacuccuggcggggcucagccacgcugguucaucgccaaccugaguuucgaca

SEQ ID NO: 291 - RNA, 100 nt, Artificial Sequence
  Description of Artificial Sequence Synthetic polynucleotide
uccugucccaauacccgucauucccaggacacccuuccaucacuucgaacuccuggcggggcucagccacgcugguucaucgccaaccugauagucgaca

SEQ ID NO: 292 - RNA, 100 nt, Artificial Sequence
  Description of Artificial Sequence Synthetic polynucleotide
uccuguccacaaacccgcuggucccaucagcuucuuccaucacuucgaacuccuggcggggcucagccacgcugguucacccugaaccugaacuucgaca

SEQ ID NO: 293 - RNA, 100 nt, Artificial Sequence
  Description of Artificial Sequence Synthetic polynucleotide
uccuguccacaaacccgcuggucccaucagcuucuuccaucacuucgaacuccuggcggggcucagccacgcugguucacccugaaccugauagucgaca

SEQ ID NO: 294 - RNA, 100 nt, Artificial Sequence
  Description of Artificial Sequence Synthetic polynucleotide
uccugucgggcaaccccgccgucccucucuuaucuuccaucacuucgaacuccuggcggggcucagccacgcugguucacuacuaaccugaaccucgaca

SEQ ID NO: 295 - RNA, 100 nt, Artificial Sequence
  Description of Artificial Sequence Synthetic polynucleotide
uccugucgggcaaccccgccgucccucucuuaucuuccaucacuucgaacuccuggcggggcucagccacgcugguucacuacuaaccugauagucgaca

SEQ ID NO: 296 - RNA, 100 nt, Artificial Sequence
  Description of Artificial Sequence Synthetic polynucleotide
uccuguccagaaacccgguagucccauacugaucucagaucacucacuuguccuggcggggcucagccacgcugguucaaccgaauccugauuuccgaca

SEQ ID NO: 297 - RNA, 100 nt, Artificial Sequence
  Description of Artificial Sequence Synthetic polynucleotide
uccuguccagaaacccgguagucccauacugaucucagaucacucacuuguccuggcggggcucagccacgcugguucaaccgaauccugauagucgaca

SEQ ID NO: 298 - RNA, 100 nt, Artificial Sequence
  Description of Artificial Sequence Synthetic polynucleotide
uccugucgggcaacccuaucguccccaagugaucgagcaucacaguauccuccuggcggggcucagccacgcugguucaauuacguccugauuugcgaca

SEQ ID NO: 299 - RNA, 100 nt, Artificial Sequence
  Description of Artificial Sequence Synthetic polynucleotide
uccugucgggcaacccuaucguccccaagugaucgagcaucacaguauccuccuggcggggcucagccacgcugguucaauuacguccugauagucgaca

SEQ ID NO: 300 - RNA, 100 nt, Artificial Sequence
  Description of Artificial Sequence Synthetic polynucleotide
uccuguggaccaaccgaaacguccaaccgugauaugccaucagcgguuacuccuggcggggcucagccacgcugguucaaccgcccccugauagucgaca

SEQ ID NO: 301 - RNA, 100 nt, Artificial Sequence
  Description of Artificial Sequence Synthetic polynucleotide
uccugcggcccaacggcuacgucagugcgugacgauccaucuuccacaacuccuggcggggcucagccacgcugguucaacuaauuccugaucuccgaca

SEQ ID NO: 302 - RNA, 100 nt, Artificial Sequence
  Description of Artificial Sequence Synthetic polynucleotide
uccugcggcccaacggcuacgucagugcgugacgauccaucuuccacaacuccuggcggggcucagccacgcugguucaacuaauuccugauagucgaca

SEQ ID NO: 303 - RNA, 100 nt, Artificial Sequence
  Description of Artificial Sequence Synthetic polynucleotide
uccuacgccccaagggguacguggaagcgugcaguuccauagaucagaacuccuggcggggcucagccacgcugguucaauaccguccugaucaccgaca

SEQ ID NO: 304 - RNA, 100 nt, Artificial Sequence
  Description of Artificial Sequence Synthetic polynucleotide
uccuacgccccaagggguacguggaagcgugcaguuccauagaucagaacuccuggcggggcucagccacgcugguucaauaccguccugauagucgaca

SEQ ID NO: 305 - RNA, 100 nt, Artificial Sequence
  Description of Artificial Sequence Synthetic polynucleotide
uccaagcccccauggcguacgaagcagcguuaccuuccaggugagcgaacuccuggcggggcucagccacgcugguucaauuccauccugauucacgaca

SEQ ID NO: 306 - RNA, 100 nt, Artificial Sequence
  Description of Artificial Sequence Synthetic polynucleotide
uccaagcccccauggcguacgaagcagcguuaccuuccaggugagcgaacuccuggcggggcucagccacgcugguucaauuccauccugauagucgaca

SEQ ID NO: 307 - RNA, 100 nt, Artificial Sequence
  Description of Artificial Sequence Synthetic polynucleotide
ucaccucccccuaaccguacaaaccagcggguucuuccuaccccucgaacuccuggcggggcucagccacgcugguucaaugcgauccugauccgcgaca

SEQ ID NO: 308 - RNA, 100 nt, Artificial Sequence
  Description of Artificial Sequence Synthetic polynucleotide
ucaccucccccuaaccguacaaaccagcggguucuuccuaccccucgaacuccuggcggggcucagccacgcugguucaaugcgauccugauagucgaca

SEQ ID NO: 309 - RNA, 100 nt, Artificial Sequence
  Description of Artificial Sequence Synthetic polynucleotide
uccugucccagcacccguuuaucccagaccgaucuuaauguucuucgaacuccuggcggggcucagccacgcugguucaacuaaauccugauucgcgaca

SEQ ID NO: 310 - RNA, 100 nt, Artificial Sequence
  Description of Artificial Sequence Synthetic polynucleotide
uccugucccagcacccguuuaucccagaccgaucuuaauguucuucgaacuccuggcggggcucagccacgcugguucaacuaaauccugauagucgaca

SEQ ID NO: 311 - RNA, 100 nt, Artificial Sequence
  Description of Artificial Sequence Synthetic polynucleotide
uccuguccaaaaacccgcuggucccauuuugaucugaagcaacuucgaacuccuggcggggcucagccacgcugguucaauacaccccugauuaccgaca

SEQ ID NO: 312 - RNA, 100 nt, Artificial Sequence
  Description of Artificial Sequence Synthetic polynucleotide
uccuguccaaaaacccgcuggucccauuuugaucugaagcaacuucgaacuccuggcggggcucagccacgcugguucaauacaccccugauagucgaca

SEQ ID NO: 313 - RNA, 100 nt, Artificial Sequence
  Description of Artificial Sequence Synthetic polynucleotide
uccugucaggcaacccagccguccccuagugaucgaacugcacuucgaacuccuggcggggcucagccacgcugguucaaccgaauccugauccgcgaca

SEQ ID NO: 314 - RNA, 100 nt, Artificial Sequence
  Description of Artificial Sequence Synthetic polynucleotide
uccugucaggcaacccagccguccccuagugaucgaacugcacuucgaacuccuggcggggcucagccacgcugguucaaccgaauccugauagucgaca

SEQ ID NO: 315 - RNA, 100 nt, Artificial Sequence
  Description of Artificial Sequence Synthetic polynucleotide
uccuguaggccaaccgagacguccauacgugaugcuauuucacuucgaacuccuggcggggcucagccacgcugguucaacaucccccugaucaccgaca

SEQ ID NO: 316 - RNA, 100 nt, Artificial Sequence
  Description of Artificial Sequence Synthetic polynucleotide
uccuguaggccaaccgagacguccauacgugaugcuauuucacuucgaacuccuggcggggcucagccacgcugguucaacaucccccugauagucgaca

SEQ ID NO: 317 - RNA, 100 nt, Artificial Sequence
  Description of Artificial Sequence Synthetic polynucleotide
uccugaagcccaacgguuacgucggugcgugacaggaaaucacuucgaacuccuggcggggcucagccacgcugguucaaccggguccugauccacgaca

SEQ ID NO: 318 - RNA, 100 nt, Artificial Sequence
  Description of Artificial Sequence Synthetic polynucleotide
uccugaagcccaacgguuacgucggugcgugacaggaaaucacuucgaacuccuggcggggcucagccacgcugguucaaccggguccugauagucgaca

SEQ ID NO: 319 - RNA, 100 nt, Artificial Sequence
  Description of Artificial Sequence Synthetic polynucleotide
uccucggccccaagggguacgugaaagcguguggccacaucacuucgaacuccuggcggggcucagccacgcugguucaauagacuccugauuaacgaca

SEQ ID NO: 320 - RNA, 100 nt, Artificial Sequence
  Description of Artificial Sequence Synthetic polynucleotide
uccucggccccaagggguacgugaaagcguguggccacaucacuucgaacuccuggcggggcucagccacgcugguucaauagacuccugauagucgaca

SEQ ID NO: 321 - RNA, 100 nt, Artificial Sequence
  Description of Artificial Sequence Synthetic polynucleotide
uccgcgcccccacaacguacgguacagcgucgcgaaccaucacuucgaacuccuggcggggcucagccacgcugguucaaccucuuccugaucaacgaca

SEQ ID NO: 322 - RNA, 100 nt, Artificial Sequence
  Description of Artificial Sequence Synthetic polynucleotide
ucgaaucccccuaaccguacuagccagcggcaaaguccaucacuucgaacuccuggcggggcucagccacgcugguucaauuagauccugaucuccgaca

SEQ ID NO: 323 - RNA, 100 nt, Artificial Sequence
  Description of Artificial Sequence Synthetic polynucleotide
ucgaaucccccuaaccguacuagccagcggcaaaguccaucacuucgaacuccuggcggggcucagccacgcugguucaauuagauccugauagucgaca

SEQ ID NO: 324 - RNA, 100 nt, Artificial Sequence
  Description of Artificial Sequence Synthetic polynucleotide
uccugucccgguacccguuucucccagaucagccuuccaucacuucgaacuccuggcggggcucagccacgcugguucaaccucccccugaucuacgaca

SEQ ID NO: 325 - RNA, 100 nt, Artificial Sequence
  Description of Artificial Sequence Synthetic polynucleotide
uccugucccgguacccguuucucccagaucagccuuccaucacuucgaacuccuggcggggcucagccacgcugguucaaccucccccugauagucgaca

SEQ ID NO: 326 - RNA, 100 nt, Artificial Sequence
  Description of Artificial Sequence Synthetic polynucleotide
uccuguccaagaacccgguggucccacacaucucuuccaucacuucgaacuccuggcggggcucagccacgcugguucaaucuaguccugaucuccgaca

SEQ ID NO: 327 - RNA, 100 nt, Artificial Sequence
  Description of Artificial Sequence Synthetic polynucleotide
uccuguccaagaacccgguggucccacacaucucuuccaucacuucgaacuccuggcggggcucagccacgcugguucaaucuaguccugauagucgaca

SEQ ID NO: 328 - RNA, 100 nt, Artificial Sequence
  Description of Artificial Sequence Synthetic polynucleotide
uccugucaggcaacccauccguccccauuucaucuuccaucacuucgaacuccuggcggggcucagccacgcugguucaaugacauccugaucaacgaca

SEQ ID NO: 329 - RNA, 100 nt, Artificial Sequence
  Description of Artificial Sequence Synthetic polynucleotide
uccugucaggcaacccauccguccccauuucaucuuccaucacuucgaacuccuggcggggcucagccacgcugguucaaugacauccugauagucgaca

SEQ ID NO: 330 - RNA, 100 nt, Artificial Sequence
  Description of Artificial Sequence Synthetic polynucleotide
uccugucaaacaacccuaucgucccagcgugaucuuccaucacuucgaacuccuggcggggcucagccacgcugguucaacuacuuccugauccccgaca

SEQ ID NO: 331 - RNA, 100 nt, Artificial Sequence
  Description of Artificial Sequence Synthetic polynucleotide
uccugucaaacaacccuaucgucccagcgugaucuuccaucacuucgaacuccuggcggggcucagccacgcugguucaacuacuuccugauagucgaca

SEQ ID NO: 332 - RNA, 100 nt, Artificial Sequence
  Description of Artificial Sequence Synthetic polynucleotide
uccuguccgggaacccgcuggucccauaaugaucugggaucacugcuguauccuggcggggcucagccacgcugguucaaaacuagacugauaaaggaca

SEQ ID NO: 333 - RNA, 100 nt, Artificial Sequence
  Description of Artificial Sequence Synthetic polynucleotide
uccuguccgggaacccgcuggucccauaaugaucugggaucacugcuguauccuggcggggcucagccacgcugguucaaaacuagacugauagucgaca

SEQ ID NO: 334 - RNA, 100 nt, Artificial Sequence
  Description of Artificial Sequence Synthetic polynucleotide
uccugugggccaaccgagacguccauccgugauggcccaucaaacuauacuccuggcggggcucagccacgcugguucaaauaagugcugauaccggaca

SEQ ID NO: 335 - RNA, 100 nt, Artificial Sequence
  Description of Artificial Sequence Synthetic polynucleotide
uccugugggccaaccgagacguccauccgugauggcccaucaaacuauacuccuggcggggcucagccacgcugguucaaauaagugcugauagucgaca

SEQ ID NO: 336 - RNA, 100 nt, Artificial Sequence
  Description of Artificial Sequence Synthetic polynucleotide
uccugaaacccaacggauacgucaacgcgugaagcuccauccccuauaacuccuggcggggcucagccacgcugguucaaaaaggugcugauaagggaca

SEQ ID NO: 337 - RNA, 100 nt, Artificial Sequence
  Description of Artificial Sequence Synthetic polynucleotide
uccugaaacccaacggauacgucaacgcgugaagcuccauccccuauaacuccuggcggggcucagccacgcugguucaaaaaggugcugauagucgaca

SEQ ID NO: 338 - RNA, 100 nt, Artificial Sequence
  Description of Artificial Sequence Synthetic polynucleotide
uccuuuaccccaagggguacguagaagcgugccguuccauguagcggaacuccuggcggggcucagccacgcugguucaaaacaaaacugauaugggaca

SEQ ID NO: 339 - RNA, 100 nt, Artificial Sequence
  Description of Artificial Sequence Synthetic polynucleotide
uccuuuaccccaagggguacguagaagcgugccguuccauguagcggaacuccuggcggggcucagccacgcugguucaaaacaaaacugauagucgaca

SEQ ID NO: 340 - RNA, 100 nt, Artificial Sequence
  Description of Artificial Sequence Synthetic polynucleotide
uccacgcccccaugacguacgacacagcguuugcuuccaaaaaaccgaacuccuggcggggcucagccacgcugguucaaaacuguacugauaccggaca

SEQ ID NO: 341 - RNA, 100 nt, Artificial Sequence
  Description of Artificial Sequence Synthetic polynucleotide
uccacgcccccaugacguacgacacagcguuugcuuccaaaaaaccgaacuccuggcggggcucagccacgcugguucaaaacuguacugauagucgaca

SEQ ID NO: 342 - RNA, 100 nt, Artificial Sequence
  Description of Artificial Sequence Synthetic polynucleotide
uccugucccgacacccgucaaucccagaaagaucuugguaaccuucgaacuccuggcggggcucagccacgcugguucaaaugggugcugauauuagaca

SEQ ID NO: 343 - RNA, 100 nt, Artificial Sequence
  Description of Artificial Sequence Synthetic polynucleotide
uccugucccgacacccgucaaucccagaaagaucuugguaaccuucgaacuccuggcggggcucagccacgcugguucaaaugggugcugauagucgaca

SEQ ID NO: 344 - RNA, 100 nt, Artificial Sequence
  Description of Artificial Sequence Synthetic polynucleotide
uccugucaaacaacccauccgucccccggugaucacauugcacuucgaacuccuggcggggcucagccacgcugguucaaauaagugcugauaacagaca

SEQ ID NO: 345 - RNA, 100 nt, Artificial Sequence
  Description of Artificial Sequence Synthetic polynucleotide
uccugucaaacaacccauccgucccccggugaucacauugcacuucgaacuccuggcggggcucagccacgcugguucaaauaagugcugauagucgaca

SEQ ID NO: 346 - RNA, 100 nt, Artificial Sequence
  Description of Artificial Sequence Synthetic polynucleotide
uccugugagccaaccgacacguccgcucgugaugacgguucacuucgaacuccuggcggggcucagccacgcugguucaaauaaccgcugauauaggaca

SEQ ID NO: 347 - RNA, 100 nt, Artificial Sequence
  Description of Artificial Sequence Synthetic polynucleotide
uccugugagccaaccgacacguccgcucgugaugacgguucacuucgaacuccuggcggggcucagccacgcugguucaaauaaccgcugauagucgaca

SEQ ID NO: 348 - RNA, 100 nt, Artificial Sequence
  Description of Artificial Sequence Synthetic polynucleotide
uccuggagcccaacgguuacgucaaugcgugacguagaaucacuucgaacuccuggcggggcucagccacgcugguucaaauggagacugauaugagaca

SEQ ID NO: 349 - RNA, 100 nt, Artificial Sequence
  Description of Artificial Sequence Synthetic polynucleotide
uccuggagcccaacgguuacgucaaugcgugacguagaaucacuucgaacuccuggcggggcucagccacgcugguucaaauggagacugauagucgaca

SEQ ID NO: 350 - RNA, 100 nt, Artificial Sequence
  Description of Artificial Sequence Synthetic polynucleotide
uccuuggccccaagagguacgugaaagcgugccguggcaucacuucgaacuccuggcggggcucagccacgcugguucaaacaggugcugauaaaggaca

SEQ ID NO: 351 - RNA, 100 nt, Artificial Sequence
  Description of Artificial Sequence Synthetic polynucleotide
uccuuggccccaagagguacgugaaagcgugccguggcaucacuucgaacuccuggcggggcucagccacgcugguucaaacaggugcugauagucgaca

SEQ ID NO: 352 - RNA, 100 nt, Artificial Sequence
  Description of Artificial Sequence Synthetic polynucleotide
uccgaacccccacaacguacgauacagcguuccacaccaucacuucgaacuccuggcggggcucagccacgcugguucaaaccgggacugauacuagaca

SEQ ID NO: 353 - RNA, 100 nt, Artificial Sequence
  Description of Artificial Sequence Synthetic polynucleotide
uccgaacccccacaacguacgauacagcguuccacaccaucacuucgaacuccuggcggggcucagccacgcugguucaaaccgggacugauagucgaca

SEQ ID NO: 354 - RNA, 100 nt, Artificial Sequence
  Description of Artificial Sequence Synthetic polynucleotide
uccugucccaguacccguuaaucccaggcaacccuuccaucacuucgaacuccuggcggggcucagccacgcugguucaaauacccgcugauaacagaca

SEQ ID NO: 355 - RNA, 100 nt, Artificial Sequence
  Description of Artificial Sequence Synthetic polynucleotide
uccugucccaguacccguuaaucccaggcaacccuuccaucacuucgaacuccuggcggggcucagccacgcugguucaaauacccgcugauagucgaca

SEQ ID NO: 356 - RNA, 100 nt, Artificial Sequence
  Description of Artificial Sequence Synthetic polynucleotide
uccuguccaagaacccggaagucccacaacuuucuuccaucacuucgaacuccuggcggggcucagccacgcugguucaaacuaccacugauacgggaca

SEQ ID NO: 357 - RNA, 100 nt, Artificial Sequence
  Description of Artificial Sequence Synthetic polynucleotide
uccuguccaagaacccggaagucccacaacuuucuuccaucacuucgaacuccuggcggggcucagccacgcugguucaaacuaccacugauagucgaca

SEQ ID NO: 358 - RNA, 100 nt, Artificial Sequence
  Description of Artificial Sequence Synthetic polynucleotide
uccugucaagcaacccacccgucccagcgugaucuuccaucacuucgaacuccuggcggggcucagccacgcugguucaaacccguacugauaauagaca

SEQ ID NO: 359 - RNA, 100 nt, Artificial Sequence
  Description of Artificial Sequence Synthetic polynucleotide
uccugucaagcaacccacccgucccagcgugaucuuccaucacuucgaacuccuggcggggcucagccacgcugguucaaacccguacugauagucgaca

SEQ ID NO: 360 - RNA, 100 nt, Artificial Sequence
  Description of Artificial Sequence Synthetic polynucleotide
uccuguccaggaacccggcagucccaaucugaucugaaaucacuaguaaauccuggcggggcucagccacgcugguucaaagagucuaugauaggguaca

SEQ ID NO: 361 - RNA, 100 nt, Artificial Sequence
  Description of Artificial Sequence Synthetic polynucleotide
uccuguccaggaacccggcagucccaaucugaucugaaaucacuaguaaauccuggcggggcucagccacgcugguucaaagagucuaugauagucgaca

SEQ ID NO: 362 - RNA, 100 nt, Artificial Sequence
  Description of Artificial Sequence Synthetic polynucleotide
uccugucacacaaccccuucgucccucagugaucacacaucacggaugucuccuggcggggcucagccacgcugguucaaaggcuuggugauaggguaca

SEQ ID NO: 363 - RNA, 100 nt, Artificial Sequence
  Description of Artificial Sequence Synthetic polynucleotide
uccugucacacaaccccuucgucccucagugaucacacaucacggaugucuccuggcggggcucagccacgcugguucaaaggcuuggugauagucgaca

SEQ ID NO: 364 - RNA, 100 nt, Artificial Sequence
  Description of Artificial Sequence Synthetic polynucleotide
uccugugggccaaccgugacguccguccgugauaggccaucaaggguuacuccuggcggggcucagccacgcugguucaaagcaggaaugauaggguaca

SEQ ID NO: 365 - RNA, 100 nt, Artificial Sequence
  Description of Artificial Sequence Synthetic polynucleotide
uccugugggccaaccgugacguccguccgugauaggccaucaaggguuacuccuggcggggcucagccacgcugguucaaagcaggaaugauagucgaca

SEQ ID NO: 366 - RNA, 100 nt, Artificial Sequence
  Description of Artificial Sequence Synthetic polynucleotide
uccugaagcccaacgaauacgucacugcgugacgcuccaucugggauaacuccuggcggggcucagccacgcugguucaaagcggugaugauaggguaca

SEQ ID NO: 367 - RNA, 100 nt, Artificial Sequence
  Description of Artificial Sequence Synthetic polynucleotide
uccugaagcccaacgaauacgucacugcgugacgcuccaucugggauaacuccuggcggggcucagccacgcugguucaaagcggugaugauagucgaca

SEQ ID NO: 368 - RNA, 100 nt, Artificial Sequence
  Description of Artificial Sequence Synthetic polynucleotide
uccuccgccccaaagaguacguggaagcgugcgauuccauguucaggaacuccuggcggggcucagccacgcugguucaaaggagugaugauaggguaca

SEQ ID NO: 369 - RNA, 100 nt, Artificial Sequence
  Description of Artificial Sequence Synthetic polynucleotide
uccuccgccccaaagaguacguggaagcgugcgauuccauguucaggaacuccuggcggggcucagccacgcugguucaaaggagugaugauagucgaca

SEQ ID NO: 370 - RNA, 100 nt, Artificial Sequence
  Description of Artificial Sequence Synthetic polynucleotide
uccaggcccccauggcguacgcggcagcguacgcuuccacuuguacgaacuccuggcggggcucagccacgcugguucaaagcaugaaugauaggaaaca

SEQ ID NO: 371 - RNA, 100 nt, Artificial Sequence
  Description of Artificial Sequence Synthetic polynucleotide
uccaggcccccauggcguacgcggcagcguacgcuuccacuuguacgaacuccuggcggggcucagccacgcugguucaaagcaugaaugauagucgaca

SEQ ID NO: 372 - RNA, 100 nt, Artificial Sequence
  Description of Artificial Sequence Synthetic polynucleotide
ucaguuccccccaaccguacuaaccagcggucucuucccaagggucgaacuccuggcggggcucagccacgcugguucaaaggguuagugauaggauaca

SEQ ID NO: 373 - RNA, 100 nt, Artificial Sequence
  Description of Artificial Sequence Synthetic polynucleotide
ucaguuccccccaaccguacuaaccagcggucucuucccaagggucgaacuccuggcggggcucagccacgcugguucaaaggguuagugauagucgaca

SEQ ID NO: 374 - RNA, 100 nt, Artificial Sequence
  Description of Artificial Sequence Synthetic polynucleotide
uccugucccagcacccgucucucccaggacgaucuugacgaucuucgaacuccuggcggggcucagccacgcugguucaaagccaagaugauaggguaca

SEQ ID NO: 375 - RNA, 100 nt, Artificial Sequence
  Description of Artificial Sequence Synthetic polynucleotide
uccugucccagcacccgucucucccaggacgaucuugacgaucuucgaacuccuggcggggcucagccacgcugguucaaagccaagaugauagucgaca

SEQ ID NO: 376 - RNA, 100 nt, Artificial Sequence
  Description of Artificial Sequence Synthetic polynucleotide
uccuguccggaaacccgaaagucccaaaaugaucucaagggacuucgaacuccuggcggggcucagccacgcugguucaaagcgauggugauaggguaca

SEQ ID NO: 377 - RNA, 100 nt, Artificial Sequence
  Description of Artificial Sequence Synthetic polynucleotide
uccuguccggaaacccgaaagucccaaaaugaucucaagggacuucgaacuccuggcggggcucagccacgcugguucaaagcgauggugauagucgaca

SEQ ID NO: 378 - RNA, 100 nt, Artificial Sequence
  Description of Artificial Sequence Synthetic polynucleotide
uccuguggaccaaccaucacguccaaacgugaugccuuuucacuucgaacuccuggcggggcucagccacgcugguucaaagcuaaaaugauaggguaca

SEQ ID NO: 379 - RNA, 100 nt, Artificial Sequence
  Description of Artificial Sequence Synthetic polynucleotide
uccuguggaccaaccaucacguccaaacgugaugccuuuucacuucgaacuccuggcggggcucagccacgcugguucaaagcuaaaaugauagucgaca

SEQ ID NO: 380 - RNA, 100 nt, Artificial Sequence
  Description of Artificial Sequence Synthetic polynucleotide
uccugcggcccaacaacuacgucggcgcgugaccacggaucacuucgaacuccuggcggggcucagccacgcugguucaaaggcugggugauaggguaca

SEQ ID NO: 381 - RNA, 100 nt, Artificial Sequence
  Description of Artificial Sequence Synthetic polynucleotide
uccugcggcccaacaacuacgucggcgcgugaccacggaucacuucgaacuccuggcggggcucagccacgcugguucaaaggcugggugauagucgaca

SEQ ID NO: 382 - RNA, 100 nt, Artificial Sequence
  Description of Artificial Sequence Synthetic polynucleotide
uccucagccccaagggguacguaaaagcgugcccccacaucacuucgaacuccuggcggggcucagccacgcugguucaaagaugcaaugauaggguaca

SEQ ID NO: 383 - RNA, 100 nt, Artificial Sequence
  Description of Artificial Sequence Synthetic polynucleotide
uccucagccccaagggguacguaaaagcgugcccccacaucacuucgaacuccuggcggggcucagccacgcugguucaaagaugcaaugauagucgaca

SEQ ID NO: 384 - RNA, 100 nt, Artificial Sequence
  Description of Artificial Sequence Synthetic polynucleotide
uccgcgcccccauagcguacgcaacagcgucugaccccaucacuucgaacuccuggcggggcucagccacgcugguucaaaggguggaugauagucgaca

SEQ ID NO: 385 - RNA, 100 nt, Artificial Sequence
  Description of Artificial Sequence Synthetic polynucleotide
ucaucuccccccuaccguaccggccagcgcauagguccaucacuucgaacuccuggcggggcucagccacgcugguucaaaggaaagaugauaggguaca

SEQ ID NO: 386 - RNA, 100 nt, Artificial Sequence
  Description of Artificial Sequence Synthetic polynucleotide
ucaucuccccccuaccguaccggccagcgcauagguccaucacuucgaacuccuggcggggcucagccacgcugguucaaaggaaagaugauagucgaca

SEQ ID NO: 387 - RNA, 100 nt, Artificial Sequence
  Description of Artificial Sequence Synthetic polynucleotide
uccugucccaacacccgucauucccagauuacgcuuccaucacuucgaacuccuggcggggcucagccacgcugguucaaagcaauagugauaggguaca

SEQ ID NO: 388 - RNA, 100 nt, Artificial Sequence
  Description of Artificial Sequence Synthetic polynucleotide
uccuguccgagaacccggcagucccacucuauucuuccaucacuucgaacuccuggcggggcucagccacgcugguucaaagcuguggugauaggguaca

SEQ ID NO: 389 - RNA, 100 nt, Artificial Sequence
  Description of Artificial Sequence Synthetic polynucleotide
uccuguccgagaacccggcagucccacucuauucuuccaucacuucgaacuccuggcggggcucagccacgcugguucaaagcuguggugauagucgaca

SEQ ID NO: 390 - RNA, 100 nt, Artificial Sequence
  Description of Artificial Sequence Synthetic polynucleotide
uccugucaagcaaccccuucgucccagcgugaucuuccaucacuucgaacuccuggcggggcucagccacgcugguucaaagaauugaugauaggguaca

SEQ ID NO: 391 - RNA, 100 nt, Artificial Sequence
  Description of Artificial Sequence Synthetic polynucleotide
uccugucaagcaaccccuucgucccagcgugaucuuccaucacuucgaacuccuggcggggcucagccacgcugguucaaagaauugaugauagucgaca

SEQ ID NO: 392 - RNA, 100 nt, Artificial Sequence
  Description of Artificial Sequence Synthetic polynucleotide
ucaauucccccuaaccguaccuaccagcgccuucuuccuaguucucgaacuccuggcggggcucagccacgcuaacuauaaguucuaaugauaggauaca

SEQ ID NO: 393 - RNA, 100 nt, Artificial Sequence
  Description of Artificial Sequence Synthetic polynucleotide
uccuguccggaaacccggcggucccaauuugaucuaaacgaacuucgaacuccuggcggggcucagccacgcuuacccuaaguucuuaugauagguaaca

SEQ ID NO: 394 - RNA, 100 nt, Artificial Sequence
  Description of Artificial Sequence Synthetic polynucleotide
uccugugggccaaccaagacguccauacgugaugcgaguucacuucgaacuccuggcggggcucagccacgcuaagcccaaguuccggugauaggguaca

SEQ ID NO: 395 - RNA, 100 nt, Artificial Sequence
  Description of Artificial Sequence Synthetic polynucleotide
uccuacaccccaagggguacguacaagcgugcgugcgcaucacuucgaacuccuggcggggcucagccacgcuuacuauaaguucugaugauaggguaca

SEQ ID NO: 396 - RNA, 100 nt, Artificial Sequence
  Description of Artificial Sequence Synthetic polynucleotide
uccuguccgaaaacccgguggucccaaucucuucuuccaucacuucgaacuccuggcggggcucagccacgcuagcuacaaguucugaugauaggguaca

SEQ ID NO: 397 - RNA, 100 nt, Artificial Sequence
  Description of Artificial Sequence Synthetic polynucleotide
uccugucgggcaaccccuucguccccaagugaucggacaucacgaaaaucuccuggcggggcucagccacgcuguugauuaguucagaagauagucgaca

SEQ ID NO: 398 - RNA, 100 nt, Artificial Sequence
  Description of Artificial Sequence Synthetic polynucleotide
uccugucgggcaaccccuucguccccaagugaucggacaucacgaaaaucuccuggcggggcucagccacgcuguugauuaguucaccugauagucgaca

SEQ ID NO: 399 - RNA, 100 nt, Artificial Sequence
  Description of Artificial Sequence Synthetic polynucleotide
uccuguggaccaaccaucacguccgcccgugauaugccaucaggagcuacuccuggcggggcucagccacgcuguaauauaguucagaggauagucgaca

SEQ ID NO: 400 - RNA, 100 nt, Artificial Sequence
  Description of Artificial Sequence Synthetic polynucleotide
uccuguggaccaaccaucacguccgcccgugauaugccaucaggagcuacuccuggcggggcucagccacgcuguaauauaguucaccugauagucgaca

SEQ ID NO: 401 - RNA, 100 nt, Artificial Sequence
  Description of Artificial Sequence Synthetic polynucleotide
uccuggcacccaacgaauacgucgaugcgugagacuccaucugggauaacuccuggcggggcucagccacgcuguuaauuaguucaaaagauagucgaca

SEQ ID NO: 402 - RNA, 100 nt, Artificial Sequence
  Description of Artificial Sequence Synthetic polynucleotide
uccuggcacccaacgaauacgucgaugcgugagacuccaucugggauaacuccuggcggggcucagccacgcuguuaauuaguucaccugauagucgaca

SEQ ID NO: 403 - RNA, 100 nt, Artificial Sequence
  Description of Artificial Sequence Synthetic polynucleotide
uccuagaccccaaacaguacguggaagcgugcaauuccauguuugggaacuccuggcggggcucagccacgcugaccuguaguucaggcgauagucgaca

SEQ ID NO: 404 - RNA, 100 nt, Artificial Sequence
  Description of Artificial Sequence Synthetic polynucleotide
ucccaacccccacggcguacggggcagcgucugcuuccacgugcacgaacuccuggcggggcucagccacgcuguauaccaguucagacgauagucgaca

SEQ ID NO: 405 - RNA, 100 nt, Artificial Sequence
  Description of Artificial Sequence Synthetic polynucleotide
ucccaacccccacggcguacggggcagcgucugcuuccacgugcacgaacuccuggcggggcucagccacgcuguauaccaguucaccugauagucgaca

SEQ ID NO: 406 - RNA, 100 nt, Artificial Sequence
  Description of Artificial Sequence Synthetic polynucleotide
uccugucccgauacccgucaaucccagaacgaucuuagugaucuucgaacuccuggcggggcucagccacgcuguaaacuaguucaggagauagucgaca

SEQ ID NO: 407 - RNA, 100 nt, Artificial Sequence
  Description of Artificial Sequence Synthetic polynucleotide
uccuguccgaaaacccgcuggucccaagaugaucugugcgaacuucgaacuccuggcggggcucagccacgcuguuguguaguucaaaggauagucgaca

SEQ ID NO: 408 - RNA, 100 nt, Artificial Sequence
  Description of Artificial Sequence Synthetic polynucleotide
uccuguccgaaaacccgcuggucccaagaugaucugugcgaacuucgaacuccuggcggggcucagccacgcuguuguguaguucaccugauagucgaca

SEQ ID NO: 409 - RNA, 100 nt, Artificial Sequence
  Description of Artificial Sequence Synthetic polynucleotide
uccuguacaccaaccacaacguccauccgugaugcauauucacuucgaacuccuggcggggcucagccacgcugugcccuaguucaaacgauagucgaca

SEQ ID NO: 410 - RNA, 100 nt, Artificial Sequence
  Description of Artificial Sequence Synthetic polynucleotide
uccuguacaccaaccacaacguccauccgugaugcauauucacuucgaacuccuggcggggcucagccacgcugugcccuaguucaccugauagucgaca

SEQ ID NO: 411 - RNA, 100 nt, Artificial Sequence
  Description of Artificial Sequence Synthetic polynucleotide
uccuggagcccaacagcuacgucaaugcgugaaaaguaaucacuucgaacuccuggcggggcucagccacgcugauagccaguucaaacgauagucgaca

SEQ ID NO: 412 - RNA, 100 nt, Artificial Sequence
  Description of Artificial Sequence Synthetic polynucleotide
uccuggagcccaacagcuacgucaaugcgugaaaaguaaucacuucgaacuccuggcggggcucagccacgcugauagccaguucaccugauagucgaca

SEQ ID NO: 413 - RNA, 100 nt, Artificial Sequence
  Description of Artificial Sequence Synthetic polynucleotide
uccucggccccaaagaguacguaagagcgugccggaacaucacuucgaacuccuggcggggcucagccacgcugccagguaguucaacagauagucgaca

SEQ ID NO: 414 - RNA, 100 nt, Artificial Sequence
  Description of Artificial Sequence Synthetic polynucleotide
uccaagcccccauagcguacggagcagcguacuaggccaucacuucgaacuccuggcggggcucagccacgcugccuaauaguucaacggauagucgaca

SEQ ID NO: 415 - RNA, 100 nt, Artificial Sequence
  Description of Artificial Sequence Synthetic polynucleotide
uccaagcccccauagcguacggagcagcguacuaggccaucacuucgaacuccuggcggggcucagccacgcugccuaauaguucaccugauagucgaca

SEQ ID NO: 416 - RNA, 100 nt, Artificial Sequence
  Description of Artificial Sequence Synthetic polynucleotide
uccugucccaguacccgucuaucccaggaccagcuuccaucacuucgaacuccuggcggggcucagccacgcugcgaaacaguucaagcgauagucgaca

SEQ ID NO: 417 - RNA, 100 nt, Artificial Sequence
  Description of Artificial Sequence Synthetic polynucleotide
uccuguccgggaacccgccggucccauaagccucuuccaucacuucgaacuccuggcggggcucagccacgcugcuauuuaguucaggggauagucgaca

SEQ ID NO: 418 - RNA, 100 nt, Artificial Sequence
  Description of Artificial Sequence Synthetic polynucleotide
uccugucgggcaaccccgccgucccuuuucuaucuuccaucacuucgaacuccuggcggggcucagccacgcugaccugcaguucaagcgauagucgaca

SEQ ID NO: 419 - RNA, 100 nt, Artificial Sequence
  Description of Artificial Sequence Synthetic polynucleotide
uccugucgaacaacccuaucgucccuuggugaucaggcaucaccgucuccuccuggcggggcucagccacgcuggcuagguguucacaauauagucgaca

SEQ ID NO: 420 - RNA, 100 nt, Artificial Sequence
  Description of Artificial Sequence Synthetic polynucleotide
uccugucgaacaacccuaucgucccuuggugaucaggcaucaccgucuccuccuggcggggcucagccacgcuggcuagguguucaccugauagucgaca

SEQ ID NO: 421 - RNA, 100 nt, Artificial Sequence
  Description of Artificial Sequence Synthetic polynucleotide
uccuguggaccaaccgaaacguccauucgugauggcccaucaacguauacuccuggcggggcucagccacgcugggcugccguucacgaaauagucgaca

SEQ ID NO: 422 - RNA, 100 nt, Artificial Sequence
  Description of Artificial Sequence Synthetic polynucleotide
uccuguggaccaaccgaaacguccauucgugauggcccaucaacguauacuccuggcggggcucagccacgcugggcugccguucaccugauagucgaca

SEQ ID NO: 423 - RNA, 100 nt, Artificial Sequence
  Description of Artificial Sequence Synthetic polynucleotide
uccugcagcccaacaguuacgucaaugcgugacaauccaucugucuaaacuccuggcggggcucagccacgcugggguuauguucacgacauagucgaca

SEQ ID NO: 424 - RNA, 100 nt, Artificial Sequence
  Description of Artificial Sequence Synthetic polynucleotide
uccugcagcccaacaguuacgucaaugcgugacaauccaucugucuaaacuccuggcggggcucagccacgcugggguuauguucaccugauagucgaca

SEQ ID NO: 425 - RNA, 100 nt, Artificial Sequence
  Description of Artificial Sequence Synthetic polynucleotide
uccuaaaccccaagggguacgugagagcgugccguuccauaccaaggaacuccuggcggggcucagccacgcuggggguacguucacagcauagucgaca

SEQ ID NO: 426 - RNA, 100 nt, Artificial Sequence
  Description of Artificial Sequence Synthetic polynucleotide
uccuaaaccccaagggguacgugagagcgugccguuccauaccaaggaacuccuggcggggcucagccacgcuggggguacguucaccugauagucgaca

SEQ ID NO: 427 - RNA, 100 nt, Artificial Sequence
  Description of Artificial Sequence Synthetic polynucleotide
uccgaccccccacggcguacgacacagcguuagcuuccagggacgcgaacuccuggcggggcucagccacgcugggaauauguucacaaaauagucgaca

SEQ ID NO: 428 - RNA, 100 nt, Artificial Sequence
  Description of Artificial Sequence Synthetic polynucleotide
uccgaccccccacggcguacgacacagcguuagcuuccagggacgcgaacuccuggcggggcucagccacgcugggaauauguucaccugauagucgaca

SEQ ID NO: 429 - RNA, 100 nt, Artificial Sequence
  Description of Artificial Sequence Synthetic polynucleotide
ucaccuccccccuaccguaccgaccagcgaccucuuccuaguaaucgaacuccuggcggggcucagccacgcuggagugcuguucacgguauagucgaca

SEQ ID NO: 430 - RNA, 100 nt, Artificial Sequence
  Description of Artificial Sequence Synthetic polynucleotide
ucaccuccccccuaccguaccgaccagcgaccucuuccuaguaaucgaacuccuggcggggcucagccacgcuggagugcuguucaccugauagucgaca

SEQ ID NO: 431 - RNA, 100 nt, Artificial Sequence
  Description of Artificial Sequence Synthetic polynucleotide
uccugucccaacacccguuauucccagacagaucuuauaccccuucgaacuccuggcggggcucagccacgcuggcaguccguucacaaaauagucgaca

SEQ ID NO: 432 - RNA, 100 nt, Artificial Sequence
  Description of Artificial Sequence Synthetic polynucleotide
uccuguccaaaaacccgguagucccauauugaucuggggaaacuucgaacuccuggcggggcucagccacgcuggaauaucguucacacaauagucgaca

SEQ ID NO: 433 - RNA, 100 nt, Artificial Sequence
  Description of Artificial Sequence Synthetic polynucleotide
uccuguccaaaaacccgguagucccauauugaucuggggaaacuucgaacuccuggcggggcucagccacgcuggaauaucguucaccugauagucgaca

SEQ ID NO: 434 - RNA, 100 nt, Artificial Sequence
  Description of Artificial Sequence Synthetic polynucleotide
uccugucgggcaacccauucgucccuuagugauccggaaccacuucgaacuccuggcggggcucagccacgcuggccgguuguucacgguauagucgaca

SEQ ID NO: 435 - RNA, 100 nt, Artificial Sequence
  Description of Artificial Sequence Synthetic polynucleotide
uccugucgggcaacccauucgucccuuagugauccggaaccacuucgaacuccuggcggggcucagccacgcuggccgguuguucaccugauagucgaca

SEQ ID NO: 436 - RNA, 100 nt, Artificial Sequence
  Description of Artificial Sequence Synthetic polynucleotide
uccuguaagccaaccaagacguccauccgugauagcgacucacuucgaacuccuggcggggcucagccacgcuggaggcuuguucacaccauagucgaca

SEQ ID NO: 437 - RNA, 100 nt, Artificial Sequence
  Description of Artificial Sequence Synthetic polynucleotide
uccuguaagccaaccaagacguccauccgugauagcgacucacuucgaacuccuggcggggcucagccacgcuggaggcuuguucaccugauagucgaca

SEQ ID NO: 438 - RNA, 100 nt, Artificial Sequence
  Description of Artificial Sequence Synthetic polynucleotide
uccugggacccaacaacuacgucgacgcgugaauauggaucacuucgaacuccuggcggggcucagccacgcuggccccuuguucacgacauagucgaca

SEQ ID NO: 439 - RNA, 100 nt, Artificial Sequence
  Description of Artificial Sequence Synthetic polynucleotide
uccugggacccaacaacuacgucgacgcgugaauauggaucacuucgaacuccuggcggggcucagccacgcuggccccuuguucaccugauagucgaca

SEQ ID NO: 440 - RNA, 100 nt, Artificial Sequence
  Description of Artificial Sequence Synthetic polynucleotide
uccuugaccccaagaaguacgugggagcgugccuccgcaucacuucgaacuccuggcggggcucagccacgcuggaagguuguucacgauauagucgaca

SEQ ID NO: 441 - RNA, 100 nt, Artificial Sequence
  Description of Artificial Sequence Synthetic polynucleotide
uccuugaccccaagaaguacgugggagcgugccuccgcaucacuucgaacuccuggcggggcucagccacgcuggaagguuguucaccugauagucgaca

SEQ ID NO: 442 - RNA, 100 nt, Artificial Sequence
  Description of Artificial Sequence Synthetic polynucleotide
uccauccccccauggcguacgcaacagcguacgggcccaucacuucgaacuccuggcggggcucagccacgcuggggauccguucacggcauagucgaca

SEQ ID NO: 443 - RNA, 100 nt, Artificial Sequence
  Description of Artificial Sequence Synthetic polynucleotide
uccauccccccauggcguacgcaacagcguacgggcccaucacuucgaacuccuggcggggcucagccacgcuggggauccguucaccugauagucgaca

SEQ ID NO: 444 - RNA, 100 nt, Artificial Sequence
  Description of Artificial Sequence Synthetic polynucleotide
ucaguuccccccugccguacuagccagcgguugcguccaucacuucgaacuccuggcggggcucagccacgcuggaacuguguucacauuauagucgaca

SEQ ID NO: 445 - RNA, 100 nt, Artificial Sequence
  Description of Artificial Sequence Synthetic polynucleotide
ucaguuccccccugccguacuagccagcgguugcguccaucacuucgaacuccuggcggggcucagccacgcuggaacuguguucaccugauagucgaca

SEQ ID NO: 446 - RNA, 100 nt, Artificial Sequence
  Description of Artificial Sequence Synthetic polynucleotide
uccugucccgauacccguuaaucccaggagccccuuccaucacuucgaacuccuggcggggcucagccacgcuggcggcccguucacaucauagucgaca

SEQ ID NO: 447 - RNA, 100 nt, Artificial Sequence
  Description of Artificial Sequence Synthetic polynucleotide
uccugucccgauacccguuaaucccaggagccccuuccaucacuucgaacuccuggcggggcucagccacgcuggcggcccguucaccugauagucgaca

SEQ ID NO: 448 - RNA, 100 nt, Artificial Sequence
  Description of Artificial Sequence Synthetic polynucleotide
uccuguccaggaacccggcggucccaaauccuucuuccaucacuucgaacuccuggcggggcucagccacgcuggagauguguucacaccauagucgaca

SEQ ID NO: 449 - RNA, 100 nt, Artificial Sequence
  Description of Artificial Sequence Synthetic polynucleotide
uccuguccaggaacccggcggucccaaauccuucuuccaucacuucgaacuccuggcggggcucagccacgcuggagauguguucaccugauagucgaca

SEQ ID NO: 450 - RNA, 100 nt, Artificial Sequence
  Description of Artificial Sequence Synthetic polynucleotide
uccugucgggcaacccaaucgucccagcgugaucuuccaucacuucgaacuccuggcggggcucagccacgcugggagacuguucacaaaauagucgaca

SEQ ID NO: 451 - RNA, 100 nt, Artificial Sequence
  Description of Artificial Sequence Synthetic polynucleotide
uccugucgggcaacccaaucgucccagcgugaucuuccaucacuucgaacuccuggcggggcucagccacgcugggagacuguucaccugauagucgaca

SEQ ID NO: 452 - RNA, 100 nt, Artificial Sequence
  Description of Artificial Sequence Synthetic polynucleotide
uccuguccacaaacccgguagucccauuuugaucucgaaucacuacaauguccuggcggggcucagccacgcugguaacauuuucaccgcuuagucgaca

SEQ ID NO: 453 - RNA, 100 nt, Artificial Sequence
  Description of Artificial Sequence Synthetic polynucleotide
uccuguccacaaacccgguagucccauuuugaucucgaaucacuacaauguccuggcggggcucagccacgcugguaacauuuucaccugauagucgaca

SEQ ID NO: 454 - RNA, 100 nt, Artificial Sequence
  Description of Artificial Sequence Synthetic polynucleotide
uccugucacacaacccauccgucccucggugaucguacaucaccaaugccuccuggcggggcucagccacgcugguaaugcuuucaccguuuagucgaca

SEQ ID NO: 455 - RNA, 100 nt, Artificial Sequence
  Description of Artificial Sequence Synthetic polynucleotide
uccugucacacaacccauccgucccucggugaucguacaucaccaaugccuccuggcggggcucagccacgcugguaaugcuuucaccugauagucgaca

SEQ ID NO: 456 - RNA, 100 nt, Artificial Sequence
  Description of Artificial Sequence Synthetic polynucleotide
uccuguaggccaaccauaacguccauucgugaugcgccaucaauacauacuccuggcggggcucagccacgcugguauacucuucacccccuagucgaca

SEQ ID NO: 457 - RNA, 100 nt, Artificial Sequence
  Description of Artificial Sequence Synthetic polynucleotide
uccuguaggccaaccauaacguccauucgugaugcgccaucaauacauacuccuggcggggcucagccacgcugguauacucuucaccugauagucgaca

SEQ ID NO: 458 - RNA, 100 nt, Artificial Sequence
  Description of Artificial Sequence Synthetic polynucleotide
uccugcgacccaacgaauacgucgacgcgugagaauccaucugggaaaacuccuggcggggcucagccacgcuggugauacauucacccccuagucgaca

SEQ ID NO: 459 - RNA, 100 nt, Artificial Sequence
  Description of Artificial Sequence Synthetic polynucleotide
uccugcgacccaacgaauacgucgacgcgugagaauccaucugggaaaacuccuggcggggcucagccacgcuggugauacauucaccugauagucgaca

SEQ ID NO: 460 - RNA, 100 nt, Artificial Sequence
  Description of Artificial Sequence Synthetic polynucleotide
uccuuaaccccaagagguacguaagagcguguaauuccauaugggggaacuccuggcggggcucagccacgcuggugggcaauucacccacuagucgaca

SEQ ID NO: 461 - RNA, 100 nt, Artificial Sequence
  Description of Artificial Sequence Synthetic polynucleotide
uccuuaaccccaagagguacguaagagcguguaauuccauaugggggaacuccuggcggggcucagccacgcuggugggcaauucaccugauagucgaca

SEQ ID NO: 462 - RNA, 100 nt, Artificial Sequence
  Description of Artificial Sequence Synthetic polynucleotide
uccccacccccauagcguacgcaacagcguacccuuccaaaguagcgaacuccuggcggggcucagccacgcugguacccuuuucaccgccuagucgaca

SEQ ID NO: 463 - RNA, 100 nt, Artificial Sequence
  Description of Artificial Sequence Synthetic polynucleotide
uccccacccccauagcguacgcaacagcguacccuuccaaaguagcgaacuccuggcggggcucagccacgcugguacccuuuucaccugauagucgaca

SEQ ID NO: 464 - RNA, 100 nt, Artificial Sequence
  Description of Artificial Sequence Synthetic polynucleotide
ucgaaucccccucgccguacacaccagcgaucucuucccuagugucgaacuccuggcggggcucagccacgcugguauuaccuucaccgucuagucgaca

SEQ ID NO: 465 - RNA, 100 nt, Artificial Sequence
  Description of Artificial Sequence Synthetic polynucleotide
ucgaaucccccucgccguacacaccagcgaucucuucccuagugucgaacuccuggcggggcucagccacgcugguauuaccuucaccugauagucgaca

SEQ ID NO: 466 - RNA, 100 nt, Artificial Sequence
  Description of Artificial Sequence Synthetic polynucleotide
uccugucccaauacccgucaaucccaggcagaucuuggccuucuucgaacuccuggcggggcucagccacgcugguaauuacuucacccuuuagucgaca

SEQ ID NO: 467 - RNA, 100 nt, Artificial Sequence
  Description of Artificial Sequence Synthetic polynucleotide
uccugucccaauacccgucaaucccaggcagaucuuggccuucuucgaacuccuggcggggcucagccacgcugguaauuacuucaccugauagucgaca

SEQ ID NO: 468 - RNA, 100 nt, Artificial Sequence
  Description of Artificial Sequence Synthetic polynucleotide
uccuguccacaaacccggcggucccauauugaucuagguuaacuucgaacuccuggcggggcucagccacgcuggugaacuauucaccccuuagucgaca

SEQ ID NO: 469 - RNA, 100 nt, Artificial Sequence
  Description of Artificial Sequence Synthetic polynucleotide
uccuguccacaaacccggcggucccauauugaucuagguuaacuucgaacuccuggcggggcucagccacgcuggugaacuauucaccugauagucgaca

SEQ ID NO: 470 - RNA, 100 nt, Artificial Sequence
  Description of Artificial Sequence Synthetic polynucleotide
uccugucgggcaacccagccguccccuagugaucaguagacacuucgaacuccuggcggggcucagccacgcuggugccuacuucacccauuagucgaca

SEQ ID NO: 471 - RNA, 100 nt, Artificial Sequence
  Description of Artificial Sequence Synthetic polynucleotide
uccugucgggcaacccagccguccccuagugaucaguagacacuucgaacuccuggcggggcucagccacgcuggugccuacuucaccugauagucgaca

SEQ ID NO: 472 - RNA, 100 nt, Artificial Sequence
  Description of Artificial Sequence Synthetic polynucleotide
uccugugaaccaaccauaacguccauacgugauaguuacucacuucgaacuccuggcggggcucagccacgcugguagcuccuucaccaucuagucgaca

SEQ ID NO: 473 - RNA, 100 nt, Artificial Sequence
  Description of Artificial Sequence Synthetic polynucleotide
uccugugaaccaaccauaacguccauacgugauaguuacucacuucgaacuccuggcggggcucagccacgcugguagcuccuucaccugauagucgaca

SEQ ID NO: 474 - RNA, 100 nt, Artificial Sequence
  Description of Artificial Sequence Synthetic polynucleotide
uccugggacccaacauauacgucgaugcgugagagacaaucacuucgaacuccuggcggggcucagccacgcuggucccugcuucacccauuagucgaca

SEQ ID NO: 475 - RNA, 100 nt, Artificial Sequence
  Description of Artificial Sequence Synthetic polynucleotide
uccugggacccaacauauacgucgaugcgugagagacaaucacuucgaacuccuggcggggcucagccacgcuggucccugcuucaccugauagucgaca

SEQ ID NO: 476 - RNA, 100 nt, Artificial Sequence
  Description of Artificial Sequence Synthetic polynucleotide
uccuugaccccaagggguacgugaaagcgugcuggcgcaucacuucgaacuccuggcggggcucagccacgcuggugacuucuucacccuuuagucgaca

SEQ ID NO: 477 - RNA, 100 nt, Artificial Sequence
  Description of Artificial Sequence Synthetic polynucleotide
uccuugaccccaagggguacgugaaagcgugcuggcgcaucacuucgaacuccuggcggggcucagccacgcuggugacuucuucaccugauagucgaca

SEQ ID NO: 478 - RNA, 100 nt, Artificial Sequence
  Description of Artificial Sequence Synthetic polynucleotide
uccccccccccauaacguacggcacagcguucgaaaccaucacuucgaacuccuggcggggcucagccacgcugguaaccucuucaccgccuagucgaca

SEQ ID NO: 479 - RNA, 100 nt, Artificial Sequence
  Description of Artificial Sequence Synthetic polynucleotide
uccccccccccauaacguacggcacagcguucgaaaccaucacuucgaacuccuggcggggcucagccacgcugguaaccucuucaccugauagucgaca

SEQ ID NO: 480 - RNA, 100 nt, Artificial Sequence
  Description of Artificial Sequence Synthetic polynucleotide
ucaaaucccccucgccguacuagccagcgcaggaguccaucacuucgaacuccuggcggggcucagccacgcuggugaaccauucaccaccuagucgaca

SEQ ID NO: 481 - RNA, 100 nt, Artificial Sequence
  Description of Artificial Sequence Synthetic polynucleotide
ucaaaucccccucgccguacuagccagcgcaggaguccaucacuucgaacuccuggcggggcucagccacgcuggugaaccauucaccugauagucgaca

SEQ ID NO: 482 - RNA, 100 nt, Artificial Sequence
  Description of Artificial Sequence Synthetic polynucleotide
uccugucccggcacccguuucucccagauauuccuuccaucacuucgaacuccuggcggggcucagccacgcugguguucuuuucaccgguuagucgaca

SEQ ID NO: 483 - RNA, 100 nt, Artificial Sequence
  Description of Artificial Sequence Synthetic polynucleotide
uccuguccacaaacccgaaagucccaaaucucucuuccaucacuucgaacuccuggcggggcucagccacgcugguauuguuuucaccgguuagucgaca

SEQ ID NO: 484 - RNA, 100 nt, Artificial Sequence
  Description of Artificial Sequence Synthetic polynucleotide
uccuguccacaaacccgaaagucccaaaucucucuuccaucacuucgaacuccuggcggggcucagccacgcugguauuguuuucaccugauagucgaca

SEQ ID NO: 485 - RNA, 100 nt, Artificial Sequence
  Description of Artificial Sequence Synthetic polynucleotide
uccugucaagcaacccauucguccccuucacaucuuccaucacuucgaacuccuggcggggcucagccacgcuggugcccguuucaccauuuagucgaca

SEQ ID NO: 486 - RNA, 100 nt, Artificial Sequence
  Description of Artificial Sequence Synthetic polynucleotide
uccugucaagcaacccauucguccccuucacaucuuccaucacuucgaacuccuggcggggcucagccacgcuggugcccguuucaccugauagucgaca

SEQ ID NO: 487 - RNA, 100 nt, Artificial Sequence
  Description of Artificial Sequence Synthetic polynucleotide
uccugucagacaacccauucgucccagcgugaucuuccaucacuucgaacuccuggcggggcucagccacgcuggugcuuacuucaccgucuagucgaca

SEQ ID NO: 488 - RNA, 100 nt, Artificial Sequence
  Description of Artificial Sequence Synthetic polynucleotide
uccugucagacaacccauucgucccagcgugaucuuccaucacuucgaacuccuggcggggcucagccacgcuggugcuuacuucaccugauagucgaca

SEQ ID NO: 489 - RNA, 100 nt, Artificial Sequence
  Description of Artificial Sequence Synthetic polynucleotide
uccugucaggcaacccccccgucccccagugaucauacaucacgcucaucuccuggcggggcucagccacgcugguugcguucucaccuaucagucgaca

SEQ ID NO: 490 - RNA, 100 nt, Artificial Sequence
  Description of Artificial Sequence Synthetic polynucleotide
uccugucaggcaacccccccgucccccagugaucauacaucacgcucaucuccuggcggggcucagccacgcugguugcguucucaccugauagucgaca

SEQ ID NO: 491 - RNA, 100 nt, Artificial Sequence
  Description of Artificial Sequence Synthetic polynucleotide
uccugugggccaaccgacacguccguucgugaugagccaucagacaacacuccuggcggggcucagccacgcugguugcuccaucaccuuacagucgaca

SEQ ID NO: 492 - RNA, 100 nt, Artificial Sequence
  Description of Artificial Sequence Synthetic polynucleotide
uccugugggccaaccgacacguccguucgugaugagccaucagacaacacuccuggcggggcucagccacgcugguugcuccaucaccugauagucgaca

SEQ ID NO: 493 - RNA, 100 nt, Artificial Sequence
  Description of Artificial Sequence Synthetic polynucleotide
uccugcaacccaacagcuacgucaaugcgugaaaguccaucugacaaaacuccuggcggggcucagccacgcugguuagcgaaucaccuaacagucgaca

SEQ ID NO: 494 - RNA, 100 nt, Artificial Sequence
  Description of Artificial Sequence Synthetic polynucleotide
uccugcaacccaacagcuacgucaaugcgugaaaguccaucugacaaaacuccuggcggggcucagccacgcugguuagcgaaucaccugauagucgaca

SEQ ID NO: 495 - RNA, 100 nt, Artificial Sequence
  Description of Artificial Sequence Synthetic polynucleotide
uccggacccccaugacguacgaaacagcguuaccuuccaauuaaacgaacuccuggcggggcucagccacgcugguugcgcccucaccuaccagucgaca

SEQ ID NO: 496 - RNA, 100 nt, Artificial Sequence
  Description of Artificial Sequence Synthetic polynucleotide
uccggacccccaugacguacgaaacagcguuaccuuccaauuaaacgaacuccuggcggggcucagccacgcugguugcgcccucaccugauagucgaca

SEQ ID NO: 497 - RNA, 100 nt, Artificial Sequence
  Description of Artificial Sequence Synthetic polynucleotide
ucaaauccccccaaccguacugaccagcgcauucuuccccgugaucgaacuccuggcggggcucagccacgcugguuacucucucaccuuccagucgaca

SEQ ID NO: 498 - RNA, 100 nt, Artificial Sequence
  Description of Artificial Sequence Synthetic polynucleotide
ucaaauccccccaaccguacugaccagcgcauucuuccccgugaucgaacuccuggcggggcucagccacgcugguuacucucucaccugauagucgaca

SEQ ID NO: 499 - RNA, 100 nt, Artificial Sequence
  Description of Artificial Sequence Synthetic polynucleotide
uccugucccagcacccgucauucccagaccgaucuuaauggucuucgaacuccuggcggggcucagccacgcugguugcucacucaccuacaagucgaca

SEQ ID NO: 500 - RNA, 100 nt, Artificial Sequence
  Description of Artificial Sequence Synthetic polynucleotide
uccugucccagcacccgucauucccagaccgaucuuaauggucuucgaacuccuggcggggcucagccacgcugguugcucacucaccugauagucgaca

SEQ ID NO: 501 - RNA, 100 nt, Artificial Sequence
  Description of Artificial Sequence Synthetic polynucleotide
uccuguccaagaacccggaagucccacacugaucuagauggacuucgaacuccuggcggggcucagccacgcugguuacgcaaucaccuaacagucgaca

SEQ ID NO: 502 - RNA, 100 nt, Artificial Sequence
  Description of Artificial Sequence Synthetic polynucleotide
uccuguccaagaacccggaagucccacacugaucuagauggacuucgaacuccuggcggggcucagccacgcugguuacgcaaucaccugauagucgaca

SEQ ID NO: 503 - RNA, 100 nt, Artificial Sequence
  Description of Artificial Sequence Synthetic polynucleotide
uccugucaggcaacccuaucgucccuaagugaucgaaggacacuucgaacuccuggcggggcucagccacgcugguugcucacucaccuacgagucgaca

SEQ ID NO: 504 - RNA, 100 nt, Artificial Sequence
  Description of Artificial Sequence Synthetic polynucleotide
uccugucaggcaacccuaucgucccuaagugaucgaaggacacuucgaacuccuggcggggcucagccacgcugguugcucacucaccugauagucgaca

SEQ ID NO: 505 - RNA, 100 nt, Artificial Sequence
  Description of Artificial Sequence Synthetic polynucleotide
uccuguaggccaaccaccacguccauucgugauaaaauuucacuucgaacuccuggcggggcucagccacgcugguuaauccaucaccuaucagucgaca

SEQ ID NO: 506 - RNA, 100 nt, Artificial Sequence
  Description of Artificial Sequence Synthetic polynucleotide
uccuguaggccaaccaccacguccauucgugauaaaauuucacuucgaacuccuggcggggcucagccacgcugguuaauccaucaccugauagucgaca

SEQ ID NO: 507 - RNA, 100 nt, Artificial Sequence
  Description of Artificial Sequence Synthetic polynucleotide
uccugaagcccaacggauacgucggcgcgugacagcgaaucacuucgaacuccuggcggggcucagccacgcugguuguucagucaccuacaagucgaca

SEQ ID NO: 508 - RNA, 100 nt, Artificial Sequence
  Description of Artificial Sequence Synthetic polynucleotide
uccugaagcccaacggauacgucggcgcgugacagcgaaucacuucgaacuccuggcggggcucagccacgcugguuguucagucaccugauagucgaca

SEQ ID NO: 509 - RNA, 100 nt, Artificial Sequence
  Description of Artificial Sequence Synthetic polynucleotide
uccuccaccccaagaaguacguggaagcgugcggaagcaucacuucgaacuccuggcggggcucagccacgcugguuaucgcgucaccuuucagucgaca

SEQ ID NO: 510 - RNA, 100 nt, Artificial Sequence
  Description of Artificial Sequence Synthetic polynucleotide
uccuccaccccaagaaguacguggaagcgugcggaagcaucacuucgaacuccuggcggggcucagccacgcugguuaucgcgucaccugauagucgaca

SEQ ID NO: 511 - RNA, 100 nt, Artificial Sequence
  Description of Artificial Sequence Synthetic polynucleotide
uccgcccccccacagcguacgguacagcguccugagccaucacuucgaacuccuggcggggcucagccacgcugguugcgcuaucaccuaacagucgaca

SEQ ID NO: 512 - RNA, 100 nt, Artificial Sequence
  Description of Artificial Sequence Synthetic polynucleotide
ucaacuccccccugccguacuggccagcgcaacuauccaucacuucgaacuccuggcggggcucagccacgcugguuguucggucaccuuacagucgaca

SEQ ID NO: 513 - RNA, 100 nt, Artificial Sequence
  Description of Artificial Sequence Synthetic polynucleotide
ucaacuccccccugccguacuggccagcgcaacuauccaucacuucgaacuccuggcggggcucagccacgcugguuguucggucaccugauagucgaca

SEQ ID NO: 514 - RNA, 100 nt, Artificial Sequence
  Description of Artificial Sequence Synthetic polynucleotide
uccugucccaacacccguuaaucccaggaccugcuuccaucacuucgaacuccuggcggggcucagccacgcugguuauccugucaccuaugagucgaca

SEQ ID NO: 515 - RNA, 100 nt, Artificial Sequence
  Description of Artificial Sequence Synthetic polynucleotide
uccugucccaacacccguuaaucccaggaccugcuuccaucacuucgaacuccuggcggggcucagccacgcugguuauccugucaccugauagucgaca

SEQ ID NO: 516 - RNA, 100 nt, Artificial Sequence
  Description of Artificial Sequence Synthetic polynucleotide
uccuguccaggaacccggcggucccaaauaucucuuccaucacuucgaacuccuggcggggcucagccacgcugguuacguccucaccuuucagucgaca

SEQ ID NO: 517 - RNA, 100 nt, Artificial Sequence
  Description of Artificial Sequence Synthetic polynucleotide
uccuguccaggaacccggcggucccaaauaucucuuccaucacuucgaacuccuggcggggcucagccacgcugguuacguccucaccugauagucgaca

SEQ ID NO: 518 - RNA, 100 nt, Artificial Sequence
  Description of Artificial Sequence Synthetic polynucleotide
uccugucgggcaacccucucguccccugaucaucuuccaucacuucgaacuccuggcggggcucagccacgcugguuaagcucucaccucugagucgaca

SEQ ID NO: 519 - RNA, 100 nt, Artificial Sequence
  Description of Artificial Sequence Synthetic polynucleotide
uccugucgggcaacccucucguccccugaucaucuuccaucacuucgaacuccuggcggggcucagccacgcugguuaagcucucaccugauagucgaca

SEQ ID NO: 520 - RNA, 100 nt, Artificial Sequence
  Description of Artificial Sequence Synthetic polynucleotide
uccugucaaacaaccccaucgucccagcgugaucuuccaucacuucgaacuccuggcggggcucagccacgcugguugccaccucaccuccgagucgaca

SEQ ID NO: 521 - RNA, 100 nt, Artificial Sequence
  Description of Artificial Sequence Synthetic polynucleotide
uccugucaaacaaccccaucgucccagcgugaucuuccaucacuucgaacuccuggcggggcucagccacgcugguugccaccucaccugauagucgaca

SEQ ID NO: 522 - RNA, 100 nt, Artificial Sequence
  Description of Artificial Sequence Synthetic polynucleotide
uccuguccacaaacccgaaagucccacauugaucuggaaucacuccuucauccuggcggggcucagccacgcugguuccgcccgcaccugcgugucgaca

SEQ ID NO: 523 - RNA, 100 nt, Artificial Sequence
  Description of Artificial Sequence Synthetic polynucleotide
uccuguccacaaacccgaaagucccacauugaucuggaaucacuccuucauccuggcggggcucagccacgcugguuccgcccgcaccugauagucgaca

SEQ ID NO: 524 - RNA, 100 nt, Artificial Sequence
  Description of Artificial Sequence Synthetic polynucleotide
uccugucgagcaacccaaccgucccuuagugaucgcgcaucacaggugucuccuggcggggcucagccacgcugguuccggacacaccugcuugucgaca

SEQ ID NO: 525 - RNA, 100 nt, Artificial Sequence
  Description of Artificial Sequence Synthetic polynucleotide
uccugucgagcaacccaaccgucccuuagugaucgcgcaucacaggugucuccuggcggggcucagccacgcugguuccggacacaccugauagucgaca

SEQ ID NO: 526 - RNA, 100 nt, Artificial Sequence
  Description of Artificial Sequence Synthetic polynucleotide
uccugugaaccaaccacaacguccgcccgugauaaaccaucagccaucacuccuggcggggcucagccacgcugguucugaugacaccugcgcgucgaca

SEQ ID NO: 527 - RNA, 100 nt, Artificial Sequence
  Description of Artificial Sequence Synthetic polynucleotide
uccugugaaccaaccacaacguccgcccgugauaaaccaucagccaucacuccuggcggggcucagccacgcugguucugaugacaccugauagucgaca

SEQ ID NO: 528 - RNA, 100 nt, Artificial Sequence
  Description of Artificial Sequence Synthetic polynucleotide
uccugcaacccaacgaauacgucaacgcgugacgguccaucuccguaaacuccuggcggggcucagccacgcugguucuuuuuccaccugugcgucgaca

SEQ ID NO: 529 - RNA, 100 nt, Artificial Sequence
  Description of Artificial Sequence Synthetic polynucleotide
uccugcaacccaacgaauacgucaacgcgugacgguccaucuccguaaacuccuggcggggcucagccacgcugguucuuuuuccaccugauagucgaca

SEQ ID NO: 530 - RNA, 100 nt, Artificial Sequence
  Description of Artificial Sequence Synthetic polynucleotide
uccaagcccccaugacguacggcacagcgucuacuuccagguguccgaacuccuggcggggcucagccacgcugguucuauaugcaccugccugucgaca

SEQ ID NO: 531 - RNA, 100 nt, Artificial Sequence
  Description of Artificial Sequence Synthetic polynucleotide
uccaagcccccaugacguacggcacagcgucuacuuccagguguccgaacuccuggcggggcucagccacgcugguucuauaugcaccugauagucgaca

SEQ ID NO: 532 - RNA, 100 nt, Artificial Sequence
  Description of Artificial Sequence Synthetic polynucleotide
ucaauucccccucaccguacaggccagcgcauucuuccugcgggucgaacuccuggcggggcucagccacgcugguucccgucacaccugcucgucgaca

SEQ ID NO: 533 - RNA, 100 nt, Artificial Sequence
  Description of Artificial Sequence Synthetic polynucleotide
ucaauucccccucaccguacaggccagcgcauucuuccugcgggucgaacuccuggcggggcucagccacgcugguucccgucacaccugauagucgaca

SEQ ID NO: 534 - RNA, 100 nt, Artificial Sequence
  Description of Artificial Sequence Synthetic polynucleotide
uccugucccgauacccguuauucccaggaagaucuugagccucuucgaacuccuggcggggcucagccacgcugguucuccucgcaccugugcgucgaca

SEQ ID NO: 535 - RNA, 100 nt, Artificial Sequence
  Description of Artificial Sequence Synthetic polynucleotide
uccugucccgauacccguuauucccaggaagaucuugagccucuucgaacuccuggcggggcucagccacgcugguucuccucgcaccugauagucgaca

SEQ ID NO: 536 - RNA, 100 nt, Artificial Sequence
  Description of Artificial Sequence Synthetic polynucleotide
uccuguccaaaaacccgccggucccaaauugaucuaaggcgacuucgaacuccuggcggggcucagccacgcugguuccugccgcaccugcaugucgaca

SEQ ID NO: 537 - RNA, 100 nt, Artificial Sequence
  Description of Artificial Sequence Synthetic polynucleotide
uccuguccaaaaacccgccggucccaaauugaucuaaggcgacuucgaacuccuggcggggcucagccacgcugguuccugccgcaccugauagucgaca

SEQ ID NO: 538 - RNA, 100 nt, Artificial Sequence
  Description of Artificial Sequence Synthetic polynucleotide
uccugucaggcaaccccgccgucccccggugaucgaggaacacuucgaacuccuggcggggcucagccacgcugguuccuucggcaccuguccgucgaca

SEQ ID NO: 539 - RNA, 100 nt, Artificial Sequence
  Description of Artificial Sequence Synthetic polynucleotide
uccugucaggcaaccccgccgucccccggugaucgaggaacacuucgaacuccuggcggggcucagccacgcugguuccuucggcaccugauagucgaca

SEQ ID NO: 540 - RNA, 100 nt, Artificial Sequence
  Description of Artificial Sequence Synthetic polynucleotide
uccugugaaccaaccaucacguccaaccgugauacuaauucacuucgaacuccuggcggggcucagccacgcugguucccuugccaccugugcgucgaca

SEQ ID NO: 541 - RNA, 100 nt, Artificial Sequence
  Description of Artificial Sequence Synthetic polynucleotide
uccugugaaccaaccaucacguccaaccgugauacuaauucacuucgaacuccuggcggggcucagccacgcugguucccuugccaccugauagucgaca

SEQ ID NO: 542 - RNA, 100 nt, Artificial Sequence
  Description of Artificial Sequence Synthetic polynucleotide
uccuggaacccaacaauuacgucggugcgugagccaagaucacuucgaacuccuggcggggcucagccacgcugguucccacggcaccugcuugucgaca

SEQ ID NO: 543 - RNA, 100 nt, Artificial Sequence
  Description of Artificial Sequence Synthetic polynucleotide
uccuggaacccaacaauuacgucggugcgugagccaagaucacuucgaacuccuggcggggcucagccacgcugguucccacggcaccugauagucgaca

SEQ ID NO: 544 - RNA, 100 nt, Artificial Sequence
  Description of Artificial Sequence Synthetic polynucleotide
uccuuagccccaaaaaguacgugaaagcguguagaagcaucacuucgaacuccuggcggggcucagccacgcugguucuuucaacaccugauagucgaca

SEQ ID NO: 545 - RNA, 100 nt, Artificial Sequence
  Description of Artificial Sequence Synthetic polynucleotide
uccgggcccccaucacguacggcacagcguuccacaccaucacuucgaacuccuggcggggcucagccacgcugguucucuuugcaccugcaugucgaca

SEQ ID NO: 546 - RNA, 100 nt, Artificial Sequence
  Description of Artificial Sequence Synthetic polynucleotide
uccgggcccccaucacguacggcacagcguuccacaccaucacuucgaacuccuggcggggcucagccacgcugguucucuuugcaccugauagucgaca

SEQ ID NO: 547 - RNA, 100 nt, Artificial Sequence
  Description of Artificial Sequence Synthetic polynucleotide
uccugucccgacacccgucucucccagagcucacuuccaucacuucgaacuccuggcggggcucagccacgcugguuccccucacaccuguaugucgaca

SEQ ID NO: 548 - RNA, 100 nt, Artificial Sequence
  Description of Artificial Sequence Synthetic polynucleotide
uccugucccgacacccgucucucccagagcucacuuccaucacuucgaacuccuggcggggcucagccacgcugguuccccucacaccugauagucgaca

SEQ ID NO: 549 - RNA, 100 nt, Artificial Sequence
  Description of Artificial Sequence Synthetic polynucleotide
uccuguccaagaacccgauggucccauaagauucuuccaucacuucgaacuccuggcggggcucagccacgcugguuccucaagcaccugcgcgucgaca

SEQ ID NO: 550 - RNA, 100 nt, Artificial Sequence
  Description of Artificial Sequence Synthetic polynucleotide
uccuguccaagaacccgauggucccauaagauucuuccaucacuucgaacuccuggcggggcucagccacgcugguuccucaagcaccugauagucgaca

SEQ ID NO: 551 - RNA, 100 nt, Artificial Sequence
  Description of Artificial Sequence Synthetic polynucleotide
uccugucaaacaacccacccguccccuuauuaucuuccaucacuucgaacuccuggcggggcucagccacgcugguucucgcggcaccugcacgucgaca

SEQ ID NO: 552 - RNA, 100 nt, Artificial Sequence
  Description of Artificial Sequence Synthetic polynucleotide
uccugucaaacaacccacccguccccuuauuaucuuccaucacuucgaacuccuggcggggcucagccacgcugguucucgcggcaccugauagucgaca

SEQ ID NO: 553 - RNA, 100 nt, Artificial Sequence
  Description of Artificial Sequence Synthetic polynucleotide
uccugucgggcaaccccaucgucccagcgugaucuuccaucacuucgaacuccuggcggggcucagccacgcugguucucuagccaccuguucgucgaca

SEQ ID NO: 554 - RNA, 100 nt, Artificial Sequence
  Description of Artificial Sequence Synthetic polynucleotide
uccugucgggcaaccccaucgucccagcgugaucuuccaucacuucgaacuccuggcggggcucagccacgcugguucucuagccaccugauagucgaca

SEQ ID NO: 555 - RNA, 100 nt, Artificial Sequence
  Description of Artificial Sequence Synthetic polynucleotide
uccuguccagaaacccgcuagucccacaaugaucucggaucacucccauguccuggcggggcucagccacgcugguucaaaguucaccugauagucgaca

SEQ ID NO: 556 - RNA, 100 nt, Artificial Sequence
  Description of Artificial Sequence Synthetic polynucleotide
uccugucgagcaacccauucguccccaagugaucgcgcaucacacuagccuccuggcggggcucagccacgcuggggaaaaguuguacugauaagggaca

SEQ ID NO: 557 - RNA, 100 nt, Artificial Sequence
  Description of Artificial Sequence Synthetic polynucleotide
uccugucgagcaacccauucguccccaagugaucgcgcaucacacuagccuccuggcggggcucagccacgcugguucaaaguucaccugauagucgaca

SEQ ID NO: 558 - RNA, 100 nt, Artificial Sequence
  Description of Artificial Sequence Synthetic polynucleotide
uccugugggccaaccgcaacguccaaucgugauggaccaucaaucaccacuccuggcggggcucagccacgcuggccaaaaguugcgcugauaacagaca

SEQ ID NO: 559 - RNA, 100 nt, Artificial Sequence
  Description of Artificial Sequence Synthetic polynucleotide
uccugugggccaaccgcaacguccaaucgugauggaccaucaaucaccacuccuggcggggcucagccacgcugguucaaaguucaccugauagucgaca

SEQ ID NO: 560 - RNA, 100 nt, Artificial Sequence
  Description of Artificial Sequence Synthetic polynucleotide
uccuggaacccaacaacuacgucagugcgugaguauccauccauauuaacuccuggcggggcucagccacgcugggaaaaaguugcacugauaugagaca

SEQ ID NO: 561 - RNA, 100 nt, Artificial Sequence
  Description of Artificial Sequence Synthetic polynucleotide
uccuggaacccaacaacuacgucagugcgugaguauccauccauauuaacuccuggcggggcucagccacgcugguucaaaguucaccugauagucgaca

SEQ ID NO: 562 - RNA, 100 nt, Artificial Sequence
  Description of Artificial Sequence Synthetic polynucleotide
ucaccucccccccgccguacaggccagcggucucuuccuuguugucgaacuccuggcggggcucagccacgcuggcaaaaaguugcacugauaaaggaca

SEQ ID NO: 563 - RNA, 100 nt, Artificial Sequence
  Description of Artificial Sequence Synthetic polynucleotide
ucaccucccccccgccguacaggccagcggucucuuccuuguugucgaacuccuggcggggcucagccacgcugguucaaaguucaccugauagucgaca

SEQ ID NO: 564 - RNA, 100 nt, Artificial Sequence
  Description of Artificial Sequence Synthetic polynucleotide
uccugucccaguacccguuucucccagaucgaucuugaagaccuucgaacuccuggcggggcucagccacgcuggcagaaaguuguacugauaugggaca

SEQ ID NO: 565 - RNA, 100 nt, Artificial Sequence
  Description of Artificial Sequence Synthetic polynucleotide
uccuguccgagaacccgguggucccauacugaucuggugcaacuucgaacuccuggcggggcucagccacgcugggggaaaguuaaacugauacaagaca

SEQ ID NO: 566 - RNA, 100 nt, Artificial Sequence
  Description of Artificial Sequence Synthetic polynucleotide
uccuguccgagaacccgguggucccauacugaucuggugcaacuucgaacuccuggcggggcucagccacgcugguucaaaguucaccugauagucgaca

SEQ ID NO: 567 - RNA, 100 nt, Artificial Sequence
  Description of Artificial Sequence Synthetic polynucleotide
uccugucggacaacccuuccgucccccagugaucgauuuccacuucgaacuccuggcggggcucagccacgcuggcuaaaaguugcgcugauauaggaca

SEQ ID NO: 568 - RNA, 100 nt, Artificial Sequence
  Description of Artificial Sequence Synthetic polynucleotide
uccuguggaccaaccaaaacguccaaucgugauaggguuucacuucgaacuccuggcggggcucagccacgcuggggaaaaguugcgcugauauuagaca

SEQ ID NO: 569 - RNA, 100 nt, Artificial Sequence
  Description of Artificial Sequence Synthetic polynucleotide
uccuguggaccaaccaaaacguccaaucgugauaggguuucacuucgaacuccuggcggggcucagccacgcugguucaaaguucaccugauagucgaca

SEQ ID NO: 570 - RNA, 100 nt, Artificial Sequence
  Description of Artificial Sequence Synthetic polynucleotide
uccugcggcccaacaaauacgucacugcgugaauaaggaucacuucgaacuccuggcggggcucagccacgcuggaggaaaguugcacugauauaggaca

SEQ ID NO: 571 - RNA, 100 nt, Artificial Sequence
  Description of Artificial Sequence Synthetic polynucleotide
uccugcggcccaacaaauacgucacugcgugaauaaggaucacuucgaacuccuggcggggcucagccacgcugguucaaaguucaccugauagucgaca

SEQ ID NO: 572 - RNA, 100 nt, Artificial Sequence
  Description of Artificial Sequence Synthetic polynucleotide
uccuaggccccaagaaguacgugagagcgugcggccacaucacuucgaacuccuggcggggcucagccacgcugggcgaaaguugugcugauaagggaca

SEQ ID NO: 573 - RNA, 100 nt, Artificial Sequence
  Description of Artificial Sequence Synthetic polynucleotide
uccuaggccccaagaaguacgugagagcgugcggccacaucacuucgaacuccuggcggggcucagccacgcugguucaaaguucaccugauagucgaca

SEQ ID NO: 574 - RNA, 100 nt, Artificial Sequence
  Description of Artificial Sequence Synthetic polynucleotide
uccgaccccccauggcguacgacacagcgucucauaccaucacuucgaacuccuggcggggcucagccacgcugggaaaaaguuguacugauacgggaca

SEQ ID NO: 575 - RNA, 100 nt, Artificial Sequence
  Description of Artificial Sequence Synthetic polynucleotide
uccgaccccccauggcguacgacacagcgucucauaccaucacuucgaacuccuggcggggcucagccacgcugguucaaaguucaccugauagucgaca

SEQ ID NO: 576 - RNA, 100 nt, Artificial Sequence
  Description of Artificial Sequence Synthetic polynucleotide
uccugucccaacacccguuaaucccagauacuccuuccaucacuucgaacuccuggcggggcucagccacgcuggaagaaaguugugcugauaagggaca

SEQ ID NO: 577 - RNA, 100 nt, Artificial Sequence
  Description of Artificial Sequence Synthetic polynucleotide
uccuguccggaaacccgccagucccaagaccuucuuccaucacuucgaacuccuggcggggcucagccacgcuggauaaaaguuaugcugauaauagaca

SEQ ID NO: 578 - RNA, 100 nt, Artificial Sequence
  Description of Artificial Sequence Synthetic polynucleotide
uccugucaggcaacccccucgucccuaucuuaucuuccaucacuucgaacuccuggcggggcucagccacgcuggccgaaaguugugcugauacgggaca

SEQ ID NO: 579 - RNA, 100 nt, Artificial Sequence
  Description of Artificial Sequence Synthetic polynucleotide
uccugucaggcaacccccucgucccuaucuuaucuuccaucacuucgaacuccuggcggggcucagccacgcugguucaaaguucaccugauagucgaca

SEQ ID NO: 580 - RNA, 100 nt, Artificial Sequence
  Description of Artificial Sequence Synthetic polynucleotide
ucacauccccccugccguaccuaccagcgauuucuuccccgggaucgaaguccuggugggacucagccacgcuguuucaaaguucaccuaucgcccgaca

SEQ ID NO: 581 - RNA, 100 nt, Artificial Sequence
  Description of Artificial Sequence Synthetic polynucleotide
uccugucgagcaacccagccgucccagcgugaucuuccaucacuucgaaguccuggugggacucagccacgcugugagaaaguugugcugauauaagaca

SEQ ID NO: 582 - RNA, 100 nt, Artificial Sequence
  Description of Artificial Sequence Synthetic polynucleotide
uccugucgagcaacccagccgucccagcgugaucuuccaucacuucgaaguccuggugggacucagccacgcuguuucaaaguucaccugauagucgaca

SEQ ID NO: 583 - RNA, 100 nt, Artificial Sequence
  Description of Artificial Sequence Synthetic polynucleotide
uccugucccccaacccguacgucccagcgugaucuucccaauggucgaacuucuggcgagguucagccacgcuuguucaaaguucaccuaccucgcgaca

SEQ ID NO: 584 - RNA, 100 nt, Artificial Sequence
  Description of Artificial Sequence Synthetic polynucleotide
ucagaucccccccgccguacuggccagcgcccucuucccaauggucgaacuucuggcgagguucagccacgcuuguucaaaguucaccuaccucgcgaca

SEQ ID NO: 585 - RNA, 100 nt, Artificial Sequence
  Description of Artificial Sequence Synthetic polynucleotide
uccugucccccaacccguacgucccagcguguuucaacaucacuucgaacuucuggcgagguucagccacgcuuguuccucgcacaccugauagucgaca

SEQ ID NO: 586 - RNA, 100 nt, Artificial Sequence
  Description of Artificial Sequence Synthetic polynucleotide
uccuaggccccaagggguacgugagagcguguuucaacaucacuucgaacuucuggcgagguucagccacgcuuguuccucgcacaccugcaugucgaca

SEQ ID NO: 587 - RNA, 100 nt, Artificial Sequence
  Description of Artificial Sequence Synthetic polynucleotide
uccuaggccccaagggguacgugagagcguguuucaacaucacuucgaacuucuggcgagguucagccacgcuuguuccucgcacaccugauagucgaca

SEQ ID NO: 588 - RNA, 100 nt, Artificial Sequence
  Description of Artificial Sequence Synthetic polynucleotide
uccuuaaccccaagggguacguggaagcgugaucuuccaucacuucgaacuucuggcgagguucagccacgcuuguucaaaguucaccugauagucgaca

SEQ ID NO: 589 - RNA, 100 nt, Artificial Sequence
  Description of Artificial Sequence Synthetic polynucleotide
ucaacucccccucgccguacacaccagcgcucucuucccuguggucgaacuccuggcggugcuuagccacgcugauucaauguucaccuucuuugcgaca

SEQ ID NO: 590 - RNA, 100 nt, Artificial Sequence
  Description of Artificial Sequence Synthetic polynucleotide
uccugucacacaaccccuccgucccucagugauccguaaccacuucgaacuccuggcggugcuuagccacgcugaaggaucguucacgacauagucgaca

SEQ ID NO: 591 - RNA, 100 nt, Artificial Sequence
  Description of Artificial Sequence Synthetic polynucleotide
uccugucacacaaccccuccgucccucagugauccguaaccacuucgaacuccuggcggugcuuagccacgcugaaggaucguucaccugauagucgaca

SEQ ID NO: 592 - RNA, 100 nt, Artificial Sequence
  Description of Artificial Sequence Synthetic polynucleotide
uccuaaaccccaagaaguacguaggagcguguuucaacaucacuucgaacuccuggcggugcuuagccacgcugauuccauacacaccuguuugucgaca

SEQ ID NO: 593 - RNA, 100 nt, Artificial Sequence
  Description of Artificial Sequence Synthetic polynucleotide
uccuaaaccccaagaaguacguaggagcguguuucaacaucacuucgaacuccuggcggugcuuagccacgcugauuccauacacaccugauagucgaca

SEQ ID NO: 594 - RNA, 100 nt, Artificial Sequence
  Description of Artificial Sequence Synthetic polynucleotide
uccugucccccaacccguacgucccagcgugaucuuccaucacuucgaacuccuggcggugcuuagccacgcugauucaauguucaccugauagucgaca

SEQ ID NO: 595 - RNA, 100 nt, Artificial Sequence
  Description of Artificial Sequence Synthetic polynucleotide
uccuuagccccaaacaguacgugaaagcgugaucuuccaucacuucgaacuccuggcggugcuuagccacgcugaauaaauguuaugcugauaagggaca

SEQ ID NO: 596 - RNA, 100 nt, Artificial Sequence
  Description of Artificial Sequence Synthetic polynucleotide
uccuuagccccaaacaguacgugaaagcgugaucuuccaucacuucgaacuccuggcggugcuuagccacgcugauucaauguucaccugauagucgaca

SEQ ID NO: 597 - RNA, 100 nt, Artificial Sequence
  Description of Artificial Sequence Synthetic polynucleotide
ucgauuccccccuaccguaccaaccagcgaucucuucccguaggucgaacuccugggggggcucagccacgcuguuucaaaguucaccuucauaccgaca

SEQ ID NO: 598 - RNA, 100 nt, Artificial Sequence
  Description of Artificial Sequence Synthetic polynucleotide
uccugucgaacaacccuaucgucccuaagugaucaguggacacuucgaacuccugggggggcucagccacgcugugaggcuguucaccugauagucgaca

SEQ ID NO: 599 - RNA, 100 nt, Artificial Sequence
  Description of Artificial Sequence Synthetic polynucleotide
uccuccgccccaaaggguacguggaagcguguuucaacaucacuucgaacuccugggggggcucagccacgcuguuucucuagacaccugcgcgucgaca

SEQ ID NO: 600 - RNA, 100 nt, Artificial Sequence
  Description of Artificial Sequence Synthetic polynucleotide
uccuccgccccaaaggguacguggaagcguguuucaacaucacuucgaacuccugggggggcucagccacgcuguuucucuagacaccugauagucgaca

SEQ ID NO: 601 - RNA, 100 nt, Artificial Sequence
  Description of Artificial Sequence Synthetic polynucleotide
uccuacgccccaagagguacguaaaagcgugaucuuccaucacuucgaacuccugggggggcucagccacgcugugcaaaaguuaugcugauaacagaca

SEQ ID NO: 602 - RNA, 100 nt, Artificial Sequence
  Description of Artificial Sequence Synthetic polynucleotide
uccuacgccccaagagguacguaaaagcgugaucuuccaucacuucgaacuccugggggggcucagccacgcuguuucaaaguucaccugauagucgaca

SEQ ID NO: 603 - RNA, 100 nt, Artificial Sequence
  Description of Artificial Sequence Synthetic polynucleotide
ucaguucccccuuaccguacacgccagcgccuucuuccuaugaaucgaacuccuggcggggcucauccacgcuguaucaaaguucacuuagcgcccgaca

SEQ ID NO: 604 - RNA, 100 nt, Artificial Sequence
  Description of Artificial Sequence Synthetic polynucleotide
uccugucagacaacccauucguccccuggugaucaauuggcacuucgaacuccuggcggggcucauccacgcuguaugcucguucacaauauagucgaca

SEQ ID NO: 605 - RNA, 100 nt, Artificial Sequence
  Description of Artificial Sequence Synthetic polynucleotide
uccugucagacaacccauucguccccuggugaucaauuggcacuucgaacuccuggcggggcucauccacgcuguaugcucguucaccugauagucgaca

SEQ ID NO: 606 - RNA, 100 nt, Artificial Sequence
  Description of Artificial Sequence Synthetic polynucleotide
uccucuaccccaagggguacguaggagcguguuucaacaucacuucgaacuccuggcggggcucauccacgcuguauccuaccccaccuguuugucgaca

SEQ ID NO: 607 - RNA, 100 nt, Artificial Sequence
  Description of Artificial Sequence Synthetic polynucleotide
uccucuaccccaagggguacguaggagcguguuucaacaucacuucgaacuccuggcggggcucauccacgcuguauccuaccccaccugauagucgaca

SEQ ID NO: 608 - RNA, 100 nt, Artificial Sequence
  Description of Artificial Sequence Synthetic polynucleotide
uccucagccccaaggaguacguaagagcgugaucuuccaucacuucgaacuccuggcggggcucauccacgcuguaggaaaguugcguugauaugggaca

SEQ ID NO: 609 - RNA, 100 nt, Artificial Sequence
  Description of Artificial Sequence Synthetic polynucleotide
uccucagccccaaggaguacguaagagcgugaucuuccaucacuucgaacuccuggcggggcucauccacgcuguaucaaaguucacuugauagucgaca

SEQ ID NO: 610 - RNA, 100 nt, Artificial Sequence
  Description of Artificial Sequence Synthetic polynucleotide
uccugucccccaacccguacgucccagcgugaucuucccaauugucgaauuucuggcgggccucagccacgcugauucaaaguucaccuaagccacgaca

SEQ ID NO: 611 - RNA, 100 nt, Artificial Sequence
  Description of Artificial Sequence Synthetic polynucleotide
ucagaucccccuuaccguacaggccagcgcccucuucccaauugucgaauuucuggcgggccucagccacgcugauucaaaguucaccuaagccacgaca

SEQ ID NO: 612 - RNA, 100 nt, Artificial Sequence
  Description of Artificial Sequence Synthetic polynucleotide
uccugucgagcaacccuuucgucccuuggugauccaauuacacuucgaauuucuggcgggccucagccacgcugaaagcucguucacggaauagucgaca

SEQ ID NO: 613 - RNA, 100 nt, Artificial Sequence
  Description of Artificial Sequence Synthetic polynucleotide
uccugucgagcaacccuuucgucccuuggugauccaauuacacuucgaauuucuggcgggccucagccacgcugaaagcucguucaccugauagucgaca

SEQ ID NO: 614 - RNA, 100 nt, Artificial Sequence
  Description of Artificial Sequence Synthetic polynucleotide
uccucggccccaagggguacgugggagcguguuucaacaucacuucgaauuucuggcgggccucagccacgcugauucuauauccaccugcucgucgaca

SEQ ID NO: 615 - RNA, 100 nt, Artificial Sequence
  Description of Artificial Sequence Synthetic polynucleotide
uccucggccccaagggguacgugggagcguguuucaacaucacuucgaauuucuggcgggccucagccacgcugauucuauauccaccugauagucgaca

SEQ ID NO: 616 - RNA, 100 nt, Artificial Sequence
  Description of Artificial Sequence Synthetic polynucleotide
uccuuagccccaagaaguacguaagagcgugaucuuccaucacuucgaauuucuggcgggccucagccacgcugauagaaaguugcgcugauacgggaca

SEQ ID NO: 617 - RNA, 100 nt, Artificial Sequence
  Description of Artificial Sequence Synthetic polynucleotide
uccuuagccccaagaaguacguaagagcgugaucuuccaucacuucgaauuucuggcgggccucagccacgcugauucaaaguucaccugauagucgaca

SEQ ID NO: 618 - RNA, 100 nt, Artificial Sequence
  Description of Artificial Sequence Synthetic polynucleotide
ucaauucccccucaccguacauaccagcgcauucuuccuaagucucgaacugcuggcggagcucagccacgcuguuucaaaguucaccuaaguugcgaca

SEQ ID NO: 619 - RNA, 100 nt, Artificial Sequence
  Description of Artificial Sequence Synthetic polynucleotide
uccugucgggcaaccccuccgucccccggugaucguaaaacacuucgaacugcuggcggagcucagccacgcuguaauguuguucacaccauagucgaca

SEQ ID NO: 620 - RNA, 100 nt, Artificial Sequence
  Description of Artificial Sequence Synthetic polynucleotide
uccugucgggcaaccccuccgucccccggugaucguaaaacacuucgaacugcuggcggagcucagccacgcuguaauguuguucaccugauagucgaca

SEQ ID NO: 621 - RNA, 100 nt, Artificial Sequence
  Description of Artificial Sequence Synthetic polynucleotide
uccuauaccccaaagaguacgugggagcguguuucaacaucacuucgaacugcuggcggagcucagccacgcuguuuccccuagcaccuguuugucgaca

SEQ ID NO: 622 - RNA, 100 nt, Artificial Sequence
  Description of Artificial Sequence Synthetic polynucleotide
uccuauaccccaaagaguacgugggagcguguuucaacaucacuucgaacugcuggcggagcucagccacgcuguuuccccuagcaccugauagucgaca

SEQ ID NO: 623 - RNA, 100 nt, Artificial Sequence
  Description of Artificial Sequence Synthetic polynucleotide
uccuacaccccaaggaguacguaggagcgugaucuuccaucacuucgaacugcuggcggagcucagccacgcuguuuaaaaguuaaacugauacaggaca

SEQ ID NO: 624 - RNA, 100 nt, Artificial Sequence
  Description of Artificial Sequence Synthetic polynucleotide
uccuacaccccaaggaguacguaggagcgugaucuuccaucacuucgaacugcuggcggagcucagccacgcuguuucaaaguucaccugauagucgaca

SEQ ID NO: 625 - RNA, 100 nt, Artificial Sequence
  Description of Artificial Sequence Synthetic polynucleotide
uccugucccccaacccguacgucccagcgugaucuuccaucacuucgaacuccuagaagggcucagccacgcugcuucaagguucaccugauagucgaca

SEQ ID NO: 626 - RNA, 100 nt, Artificial Sequence
  Description of Artificial Sequence Synthetic polynucleotide
uccuuaaccccaaaagguacguacaagcgugaucuuccaucacuucgaacuccuagaagggcucagccacgcugcuuaaagguuauacugauaagggaca

SEQ ID NO: 627 - RNA, 100 nt, Artificial Sequence
  Description of Artificial Sequence Synthetic polynucleotide
uccuuaaccccaaaagguacguacaagcgugaucuuccaucacuucgaacuccuagaagggcucagccacgcugcuucaagguucaccugauagucgaca

SEQ ID NO: 628 - RNA, 100 nt, Artificial Sequence
  Description of Artificial Sequence Synthetic polynucleotide
ucaauucccccccgccguacaggccagcgaauucuuccuacgucucgaauuccuggcggaacuuagccacgcugcuucaaaguucaccuaagcugcgaca

SEQ ID NO: 629 - RNA, 100 nt, Artificial Sequence
  Description of Artificial Sequence Synthetic polynucleotide
uccugucgagcaacccauccgucccuuagugauccagaaacacuucgaauuccuggcggaacuuagccacgcugcagaccuguucacaaaauagucgaca

SEQ ID NO: 630 - RNA, 100 nt, Artificial Sequence
  Description of Artificial Sequence Synthetic polynucleotide
uccugucgagcaacccauccgucccuuagugauccagaaacacuucgaauuccuggcggaacuuagccacgcugcagaccuguucaccugauagucgaca

SEQ ID NO: 631 - RNA, 100 nt, Artificial Sequence
  Description of Artificial Sequence Synthetic polynucleotide
uccugucccccaacccguacgucccagcguguuucaacaucacuucgaauuccuggcggaacuuagccacgcugcuucugccugcaccugauagucgaca

SEQ ID NO: 632 - RNA, 100 nt, Artificial Sequence
  Description of Artificial Sequence Synthetic polynucleotide
uccuuagccccaagggguacgugagagcguguuucaacaucacuucgaauuccuggcggaacuuagccacgcugcuucugccugcaccugauagucgaca

SEQ ID NO: 633 - RNA, 100 nt, Artificial Sequence
  Description of Artificial Sequence Synthetic polynucleotide
uccuccaccccaagaaguacguaagagcgugaucuuccaucacuucgaauuccuggcggaacuuagccacgcugcaggaaaguugcgcugauaugggaca

SEQ ID NO: 634 - RNA, 100 nt, Artificial Sequence
  Description of Artificial Sequence Synthetic polynucleotide
uccuccaccccaagaaguacguaagagcgugaucuuccaucacuucgaauuccuggcggaacuuagccacgcugcuucaaaguucaccugauagucgaca

SEQ ID NO: 635 - RNA, 100 nt, Artificial Sequence
  Description of Artificial Sequence Synthetic polynucleotide
uccugucccccaacccguacgucccagcgugaucuuccugccggucgaacuccuggcggagcuuagccacgcuguuucaaaguucaccuuuguaccgaca

SEQ ID NO: 636 - RNA, 100 nt, Artificial Sequence
  Description of Artificial Sequence Synthetic polynucleotide
ucaccucccccuaaccguacccgccagcgcccucuuccugccggucgaacuccuggcggagcuuagccacgcuguuucaaaguucaccuuuguaccgaca

SEQ ID NO: 637 - RNA, 100 nt, Artificial Sequence
  Description of Artificial Sequence Synthetic polynucleotide
uccugucacacaacccccucgucccucggugaucgcauggcacuucgaacuccuggcggagcuuagccacgcuguugucccguucacgcaauagucgaca

SEQ ID NO: 638 - RNA, 100 nt, Artificial Sequence
  Description of Artificial Sequence Synthetic polynucleotide
uccugucacacaacccccucgucccucggugaucgcauggcacuucgaacuccuggcggagcuuagccacgcuguugucccguucaccugauagucgaca

SEQ ID NO: 639 - RNA, 100 nt, Artificial Sequence
  Description of Artificial Sequence Synthetic polynucleotide
uccuugaccccaaggaguacgugggagcguguuucaacaucacuucgaacuccuggcggagcuuagccacgcuguuucuucuggcaccuguuugucgaca

SEQ ID NO: 640 - RNA, 100 nt, Artificial Sequence
  Description of Artificial Sequence Synthetic polynucleotide
uccuugaccccaaggaguacgugggagcguguuucaacaucacuucgaacuccuggcggagcuuagccacgcuguuucuucuggcaccugauagucgaca

SEQ ID NO: 641 - RNA, 100 nt, Artificial Sequence
  Description of Artificial Sequence Synthetic polynucleotide
uccuuaaccccaaaaaguacguaggagcgugaucuuccaucacuucgaacuccuggcggagcuuagccacgcuguuagaaaguugcacugauaugggaca

SEQ ID NO: 642 - RNA, 100 nt, Artificial Sequence
  Description of Artificial Sequence Synthetic polynucleotide
uccuuaaccccaaaaaguacguaggagcgugaucuuccaucacuucgaacuccuggcggagcuuagccacgcuguuucaaaguucaccugauagucgaca

SEQ ID NO: 643 - RNA, 100 nt, Artificial Sequence
  Description of Artificial Sequence Synthetic polynucleotide
uccuacgccccaagggguacguaaaagcguguuucaacaucacuucgaacuccuggcggugcuaagccacgcuguuuccgccggcaccugugugucgaca

SEQ ID NO: 644 - RNA, 100 nt, Artificial Sequence
  Description of Artificial Sequence Synthetic polynucleotide
uccuacgccccaagggguacguaaaagcguguuucaacaucacuucgaacuccuggcggugcuaagccacgcuguuuccgccggcaccugauagucgaca

SEQ ID NO: 645 - RNA, 100 nt, Artificial Sequence
  Description of Artificial Sequence Synthetic polynucleotide
uccuccgccccaagagguacguacaagcgugaucuuccaucacuucgaacuccuggcggugcuaagccacgcuguuaaaaaguugcgcugauauuagaca

SEQ ID NO: 646 - RNA, 100 nt, Artificial Sequence
  Description of Artificial Sequence Synthetic polynucleotide
uccuccgccccaagagguacguacaagcgugaucuuccaucacuucgaacuccuggcggugcuaagccacgcuguuucaaaguucaccugauagucgaca

SEQ ID NO: 647 - RNA, 100 nt, Artificial Sequence
  Description of Artificial Sequence Synthetic polynucleotide
ucgacucccccccgccguacuagccagcgaccucuuccuguucgucgaaguccuggcgggguucagccacgcuuguucaaaguucaccuuucaaacgaca

SEQ ID NO: 648 - RNA, 100 nt, Artificial Sequence
  Description of Artificial Sequence Synthetic polynucleotide
uccuagaccccaagagguacgugggagcguguuucaacaucacuucgaaguccuggcgggguucagccacgcuuguucuauccacaccugcuugucgaca

SEQ ID NO: 649 - RNA, 100 nt, Artificial Sequence
  Description of Artificial Sequence Synthetic polynucleotide
uccuagaccccaagagguacgugggagcguguuucaacaucacuucgaaguccuggcgggguucagccacgcuuguucuauccacaccugauagucgaca

SEQ ID NO: 650 - RNA, 100 nt, Artificial Sequence
  Description of Artificial Sequence Synthetic polynucleotide
uccucagccccaaggaguacgugagagcgugaucuuccaucacuucgaaguccuggcgggguucagccacgcuuguucaaaguucaccugauagucgaca

SEQ ID NO: 651 - RNA, 100 nt, Artificial Sequence
  Description of Artificial Sequence Synthetic polynucleotide
ucgcaucccccuaaccguaccuaccagcgcuuucuuccuaccccucgaauuccuggcgcgguucagccacgcugauucaaaguucaccuuucacacgaca

SEQ ID NO: 652 - RNA, 100 nt, Artificial Sequence
  Description of Artificial Sequence Synthetic polynucleotide
uccugucgggcaacccagccguccccuagugaucauauuccacuucgaauuccuggcgcgguucagccacgcugaaggacuguucacgaaauagucgaca

SEQ ID NO: 653 - RNA, 100 nt, Artificial Sequence
  Description of Artificial Sequence Synthetic polynucleotide
uccugucgggcaacccagccguccccuagugaucauauuccacuucgaauuccuggcgcgguucagccacgcugaaggacuguucaccugauagucgaca

SEQ ID NO: 654 - RNA, 100 nt, Artificial Sequence
  Description of Artificial Sequence Synthetic polynucleotide
uccucggccccaaaaaguacguaagagcguguuucaacaucacuucgaauuccuggcgcgguucagccacgcugauucucuaagcaccuguucgucgaca

SEQ ID NO: 655 - RNA, 100 nt, Artificial Sequence
  Description of Artificial Sequence Synthetic polynucleotide
uccucggccccaaaaaguacguaagagcguguuucaacaucacuucgaauuccuggcgcgguucagccacgcugauucucuaagcaccugauagucgaca

SEQ ID NO: 656 - RNA, 100 nt, Artificial Sequence
  Description of Artificial Sequence Synthetic polynucleotide
uccucagccccaaagaguacguagaagcgugaucuuccaucacuucgaauuccuggcgcgguucagccacgcugaacgaaaguuauacugauaauagaca

SEQ ID NO: 657 - RNA, 100 nt, Artificial Sequence
  Description of Artificial Sequence Synthetic polynucleotide
uccucagccccaaagaguacguagaagcgugaucuuccaucacuucgaauuccuggcgcgguucagccacgcugauucaaaguucaccugauagucgaca

SEQ ID NO: 658 - RNA, 100 nt, Artificial Sequence
  Description of Artificial Sequence Synthetic polynucleotide
ucgguuccccccugccguacaaaccagcggucucuuccccuguaucgaacugcuggcgggguucagucacgcuguuucaaaauucaccuagcuuccgaca

SEQ ID NO: 659 - RNA, 100 nt, Artificial Sequence
  Description of Artificial Sequence Synthetic polynucleotide
uccuuggccccaagggguacguggaagcguguuucaacaucacuucgaacugcuggcgggguucagucacgcuguuuccuuucccaccugauagucgaca

SEQ ID NO: 660 - RNA, 100 nt, Artificial Sequence
  Description of Artificial Sequence Synthetic polynucleotide
uccuacaccccaagaaguacguacaagcgugaucuuccaucacuucgaacugcuggcgggguucagucacgcuguuaaaaaauugugcugauaagggaca

SEQ ID NO: 661 - RNA, 100 nt, Artificial Sequence
  Description of Artificial Sequence Synthetic polynucleotide
uccuacaccccaagaaguacguacaagcgugaucuuccaucacuucgaacugcuggcgggguucagucacgcuguuucaaaauucaccugauagucgaca

SEQ ID NO: 662 - RNA, 100 nt, Artificial Sequence
  Description of Artificial Sequence Synthetic polynucleotide
ucggaucccccccaccguacaaaccagcgcccucuuccugcgggucgaacuccuggcgggguucagccacgcuugugcaaaguucaccuucgaaacgaca

SEQ ID NO: 663 - RNA, 100 nt, Artificial Sequence
  Description of Artificial Sequence Synthetic polynucleotide
uccuacgccccaaaagguacguggaagcgugaucuuccaucacuucgaacuccuggcgggguucagccacgcuugugcaaaguucaccugauagucgaca

SEQ ID NO: 664 - RNA, 100 nt, Artificial Sequence
  Description of Artificial Sequence Synthetic polynucleotide
ucgaaucccccucgccguacccgccagcgccuucuuccuguggaucgaacuccuggcggggggcagucacgcugauucaaaguucaccuaccacccgaca

SEQ ID NO: 665 - RNA, 100 nt, Artificial Sequence
  Description of Artificial Sequence Synthetic polynucleotide
uccuacgccccaagagguacguaagagcguguuucaacaucacuucgaacuccuggcggggggcagucacgcugauuccuacggcaccugauagucgaca

SEQ ID NO: 666 - RNA, 100 nt, Artificial Sequence
  Description of Artificial Sequence Synthetic polynucleotide
ucgauuccccccaaccguacacaccagcgaauucuucccgauucucgaaccccuggcgggccucagccacgcuguuucaaaguucaccucccccccgaca

SEQ ID NO: 667 - RNA, 100 nt, Artificial Sequence
  Description of Artificial Sequence Synthetic polynucleotide
uccucggccccaagaaguacgugaaagcgugaucuuccaucacuucgaaccccuggcgggccucagccacgcuguccgaaaguugcgcugauaagggaca

SEQ ID NO: 668 - RNA, 100 nt, Artificial Sequence
  Description of Artificial Sequence Synthetic polynucleotide
uccucggccccaagaaguacgugaaagcgugaucuuccaucacuucgaaccccuggcgggccucagccacgcuguuucaaaguucaccugauagucgaca

SEQ ID NO: 669 - RNA, 100 nt, Artificial Sequence
  Description of Artificial Sequence Synthetic polynucleotide
ucgcaucccccuaaccguacauaccagcgccuucuuccuguuggucgaacuccaggcgggguucagccacgcuuguacaaaguucaccucaaccccgaca

SEQ ID NO: 670 - RNA, 100 nt, Artificial Sequence
  Description of Artificial Sequence Synthetic polynucleotide
uccuuaaccccaaggaguacgugaaagcgugaucuuccaucacuucgaacuccaggcgggguucagccacgcuuguacaaaguucaccugauagucgaca

SEQ ID NO: 671 - RNA, 100 nt, Artificial Sequence
  Description of Artificial Sequence Synthetic polynucleotide
uccucggccccaaagaguacguaggagcgugaucuuccaucacuucgaacucgugucgggguucugccacgcugcuccaaaguucaccugauagucgaca

SEQ ID NO: 672 - RNA, 100 nt, Artificial Sequence
  Description of Artificial Sequence Synthetic polynucleotide
ucauuucccccucaccguacugaccagcgcuuucuucccugugaucgaaccccuggcguagcucagccacgcuuguccaaaguucaccuaaccaccgaca

SEQ ID NO: 673 - RNA, 100 nt, Artificial Sequence
  Description of Artificial Sequence Synthetic polynucleotide
uccuaggccccaaaggguacgugggagcguguuucaacaucacuucgaaccccuggcguagcucagccacgcuuguccucuaugcaccugauagucgaca

SEQ ID NO: 674 - RNA, 100 nt, Artificial Sequence
  Description of Artificial Sequence Synthetic polynucleotide
uccuccaccccaagagguacguggaagcgugaucuuccaucacuucgaaccccuggcguagcucagccacgcuuguccaaaguucaccugauagucgaca

SEQ ID NO: 675 - RNA, 100 nt, Artificial Sequence
  Description of Artificial Sequence Synthetic polynucleotide
uccugucccccaacccguacgucccagcgugaucuuccuacgagucgaaguccuggccggguucagccacgcugcuucaaaguucaccuugccuccgaca

SEQ ID NO: 676 - RNA, 100 nt, Artificial Sequence
  Description of Artificial Sequence Synthetic polynucleotide
ucaacucccccccaccguaccggccagcgaucucuuccuacgagucgaaguccuggccggguucagccacgcugcuucaaaguucaccuugccuccgaca

SEQ ID NO: 677 - RNA, 100 nt, Artificial Sequence
  Description of Artificial Sequence Synthetic polynucleotide
uccugucaaacaacccuuucgucccucagugaucacaggccacuucgaaguccuggccggguucagccacgcugcacuacuguucacaaaauagucgaca

SEQ ID NO: 678 - RNA, 100 nt, Artificial Sequence
  Description of Artificial Sequence Synthetic polynucleotide
uccugucaaacaacccuuucgucccucagugaucacaggccacuucgaaguccuggccggguucagccacgcugcacuacuguucaccugauagucgaca

SEQ ID NO: 679 - RNA, 100 nt, Artificial Sequence
  Description of Artificial Sequence Synthetic polynucleotide
uccugucccccaacccguacgucccagcguguuucaacaucacuucgaaguccuggccggguucagccacgcugcuuccccccgcaccugauagucgaca

SEQ ID NO: 680 - RNA, 100 nt, Artificial Sequence
  Description of Artificial Sequence Synthetic polynucleotide
uccucgaccccaaaagguacguaaaagcguguuucaacaucacuucgaaguccuggccggguucagccacgcugcuuccccccgcaccuguucgucgaca

SEQ ID NO: 681 - RNA, 100 nt, Artificial Sequence
  Description of Artificial Sequence Synthetic polynucleotide
uccucgaccccaaaagguacguaaaagcguguuucaacaucacuucgaaguccuggccggguucagccacgcugcuuccccccgcaccugauagucgaca

SEQ ID NO: 682 - RNA, 100 nt, Artificial Sequence
  Description of Artificial Sequence Synthetic polynucleotide
uccuuaaccccaaggaguacguggaagcgugaucuuccaucacuucgaaguccuggccggguucagccacgcugccuaaaaguugcgcugauauaggaca

SEQ ID NO: 683 - RNA, 100 nt, Artificial Sequence
  Description of Artificial Sequence Synthetic polynucleotide
uccuuaaccccaaggaguacguggaagcgugaucuuccaucacuucgaaguccuggccggguucagccacgcugcuucaaaguucaccugauagucgaca

SEQ ID NO: 684 - RNA, 100 nt, Artificial Sequence
  Description of Artificial Sequence Synthetic polynucleotide
uccugucaaacaacccucucguccccaagugauccauuggcacuucgaacugcuggcguggcucagcuacgcugacaguauguucacaacauagucgaca

SEQ ID NO: 685 - RNA, 100 nt, Artificial Sequence
  Description of Artificial Sequence Synthetic polynucleotide
uccugucaaacaacccucucguccccaagugauccauuggcacuucgaacugcuggcguggcucagcuacgcugacaguauguucaccugauagucgaca

SEQ ID NO: 686 - RNA, 100 nt, Artificial Sequence
  Description of Artificial Sequence Synthetic polynucleotide
uccugucccccaacccguacgucccagcguguuucaacaucacuucgaacugcuggcguggcucagcuacgcugauucuucaggcaccugauagucgaca

SEQ ID NO: 687 - RNA, 100 nt, Artificial Sequence
  Description of Artificial Sequence Synthetic polynucleotide
uccucuaccccaaggaguacguaagagcguguuucaacaucacuucgaacugcuggcguggcucagcuacgcugauucuucaggcaccuguaugucgaca

SEQ ID NO: 688 - RNA, 100 nt, Artificial Sequence
  Description of Artificial Sequence Synthetic polynucleotide
uccucuaccccaaggaguacguaagagcguguuucaacaucacuucgaacugcuggcguggcucagcuacgcugauucuucaggcaccugauagucgaca

SEQ ID NO: 689 - RNA, 100 nt, Artificial Sequence
  Description of Artificial Sequence Synthetic polynucleotide
uccuaggccccaaggaguacguaggagcgugaucuuccaucacuucgaacugcuggcguggcucagcuacgcugauuaaaaguugcgcugauauuagaca

SEQ ID NO: 690 - RNA, 100 nt, Artificial Sequence
  Description of Artificial Sequence Synthetic polynucleotide
uccuaggccccaaggaguacguaggagcgugaucuuccaucacuucgaacugcuggcguggcucagcuacgcugauucaaaguucaccugauagucgaca

SEQ ID NO: 691 - RNA, 100 nt, Artificial Sequence
  Description of Artificial Sequence Synthetic polynucleotide
uccugucacacaaccccaccguccccuagugaucuuccaucacuucgaacuccugucgggucucagccacgcuguuucaaaguucaccugauagucgaca

SEQ ID NO: 692 - RNA, 100 nt, Artificial Sequence
  Description of Artificial Sequence Synthetic polynucleotide
ucgaaucccccuugccguacuagccagcgcauucuucccggccgucgaacuccuggcggggcuuagcuacgcugcuucaaaguucaccuccagaacgaca

SEQ ID NO: 693 - RNA, 100 nt, Artificial Sequence
  Description of Artificial Sequence Synthetic polynucleotide
uccugucgagcaacccacccguccccaagugauccuagugcacuucgaacuccuggcggggcuuagcuacgcugcgccucuguucacagaauagucgaca

SEQ ID NO: 694 - RNA, 100 nt, Artificial Sequence
  Description of Artificial Sequence Synthetic polynucleotide
uccugucgagcaacccacccguccccaagugauccuagugcacuucgaacuccuggcggggcuuagcuacgcugcgccucuguucaccugauagucgaca

SEQ ID NO: 695 - RNA, 100 nt, Artificial Sequence
  Description of Artificial Sequence Synthetic polynucleotide
uccuuagccccaaagaguacguaagagcguguuucaacaucacuucgaacuccuggcggggcuuagcuacgcugcuucccucgacaccugcuugucgaca

SEQ ID NO: 696 - RNA, 100 nt, Artificial Sequence
  Description of Artificial Sequence Synthetic polynucleotide
uccuuagccccaaagaguacguaagagcguguuucaacaucacuucgaacuccuggcggggcuuagcuacgcugcuucccucgacaccugauagucgaca

SEQ ID NO: 697 - RNA, 100 nt, Artificial Sequence
  Description of Artificial Sequence Synthetic polynucleotide
uccuccgccccaagggguacguaggagcgugaucuuccaucacuucgaacuccuggcggggcuuagcuacgcugcuaaaaaguuagacugauaugggaca

SEQ ID NO: 698 - RNA, 100 nt, Artificial Sequence
  Description of Artificial Sequence Synthetic polynucleotide
uccuccgccccaagggguacguaggagcgugaucuuccaucacuucgaacuccuggcggggcuuagcuacgcugcuucaaaguucaccugauagucgaca

SEQ ID NO: 699 - RNA, 100 nt, Artificial Sequence
  Description of Artificial Sequence Synthetic polynucleotide
uccugucccccaacccguacgucccagcgugaucuuccccggagucgaauuccuggcggagcucagccacgcugauucaaaguucaccucgcucccgaca

SEQ ID NO: 700 - RNA, 100 nt, Artificial Sequence
  Description of Artificial Sequence Synthetic polynucleotide
ucagaucccccuuaccguaccgaccagcggccucuuccccggagucgaauuccuggcggagcucagccacgcugauucaaaguucaccucgcucccgaca

SEQ ID NO: 701 - RNA, 100 nt, Artificial Sequence
  Description of Artificial Sequence Synthetic polynucleotide
uccugucgaacaacccauucgucccuaagugauccgguugcacuucgaauuccuggcggagcucagccacgcugaagugccguucacauuauagucgaca

SEQ ID NO: 702 - RNA, 100 nt, Artificial Sequence
  Description of Artificial Sequence Synthetic polynucleotide
uccugucgaacaacccauucgucccuaagugauccgguugcacuucgaauuccuggcggagcucagccacgcugaagugccguucaccugauagucgaca

SEQ ID NO: 703 - RNA, 100 nt, Artificial Sequence
  Description of Artificial Sequence Synthetic polynucleotide
uccugucccccaacccguacgucccagcguguuucaacaucacuucgaauuccuggcggagcucagccacgcugauuccuucggcaccugauagucgaca

SEQ ID NO: 704 - RNA, 100 nt, Artificial Sequence
  Description of Artificial Sequence Synthetic polynucleotide
uccuuggccccaaggaguacguaagagcguguuucaacaucacuucgaauuccuggcggagcucagccacgcugauuccuucggcaccugcucgucgaca

SEQ ID NO: 705 - RNA, 100 nt, Artificial Sequence
  Description of Artificial Sequence Synthetic polynucleotide
uccuuggccccaaggaguacguaagagcguguuucaacaucacuucgaauuccuggcggagcucagccacgcugauuccuucggcaccugauagucgaca

SEQ ID NO: 706 - RNA, 100 nt, Artificial Sequence
  Description of Artificial Sequence Synthetic polynucleotide
uccuaagccccaaggaguacguaaaagcgugaucuuccaucacuucgaauuccuggcggagcucagccacgcugauaaaaaguugugcugauaccggaca

SEQ ID NO: 707 - RNA, 100 nt, Artificial Sequence
  Description of Artificial Sequence Synthetic polynucleotide
uccuaagccccaaggaguacguaaaagcgugaucuuccaucacuucgaauuccuggcggagcucagccacgcugauucaaaguucaccugauagucgaca

SEQ ID NO: 708 - RNA, 100 nt, Artificial Sequence
  Description of Artificial Sequence Synthetic polynucleotide
uccugucccccaacccguacgucccagcgugaucuucccuagagucgaacuccuggcggagcucagccacgcuuguucaaaguucaccuucaccgcgaca

SEQ ID NO: 709 - RNA, 100 nt, Artificial Sequence
  Description of Artificial Sequence Synthetic polynucleotide
ucauuucccccuaaccguacccaccagcgcucucuucccuagagucgaacuccuggcggagcucagccacgcuuguucaaaguucaccuucaccgcgaca

SEQ ID NO: 710 - RNA, 100 nt, Artificial Sequence
  Description of Artificial Sequence Synthetic polynucleotide
uccuaggccccaagagguacgugggagcguguuucaacaucacuucgaacuccuggcggagcucagccacgcuuguuccguacacaccugcucgucgaca

SEQ ID NO: 711 - RNA, 100 nt, Artificial Sequence
  Description of Artificial Sequence Synthetic polynucleotide
uccuaggccccaagagguacgugggagcguguuucaacaucacuucgaacuccuggcggagcucagccacgcuuguuccguacacaccugauagucgaca

SEQ ID NO: 712 - RNA, 100 nt, Artificial Sequence
  Description of Artificial Sequence Synthetic polynucleotide
uccuuaaccccaaggaguacguagaagcgugaucuuccaucacuucgaacuccuggcggagcucagccacgcuuguucaaaguucaccugauagucgaca

SEQ ID NO: 713 - RNA, 100 nt, Artificial Sequence
  Description of Artificial Sequence Synthetic polynucleotide
ucgccucccccucgccguaccuaccagcggccucuuccccuuugucgaacuccuggcggggguuagccacgcugguuccaaguucaccuucucugcgaca

SEQ ID NO: 714 - RNA, 100 nt, Artificial Sequence
  Description of Artificial Sequence Synthetic polynucleotide
uccuauaccccaaggaguacguaaaagcguguuucaacaucacuucgaacuccuggcggggguuagccacgcugguuccggccccaccuguacgucgaca

SEQ ID NO: 715 - RNA, 100 nt, Artificial Sequence
  Description of Artificial Sequence Synthetic polynucleotide
uccuauaccccaaggaguacguaaaagcguguuucaacaucacuucgaacuccuggcggggguuagccacgcugguuccggccccaccugauagucgaca

SEQ ID NO: 716 - RNA, 100 nt, Artificial Sequence
  Description of Artificial Sequence Synthetic polynucleotide
uccuccgccccaagggguacguggaagcgugaucuuccaucacuucgaacuccuggcggggguuagccacgcuggaaacaaguuaugcugauaacggaca

SEQ ID NO: 717 - RNA, 100 nt, Artificial Sequence
  Description of Artificial Sequence Synthetic polynucleotide
uccuccgccccaagggguacguggaagcgugaucuuccaucacuucgaacuccuggcggggguuagccacgcugguuccaaguucaccugauagucgaca

SEQ ID NO: 718 - RNA, 100 nt, Artificial Sequence
  Description of Artificial Sequence Synthetic polynucleotide
uccugucccccaacccguacgucccagcgugaucuucccaaggaucgaacuccugacgggacuccgccacgcugauucaaaguucaccuaaggcgcgaca

SEQ ID NO: 719 - RNA, 100 nt, Artificial Sequence
  Description of Artificial Sequence Synthetic polynucleotide
ucgauucccccuaaccguacuagccagcgaccucuucccaaggaucgaacuccugacgggacuccgccacgcugauucaaaguucaccuaaggcgcgaca

SEQ ID NO: 720 - RNA, 100 nt, Artificial Sequence
  Description of Artificial Sequence Synthetic polynucleotide
uccugucaagcaacccaaccgucccuuagugauccuggugcacuucgaacuccugacgggacuccgccacgcugaagaaccguucacgcaauagucgaca

SEQ ID NO: 721 - RNA, 100 nt, Artificial Sequence
  Description of Artificial Sequence Synthetic polynucleotide
uccugucaagcaacccaaccgucccuuagugauccuggugcacuucgaacuccugacgggacuccgccacgcugaagaaccguucaccugauagucgaca

SEQ ID NO: 722 - RNA, 100 nt, Artificial Sequence
  Description of Artificial Sequence Synthetic polynucleotide
uccucagccccaaaaaguacguagaagcguguuucaacaucacuucgaacuccugacgggacuccgccacgcugauucuggcugcaccugcgugucgaca

SEQ ID NO: 723 - RNA, 100 nt, Artificial Sequence
  Description of Artificial Sequence Synthetic polynucleotide
uccucagccccaaaaaguacguagaagcguguuucaacaucacuucgaacuccugacgggacuccgccacgcugauucuggcugcaccugauagucgaca

SEQ ID NO: 724 - RNA, 100 nt, Artificial Sequence
  Description of Artificial Sequence Synthetic polynucleotide
uccuuagccccaagagguacguaagagcgugaucuuccaucacuucgaacuccugacgggacuccgccacgcugaaggaaaguugcacugauaagggaca

SEQ ID NO: 725 - RNA, 100 nt, Artificial Sequence
  Description of Artificial Sequence Synthetic polynucleotide
uccuuagccccaagagguacguaagagcgugaucuuccaucacuucgaacuccugacgggacuccgccacgcugauucaaaguucaccugauagucgaca

SEQ ID NO: 726 - RNA, 100 nt, Artificial Sequence
  Description of Artificial Sequence Synthetic polynucleotide
uccugucgagcaacccacucgucccuuagugauccauuagcacuucgaacuccuggcggggcuuagccacgcugaccccuuguucacaauauagucgaca

SEQ ID NO: 727 - RNA, 100 nt, Artificial Sequence
  Description of Artificial Sequence Synthetic polynucleotide
uccugucgagcaacccacucgucccuuagugauccauuagcacuucgaacuccuggcggggcuuagccacgcugaccccuuguucaccugauagucgaca

SEQ ID NO: 728 - RNA, 100 nt, Artificial Sequence
  Description of Artificial Sequence Synthetic polynucleotide
uccuccaccccaaacaguacgugagagcguguuucaacaucacuucgaacuccuggcggggcuuagccacgcugauuccugcagcaccuguccgucgaca

SEQ ID NO: 729 - RNA, 100 nt, Artificial Sequence
  Description of Artificial Sequence Synthetic polynucleotide
uccuccaccccaaacaguacgugagagcguguuucaacaucacuucgaacuccuggcggggcuuagccacgcugauuccugcagcaccugauagucgaca

SEQ ID NO: 730 - RNA, 100 nt, Artificial Sequence
  Description of Artificial Sequence Synthetic polynucleotide
uccuuuaccccaagaaguacguaggagcgugaucuuccaucacuucgaacuccuggcggggcuuagccacgcugacggaaaguugucaugauaacagaca

SEQ ID NO: 731 - RNA, 100 nt, Artificial Sequence
  Description of Artificial Sequence Synthetic polynucleotide
uccuuuaccccaagaaguacguaggagcgugaucuuccaucacuucgaacuccuggcggggcuuagccacgcugauucaaaguucacaugauagucgaca

SEQ ID NO: 732 - RNA, 100 nt, Artificial Sequence
  Description of Artificial Sequence Synthetic polynucleotide
ucggaucccccuugccguacuaaccagcgaccucuuccccaggaucgaauuccuggcgggucuaagccacgcugguccaaaguucaccuaaauuacgaca

SEQ ID NO: 733 - RNA, 100 nt, Artificial Sequence
  Description of Artificial Sequence Synthetic polynucleotide
uccugucaggcaacccuaucguccccuggugauccuggugcacuucgaauuccuggcgggucuaagccacgcuggacucucguucacagaauagucgaca

SEQ ID NO: 734 - RNA, 100 nt, Artificial Sequence
  Description of Artificial Sequence Synthetic polynucleotide
uccugucaggcaacccuaucguccccuggugauccuggugcacuucgaauuccuggcgggucuaagccacgcuggacucucguucaccugauagucgaca

SEQ ID NO: 735 - RNA, 100 nt, Artificial Sequence
  Description of Artificial Sequence Synthetic polynucleotide
uccuauaccccaagggguacguaggagcguguuucaacaucacuucgaauuccuggcgggucuaagccacgcugguccuccgcacaccuguuugucgaca

SEQ ID NO: 736 - RNA, 100 nt, Artificial Sequence
  Description of Artificial Sequence Synthetic polynucleotide
uccuauaccccaagggguacguaggagcguguuucaacaucacuucgaauuccuggcgggucuaagccacgcugguccuccgcacaccugauagucgaca

SEQ ID NO: 737 - RNA, 100 nt, Artificial Sequence
  Description of Artificial Sequence Synthetic polynucleotide
uccuauaccccaaaaaguacguaaaagcgugaucuuccaucacuucgaauuccuggcgggucuaagccacgcuggccgaaaguuaugcugauauaagaca

SEQ ID NO: 738 - RNA, 100 nt, Artificial Sequence
  Description of Artificial Sequence Synthetic polynucleotide
uccuauaccccaaaaaguacguaaaagcgugaucuuccaucacuucgaauuccuggcgggucuaagccacgcugguccaaaguucaccugauagucgaca

SEQ ID NO: 739 - RNA, 100 nt, Artificial Sequence
  Description of Artificial Sequence Synthetic polynucleotide
ucgauucccccccgccguacuggccagcgcccucuuccuaccgaucgaacuucugguggggcucagccacgcugguccaaagcucaccucgccgacgaca

SEQ ID NO: 740 - RNA, 100 nt, Artificial Sequence
  Description of Artificial Sequence Synthetic polynucleotide
uccugucaagcaaccccuccgucccuaagugauccguaagcacuucgaacuucugguggggcucagccacgcugggcuccuguucacgguauagucgaca

SEQ ID NO: 741 - RNA, 100 nt, Artificial Sequence
  Description of Artificial Sequence Synthetic polynucleotide
uccugucaagcaaccccuccgucccuaagugauccguaagcacuucgaacuucugguggggcucagccacgcugggcuccuguucaccugauagucgaca

SEQ ID NO: 742 - RNA, 100 nt, Artificial Sequence
  Description of Artificial Sequence Synthetic polynucleotide
uccuuuaccccaaaggguacgugaaagcguguuucaacaucacuucgaacuucugguggggcucagccacgcugguccuugacccaccugcacgucgaca

SEQ ID NO: 743 - RNA, 100 nt, Artificial Sequence
  Description of Artificial Sequence Synthetic polynucleotide
uccuuuaccccaaaggguacgugaaagcguguuucaacaucacuucgaacuucugguggggcucagccacgcugguccuugacccaccugauagucgaca

SEQ ID NO: 744 - RNA, 100 nt, Artificial Sequence
  Description of Artificial Sequence Synthetic polynucleotide
uccuuggccccaaaaaguacguaggagcgugaucuuccaucacuucgaacuucugguggggcucagccacgcugguccaaagcucaccugauagucgaca

SEQ ID NO: 745 - RNA, 100 nt, Artificial Sequence
  Description of Artificial Sequence Synthetic polynucleotide
ucgguucccccuuaccguacuagccagcgcucucuuccuggaaaucgaacuccuggcgggccucagccacgcugcuccaaaguucaccucacauccgaca

SEQ ID NO: 746 - RNA, 100 nt, Artificial Sequence
  Description of Artificial Sequence Synthetic polynucleotide
uccugucagacaacccaaucgucccuuggugaucaagugacacuucgaacuccuggcgggccucagccacgcugcuagucuguucacaguauagucgaca

SEQ ID NO: 747 - RNA, 100 nt, Artificial Sequence
  Description of Artificial Sequence Synthetic polynucleotide
uccugucagacaacccaaucgucccuuggugaucaagugacacuucgaacuccuggcgggccucagccacgcugcuagucuguucaccugauagucgaca

SEQ ID NO: 748 - RNA, 100 nt, Artificial Sequence
  Description of Artificial Sequence Synthetic polynucleotide
uccuacaccccaaaaaguacguaagagcgugaucuuccaucacuucgaacuccuggcgggccucagccacgcugcucaaaaguugcacugauaauagaca

SEQ ID NO: 749 - RNA, 100 nt, Artificial Sequence
  Description of Artificial Sequence Synthetic polynucleotide
uccuacaccccaaaaaguacguaagagcgugaucuuccaucacuucgaacuccuggcgggccucagccacgcugcuccaaaguucaccugauagucgaca

SEQ ID NO: 750 - RNA, 100 nt, Artificial Sequence
  Description of Artificial Sequence Synthetic polynucleotide
ucaucucccccccaccguacuagccagcggguucuuccuguuuaucgaacuccuggcggggauuagccacgcugauucaaaguucaccuuuauaccgaca

SEQ ID NO: 751 - RNA, 100 nt, Artificial Sequence
  Description of Artificial Sequence Synthetic polynucleotide
uccugucaggcaaccccuucgucccuuggugaucggagagcacuucgaacuccuggcggggauuagccacgcugauauguuguucacaucauagucgaca

SEQ ID NO: 752 - RNA, 100 nt, Artificial Sequence
  Description of Artificial Sequence Synthetic polynucleotide
uccugucaggcaaccccuucgucccuuggugaucggagagcacuucgaacuccuggcggggauuagccacgcugauauguuguucaccugauagucgaca

SEQ ID NO: 753 - RNA, 100 nt, Artificial Sequence
  Description of Artificial Sequence Synthetic polynucleotide
uccuacaccccaagggguacguaagagcguguuucaacaucacuucgaacuccuggcggggauuagccacgcugauucuauacgcaccugcuugucgaca

SEQ ID NO: 754 - RNA, 100 nt, Artificial Sequence
  Description of Artificial Sequence Synthetic polynucleotide
uccuacaccccaagggguacguaagagcguguuucaacaucacuucgaacuccuggcggggauuagccacgcugauucuauacgcaccugauagucgaca

SEQ ID NO: 755 - RNA, 100 nt, Artificial Sequence
  Description of Artificial Sequence Synthetic polynucleotide
uccuccgccccaagggguacguggaagcgugaucuuccaucacuucgaacuccuggcggggauuagccacgcugauaaaaaguugcacugauaaaggaca

SEQ ID NO: 756 - RNA, 100 nt, Artificial Sequence
  Description of Artificial Sequence Synthetic polynucleotide
uccuccgccccaagggguacguggaagcgugaucuuccaucacuucgaacuccuggcggggauuagccacgcugauucaaaguucaccugauagucgaca

SEQ ID NO: 757 - RNA, 100 nt, Artificial Sequence
  Description of Artificial Sequence Synthetic polynucleotide
ucgauucccccuuaccguacuaaccagcgcauucuucccaagaaucgaacuucuggcgggucucagccacgcucguucaaaguucaccuccaccacgaca

SEQ ID NO: 758 - RNA, 100 nt, Artificial Sequence
  Description of Artificial Sequence Synthetic polynucleotide
uccuacgccccaaacaguacguggaagcguguuucaacaucacuucgaacuucuggcgggucucagccacgcucguuccucaaccaccuguucgucgaca

SEQ ID NO: 759 - RNA, 100 nt, Artificial Sequence
  Description of Artificial Sequence Synthetic polynucleotide
uccuacgccccaaacaguacguggaagcguguuucaacaucacuucgaacuucuggcgggucucagccacgcucguuccucaaccaccugauagucgaca

SEQ ID NO: 760 - RNA, 100 nt, Artificial Sequence
  Description of Artificial Sequence Synthetic polynucleotide
uccuaaaccccaaacaguacguaaaagcgugaucuuccaucacuucgaacuucuggcgggucucagccacgcucgcggaaaguuaaacugauauuagaca

SEQ ID NO: 761 - RNA, 100 nt, Artificial Sequence
  Description of Artificial Sequence Synthetic polynucleotide
uccuaaaccccaaacaguacguaaaagcgugaucuuccaucacuucgaacuucuggcgggucucagccacgcucguucaaaguucaccugauagucgaca

SEQ ID NO: 762 - RNA, 100 nt, Artificial Sequence
  Description of Artificial Sequence Synthetic polynucleotide
ucgaaucccccccgccguacuagccagcgaauucuuccugugaaucgaauuccuggcggugcucagccacgcugguacaaaguucaccuccucaacgaca

SEQ ID NO: 763 - RNA, 100 nt, Artificial Sequence
  Description of Artificial Sequence Synthetic polynucleotide
uccugucaaacaacccuuccgucccucagugaucgcuuuccacuucgaauuccuggcggugcucagccacgcuggccgcacguucacaauauagucgaca

SEQ ID NO: 764 - RNA, 100 nt, Artificial Sequence
  Description of Artificial Sequence Synthetic polynucleotide
uccugucaaacaacccuuccgucccucagugaucgcuuuccacuucgaauuccuggcggugcucagccacgcuggccgcacguucaccugauagucgaca

SEQ ID NO: 765 - RNA, 100 nt, Artificial Sequence
  Description of Artificial Sequence Synthetic polynucleotide
uccuuuaccccaagagguacguaggagcguguuucaacaucacuucgaauuccuggcggugcucagccacgcugguacucgcgacaccugcaugucgaca

SEQ ID NO: 766 - RNA, 100 nt, Artificial Sequence
  Description of Artificial Sequence Synthetic polynucleotide
uccuuuaccccaagagguacguaggagcguguuucaacaucacuucgaauuccuggcggugcucagccacgcugguacucgcgacaccugauagucgaca

SEQ ID NO: 767 - RNA, 100 nt, Artificial Sequence
  Description of Artificial Sequence Synthetic polynucleotide
uccuauaccccaagggguacgugaaagcgugaucuuccaucacuucgaauuccuggcggugcucagccacgcuggcagaaaguuaugcugauaauagaca

SEQ ID NO: 768 - RNA, 100 nt, Artificial Sequence
  Description of Artificial Sequence Synthetic polynucleotide
uccuauaccccaagggguacgugaaagcgugaucuuccaucacuucgaauuccuggcggugcucagccacgcugguacaaaguucaccugauagucgaca

SEQ ID NO: 769 - RNA, 100 nt, Artificial Sequence
  Description of Artificial Sequence Synthetic polynucleotide
ucaucucccccucaccguacacgccagcgcucucuuccuuuguaucgcacuccuggcgggugucagccacgcuggugcaaaguucaccuucggcgcgaca

SEQ ID NO: 770 - RNA, 100 nt, Artificial Sequence
  Description of Artificial Sequence Synthetic polynucleotide
uccugucacacaaccccuccgucccucagugaucacacugcacuucgcacuccuggcgggugucagccacgcuggcgucccguucacaauauagucgaca

SEQ ID NO: 771 - RNA, 100 nt, Artificial Sequence
  Description of Artificial Sequence Synthetic polynucleotide
uccugucacacaaccccuccgucccucagugaucacacugcacuucgcacuccuggcgggugucagccacgcuggcgucccguucaccugauagucgaca

SEQ ID NO: 772 - RNA, 100 nt, Artificial Sequence
  Description of Artificial Sequence Synthetic polynucleotide
uccuagaccccaaaggguacguggaagcgugaucuuccaucacuucgcacuccuggcgggugucagccacgcuggggaaaaguuaugcugauaacggaca

SEQ ID NO: 773 - RNA, 100 nt, Artificial Sequence
  Description of Artificial Sequence Synthetic polynucleotide
uccuagaccccaaaggguacguggaagcgugaucuuccaucacuucgcacuccuggcgggugucagccacgcuggugcaaaguucaccugauagucgaca

SEQ ID NO: 774 - RNA, 100 nt, Artificial Sequence
  Description of Artificial Sequence Synthetic polynucleotide
ucgguucccccuuaccguaccagccagcgaauucuuccccccggucgaacuucuggcgggggucagcuacgcuguuacaaaguucaccucuccaccgaca

SEQ ID NO: 775 - RNA, 100 nt, Artificial Sequence
  Description of Artificial Sequence Synthetic polynucleotide
uccugucaggcaacccuaucgucccuuagugaucaaauugcacuucgaacuucuggcgggggucagcuacgcugucgccgcguucacgccauagucgaca

SEQ ID NO: 776 - RNA, 100 nt, Artificial Sequence
  Description of Artificial Sequence Synthetic polynucleotide
uccugucaggcaacccuaucgucccuuagugaucaaauugcacuucgaacuucuggcgggggucagcuacgcugucgccgcguucaccugauagucgaca

SEQ ID NO: 777 - RNA, 100 nt, Artificial Sequence
  Description of Artificial Sequence Synthetic polynucleotide
uccugucgagcaaccccuucgucccuaagugaucuuccaucacuucgaacuucuggcgggggucagcuacgcuguuaaaaaguuaaacugauacuagaca

SEQ ID NO: 778 - RNA, 100 nt, Artificial Sequence
  Description of Artificial Sequence Synthetic polynucleotide
uccugucgagcaaccccuucgucccuaagugaucuuccaucacuucgaacuucuggcgggggucagcuacgcuguuacaaaguucaccugauagucgaca

SEQ ID NO: 779 - RNA, 100 nt, Artificial Sequence
  Description of Artificial Sequence Synthetic polynucleotide
ucaccucccccccgccguacugaccagcggguucuucccagaggucgaaauccugacgggguuaagccacgcugcuacaaaguucaccuucgccgcgaca

SEQ ID NO: 780 - RNA, 100 nt, Artificial Sequence
  Description of Artificial Sequence Synthetic polynucleotide
uccugucaggcaaccccuucgucccuaagugaucgcaugccacuucgaaauccugacgggguuaagccacgcugcgaguacguucacaauauagucgaca

SEQ ID NO: 781 - RNA, 100 nt, Artificial Sequence
  Description of Artificial Sequence Synthetic polynucleotide
uccugucaggcaaccccuucgucccuaagugaucgcaugccacuucgaaauccugacgggguuaagccacgcugcgaguacguucaccugauagucgaca

SEQ ID NO: 782 - RNA, 100 nt, Artificial Sequence
  Description of Artificial Sequence Synthetic polynucleotide
uccugucaaacaacccauucguccccuggugaucuuccaucacuucgaaauccugacgggguuaagccacgcugcuagaaaguugcgcugauacuagaca

SEQ ID NO: 783 - RNA, 100 nt, Artificial Sequence
  Description of Artificial Sequence Synthetic polynucleotide
uccugucaaacaacccauucguccccuggugaucuuccaucacuucgaaauccugacgggguuaagccacgcugcuacaaaguucaccugauagucgaca

SEQ ID NO: 784 - RNA, 100 nt, Artificial Sequence
  Description of Artificial Sequence Synthetic polynucleotide
ucacauccccccaaccguacaggccagcggguucuuccccuuuaucgaacuccuggcggggcuuagucacgcuggugcaaaguucaccuaacuaccgaca

SEQ ID NO: 785 - RNA, 100 nt, Artificial Sequence
  Description of Artificial Sequence Synthetic polynucleotide
uccugucgaacaaccccuccgucccccggugaucgauuggcacuucgaacuccuggcggggcuuagucacgcugggcuuguguucacaaaauagucgaca

SEQ ID NO: 786 - RNA, 100 nt, Artificial Sequence
  Description of Artificial Sequence Synthetic polynucleotide
uccugucgaacaaccccuccgucccccggugaucgauuggcacuucgaacuccuggcggggcuuagucacgcugggcuuguguucaccugauagucgaca

SEQ ID NO: 787 - RNA, 100 nt, Artificial Sequence
  Description of Artificial Sequence Synthetic polynucleotide
uccugucaaacaaccccaucguccccaagugaucuuccaucacuucgaacuccuggcggggcuuagucacgcuggcggaaaguugcgcugauacaggaca

SEQ ID NO: 788 - RNA, 100 nt, Artificial Sequence
  Description of Artificial Sequence Synthetic polynucleotide
uccugucaaacaaccccaucguccccaagugaucuuccaucacuucgaacuccuggcggggcuuagucacgcuggugcaaaguucaccugauagucgaca

SEQ ID NO: 789 - RNA, 100 nt, Artificial Sequence
  Description of Artificial Sequence Synthetic polynucleotide
uccugucccccaacccguacgucccagcgugaucuucccaugugucgaaguccugacggggcucagccacgcuuguucaaaguucaccuuacgaacgaca

SEQ ID NO: 790 - RNA, 100 nt, Artificial Sequence
  Description of Artificial Sequence Synthetic polynucleotide
ucggcucccccccaccguaccuaccagcgcucucuucccaugugucgaaguccugacggggcucagccacgcuuguucaaaguucaccuuacgaacgaca

SEQ ID NO: 791 - RNA, 100 nt, Artificial Sequence
  Description of Artificial Sequence Synthetic polynucleotide
uccuacaccccaaggaguacgugggagcguguuucaacaucacuucgaaguccugacggggcucagccacgcuuguucuucuaccaccugcucgucgaca

SEQ ID NO: 792 - RNA, 100 nt, Artificial Sequence
  Description of Artificial Sequence Synthetic polynucleotide
uccuacaccccaaggaguacgugggagcguguuucaacaucacuucgaaguccugacggggcucagccacgcuuguucuucuaccaccugauagucgaca

SEQ ID NO: 793 - RNA, 100 nt, Artificial Sequence
  Description of Artificial Sequence Synthetic polynucleotide
uccucuaccccaagggguacguacaagcgugaucuuccaucacuucgaaguccugacggggcucagccacgcuuguucaaaguucaccugauagucgaca

SEQ ID NO: 794 - RNA, 100 nt, Artificial Sequence
  Description of Artificial Sequence Synthetic polynucleotide
ucgacucccccucgccguaccagccagcggucucuucccuauagucgaacuccugacggagcucagccacgcuggaucaaaguucaccuuacuaccgaca

SEQ ID NO: 795 - RNA, 100 nt, Artificial Sequence
  Description of Artificial Sequence Synthetic polynucleotide
uccugucaagcaaccccuucguccccuagugaucgaauuccacuucgaacuccugacggagcucagccacgcugggauuguguucacauaauagucgaca

SEQ ID NO: 796 - RNA, 100 nt, Artificial Sequence
  Description of Artificial Sequence Synthetic polynucleotide
uccugucaagcaaccccuucguccccuagugaucgaauuccacuucgaacuccugacggagcucagccacgcugggauuguguucaccugauagucgaca

SEQ ID NO: 797 - RNA, 100 nt, Artificial Sequence
  Description of Artificial Sequence Synthetic polynucleotide
uccuuuaccccaagggguacguggaagcguguuucaacaucacuucgaacuccugacggagcucagccacgcuggaucuaacgccaccugcuugucgaca

SEQ ID NO: 798 - RNA, 100 nt, Artificial Sequence
  Description of Artificial Sequence Synthetic polynucleotide
uccuuuaccccaagggguacguggaagcguguuucaacaucacuucgaacuccugacggagcucagccacgcuggaucuaacgccaccugauagucgaca

SEQ ID NO: 799 - RNA, 100 nt, Artificial Sequence
  Description of Artificial Sequence Synthetic polynucleotide
uccucgaccccaagaaguacguaagagcgugaucuuccaucacuucgaacuccugacggagcucagccacgcuggagaaaaguugugcugauauuagaca

SEQ ID NO: 800 - RNA, 100 nt, Artificial Sequence
  Description of Artificial Sequence Synthetic polynucleotide
uccucgaccccaagaaguacguaagagcgugaucuuccaucacuucgaacuccugacggagcucagccacgcuggaucaaaguucaccugauagucgaca

SEQ ID NO: 801 - RNA, 100 nt, Artificial Sequence
  Description of Artificial Sequence Synthetic polynucleotide
uccugucccccaacccguacgucccagcgugaucuuccuacgggucgaacuccuggcgguguucagccacgcuuguucaaaguucaucuccuccccgaca

SEQ ID NO: 802 - RNA, 100 nt, Artificial Sequence
  Description of Artificial Sequence Synthetic polynucleotide
ucauuucccccuugccguacuagccagcgccuucuuccuacgggucgaacuccuggcgguguucagccacgcuuguucaaaguucaucuccuccccgaca

SEQ ID NO: 803 - RNA, 100 nt, Artificial Sequence
  Description of Artificial Sequence Synthetic polynucleotide
uccucggccccaagggguacguagaagcguguuucaacaucacuucgaacuccuggcgguguucagccacgcuuguucucacgccaccuguaugucgaca

SEQ ID NO: 804 - RNA, 100 nt, Artificial Sequence
  Description of Artificial Sequence Synthetic polynucleotide
uccucggccccaagggguacguagaagcguguuucaacaucacuucgaacuccuggcgguguucagccacgcuuguucucacgccaccugauagucgaca

SEQ ID NO: 805 - RNA, 100 nt, Artificial Sequence
  Description of Artificial Sequence Synthetic polynucleotide
uccugucccccaacccguacgucccagcgugaucuuccaucacuucgaacuccuggcgguguucagccacgcuuguucaaaguucaucugauagucgaca

SEQ ID NO: 806 - RNA, 100 nt, Artificial Sequence
  Description of Artificial Sequence Synthetic polynucleotide
uccugucgaacaaccccgccgucccucagugaucuuccaucacuucgaacuccuggcgguguucagccacgcuuguucaaaguucaucugauagucgaca

SEQ ID NO: 807 - RNA, 100 nt, Artificial Sequence
  Description of Artificial Sequence Synthetic polynucleotide
uccauccccccauggcguacggagcagcguaaacuuccacgugagcgaacuccuggcgggggagagccacgcugguucaugucgaaccugauagucgaca

SEQ ID NO: 808 - RNA, 100 nt, Artificial Sequence
  Description of Artificial Sequence Synthetic polynucleotide
uccugucccgauacccgucuuucccagauagaucuuauuggucuucgaacuccuggcgggggagagccacgcugguucaucgcggaccugagcuucgaca

SEQ ID NO: 809 - RNA, 100 nt, Artificial Sequence
  Description of Artificial Sequence Synthetic polynucleotide
uccugucccgauacccgucuuucccagauagaucuuauuggucuucgaacuccuggcgggggagagccacgcugguucaucgcggaccugauagucgaca

SEQ ID NO: 810 - RNA, 100 nt, Artificial Sequence
  Description of Artificial Sequence Synthetic polynucleotide
uccuguccgaaaacccgguagucccaaauugaucugaucgaacuucgaacuccuggcgggggagagccacgcugguucauuugggaccugaacuucgaca

SEQ ID NO: 811 - RNA, 100 nt, Artificial Sequence
  Description of Artificial Sequence Synthetic polynucleotide
uccuguccgaaaacccgguagucccaaauugaucugaucgaacuucgaacuccuggcgggggagagccacgcugguucauuugggaccugauagucgaca

SEQ ID NO: 812 - RNA, 100 nt, Artificial Sequence
  Description of Artificial Sequence Synthetic polynucleotide
uccugucggacaaccccgccguccccuggugaucgcuuggcacuucgaacuccuggcgggggagagccacgcugguucaucagaaaccugacucucgaca

SEQ ID NO: 813 - RNA, 100 nt, Artificial Sequence
  Description of Artificial Sequence Synthetic polynucleotide
uccugucggacaaccccgccguccccuggugaucgcuuggcacuucgaacuccuggcgggggagagccacgcugguucaucagaaaccugauagucgaca

SEQ ID NO: 814 - RNA, 100 nt, Artificial Sequence
  Description of Artificial Sequence Synthetic polynucleotide
uccugugaaccaaccaucacguccguccgugaugggggcucacuucgaacuccuggcgggggagagccacgcugguucaugaaaaaccugacuuucgaca

SEQ ID NO: 815 - RNA, 100 nt, Artificial Sequence
  Description of Artificial Sequence Synthetic polynucleotide
uccugugaaccaaccaucacguccguccgugaugggggcucacuucgaacuccuggcgggggagagccacgcugguucaugaaaaaccugauagucgaca

SEQ ID NO: 816 - RNA, 100 nt, Artificial Sequence
  Description of Artificial Sequence Synthetic polynucleotide
uccuggaacccaacgauuacgucagcgcgugaggcaggaucacuucgaacuccuggcgggggagagccacgcugguucacuaccgaccugaccaucgaca

SEQ ID NO: 817 - RNA, 100 nt, Artificial Sequence
  Description of Artificial Sequence Synthetic polynucleotide
uccuggaacccaacgauuacgucagcgcgugaggcaggaucacuucgaacuccuggcgggggagagccacgcugguucacuaccgaccugauagucgaca

SEQ ID NO: 818 - RNA, 100 nt, Artificial Sequence
  Description of Artificial Sequence Synthetic polynucleotide
uccuagaccccaaagaguacguggaagcgugugggaacaucacuucgaacuccuggcgggggagagccacgcugguucacuaaagaccugaaucucgaca

SEQ ID NO: 819 - RNA, 100 nt, Artificial Sequence
  Description of Artificial Sequence Synthetic polynucleotide
uccuagaccccaaagaguacguggaagcgugugggaacaucacuucgaacuccuggcgggggagagccacgcugguucacuaaagaccugauagucgaca

SEQ ID NO: 820 - RNA, 100 nt, Artificial Sequence
  Description of Artificial Sequence Synthetic polynucleotide
uccccacccccaugacguacgacacagcguuuggccccaucacuucgaacuccuggcgggggagagccacgcugguucaccuggaaccugacucucgaca

SEQ ID NO: 821 - RNA, 100 nt, Artificial Sequence
  Description of Artificial Sequence Synthetic polynucleotide
uccccacccccaugacguacgacacagcguuuggccccaucacuucgaacuccuggcgggggagagccacgcugguucaccuggaaccugauagucgaca

SEQ ID NO: 822 - RNA, 100 nt, Artificial Sequence
  Description of Artificial Sequence Synthetic polynucleotide
ucggauccccccugccguacugaccagcggugcacuccaucacuucgaacuccuggcgggggagagccacgcugguucauucauaaccugagauucgaca

SEQ ID NO: 823 - RNA, 100 nt, Artificial Sequence
  Description of Artificial Sequence Synthetic polynucleotide
ucggauccccccugccguacugaccagcggugcacuccaucacuucgaacuccuggcgggggagagccacgcugguucauucauaaccugauagucgaca

SEQ ID NO: 824 - RNA, 100 nt, Artificial Sequence
  Description of Artificial Sequence Synthetic polynucleotide
uccugucccgacacccgucaaucccagaaguuacuuccaucacuucgaacuccuggcgggggagagccacgcugguucaccccuaaccugaguaucgaca

SEQ ID NO: 825 - RNA, 100 nt, Artificial Sequence
  Description of Artificial Sequence Synthetic polynucleotide
uccugucccgacacccgucaaucccagaaguuacuuccaucacuucgaacuccuggcgggggagagccacgcugguucaccccuaaccugauagucgaca

SEQ ID NO: 826 - RNA, 100 nt, Artificial Sequence
  Description of Artificial Sequence Synthetic polynucleotide
uccugucgagcaacccauucgucccuauuccaucuuccaucacuucgaacuccuggcgggggagagccacgcugguucaugaaagaccugagucucgaca

SEQ ID NO: 827 - RNA, 100 nt, Artificial Sequence
  Description of Artificial Sequence Synthetic polynucleotide
uccugucgagcaacccauucgucccuauuccaucuuccaucacuucgaacuccuggcgggggagagccacgcugguucaugaaagaccugauagucgaca

SEQ ID NO: 828 - RNA, 100 nt, Artificial Sequence
  Description of Artificial Sequence Synthetic polynucleotide
uccugucagacaacccuaucgucccagcgugaucuuccaucacuucgaacuccuggcgggggagagccacgcugguucaccaagaaccugacaaucgaca

SEQ ID NO: 829 - RNA, 100 nt, Artificial Sequence
  Description of Artificial Sequence Synthetic polynucleotide
uccugucagacaacccuaucgucccagcgugaucuuccaucacuucgaacuccuggcgggggagagccacgcugguucaccaagaaccugauagucgaca

SEQ ID NO: 830 - RNA, 100 nt, Artificial Sequence
  Description of Artificial Sequence Synthetic polynucleotide
uccugucccccaacccguacgucccagcgugaucuuccacugaagcgaacuccuggcgggggagagccacgcugguucaauuaaauccugauagucgaca

SEQ ID NO: 831 - RNA, 100 nt, Artificial Sequence
  Description of Artificial Sequence Synthetic polynucleotide
uccccacccccacgacguacgacacagcguccacuuccacugaagcgaacuccuggcgggggagagccacgcugguucaauuaaauccugauagucgaca

SEQ ID NO: 832 - RNA, 100 nt, Artificial Sequence
  Description of Artificial Sequence Synthetic polynucleotide
ucgccucccccucgccguacuagccagcgaucucuuccuauggaucgaacuccuggcgggggagagccacgcugguucaauaauacccugaucaacgaca

SEQ ID NO: 833 - RNA, 100 nt, Artificial Sequence
  Description of Artificial Sequence Synthetic polynucleotide
ucgccucccccucgccguacuagccagcgaucucuuccuauggaucgaacuccuggcgggggagagccacgcugguucaauaauacccugauagucgaca

SEQ ID NO: 834 - RNA, 100 nt, Artificial Sequence
  Description of Artificial Sequence Synthetic polynucleotide
uccugucccgacacccgucaaucccagaccgaucuugaagaccuucgaacuccuggcgggggagagccacgcugguucaauaacccccugaucaacgaca

SEQ ID NO: 835 - RNA, 100 nt, Artificial Sequence
  Description of Artificial Sequence Synthetic polynucleotide
uccugucccgacacccgucaaucccagaccgaucuugaagaccuucgaacuccuggcgggggagagccacgcugguucaauaacccccugauagucgaca

SEQ ID NO: 836 - RNA, 100 nt, Artificial Sequence
  Description of Artificial Sequence Synthetic polynucleotide
uccuguccagaaacccggcagucccacauugaucucagugaacuucgaacuccuggcgggggagagccacgcugguucaacauccuccugauuuccgaca

SEQ ID NO: 837 - RNA, 100 nt, Artificial Sequence
  Description of Artificial Sequence Synthetic polynucleotide
uccuguccagaaacccggcagucccacauugaucucagugaacuucgaacuccuggcgggggagagccacgcugguucaacauccuccugauagucgaca

SEQ ID NO: 838 - RNA, 100 nt, Artificial Sequence
  Description of Artificial Sequence Synthetic polynucleotide
uccugucgagcaacccuaucgucccccagugaucgaaagacacuucgaacuccuggcgggggagagccacgcugguucaacuauauccugauucgcgaca

SEQ ID NO: 839 - RNA, 100 nt, Artificial Sequence
  Description of Artificial Sequence Synthetic polynucleotide
uccugucgagcaacccuaucgucccccagugaucgaaagacacuucgaacuccuggcgggggagagccacgcugguucaacuauauccugauagucgaca

SEQ ID NO: 840 - RNA, 100 nt, Artificial Sequence
  Description of Artificial Sequence Synthetic polynucleotide
uccuguggaccaaccaugacguccgcucgugaugcaaacucacuucgaacuccuggcgggggagagccacgcugguucaauacaccccugauucacgaca

SEQ ID NO: 841 - RNA, 100 nt, Artificial Sequence
  Description of Artificial Sequence Synthetic polynucleotide
uccuguggaccaaccaugacguccgcucgugaugcaaacucacuucgaacuccuggcgggggagagccacgcugguucaauacaccccugauagucgaca

SEQ ID NO: 842 - RNA, 100 nt, Artificial Sequence
  Description of Artificial Sequence Synthetic polynucleotide
uccuggaacccaacaauuacgucgaugcgugaagcagaaucacuucgaacuccuggcgggggagagccacgcugguucaaugauacccugauucacgaca

SEQ ID NO: 843 - RNA, 100 nt, Artificial Sequence
  Description of Artificial Sequence Synthetic polynucleotide
uccuggaacccaacaauuacgucgaugcgugaagcagaaucacuucgaacuccuggcgggggagagccacgcugguucaaugauacccugauagucgaca

SEQ ID NO: 844 - RNA, 100 nt, Artificial Sequence
  Description of Artificial Sequence Synthetic polynucleotide
uccuagaccccaaaggguacgugggagcgugcgagagcaucacuucgaacuccuggcgggggagagccacgcugguucaacacguuccugauucgcgaca

SEQ ID NO: 845 - RNA, 100 nt, Artificial Sequence
  Description of Artificial Sequence Synthetic polynucleotide
uccuagaccccaaaggguacgugggagcgugcgagagcaucacuucgaacuccuggcgggggagagccacgcugguucaacacguuccugauagucgaca

SEQ ID NO: 846 - RNA, 100 nt, Artificial Sequence
  Description of Artificial Sequence Synthetic polynucleotide
ucccagcccccacagcguacgaggcagcgucccgggccaucacuucgaacuccuggcgggggagagccacgcugguucaauuggguccugaucaccgaca

SEQ ID NO: 847 - RNA, 100 nt, Artificial Sequence
  Description of Artificial Sequence Synthetic polynucleotide
ucccagcccccacagcguacgaggcagcgucccgggccaucacuucgaacuccuggcgggggagagccacgcugguucaauuggguccugauagucgaca

SEQ ID NO: 848 - RNA, 100 nt, Artificial Sequence
  Description of Artificial Sequence Synthetic polynucleotide
ucaguucccccucgccguacccgccagcgggcguguccaucacuucgaacuccuggcgggggagagccacgcugguucaauuagauccugauucacgaca

SEQ ID NO: 849 - RNA, 100 nt, Artificial Sequence
  Description of Artificial Sequence Synthetic polynucleotide
ucaguucccccucgccguacccgccagcgggcguguccaucacuucgaacuccuggcgggggagagccacgcugguucaauuagauccugauagucgaca

SEQ ID NO: 850 - RNA, 100 nt, Artificial Sequence
  Description of Artificial Sequence Synthetic polynucleotide
uccugucccaacacccguuuuucccagaucucacuuccaucacuucgaacuccuggcgggggagagccacgcugguucaaucggauccugauuaacgaca

SEQ ID NO: 851 - RNA, 100 nt, Artificial Sequence
  Description of Artificial Sequence Synthetic polynucleotide
uccugucccaacacccguuuuucccagaucucacuuccaucacuucgaacuccuggcgggggagagccacgcugguucaaucggauccugauagucgaca

SEQ ID NO: 852 - RNA, 100 nt, Artificial Sequence
  Description of Artificial Sequence Synthetic polynucleotide
uccuguccgaaaacccgcuagucccaccaaguucuuccaucacuucgaacuccuggcgggggagagccacgcugguucaaccugguccugauuuccgaca

SEQ ID NO: 853 - RNA, 100 nt, Artificial Sequence
  Description of Artificial Sequence Synthetic polynucleotide
uccuguccgaaaacccgcuagucccaccaaguucuuccaucacuucgaacuccuggcgggggagagccacgcugguucaaccugguccugauagucgaca

SEQ ID NO: 854 - RNA, 100 nt, Artificial Sequence
  Description of Artificial Sequence Synthetic polynucleotide
uccugucagacaacccauucgucccccuuucaucuuccaucacuucgaacuccuggcgggggagagccacgcugguucaauaucuuccugauccacgaca

SEQ ID NO: 855 - RNA, 100 nt, Artificial Sequence
  Description of Artificial Sequence Synthetic polynucleotide
uccugucagacaacccauucgucccccuuucaucuuccaucacuucgaacuccuggcgggggagagccacgcugguucaauaucuuccugauagucgaca

SEQ ID NO: 856 - RNA, 100 nt, Artificial Sequence
  Description of Artificial Sequence Synthetic polynucleotide
uccacccccccaucacguacgcaacagcguucccuuccaggcaaccgaacuccuggcgggggagagccacgcugguucaaaucuuugcugauacaagaca

SEQ ID NO: 857 - RNA, 100 nt, Artificial Sequence
  Description of Artificial Sequence Synthetic polynucleotide
uccacccccccaucacguacgcaacagcguucccuuccaggcaaccgaacuccuggcgggggagagccacgcugguucaaaucuuugcugauagucgaca

SEQ ID NO: 858 - RNA, 100 nt, Artificial Sequence
  Description of Artificial Sequence Synthetic polynucleotide
ucggauccccccaaccguaccggccagcggccucuuccugagugucgaacuccuggcgggggagagccacgcugguucaaaaucuugcugauaccagaca

SEQ ID NO: 859 - RNA, 100 nt, Artificial Sequence
  Description of Artificial Sequence Synthetic polynucleotide
ucggauccccccaaccguaccggccagcggccucuuccugagugucgaacuccuggcgggggagagccacgcugguucaaaaucuugcugauagucgaca

SEQ ID NO: 860 - RNA, 100 nt, Artificial Sequence
  Description of Artificial Sequence Synthetic polynucleotide
uccugucccgguacccgucauucccagaaagaucuuggcgaucuucgaacuccuggcgggggagagccacgcugguucaaauauggacugauacaagaca

SEQ ID NO: 861 - RNA, 100 nt, Artificial Sequence
  Description of Artificial Sequence Synthetic polynucleotide
uccugucccgguacccgucauucccagaaagaucuuggcgaucuucgaacuccuggcgggggagagccacgcugguucaaauauggacugauagucgaca

SEQ ID NO: 862 - RNA, 100 nt, Artificial Sequence
  Description of Artificial Sequence Synthetic polynucleotide
uccuguccaagaacccggaagucccaauuugaucucaagggacuucgaacuccuggcgggggagagccacgcugguucaaaaaggugcugauauuagaca

SEQ ID NO: 863 - RNA, 100 nt, Artificial Sequence
  Description of Artificial Sequence Synthetic polynucleotide
uccuguccaagaacccggaagucccaauuugaucucaagggacuucgaacuccuggcgggggagagccacgcugguucaaaaaggugcugauagucgaca

SEQ ID NO: 864 - RNA, 100 nt, Artificial Sequence
  Description of Artificial Sequence Synthetic polynucleotide
uccugucacacaacccuaucguccccaagugaucguagaacacuucgaacuccuggcgggggagagccacgcugguucaaacaaaugcugauacaggaca

SEQ ID NO: 865 - RNA, 100 nt, Artificial Sequence
  Description of Artificial Sequence Synthetic polynucleotide
uccugucacacaacccuaucguccccaagugaucguagaacacuucgaacuccuggcgggggagagccacgcugguucaaacaaaugcugauagucgaca

SEQ ID NO: 866 - RNA, 100 nt, Artificial Sequence
  Description of Artificial Sequence Synthetic polynucleotide
uccuguaggccaaccauaacguccacucgugauggcauuucacuucgaacuccuggcgggggagagccacgcugguucaaaucuauacugauacgggaca

SEQ ID NO: 867 - RNA, 100 nt, Artificial Sequence
  Description of Artificial Sequence Synthetic polynucleotide
uccuguaggccaaccauaacguccacucgugauggcauuucacuucgaacuccuggcgggggagagccacgcugguucaaaucuauacugauagucgaca

SEQ ID NO: 868 - RNA, 100 nt, Artificial Sequence
  Description of Artificial Sequence Synthetic polynucleotide
uccugaaacccaacauauacgucagcgcgugaaaaggaaucacuucgaacuccuggcgggggagagccacgcugguucaaaccgguacugauacgggaca

SEQ ID NO: 869 - RNA, 100 nt, Artificial Sequence
  Description of Artificial Sequence Synthetic polynucleotide
uccugaaacccaacauauacgucagcgcgugaaaaggaaucacuucgaacuccuggcgggggagagccacgcugguucaaaccgguacugauagucgaca

SEQ ID NO: 870 - RNA, 100 nt, Artificial Sequence
  Description of Artificial Sequence Synthetic polynucleotide
uccccgcccccaucacguacggcacagcgucccagaccaucacuucgaacuccuggcgggggagagccacgcugguucaaacgaaugcugauaugggaca

SEQ ID NO: 871 - RNA, 100 nt, Artificial Sequence
  Description of Artificial Sequence Synthetic polynucleotide
uccccgcccccaucacguacggcacagcgucccagaccaucacuucgaacuccuggcgggggagagccacgcugguucaaacgaaugcugauagucgaca

SEQ ID NO: 872 - RNA, 100 nt, Artificial Sequence
  Description of Artificial Sequence Synthetic polynucleotide
ucggaucccccucgccguacccgccagcgagcaaauccaucacuucgaacuccuggcgggggagagccacgcugguucaaaaacccgcugauaagggaca

SEQ ID NO: 873 - RNA, 100 nt, Artificial Sequence
  Description of Artificial Sequence Synthetic polynucleotide
ucggaucccccucgccguacccgccagcgagcaaauccaucacuucgaacuccuggcgggggagagccacgcugguucaaaaacccgcugauagucgaca

SEQ ID NO: 874 - RNA, 100 nt, Artificial Sequence
  Description of Artificial Sequence Synthetic polynucleotide
uccugucccagcacccgucuaucccaggcacuccuuccaucacuucgaacuccuggcgggggagagccacgcugguucaaacuaguacugauaacggaca

SEQ ID NO: 875 - RNA, 100 nt, Artificial Sequence
  Description of Artificial Sequence Synthetic polynucleotide
uccugucccagcacccgucuaucccaggcacuccuuccaucacuucgaacuccuggcgggggagagccacgcugguucaaacuaguacugauagucgaca

SEQ ID NO: 876 - RNA, 100 nt, Artificial Sequence
  Description of Artificial Sequence Synthetic polynucleotide
uccuguccagaaacccgcuggucccacacaucucuuccaucacuucgaacuccuggcgggggagagccacgcugguucaaaagcgugcugauacgagaca

SEQ ID NO: 877 - RNA, 100 nt, Artificial Sequence
  Description of Artificial Sequence Synthetic polynucleotide
uccuguccagaaacccgcuggucccacacaucucuuccaucacuucgaacuccuggcgggggagagccacgcugguucaaaagcgugcugauagucgaca

SEQ ID NO: 878 - RNA, 100 nt, Artificial Sequence
  Description of Artificial Sequence Synthetic polynucleotide
uccugucagacaaccccaccgucccccaaauaucuuccaucacuucgaacuccuggcgggggagagccacgcugguucaaaagcaaacugauauaggaca

SEQ ID NO: 879 - RNA, 100 nt, Artificial Sequence
  Description of Artificial Sequence Synthetic polynucleotide
uccugucagacaaccccaccgucccccaaauaucuuccaucacuucgaacuccuggcgggggagagccacgcugguucaaaagcaaacugauagucgaca

SEQ ID NO: 880 - RNA, 100 nt, Artificial Sequence
  Description of Artificial Sequence Synthetic polynucleotide
ucggaucccccucgccguacuagccagcgaacucuucccccccgucgaacuccuggcgggggagagccacgcugguucaaagcuguagugauaggguaca

SEQ ID NO: 881 - RNA, 100 nt, Artificial Sequence
  Description of Artificial Sequence Synthetic polynucleotide
ucggaucccccucgccguacuagccagcgaacucuucccccccgucgaacuccuggcgggggagagccacgcugguucaaagcuguagugauagucgaca

SEQ ID NO: 882 - RNA, 100 nt, Artificial Sequence
  Description of Artificial Sequence Synthetic polynucleotide
uccugucccgguacccgucucucccagaccgaucuuagagaccuucgaacuccuggcgggggagagccacgcugguucaaagggaaggugauaggauaca

SEQ ID NO: 883 - RNA, 100 nt, Artificial Sequence
  Description of Artificial Sequence Synthetic polynucleotide
uccugucccgguacccgucucucccagaccgaucuuagagaccuucgaacuccuggcgggggagagccacgcugguucaaagggaaggugauagucgaca

SEQ ID NO: 884 - RNA, 100 nt, Artificial Sequence
  Description of Artificial Sequence Synthetic polynucleotide
uccugucgagcaacccagccgucccuaagugaucaaaaaacacuucgaacuccuggcgggggagagccacgcugguucaaaggcguggugauaggguaca

SEQ ID NO: 885 - RNA, 100 nt, Artificial Sequence
  Description of Artificial Sequence Synthetic polynucleotide
uccugucgagcaacccagccgucccuaagugaucaaaaaacacuucgaacuccuggcgggggagagccacgcugguucaaaggcguggugauagucgaca

SEQ ID NO: 886 - RNA, 100 nt, Artificial Sequence
  Description of Artificial Sequence Synthetic polynucleotide
uccuguaaaccaaccgaaacguccguacgugauggcaauucacuucgaacuccuggcgggggagagccacgcugguucaaaggaggaaugauagguuaca

SEQ ID NO: 887 - RNA, 100 nt, Artificial Sequence
  Description of Artificial Sequence Synthetic polynucleotide
uccuguaaaccaaccgaaacguccguacgugauggcaauucacuucgaacuccuggcgggggagagccacgcugguucaaaggaggaaugauagucgaca

SEQ ID NO: 888 - RNA, 100 nt, Artificial Sequence
  Description of Artificial Sequence Synthetic polynucleotide
uccuggcacccaacgguuacgucagcgcgugagcgaggaucacuucgaacuccuggcgggggagagccacgcugguucaaagccgugaugauaggauaca

SEQ ID NO: 889 - RNA, 100 nt, Artificial Sequence
  Description of Artificial Sequence Synthetic polynucleotide
uccuggcacccaacgguuacgucagcgcgugagcgaggaucacuucgaacuccuggcgggggagagccacgcugguucaaagccgugaugauagucgaca

SEQ ID NO: 890 - RNA, 100 nt, Artificial Sequence
  Description of Artificial Sequence Synthetic polynucleotide
uccuacaccccaaaggguacguaggagcgugcgggcgcaucacuucgaacuccuggcgggggagagccacgcugguucaaaggggagaugauagggaaca

SEQ ID NO: 891 - RNA, 100 nt, Artificial Sequence
  Description of Artificial Sequence Synthetic polynucleotide
uccuacaccccaaaggguacguaggagcgugcgggcgcaucacuucgaacuccuggcgggggagagccacgcugguucaaaggggagaugauagucgaca

SEQ ID NO: 892 - RNA, 100 nt, Artificial Sequence
  Description of Artificial Sequence Synthetic polynucleotide
ucccaccccccaugacguacgauacagcguacaggaccaucacuucgaacuccuggcgggggagagccacgcugguucaaaggguuaaugauagguuaca

SEQ ID NO: 893 - RNA, 100 nt, Artificial Sequence
  Description of Artificial Sequence Synthetic polynucleotide
ucccaccccccaugacguacgauacagcguacaggaccaucacuucgaacuccuggcgggggagagccacgcugguucaaaggguuaaugauagucgaca

SEQ ID NO: 894 - RNA, 100 nt, Artificial Sequence
  Description of Artificial Sequence Synthetic polynucleotide
uccugucccgauacccgucaaucccagagcaggcuuccaucacuucgaacuccuggcgggggagagccacgcugguucaaagcaugagugauaggguaca

SEQ ID NO: 895 - RNA, 100 nt, Artificial Sequence
  Description of Artificial Sequence Synthetic polynucleotide
uccuguccggaaacccgcaagucccaaccuauucuuccaucacuucgaacuccuggcgggggagagccacgcugguucaaagcugcaaugauagucgaca

SEQ ID NO: 896 - RNA, 100 nt, Artificial Sequence
  Description of Artificial Sequence Synthetic polynucleotide
uccugucaaacaaccccuccguccccuaauuaucuuccaucacuucgaacuccuggcgggggagagccacgcugguucaaaggauucaugauaggguaca

SEQ ID NO: 897 - RNA, 100 nt, Artificial Sequence
  Description of Artificial Sequence Synthetic polynucleotide
uccugucaaacaaccccuccguccccuaauuaucuuccaucacuucgaacuccuggcgggggagagccacgcugguucaaaggauucaugauagucgaca

SEQ ID NO: 898 - RNA, 100 nt, Artificial Sequence
  Description of Artificial Sequence Synthetic polynucleotide
uccugucaggcaacccccccgucccagcgugaucuuccaucacuucgaacuccuggcgggggagagccacgcugguucaaagacaaaaugauaggguaca

SEQ ID NO: 899 - RNA, 100 nt, Artificial Sequence
  Description of Artificial Sequence Synthetic polynucleotide
uccugucaggcaacccccccgucccagcgugaucuuccaucacuucgaacuccuggcgggggagagccacgcugguucaaagacaaaaugauagucgaca

SEQ ID NO: 900 - RNA, 100 nt, Artificial Sequence
  Description of Artificial Sequence Synthetic polynucleotide
uccuguccggaaacccgaaagucccaaauugaucuggggcgacuucgaacuccuggcgggggagagccacgcuagccacaaguucaccugauagucgaca

SEQ ID NO: 901 - RNA, 100 nt, Artificial Sequence
  Description of Artificial Sequence Synthetic polynucleotide
uccugcaacccaacaaauacgucaacgcgugaauauccaucugagaaaacuccuggcgggggagagccacgcugcaaacuaguucaccugauagucgaca

SEQ ID NO: 902 - RNA, 100 nt, Artificial Sequence
  Description of Artificial Sequence Synthetic polynucleotide
uccuuagccccaaagaguacguagaagcguguaguuccauauaaaggaacuccuggcgggggagagccacgcugcaagcuaguucaccugauagucgaca

SEQ ID NO: 903 - RNA, 100 nt, Artificial Sequence
  Description of Artificial Sequence Synthetic polynucleotide
ucccaacccccacaacguacgcggcagcguauacuuccacgugagcgaacuccuggcgggggagagccacgcugugcccuaguucaccugauagucgaca

SEQ ID NO: 904 - RNA, 100 nt, Artificial Sequence
  Description of Artificial Sequence Synthetic polynucleotide
ucaauucccccccgccguaccggccagcgauuucuuccuaugagucgaacuccuggcgggggagagccacgcugugauuuaguucaggggauagucgaca

SEQ ID NO: 905 - RNA, 100 nt, Artificial Sequence
  Description of Artificial Sequence Synthetic polynucleotide
ucaauucccccccgccguaccggccagcgauuucuuccuaugagucgaacuccuggcgggggagagccacgcugugauuuaguucaccugauagucgaca

SEQ ID NO: 906 - RNA, 100 nt, Artificial Sequence
  Description of Artificial Sequence Synthetic polynucleotide
uccuguaggccaaccaacacguccguacgugauggggguucacuucgaacuccuggcgggggagagccacgcugcaaccuaguucaaaagauagucgaca

SEQ ID NO: 907 - RNA, 100 nt, Artificial Sequence
  Description of Artificial Sequence Synthetic polynucleotide
uccuguaggccaaccaacacguccguacgugauggggguucacuucgaacuccuggcgggggagagccacgcugcaaccuaguucaccugauagucgaca

SEQ ID NO: 908 - RNA, 100 nt, Artificial Sequence
  Description of Artificial Sequence Synthetic polynucleotide
uccugggacccaacgguuacgucgaugcgugacggaagaucacuucgaacuccuggcgggggagagccacgcugauccccaguucaacggauagucgaca

SEQ ID NO: 909 - RNA, 100 nt, Artificial Sequence
  Description of Artificial Sequence Synthetic polynucleotide
uccugggacccaacgguuacgucgaugcgugacggaagaucacuucgaacuccuggcgggggagagccacgcugauccccaguucaccugauagucgaca

SEQ ID NO: 910 - RNA, 100 nt, Artificial Sequence
  Description of Artificial Sequence Synthetic polynucleotide
uccuaggccccaagaaguacgugagagcguguaggggcaucacuucgaacuccuggcgggggagagccacgcuguauguuaguucaacggauagucgaca

SEQ ID NO: 911 - RNA, 100 nt, Artificial Sequence
  Description of Artificial Sequence Synthetic polynucleotide
uccuaggccccaagaaguacgugagagcguguaggggcaucacuucgaacuccuggcgggggagagccacgcuguauguuaguucaccugauagucgaca

SEQ ID NO: 912 - RNA, 100 nt, Artificial Sequence
  Description of Artificial Sequence Synthetic polynucleotide
uccauccccccauagcguacggggcagcguuucaagccaucacuucgaacuccuggcgggggagagccacgcugacaccuaguucaggagauagucgaca

SEQ ID NO: 913 - RNA, 100 nt, Artificial Sequence
  Description of Artificial Sequence Synthetic polynucleotide
uccauccccccauagcguacggggcagcguuucaagccaucacuucgaacuccuggcgggggagagccacgcugacaccuaguucaccugauagucgaca

SEQ ID NO: 914 - RNA, 100 nt, Artificial Sequence
  Description of Artificial Sequence Synthetic polynucleotide
ucggauccccccugccguacuggccagcgguaauguccaucacuucgaacuccuggcgggggagagccacgcugacgacuaguucaccugauagucgaca

SEQ ID NO: 915 - RNA, 100 nt, Artificial Sequence
  Description of Artificial Sequence Synthetic polynucleotide
uccuguccgggaacccgguagucccaaaucucucuuccaucacuucgaacuccuggcgggggagagccacgcuguacaccaguucagaagauagucgaca

SEQ ID NO: 916 - RNA, 100 nt, Artificial Sequence
  Description of Artificial Sequence Synthetic polynucleotide
uccuguccgggaacccgguagucccaaaucucucuuccaucacuucgaacuccuggcgggggagagccacgcuguacaccaguucaccugauagucgaca

SEQ ID NO: 917 - RNA, 100 nt, Artificial Sequence
  Description of Artificial Sequence Synthetic polynucleotide
uccugucagacaacccauccgucccuaauuuaucuuccaucacuucgaacuccuggcgggggagagccacgcugcccgucaguucaagagauagucgaca

SEQ ID NO: 918 - RNA, 100 nt, Artificial Sequence
  Description of Artificial Sequence Synthetic polynucleotide
uccugucagacaacccauccgucccuaauuuaucuuccaucacuucgaacuccuggcgggggagagccacgcugcccgucaguucaccugauagucgaca

SEQ ID NO: 919 - RNA, 100 nt, Artificial Sequence
  Description of Artificial Sequence Synthetic polynucleotide
uccugucgggcaacccaaccgucccucggugaucccacaucacaugugucuccuggcgggggagagccacgcuggaaguuuguucaccugauagucgaca

SEQ ID NO: 920 - RNA, 100 nt, Artificial Sequence
  Description of Artificial Sequence Synthetic polynucleotide
uccuguaggccaaccgcaacguccguucgugauggaccaucaacaguuacuccuggcgggggagagccacgcugggugcucguucaccugauagucgaca

SEQ ID NO: 921 - RNA, 100 nt, Artificial Sequence
  Description of Artificial Sequence Synthetic polynucleotide
uccugaagcccaacgaauacgucgaugcgugacaguccaucuuuaacaacuccuggcgggggagagccacgcugggagaccguucaccugauagucgaca

SEQ ID NO: 922 - RNA, 100 nt, Artificial Sequence
  Description of Artificial Sequence Synthetic polynucleotide
uccuaggccccaagggguacgugagagcguguaguuccauagugaggaacuccuggcgggggagagccacgcuggcugccuguucaccugauagucgaca

SEQ ID NO: 923 - RNA, 100 nt, Artificial Sequence
  Description of Artificial Sequence Synthetic polynucleotide
uccauccccccauggcguacggagcagcguucccuuccaauguagcgaacuccuggcgggggagagccacgcuggccaccuguucacggcauagucgaca

SEQ ID NO: 924 - RNA, 100 nt, Artificial Sequence
  Description of Artificial Sequence Synthetic polynucleotide
uccauccccccauggcguacggagcagcguucccuuccaauguagcgaacuccuggcgggggagagccacgcuggccaccuguucaccugauagucgaca

SEQ ID NO: 925 - RNA, 100 nt, Artificial Sequence
  Description of Artificial Sequence Synthetic polynucleotide
ucaacuccccccuaccguacccaccagcgccuucuucccagugaucgaacuccuggcgggggagagccacgcuggccauauguucacaccauagucgaca

SEQ ID NO: 926 - RNA, 100 nt, Artificial Sequence
  Description of Artificial Sequence Synthetic polynucleotide
ucaacuccccccuaccguacccaccagcgccuucuucccagugaucgaacuccuggcgggggagagccacgcuggccauauguucaccugauagucgaca

SEQ ID NO: 927 - RNA, 100 nt, Artificial Sequence
  Description of Artificial Sequence Synthetic polynucleotide
uccugucccaguacccgucucucccagaaggaucuugauguucuucgaacuccuggcgggggagagccacgcuggacccucguucacauaauagucgaca

SEQ ID NO: 928 - RNA, 100 nt, Artificial Sequence
  Description of Artificial Sequence Synthetic polynucleotide
uccugucccaguacccgucucucccagaaggaucuugauguucuucgaacuccuggcgggggagagccacgcuggacccucguucaccugauagucgaca

SEQ ID NO: 929 - RNA, 100 nt, Artificial Sequence
  Description of Artificial Sequence Synthetic polynucleotide
uccuguagaccaaccgcaacguccguacgugauauuaguucacuucgaacuccuggcgggggagagccacgcuggccggccguucacgacauagucgaca

SEQ ID NO: 930 - RNA, 100 nt, Artificial Sequence
  Description of Artificial Sequence Synthetic polynucleotide
uccuguagaccaaccgcaacguccguacgugauauuaguucacuucgaacuccuggcgggggagagccacgcuggccggccguucaccugauagucgaca

SEQ ID NO: 931 - RNA, 100 nt, Artificial Sequence
  Description of Artificial Sequence Synthetic polynucleotide
uccugaagcccaacagauacgucgacgcgugagaccagaucacuucgaacuccuggcgggggagagccacgcuggacauuuguucacgaaauagucgaca

SEQ ID NO: 932 - RNA, 100 nt, Artificial Sequence
  Description of Artificial Sequence Synthetic polynucleotide
uccugaagcccaacagauacgucgacgcgugagaccagaucacuucgaacuccuggcgggggagagccacgcuggacauuuguucaccugauagucgaca

SEQ ID NO: 933 - RNA, 100 nt, Artificial Sequence
  Description of Artificial Sequence Synthetic polynucleotide
uccuuaaccccaagagguacguagaagcgugcggccgcaucacuucgaacuccuggcgggggagagccacgcuggaguaccguucacaucauagucgaca

SEQ ID NO: 934 - RNA, 100 nt, Artificial Sequence
  Description of Artificial Sequence Synthetic polynucleotide
uccuuaaccccaagagguacguagaagcgugcggccgcaucacuucgaacuccuggcgggggagagccacgcuggaguaccguucaccugauagucgaca

SEQ ID NO: 935 - RNA, 100 nt, Artificial Sequence
  Description of Artificial Sequence Synthetic polynucleotide
uccacccccccacagcguacggcacagcguccuaccccaucacuucgaacuccuggcgggggagagccacgcuggcguuuuguucacgguauagucgaca

SEQ ID NO: 936 - RNA, 100 nt, Artificial Sequence
  Description of Artificial Sequence Synthetic polynucleotide
uccacccccccacagcguacggcacagcguccuaccccaucacuucgaacuccuggcgggggagagccacgcuggcguuuuguucaccugauagucgaca

SEQ ID NO: 937 - RNA, 100 nt, Artificial Sequence
  Description of Artificial Sequence Synthetic polynucleotide
ucaaaucccccccgccguacuggccagcggccaaguccaucacuucgaacuccuggcgggggagagccacgcuggagggguguucacgacauagucgaca

SEQ ID NO: 938 - RNA, 100 nt, Artificial Sequence
  Description of Artificial Sequence Synthetic polynucleotide
ucaaaucccccccgccguacuggccagcggccaaguccaucacuucgaacuccuggcgggggagagccacgcuggagggguguucaccugauagucgaca

SEQ ID NO: 939 - RNA, 100 nt, Artificial Sequence
  Description of Artificial Sequence Synthetic polynucleotide
uccugucccgauacccguuaaucccagaugcagcuuccaucacuucgaacuccuggcgggggagagccacgcugggaugccguucacaauauagucgaca

SEQ ID NO: 940 - RNA, 100 nt, Artificial Sequence
  Description of Artificial Sequence Synthetic polynucleotide
uccugucccgauacccguuaaucccagaugcagcuuccaucacuucgaacuccuggcgggggagagccacgcugggaugccguucaccugauagucgaca

SEQ ID NO: 941 - RNA, 100 nt, Artificial Sequence
  Description of Artificial Sequence Synthetic polynucleotide
uccuguccgagaacccgguggucccaugacuuucuuccaucacuucgaacuccuggcgggggagagccacgcuggacacacguucacaguauagucgaca

SEQ ID NO: 942 - RNA, 100 nt, Artificial Sequence
  Description of Artificial Sequence Synthetic polynucleotide
uccuguccgagaacccgguggucccaugacuuucuuccaucacuucgaacuccuggcgggggagagccacgcuggacacacguucaccugauagucgaca

SEQ ID NO: 943 - RNA, 100 nt, Artificial Sequence
  Description of Artificial Sequence Synthetic polynucleotide
uccugucagacaacccuuucguccccugaauaucuuccaucacuucgaacuccuggcgggggagagccacgcuggggaugcguucacggcauagucgaca

SEQ ID NO: 944 - RNA, 100 nt, Artificial Sequence
  Description of Artificial Sequence Synthetic polynucleotide
uccugucagacaacccuuucguccccugaauaucuuccaucacuucgaacuccuggcgggggagagccacgcuggggaugcguucaccugauagucgaca

SEQ ID NO: 945 - RNA, 100 nt, Artificial Sequence
  Description of Artificial Sequence Synthetic polynucleotide
uccuguaggccaaccgccacguccguucgugauagaccaucaauauauacuccuggcgggggagagccacgcugguauuccuuucaccugauagucgaca

SEQ ID NO: 946 - RNA, 100 nt, Artificial Sequence
  Description of Artificial Sequence Synthetic polynucleotide
uccuggagcccaacgacuacgucagugcgugaggauccaucuuccuaaacuccuggcgggggagagccacgcugguguacgauucaccugauagucgaca

SEQ ID NO: 947 - RNA, 100 nt, Artificial Sequence
  Description of Artificial Sequence Synthetic polynucleotide
uccuuagccccaaaggguacgugggagcgugcgguuccaugugacagaacuccuggcgggggagagccacgcuggugugguauucaccugauagucgaca

SEQ ID NO: 948 - RNA, 100 nt, Artificial Sequence
  Description of Artificial Sequence Synthetic polynucleotide
uccgggcccccacagcguacgagacagcguaugcuuccagauucacgaacuccuggcgggggagagccacgcuggugaugcuuucaccugauagucgaca

SEQ ID NO: 949 - RNA, 100 nt, Artificial Sequence
  Description of Artificial Sequence Synthetic polynucleotide
ucgacuccccccuaccguacauaccagcggcuucuuccccuguaucgaacuccuggcgggggagagccacgcugguguaccauucaccugauagucgaca

SEQ ID NO: 950 - RNA, 100 nt, Artificial Sequence
  Description of Artificial Sequence Synthetic polynucleotide
uccugucccagcacccgucaaucccagaccgaucuuauugaucuucgaacuccuggcgggggagagccacgcugguaguuuuuucacccccuagucgaca

SEQ ID NO: 951 - RNA, 100 nt, Artificial Sequence
  Description of Artificial Sequence Synthetic polynucleotide
uccugucccagcacccgucaaucccagaccgaucuuauugaucuucgaacuccuggcgggggagagccacgcugguaguuuuuucaccugauagucgaca

SEQ ID NO: 952 - RNA, 100 nt, Artificial Sequence
  Description of Artificial Sequence Synthetic polynucleotide
uccuguccaagaacccgauggucccaaaaugaucuagaggaacuucgaacuccuggcgggggagagccacgcugguagguuuuucaccgguuagucgaca

SEQ ID NO: 953 - RNA, 100 nt, Artificial Sequence
  Description of Artificial Sequence Synthetic polynucleotide
uccuguccaagaacccgauggucccaaaaugaucuagaggaacuucgaacuccuggcgggggagagccacgcugguagguuuuucaccugauagucgaca

SEQ ID NO: 954 - RNA, 100 nt, Artificial Sequence
  Description of Artificial Sequence Synthetic polynucleotide
uccugucacacaacccuaucguccccaagugaucgggggccacuucgaacuccuggcgggggagagccacgcugguauucuuuucaccgguuagucgaca

SEQ ID NO: 955 - RNA, 100 nt, Artificial Sequence
  Description of Artificial Sequence Synthetic polynucleotide
uccugucacacaacccuaucguccccaagugaucgggggccacuucgaacuccuggcgggggagagccacgcugguauucuuuucaccugauagucgaca

SEQ ID NO: 956 - RNA, 100 nt, Artificial Sequence
  Description of Artificial Sequence Synthetic polynucleotide
uccuguacaccaaccaccacguccacccgugauaagaauucacuucgaacuccuggcgggggagagccacgcugguagcuacuucaccgcuuagucgaca

SEQ ID NO: 957 - RNA, 100 nt, Artificial Sequence
  Description of Artificial Sequence Synthetic polynucleotide
uccuguacaccaaccaccacguccacccgugauaagaauucacuucgaacuccuggcgggggagagccacgcugguagcuacuucaccugauagucgaca

SEQ ID NO: 958 - RNA, 100 nt, Artificial Sequence
  Description of Artificial Sequence Synthetic polynucleotide
uccugagacccaacgguuacgucagugcgugagcagagaucacuucgaacuccuggcgggggagagccacgcugguaaacacuucaccaucuagucgaca

SEQ ID NO: 959 - RNA, 100 nt, Artificial Sequence
  Description of Artificial Sequence Synthetic polynucleotide
uccugagacccaacgguuacgucagugcgugagcagagaucacuucgaacuccuggcgggggagagccacgcugguaaacacuucaccugauagucgaca

SEQ ID NO: 960 - RNA, 100 nt, Artificial Sequence
  Description of Artificial Sequence Synthetic polynucleotide
uccuuggccccaagagguacguggaagcgugcggcgacaucacuucgaacuccuggcgggggagagccacgcugguaacccuuucaccgucuagucgaca

SEQ ID NO: 961 - RNA, 100 nt, Artificial Sequence
  Description of Artificial Sequence Synthetic polynucleotide
uccuuggccccaagagguacguggaagcgugcggcgacaucacuucgaacuccuggcgggggagagccacgcugguaacccuuucaccugauagucgaca

SEQ ID NO: 962 - RNA, 100 nt, Artificial Sequence
  Description of Artificial Sequence Synthetic polynucleotide
ucccagcccccauggcguacgcaacagcguuuagcaccaucacuucgaacuccuggcgggggagagccacgcugguagccauuucacccuuuagucgaca

SEQ ID NO: 963 - RNA, 100 nt, Artificial Sequence
  Description of Artificial Sequence Synthetic polynucleotide
ucccagcccccauggcguacgcaacagcguuuagcaccaucacuucgaacuccuggcgggggagagccacgcugguagccauuucaccugauagucgaca

SEQ ID NO: 964 - RNA, 100 nt, Artificial Sequence
  Description of Artificial Sequence Synthetic polynucleotide
uccugucccgacacccguuaaucccaggaucuccuuccaucacuucgaacuccuggcgggggagagccacgcugguguccucuucaccgguuagucgaca

SEQ ID NO: 965 - RNA, 100 nt, Artificial Sequence
  Description of Artificial Sequence Synthetic polynucleotide
uccugucccgacacccguuaaucccaggaucuccuuccaucacuucgaacuccuggcgggggagagccacgcugguguccucuucaccugauagucgaca

SEQ ID NO: 966 - RNA, 100 nt, Artificial Sequence
  Description of Artificial Sequence Synthetic polynucleotide
uccuguccaagaacccgccagucccaagacccucuuccaucacuucgaacuccuggcgggggagagccacgcugguagguucuucaccaacuagucgaca

SEQ ID NO: 967 - RNA, 100 nt, Artificial Sequence
  Description of Artificial Sequence Synthetic polynucleotide
uccuguccaagaacccgccagucccaagacccucuuccaucacuucgaacuccuggcgggggagagccacgcugguagguucuucaccugauagucgaca

SEQ ID NO: 968 - RNA, 100 nt, Artificial Sequence
  Description of Artificial Sequence Synthetic polynucleotide
uccugucaagcaacccacccgucccagcgugaucuuccaucacuucgaacuccuggcgggggagagccacgcugguaguaccuucaccaauuagucgaca

SEQ ID NO: 969 - RNA, 100 nt, Artificial Sequence
  Description of Artificial Sequence Synthetic polynucleotide
uccugucaagcaacccacccgucccagcgugaucuuccaucacuucgaacuccuggcgggggagagccacgcugguaguaccuucaccugauagucgaca

SEQ ID NO: 970 - RNA, 100 nt, Artificial Sequence
  Description of Artificial Sequence Synthetic polynucleotide
uccuguaggccaaccaccacguccguacgugaugcgccaucagcaguuacuccuggcgggggagagccacgcugguugcucuaucaccugauagucgaca

SEQ ID NO: 971 - RNA, 100 nt, Artificial Sequence
  Description of Artificial Sequence Synthetic polynucleotide
uccugacacccaacgaauacgucgacgcgugacgauccaucuaccaaaacuccuggcgggggagagccacgcugguuguuaacucaccugauagucgaca

SEQ ID NO: 972 - RNA, 100 nt, Artificial Sequence
  Description of Artificial Sequence Synthetic polynucleotide
uccucgaccccaagaaguacguggaagcgugcgauuccaugucaaggaacuccuggcgggggagagccacgcugguugugcacucaccugauagucgaca

SEQ ID NO: 973 - RNA, 100 nt, Artificial Sequence
  Description of Artificial Sequence Synthetic polynucleotide
uccaaccccccacggcguacggggcagcgucuacuuccacgugcgcgaacuccuggcgggggagagccacgcugguuaaccgcucaccuaugagucgaca

SEQ ID NO: 974 - RNA, 100 nt, Artificial Sequence
  Description of Artificial Sequence Synthetic polynucleotide
uccaaccccccacggcguacggggcagcgucuacuuccacgugcgcgaacuccuggcgggggagagccacgcugguuaaccgcucaccugauagucgaca

SEQ ID NO: 975 - RNA, 100 nt, Artificial Sequence
  Description of Artificial Sequence Synthetic polynucleotide
ucggaucccccuugccguacccgccagcgaucucuuccuuugugucgaacuccuggcgggggagagccacgcugguuaacuugucaccugauagucgaca

SEQ ID NO: 976 - RNA, 100 nt, Artificial Sequence
  Description of Artificial Sequence Synthetic polynucleotide
uccugucccaauacccgucaaucccagaaggaucuuaugaaccuucgaacuccuggcgggggagagccacgcugguugccgacucaccucaaagucgaca

SEQ ID NO: 977 - RNA, 100 nt, Artificial Sequence
  Description of Artificial Sequence Synthetic polynucleotide
uccugucccaauacccgucaaucccagaaggaucuuaugaaccuucgaacuccuggcgggggagagccacgcugguugccgacucaccugauagucgaca

SEQ ID NO: 978 - RNA, 100 nt, Artificial Sequence
  Description of Artificial Sequence Synthetic polynucleotide
uccuguccgaaaacccgguagucccauaaugaucucagcuaacuucgaacuccuggcgggggagagccacgcugguuguagacucaccuucgagucgaca

SEQ ID NO: 979 - RNA, 100 nt, Artificial Sequence
  Description of Artificial Sequence Synthetic polynucleotide
uccuguccgaaaacccgguagucccauaaugaucucagcuaacuucgaacuccuggcgggggagagccacgcugguuguagacucaccugauagucgaca

SEQ ID NO: 980 - RNA, 100 nt, Artificial Sequence
  Description of Artificial Sequence Synthetic polynucleotide
uccugucgggcaacccaaucguccccuagugauccggaaccacuucgaacuccuggcgggggagagccacgcugguugcuuugucaccuaccagucgaca

SEQ ID NO: 981 - RNA, 100 nt, Artificial Sequence
  Description of Artificial Sequence Synthetic polynucleotide
uccugucgggcaacccaaucguccccuagugauccggaaccacuucgaacuccuggcgggggagagccacgcugguugcuuugucaccugauagucgaca

SEQ ID NO: 982 - RNA, 100 nt, Artificial Sequence
  Description of Artificial Sequence Synthetic polynucleotide
uccuguaggccaaccgccacguccgcccgugaugccuuuucacuucgaacuccuggcgggggagagccacgcugguugccccgucaccuuuaagucgaca

SEQ ID NO: 983 - RNA, 100 nt, Artificial Sequence
  Description of Artificial Sequence Synthetic polynucleotide
uccuguaggccaaccgccacguccgcccgugaugccuuuucacuucgaacuccuggcgggggagagccacgcugguugccccgucaccugauagucgaca

SEQ ID NO: 984 - RNA, 100 nt, Artificial Sequence
  Description of Artificial Sequence Synthetic polynucleotide
uccugcaacccaacgaauacgucagcgcgugaggcacaaucacuucgaacuccuggcgggggagagccacgcugguuaucuaaucaccuaacagucgaca

SEQ ID NO: 985 - RNA, 100 nt, Artificial Sequence
  Description of Artificial Sequence Synthetic polynucleotide
uccugcaacccaacgaauacgucagcgcgugaggcacaaucacuucgaacuccuggcgggggagagccacgcugguuaucuaaucaccugauagucgaca

SEQ ID NO: 986 - RNA, 100 nt, Artificial Sequence
  Description of Artificial Sequence Synthetic polynucleotide
uccugucccccaacccguacgucccagcguguaaacacaucacuucgaacuccuggcgggggagagccacgcugguuguucggucaccugauagucgaca

SEQ ID NO: 987 - RNA, 100 nt, Artificial Sequence
  Description of Artificial Sequence Synthetic polynucleotide
uccuuuaccccaagaaguacguagaagcguguaaacacaucacuucgaacuccuggcgggggagagccacgcugguuguucggucaccuccgagucgaca

SEQ ID NO: 988 - RNA, 100 nt, Artificial Sequence
  Description of Artificial Sequence Synthetic polynucleotide
uccuuuaccccaagaaguacguagaagcguguaaacacaucacuucgaacuccuggcgggggagagccacgcugguuguucggucaccugauagucgaca

SEQ ID NO: 989 - RNA, 100 nt, Artificial Sequence
  Description of Artificial Sequence Synthetic polynucleotide
uccacacccccaugacguacggcacagcguacacaaccaucacuucgaacuccuggcgggggagagccacgcugguuacgcgcucaccuaaaagucgaca

SEQ ID NO: 990 - RNA, 100 nt, Artificial Sequence
  Description of Artificial Sequence Synthetic polynucleotide
uccacacccccaugacguacggcacagcguacacaaccaucacuucgaacuccuggcgggggagagccacgcugguuacgcgcucaccugauagucgaca

SEQ ID NO: 991 - RNA, 100 nt, Artificial Sequence
  Description of Artificial Sequence Synthetic polynucleotide
uccugucccagcacccguuauucccagaagucccuuccaucacuucgaacuccuggcgggggagagccacgcugguuauucggucaccucugagucgaca

SEQ ID NO: 992 - RNA, 100 nt, Artificial Sequence
  Description of Artificial Sequence Synthetic polynucleotide
uccugucccagcacccguuauucccagaagucccuuccaucacuucgaacuccuggcgggggagagccacgcugguuauucggucaccugauagucgaca

SEQ ID NO: 993 - RNA, 100 nt, Artificial Sequence
  Description of Artificial Sequence Synthetic polynucleotide
uccuguccggaaacccgccggucccauaaucuucuuccaucacuucgaacuccuggcgggggagagccacgcugguugccaacucaccuaagagucgaca

SEQ ID NO: 994 - RNA, 100 nt, Artificial Sequence
  Description of Artificial Sequence Synthetic polynucleotide
uccuguccggaaacccgccggucccauaaucuucuuccaucacuucgaacuccuggcgggggagagccacgcugguugccaacucaccugauagucgaca

SEQ ID NO: 995 - RNA, 100 nt, Artificial Sequence
  Description of Artificial Sequence Synthetic polynucleotide
uccugucaaacaacccauucgucccccauuuaucuuccaucacuucgaacuccuggcgggggagagccacgcugguugccaugucaccuccaagucgaca

SEQ ID NO: 996 - RNA, 100 nt, Artificial Sequence
  Description of Artificial Sequence Synthetic polynucleotide
uccugucaaacaacccauucgucccccauuuaucuuccaucacuucgaacuccuggcgggggagagccacgcugguugccaugucaccugauagucgaca

SEQ ID NO: 997 - RNA, 100 nt, Artificial Sequence
  Description of Artificial Sequence Synthetic polynucleotide
uccugucggacaacccuaucgucccagcgugaucuuccaucacuucgaacuccuggcgggggagagccacgcugguuagcacgucaccucaaagucgaca

SEQ ID NO: 998 - RNA, 100 nt, Artificial Sequence
  Description of Artificial Sequence Synthetic polynucleotide
uccuuggccccaaaggguacgugagagcguguaauuccauaguauagaacuccuggcgggggagagccacgcugguucuccccacaccugauagucgaca

SEQ ID NO: 999 - RNA, 100 nt, Artificial Sequence
  Description of Artificial Sequence Synthetic polynucleotide
uccauccccccauggcguacgaggcagcguacgcuuccaaaaagccgaacuccuggcgggggagagccacgcugguucccccggcaccugauagucgaca

SEQ ID NO: 1000 - RNA, 100 nt, Artificial Sequence
  Description of Artificial Sequence Synthetic polynucleotide
ucagaucccccucgccguacaaaccagcgcucucuucccaaguaucgaacuccuggcgggggagagccacgcugguuccaaucgcaccugcacgucgaca

SEQ ID NO: 1001 - RNA, 100 nt, Artificial Sequence
  Description of Artificial Sequence Synthetic polynucleotide
ucagaucccccucgccguacaaaccagcgcucucuucccaaguaucgaacuccuggcgggggagagccacgcugguuccaaucgcaccugauagucgaca

SEQ ID NO: 1002 - RNA, 100 nt, Artificial Sequence
  Description of Artificial Sequence Synthetic polynucleotide
uccugucccgacacccguuaaucccagaacgaucuuaauagucuucgaacuccuggcgggggagagccacgcugguucccauaacaccuguuugucgaca

SEQ ID NO: 1003 - RNA, 100 nt, Artificial Sequence
  Description of Artificial Sequence Synthetic polynucleotide
uccugucccgacacccguuaaucccagaacgaucuuaauagucuucgaacuccuggcgggggagagccacgcugguucccauaacaccugauagucgaca

SEQ ID NO: 1004 - RNA, 100 nt, Artificial Sequence
  Description of Artificial Sequence Synthetic polynucleotide
uccuguccgggaacccgcuggucccacaaugaucuagucggacuucgaacuccuggcgggggagagccacgcugguuccucuagcaccuguaugucgaca

SEQ ID NO: 1005 - RNA, 100 nt, Artificial Sequence
  Description of Artificial Sequence Synthetic polynucleotide
uccuguccgggaacccgcuggucccacaaugaucuagucggacuucgaacuccuggcgggggagagccacgcugguuccucuagcaccugauagucgaca

SEQ ID NO: 1006 - RNA, 100 nt, Artificial Sequence
  Description of Artificial Sequence Synthetic polynucleotide
uccugucgaacaaccccgccgucccuuagugaucgcugaccacuucgaacuccuggcgggggagagccacgcugguucuuacuccaccugucugucgaca

SEQ ID NO: 1007 - RNA, 100 nt, Artificial Sequence
  Description of Artificial Sequence Synthetic polynucleotide
uccugucgaacaaccccgccgucccuuagugaucgcugaccacuucgaacuccuggcgggggagagccacgcugguucuuacuccaccugauagucgaca

SEQ ID NO: 1008 - RNA, 100 nt, Artificial Sequence
  Description of Artificial Sequence Synthetic polynucleotide
uccuguaggccaaccacaacguccgcccgugauaacuuuucacuucgaacuccuggcgggggagagccacgcugguuccuguaccaccugcucgucgaca

SEQ ID NO: 1009 - RNA, 100 nt, Artificial Sequence
  Description of Artificial Sequence Synthetic polynucleotide
uccuguaggccaaccacaacguccgcccgugauaacuuuucacuucgaacuccuggcgggggagagccacgcugguuccuguaccaccugauagucgaca

SEQ ID NO: 1010 - RNA, 100 nt, Artificial Sequence
  Description of Artificial Sequence Synthetic polynucleotide
uccugguacccaacgguuacgucggugcgugaggugcaaucacuucgaacuccuggcgggggagagccacgcugguucucacuccaccuguucgucgaca

SEQ ID NO: 1011 - RNA, 100 nt, Artificial Sequence
  Description of Artificial Sequence Synthetic polynucleotide
uccugguacccaacgguuacgucggugcgugaggugcaaucacuucgaacuccuggcgggggagagccacgcugguucucacuccaccugauagucgaca

SEQ ID NO: 1012 - RNA, 100 nt, Artificial Sequence
  Description of Artificial Sequence Synthetic polynucleotide
uccuuagccccaaaaaguacgugaaagcguguagaagcaucacuucgaacuccuggcgggggagagccacgcugguucuuucaacaccuguaugucgaca

SEQ ID NO: 1013 - RNA, 100 nt, Artificial Sequence
  Description of Artificial Sequence Synthetic polynucleotide
uccuuagccccaaaaaguacgugaaagcguguagaagcaucacuucgaacuccuggcgggggagagccacgcugguucuuucaacaccugauagucgaca

SEQ ID NO: 1014 - RNA, 100 nt, Artificial Sequence
  Description of Artificial Sequence Synthetic polynucleotide
uccccgcccccauggcguacgcgacagcgucccucaccaucacuucgaacuccuggcgggggagagccacgcugguuccuauuacaccugucugucgaca

SEQ ID NO: 1015 - RNA, 100 nt, Artificial Sequence
  Description of Artificial Sequence Synthetic polynucleotide
uccccgcccccauggcguacgcgacagcgucccucaccaucacuucgaacuccuggcgggggagagccacgcugguuccuauuacaccugauagucgaca

SEQ ID NO: 1016 - RNA, 100 nt, Artificial Sequence
  Description of Artificial Sequence Synthetic polynucleotide
ucgauucccccucgccguacagaccagcgcuuguauccaucacuucgaacuccuggcgggggagagccacgcugguuccuuugccaccuguuugucgaca

SEQ ID NO: 1017 - RNA, 100 nt, Artificial Sequence
  Description of Artificial Sequence Synthetic polynucleotide
uccugucccaacacccgucaaucccagaaguagcuuccaucacuucgaacuccuggcgggggagagccacgcugguuccuauaacaccugcuugucgaca

SEQ ID NO: 1018 - RNA, 100 nt, Artificial Sequence
  Description of Artificial Sequence Synthetic polynucleotide
uccugucccaacacccgucaaucccagaaguagcuuccaucacuucgaacuccuggcgggggagagccacgcugguuccuauaacaccugauagucgaca

SEQ ID NO: 1019 - RNA, 100 nt, Artificial Sequence
  Description of Artificial Sequence Synthetic polynucleotide
uccuguccacaaacccgaaagucccaagacccucuuccaucacuucgaacuccuggcgggggagagccacgcugguucccacuacaccugcccgucgaca

SEQ ID NO: 1020 - RNA, 100 nt, Artificial Sequence
  Description of Artificial Sequence Synthetic polynucleotide
uccuguccacaaacccgaaagucccaagacccucuuccaucacuucgaacuccuggcgggggagagccacgcugguucccacuacaccugauagucgaca

SEQ ID NO: 1021 - RNA, 100 nt, Artificial Sequence
  Description of Artificial Sequence Synthetic polynucleotide
uccugucgagcaaccccuucguccccuauauaucuuccaucacuucgaacuccuggcgggggagagccacgcugguuccucacacaccuguuugucgaca

SEQ ID NO: 1022 - RNA, 100 nt, Artificial Sequence
  Description of Artificial Sequence Synthetic polynucleotide
uccugucgagcaaccccuucguccccuauauaucuuccaucacuucgaacuccuggcgggggagagccacgcugguuccucacacaccugauagucgaca

SEQ ID NO: 1023 - RNA, 100 nt, Artificial Sequence
  Description of Artificial Sequence Synthetic polynucleotide
uccugucccccaacccguacgucccagcgugaucuuccaucacucaaauauccuggcgggggagagccacgcugguucaaaguucaccugauagucgaca

SEQ ID NO: 1024 - RNA, 100 nt, Artificial Sequence
  Description of Artificial Sequence Synthetic polynucleotide
uccuguccacaaacccggcggucccauacugaucugggaucacucaaauauccuggcgggggagagccacgcugguucaaaguucaccugauagucgaca

SEQ ID NO: 1025 - RNA, 100 nt, Artificial Sequence
  Description of Artificial Sequence Synthetic polynucleotide
uccugucaaacaacccuuccgucccuuggugauccggcaucacguaccucuccuggcgggggagagccacgcugguucaaaguucaccugauagucgaca

SEQ ID NO: 1026 - RNA, 100 nt, Artificial Sequence
  Description of Artificial Sequence Synthetic polynucleotide
uccugugagccaaccgagacguccgcucgugaugccccaucaaauuacacuccuggcgggggagagccacgcugguucaaaguucaccugauagucgaca

SEQ ID NO: 1027 - RNA, 100 nt, Artificial Sequence
  Description of Artificial Sequence Synthetic polynucleotide
uccugaagcccaacaacuacgucgaugcgugacaguccaucucacaaaacuccuggcgggggagagccacgcugguucaaaguucaccugauagucgaca

SEQ ID NO: 1028 - RNA, 100 nt, Artificial Sequence
  Description of Artificial Sequence Synthetic polynucleotide
uccuuggccccaaaaaguacguaggagcgugccguuccauaagggggaacuccuggcgggggagagccacgcugguucaaaguucaccugauagucgaca

SEQ ID NO: 1029 - RNA, 100 nt, Artificial Sequence
  Description of Artificial Sequence Synthetic polynucleotide
uccgcccccccacggcguacgcgacagcguacgcuuccaaaugcgcgaacuccuggcgggggagagccacgcugguucaaaguucaccugauagucgaca

SEQ ID NO: 1030 - RNA, 100 nt, Artificial Sequence
  Description of Artificial Sequence Synthetic polynucleotide
uccugucccgauacccguuuaucccagaaggaucuugacugucuucgaacuccuggcgggggagagccacgcugguucaaaguucaccugauagucgaca

SEQ ID NO: 1031 - RNA, 100 nt, Artificial Sequence
  Description of Artificial Sequence Synthetic polynucleotide
uccuguccaggaacccggcggucccacaaugaucuauguagacuucgaacuccuggcgggggagagccacgcugggcaaaaguugcgcugauaagggaca

SEQ ID NO: 1032 - RNA, 100 nt, Artificial Sequence
  Description of Artificial Sequence Synthetic polynucleotide
uccuguccaggaacccggcggucccacaaugaucuauguagacuucgaacuccuggcgggggagagccacgcugguucaaaguucaccugauagucgaca

SEQ ID NO: 1033 - RNA, 100 nt, Artificial Sequence
  Description of Artificial Sequence Synthetic polynucleotide
uccugucagacaaccccaccguccccuagugaucagguuccacuucgaacuccuggcgggggagagccacgcuggacaaaaguuaaacugauacaagaca

SEQ ID NO: 1034 - RNA, 100 nt, Artificial Sequence
  Description of Artificial Sequence Synthetic polynucleotide
uccugucagacaaccccaccguccccuagugaucagguuccacuucgaacuccuggcgggggagagccacgcugguucaaaguucaccugauagucgaca

SEQ ID NO: 1035 - RNA, 100 nt, Artificial Sequence
  Description of Artificial Sequence Synthetic polynucleotide
uccuguaagccaaccaagacguccauucgugaugacauuucacuucgaacuccuggcgggggagagccacgcuggacgaaaguuaaacugauacaggaca

SEQ ID NO: 1036 - RNA, 100 nt, Artificial Sequence
  Description of Artificial Sequence Synthetic polynucleotide
uccuguaagccaaccaagacguccauucgugaugacauuucacuucgaacuccuggcgggggagagccacgcugguucaaaguucaccugauagucgaca

SEQ ID NO: 1037 - RNA, 100 nt, Artificial Sequence
  Description of Artificial Sequence Synthetic polynucleotide
uccugcgacccaacgguuacgucgacgcgugaguagagaucacuucgaacuccuggcgggggagagccacgcuggaagaaaguuauacugauaagggaca

SEQ ID NO: 1038 - RNA, 100 nt, Artificial Sequence
  Description of Artificial Sequence Synthetic polynucleotide
uccugcgacccaacgguuacgucgacgcgugaguagagaucacuucgaacuccuggcgggggagagccacgcugguucaaaguucaccugauagucgaca

SEQ ID NO: 1039 - RNA, 100 nt, Artificial Sequence
  Description of Artificial Sequence Synthetic polynucleotide
uccuauaccccaagaaguacguaaaagcgugccaccacaucacuucgaacuccuggcgggggagagccacgcuggguaaaaguugugcugauauuagaca

SEQ ID NO: 1040 - RNA, 100 nt, Artificial Sequence
  Description of Artificial Sequence Synthetic polynucleotide
uccuauaccccaagaaguacguaaaagcgugccaccacaucacuucgaacuccuggcgggggagagccacgcugguucaaaguucaccugauagucgaca

SEQ ID NO: 1041 - RNA, 100 nt, Artificial Sequence
  Description of Artificial Sequence Synthetic polynucleotide
uccauacccccauagcguacggcacagcguccugggccaucacuucgaacuccuggcgggggagagccacgcuggauaaaaguugcacugauaugggaca

SEQ ID NO: 1042 - RNA, 100 nt, Artificial Sequence
  Description of Artificial Sequence Synthetic polynucleotide
uccauacccccauagcguacggcacagcguccugggccaucacuucgaacuccuggcgggggagagccacgcugguucaaaguucaccugauagucgaca

SEQ ID NO: 1043 - RNA, 100 nt, Artificial Sequence
  Description of Artificial Sequence Synthetic polynucleotide
uccugucccgguacccgucaaucccagacuccccuuccaucacuucgaacuccuggcgggggagagccacgcugggaaaaaguuguacugauauaagaca

SEQ ID NO: 1044 - RNA, 100 nt, Artificial Sequence
  Description of Artificial Sequence Synthetic polynucleotide
uccuguccaagaacccggcggucccacccaccucuuccaucacuucgaacuccuggcgggggagagccacgcuggcagaaaguugugcugauaagggaca

SEQ ID NO: 1045 - RNA, 100 nt, Artificial Sequence
  Description of Artificial Sequence Synthetic polynucleotide
uccuguccaagaacccggcggucccacccaccucuuccaucacuucgaacuccuggcgggggagagccacgcugguucaaaguucaccugauagucgaca

SEQ ID NO: 1046 - RNA, 100 nt, Artificial Sequence
  Description of Artificial Sequence Synthetic polynucleotide
uccugucgaacaaccccaccgucccuacucaaucuuccaucacuucgaacuccuggcgggggagagccacgcugggcgaaaguugcgcugauaacagaca

SEQ ID NO: 1047 - RNA, 100 nt, Artificial Sequence
  Description of Artificial Sequence Synthetic polynucleotide
uccugucgaacaaccccaccgucccuacucaaucuuccaucacuucgaacuccuggcgggggagagccacgcugguucaaaguucaccugauagucgaca

SEQ ID NO: 1048 - RNA, 100 nt, Artificial Sequence
  Description of Artificial Sequence Synthetic polynucleotide
uccuguagaccaaccaccacguccacccgugaugacccaucagcguauacuccuggcggggcucagccaugcugguucaacucgauccugauccacgaca

SEQ ID NO: 1049 - RNA, 100 nt, Artificial Sequence
  Description of Artificial Sequence Synthetic polynucleotide
uccugacacccaacagcuacgucgaugcgugaagcuccaucucaguaaacuccuggcggggcucagccaugcugguucaaccuguuccugaucaacgaca

SEQ ID NO: 1050 - RNA, 100 nt, Artificial Sequence
  Description of Artificial Sequence Synthetic polynucleotide
uccugucaggcaacccaaccgucccuuggugaucauacaucaccccuuccuccuggcggggcucagccaugcugguucaaaaauuugcugauagucgaca

SEQ ID NO: 1051 - RNA, 100 nt, Artificial Sequence
  Description of Artificial Sequence Synthetic polynucleotide
uccuguacaccaaccgacacguccguucgugaugggccaucagagaccacuccuggcggggcucagccaugcugguucaaaacaguacugauagucgaca

SEQ ID NO: 1052 - RNA, 100 nt, Artificial Sequence
  Description of Artificial Sequence Synthetic polynucleotide
uccuggaacccaacggcuacgucaaugcgugaggcuccaucuaacauaacuccuggcggggcucagccaugcugguucaaaaagaugcugauaugagaca

SEQ ID NO: 1053 - RNA, 100 nt, Artificial Sequence
  Description of Artificial Sequence Synthetic polynucleotide
uccuggaacccaacggcuacgucaaugcgugaggcuccaucuaacauaacuccuggcggggcucagccaugcugguucaaaaagaugcugauagucgaca

SEQ ID NO: 1054 - RNA, 100 nt, Artificial Sequence
  Description of Artificial Sequence Synthetic polynucleotide
uccuguccgagaacccgaaagucccaaauugaucucgaaucacuccuccguccuggcggggcucagccaugcugguucaaagggaguaugauaggguaca

SEQ ID NO: 1055 - RNA, 100 nt, Artificial Sequence
  Description of Artificial Sequence Synthetic polynucleotide
uccugcaacccaacagauacgucgaugcgugaggauccauccauacuaacuccuggcggggcucagccaugcugguucaaagcguuagugauaggauaca

SEQ ID NO: 1056 - RNA, 100 nt, Artificial Sequence
  Description of Artificial Sequence Synthetic polynucleotide
uccugcaacccaacagauacgucgaugcgugaggauccauccauacuaacuccuggcggggcucagccaugcugguucaaagcguuagugauagucgaca

SEQ ID NO: 1057 - RNA, 100 nt, Artificial Sequence
  Description of Artificial Sequence Synthetic polynucleotide
uccaaacccccacggcguacgacacagcguacgcuuccaaguagacgaacuccuggcggggcucagccaugcugguucaaaggcaaagugauaggguaca

SEQ ID NO: 1058 - RNA, 100 nt, Artificial Sequence
  Description of Artificial Sequence Synthetic polynucleotide
uccaaacccccacggcguacgacacagcguacgcuuccaaguagacgaacuccuggcggggcucagccaugcugguucaaaggcaaagugauagucgaca

SEQ ID NO: 1059 - RNA, 100 nt, Artificial Sequence
  Description of Artificial Sequence Synthetic polynucleotide
uccucggccccaagaaguacgugggagcgugcuaccgcaucacuucgaacuccuggcggggcucagccauaagcucucaaagaagaccugagcuucgaca

SEQ ID NO: 1060 - RNA, 100 nt, Artificial Sequence
  Description of Artificial Sequence Synthetic polynucleotide
ucccagcccccaugacguacgaagcagcguaaccuuccaaccagacgaacuccuggcggggcucagccaugggccuccaaaguaguccugauuuccgaca

SEQ ID NO: 1061 - RNA, 100 nt, Artificial Sequence
  Description of Artificial Sequence Synthetic polynucleotide
ucccagcccccaugacguacgaagcagcguaaccuuccaaccagacgaacuccuggcggggcucagccaugggccuccaaaguucaccugauagucgaca

SEQ ID NO: 1062 - RNA, 100 nt, Artificial Sequence
  Description of Artificial Sequence Synthetic polynucleotide
uccuguccaggaacccggcagucccaagaugaucuagggcgacuucgaacuccuggcggggcucagccaugaucgaccaaagugguccugauccgcgaca

SEQ ID NO: 1063 - RNA, 100 nt, Artificial Sequence
  Description of Artificial Sequence Synthetic polynucleotide
uccuaaaccccaagaaguacgugggagcguguaauuccaugugggagaacuccuggcggggcucagccaugcucgaaguaaguucaccugauagucgaca

SEQ ID NO: 1064 - RNA, 100 nt, Artificial Sequence
  Description of Artificial Sequence Synthetic polynucleotide
uccugaagcccaacgguuacgucagcgcgugacaaaagaucacuucgaacuccuggcggggcucagccaugcuaaccguaaguucaccugauagucgaca

SEQ ID NO: 1065 - RNA, 100 nt, Artificial Sequence
  Description of Artificial Sequence Synthetic polynucleotide
uccugucacacaacccauccgucccucagugauccgacaucaccugucucuccuggcggggcucagccaugcugaccgguaguucagaagauagucgaca

SEQ ID NO: 1066 - RNA, 100 nt, Artificial Sequence
  Description of Artificial Sequence Synthetic polynucleotide
uccuccgccccaagaaguacguagaagcguguuuaggcaucacuucgaacuccuggcggggcucagccaugcugaugguuaguucaccugauagucgaca

SEQ ID NO: 1067 - RNA, 100 nt, Artificial Sequence
  Description of Artificial Sequence Synthetic polynucleotide
uccugucgggcaacccaaucgucccuuagugauccagcaucacaaucuucuccuggcggggcucagccaugcuggcagucuguucacgaaauagucgaca

SEQ ID NO: 1068 - RNA, 100 nt, Artificial Sequence
  Description of Artificial Sequence Synthetic polynucleotide
uccugaagcccaacgacuacgucggcgcgugacacuccaucuaagacaacuccuggcggggcucagccaugcuggagacuuguucacaccauagucgaca

SEQ ID NO: 1069 - RNA, 100 nt, Artificial Sequence
  Description of Artificial Sequence Synthetic polynucleotide
uccugucaggcaacccacccgucccuaagugauccggcaucacgaaucccuccuggcggggcucagccaugcugguuguuuaaucaccuuucagucgaca

SEQ ID NO: 1070 - RNA, 100 nt, Artificial Sequence
  Description of Artificial Sequence Synthetic polynucleotide
uccugucaggcaacccacccgucccuaagugauccggcaucacgaaucccuccuggcggggcucagccaugcugguuguuuaaucaccugauagucgaca

SEQ ID NO: 1071 - RNA, 100 nt, Artificial Sequence
  Description of Artificial Sequence Synthetic polynucleotide
uccugucgggcaacccuaucguccccuggugaucauacaucacgcuauucuccuggcggggcucagccaugcugguuccuuaaacaccuguacgucgaca

SEQ ID NO: 1072 - RNA, 100 nt, Artificial Sequence
  Description of Artificial Sequence Synthetic polynucleotide
uccugucgggcaacccuaucguccccuggugaucauacaucacgcuauucuccuggcggggcucagccaugcugguuccuuaaacaccugauagucgaca

SEQ ID NO: 1073 - RNA, 100 nt, Artificial Sequence
  Description of Artificial Sequence Synthetic polynucleotide
uccugucacacaacccccccgucccuaagugauccuacaucacggauuucuccuggcggggcucagccaugcuggacgaaaguuaaacugauacaagaca

SEQ ID NO: 1074 - RNA, 100 nt, Artificial Sequence
  Description of Artificial Sequence Synthetic polynucleotide
uccuguggaccaaccgcaacguccauucgugaugaaccaucagagaacacuccuggcggggcucagccaugcuggcaaaaaguuaugcugauauuagaca

SEQ ID NO: 1075 - RNA, 100 nt, Artificial Sequence
  Description of Artificial Sequence Synthetic polynucleotide
uccugcaacccaacaaauacgucggcgcgugaagguccaucugagacaacuccuggcggggcucagccaugcuggcagaaaguugugcugauaauagaca

SEQ ID NO: 1076 - RNA, 100 nt, Artificial Sequence
  Description of Artificial Sequence Synthetic polynucleotide
uccuguccgagaacccggcagucccaaacugaucucagaucacucauccguccuggcggggcucagccacgcugguucauuacagaccugaguuucgaca

SEQ ID NO: 1077 - RNA, 100 nt, Artificial Sequence
  Description of Artificial Sequence Synthetic polynucleotide
uccugagacccaacagauacgucacugcgugaccccuaaucacuucgaacuccuggcggggcucagccacagacaaucaaaggggaccugacucucgaca

SEQ ID NO: 1078 - RNA, 100 nt, Artificial Sequence
  Description of Artificial Sequence Synthetic polynucleotide
uccuguacaccaaccaggacguccaaccgugauacaccaucagagucuacuccuggcggggcucagccacgcucuacacaaguucuaaugauaggguaca

SEQ ID NO: 1079 - RNA, 100 nt, Artificial Sequence
  Description of Artificial Sequence Synthetic polynucleotide
uccugaaacccaacgauuacgucggcgcgugacgcuccaucucagaaaacuccuggcggggcucagccacgcucauccuaaguuccggugauaggauaca

SEQ ID NO: 1080 - RNA, 100 nt, Artificial Sequence
  Description of Artificial Sequence Synthetic polynucleotide
uccugugggccaaccaagacguccauacgugaugcgaguucacuucgaacuccuggcggggcucagccacgcuaagcccaaguucaccugauagucgaca

SEQ ID NO: 1081 - RNA, 100 nt, Artificial Sequence
  Description of Artificial Sequence Synthetic polynucleotide
uccuagaccccaaacaguacguggaagcgugcaauuccauguuugggaacuccuggcggggcucagccacgcugaccuguaguucaccugauagucgaca

SEQ ID NO: 1082 - RNA, 100 nt, Artificial Sequence
  Description of Artificial Sequence Synthetic polynucleotide
uccucggccccaaagaguacguaagagcgugccggaacaucacuucgaacuccuggcggggcucagccacgcugccagguaguucaccugauagucgaca

SEQ ID NO: 1083 - RNA, 100 nt, Artificial Sequence
  Description of Artificial Sequence Synthetic polynucleotide
uccuguccagaaacccgcuagucccacaaugaucucggaucacucccauguccuggcggggcucagccacgcuggguaaaaguugcacugauacuagaca

SEQ ID NO: 1084 - RNA, 100 nt, Artificial Sequence
  Description of Artificial Sequence Synthetic polynucleotide
uccugucaggcaacccucccguccccuagugauccaggggcacuucgaaguccuggugggacucagccacgcuguacucuuguucacagcauagucgaca

SEQ ID NO: 1085 - RNA, 100 nt, Artificial Sequence
  Description of Artificial Sequence Synthetic polynucleotide
uccuacgccccaaggaguacgugaaagcguguuucaacaucacuucgaaguccuggugggacucagccacgcuguuuccuuugacaccugcaugucgaca

SEQ ID NO: 1086 - RNA, 100 nt, Artificial Sequence
  Description of Artificial Sequence Synthetic polynucleotide
uccuacgccccaaggaguacgugaaagcguguuucaacaucacuucgaaguccuggugggacucagccacgcuguuuccuuugacaccugauagucgaca

SEQ ID NO: 1087 - RNA, 100 nt, Artificial Sequence
  Description of Artificial Sequence Synthetic polynucleotide
ucgguucccccccgccguacccaccagcgaacucuuccccuggaucgaacuccuagaagggcucagccacgcugcuucaagguucaccuauuucacgaca

SEQ ID NO: 1088 - RNA, 100 nt, Artificial Sequence
  Description of Artificial Sequence Synthetic polynucleotide
uccugucgagcaacccccccgucccucggugaucacagaacacuucgaacuccuagaagggcucagccacgcugcuaccgcguucacaaaauagucgaca

SEQ ID NO: 1089 - RNA, 100 nt, Artificial Sequence
  Description of Artificial Sequence Synthetic polynucleotide
uccugucgagcaacccccccgucccucggugaucacagaacacuucgaacuccuagaagggcucagccacgcugcuaccgcguucaccugauagucgaca

SEQ ID NO: 1090 - RNA, 100 nt, Artificial Sequence
  Description of Artificial Sequence Synthetic polynucleotide
uccuaagccccaaaaaguacguggaagcguguuucaacaucacuucgaacuccuagaagggcucagccacgcugcuucuggacccaccugugcgucgaca

SEQ ID NO: 1091 - RNA, 100 nt, Artificial Sequence
  Description of Artificial Sequence Synthetic polynucleotide
uccuaagccccaaaaaguacguggaagcguguuucaacaucacuucgaacuccuagaagggcucagccacgcugcuucuggacccaccugauagucgaca

SEQ ID NO: 1092 - RNA, 100 nt, Artificial Sequence
  Description of Artificial Sequence Synthetic polynucleotide
uccuuagccccaagggguacgugagagcguguuucaacaucacuucgaauuccuggcggaacuuagccacgcugcuucugccugcaccugugcgucgaca

SEQ ID NO: 1093 - RNA, 100 nt, Artificial Sequence
  Description of Artificial Sequence Synthetic polynucleotide
uccuuggccccaagggguacguggaagcguguuucaacaucacuucgaacugcuggcgggguucagucacgcuguuuccuuucccaccugucugucgaca

SEQ ID NO: 1094 - RNA, 100 nt, Artificial Sequence
  Description of Artificial Sequence Synthetic polynucleotide
uccugucacacaacccaaucgucccucagugaucaguaagcacuucgaacuccuggcggggggcagucacgcugacauuuuguucacaucauagucgaca

SEQ ID NO: 1095 - RNA, 100 nt, Artificial Sequence
  Description of Artificial Sequence Synthetic polynucleotide
uccugucacacaacccaaucgucccucagugaucaguaagcacuucgaacuccuggcggggggcagucacgcugacauuuuguucaccugauagucgaca

SEQ ID NO: 1096 - RNA, 100 nt, Artificial Sequence
  Description of Artificial Sequence Synthetic polynucleotide
uccuacgccccaagagguacguaagagcguguuucaacaucacuucgaacuccuggcggggggcagucacgcugauuccuacggcaccugcaugucgaca

SEQ ID NO: 1097 - RNA, 100 nt, Artificial Sequence
  Description of Artificial Sequence Synthetic polynucleotide
uccuaaaccccaagagguacguaaaagcguguuucaacaucacuucgaacuccaggcgggguucagccacgcuuguacucuuggcaccuguaugucgaca

SEQ ID NO: 1098 - RNA, 100 nt, Artificial Sequence
  Description of Artificial Sequence Synthetic polynucleotide
uccuaaaccccaagagguacguaaaagcguguuucaacaucacuucgaacuccaggcgggguucagccacgcuuguacucuuggcaccugauagucgaca

SEQ ID NO: 1099 - RNA, 100 nt, Artificial Sequence
  Description of Artificial Sequence Synthetic polynucleotide
ucauaucccccucgccguacacaccagcgcccucuuccccuuggucgaacucgugucgggguucugccacgcugcuccaaaguucaccuuucgaccgaca

SEQ ID NO: 1100 - RNA, 100 nt, Artificial Sequence
  Description of Artificial Sequence Synthetic polynucleotide
uccugucgggcaacccuuccguccccuagugaucagaggccacuucgaacucgugucgggguucugccacgcugccccuauguucacgccauagucgaca

SEQ ID NO: 1101 - RNA, 100 nt, Artificial Sequence
  Description of Artificial Sequence Synthetic polynucleotide
uccucggccccaaagaguacguaggagcgugaucuuccaucacuucgaacucgugucgggguucugccacgcugcucaaaaguugcacugauauuagaca

SEQ ID NO: 1102 - RNA, 100 nt, Artificial Sequence
  Description of Artificial Sequence Synthetic polynucleotide
uccuaggccccaaaggguacgugggagcguguuucaacaucacuucgaaccccuggcguagcucagccacgcuuguccucuaugcaccuguuugucgaca

SEQ ID NO: 1103 - RNA, 100 nt, Artificial Sequence
  Description of Artificial Sequence Synthetic polynucleotide
ucgauucccccccgccguacuagccagcggccucuucccaaggaucgaacuccugucgggucucagccacgcuguuucaaaguucaccuccucuccgaca

SEQ ID NO: 1104 - RNA, 100 nt, Artificial Sequence
  Description of Artificial Sequence Synthetic polynucleotide
uccugucaggcaacccuuucguccccuagugaucagaagccacuucgaacuccugucgggucucagccacgcugugaggcuguucaccugauagucgaca

SEQ ID NO: 1105 - RNA, 100 nt, Artificial Sequence
  Description of Artificial Sequence Synthetic polynucleotide
uccuccaccccaagaaguacguaggagcguguuucaacaucacuucgaacuccugucgggucucagccacgcuguuucuuuaaacaccuguacgucgaca

SEQ ID NO: 1106 - RNA, 100 nt, Artificial Sequence
  Description of Artificial Sequence Synthetic polynucleotide
uccuccaccccaagaaguacguaggagcguguuucaacaucacuucgaacuccugucgggucucagccacgcuguuucuuuaaacaccugauagucgaca

SEQ ID NO: 1107 - RNA, 100 nt, Artificial Sequence
  Description of Artificial Sequence Synthetic polynucleotide
uccugucacacaaccccaccguccccuagugaucuuccaucacuucgaacuccugucgggucucagccacgcugugcgaaaguuauacugauacgggaca

SEQ ID NO: 1108 - RNA, 100 nt, Artificial Sequence
  Description of Artificial Sequence Synthetic polynucleotide
ucaccucccccuaaccguacaagccagcgcccucuuccuugugaucgaacuccuuccgggguucagccacgcugguacaaaguucaccuugcgcgcgaca

SEQ ID NO: 1109 - RNA, 100 nt, Artificial Sequence
  Description of Artificial Sequence Synthetic polynucleotide
uccugucagacaacccucccguccccuggugauccguguccacuucgaacuccuuccgggguucagccacgcugggauuacguucacaucauagucgaca

SEQ ID NO: 1110 - RNA, 100 nt, Artificial Sequence
  Description of Artificial Sequence Synthetic polynucleotide
uccugucagacaacccucccguccccuggugauccguguccacuucgaacuccuuccgggguucagccacgcugggauuacguucaccugauagucgaca

SEQ ID NO: 1111 - RNA, 100 nt, Artificial Sequence
  Description of Artificial Sequence Synthetic polynucleotide
uccucggccccaaaaaguacguaaaagcguguuucaacaucacuucgaacuccuuccgggguucagccacgcugguacuacucgcaccugcuugucgaca

SEQ ID NO: 1112 - RNA, 100 nt, Artificial Sequence
  Description of Artificial Sequence Synthetic polynucleotide
uccucggccccaaaaaguacguaaaagcguguuucaacaucacuucgaacuccuuccgggguucagccacgcugguacuacucgcaccugauagucgaca

SEQ ID NO: 1113 - RNA, 100 nt, Artificial Sequence
  Description of Artificial Sequence Synthetic polynucleotide
uccuaggccccaagggguacguaaaagcgugaucuuccaucacuucgaacuccuuccgggguucagccacgcugggagaaaguuaugcugauaagagaca

SEQ ID NO: 1114 - RNA, 100 nt, Artificial Sequence
  Description of Artificial Sequence Synthetic polynucleotide
uccuaggccccaagggguacguaaaagcgugaucuuccaucacuucgaacuccuuccgggguucagccacgcugguacaaaguucaccugauagucgaca

SEQ ID NO: 1115 - RNA, 100 nt, Artificial Sequence
  Description of Artificial Sequence Synthetic polynucleotide
ucaguuccccccuaccguacuaaccagcgcauucuucccaguaaucgaacuucugucgggccucagccacgcugguccaaaguucaccuaugacacgaca

SEQ ID NO: 1116 - RNA, 100 nt, Artificial Sequence
  Description of Artificial Sequence Synthetic polynucleotide
uccucagccccaagggguacguaaaagcguguuucaacaucacuucgaacuucugucgggccucagccacgcugguccuuacagcaccugcaugucgaca

SEQ ID NO: 1117 - RNA, 100 nt, Artificial Sequence
  Description of Artificial Sequence Synthetic polynucleotide
uccucagccccaagggguacguaaaagcguguuucaacaucacuucgaacuucugucgggccucagccacgcugguccuuacagcaccugauagucgaca

SEQ ID NO: 1118 - RNA, 100 nt, Artificial Sequence
  Description of Artificial Sequence Synthetic polynucleotide
uccugucagacaacccauccgucccccggugaucuuccaucacuucgaacuucugucgggccucagccacgcuggccgaaaguuaugcugauaccagaca

SEQ ID NO: 1119 - RNA, 100 nt, Artificial Sequence
  Description of Artificial Sequence Synthetic polynucleotide
uccugucagacaacccauccgucccccggugaucuuccaucacuucgaacuucugucgggccucagccacgcugguccaaaguucaccugauagucgaca

SEQ ID NO: 1120 - RNA, 100 nt, Artificial Sequence
  Description of Artificial Sequence Synthetic polynucleotide
ucaccucccccuuaccguacccgccagcggguucuucccaauaaucgaacuccugucgggucuuagccacgcugauucaaaguucaccuccacuacgaca

SEQ ID NO: 1121 - RNA, 100 nt, Artificial Sequence
  Description of Artificial Sequence Synthetic polynucleotide
uccugucagacaacccaaccgucccucagugaucgcuugccacuucgaacuccugucgggucuuagccacgcugaaaucucguucacggcauagucgaca

SEQ ID NO: 1122 - RNA, 100 nt, Artificial Sequence
  Description of Artificial Sequence Synthetic polynucleotide
uccugucagacaacccaaccgucccucagugaucgcuugccacuucgaacuccugucgggucuuagccacgcugaaaucucguucaccugauagucgaca

SEQ ID NO: 1123 - RNA, 100 nt, Artificial Sequence
  Description of Artificial Sequence Synthetic polynucleotide
uccuuaaccccaaaagguacguacaagcguguuucaacaucacuucgaacuccugucgggucuuagccacgcugauuccugaaacaccugcgugucgaca

SEQ ID NO: 1124 - RNA, 100 nt, Artificial Sequence
  Description of Artificial Sequence Synthetic polynucleotide
uccuuaaccccaaaagguacguacaagcguguuucaacaucacuucgaacuccugucgggucuuagccacgcugauuccugaaacaccugauagucgaca

SEQ ID NO: 1125 - RNA, 100 nt, Artificial Sequence
  Description of Artificial Sequence Synthetic polynucleotide
uccuauaccccaagggguacguaagagcgugaucuuccaucacuucgaacuccugucgggucuuagccacgcugauagaaaguuguacugauaugagaca

SEQ ID NO: 1126 - RNA, 100 nt, Artificial Sequence
  Description of Artificial Sequence Synthetic polynucleotide
ucaucucccccuuaccguacaggccagcgaauucuuccccaaagucgaacuccagggagggcucagccacgcugcuccaaaguucaccuaaggaacgaca

SEQ ID NO: 1127 - RNA, 100 nt, Artificial Sequence
  Description of Artificial Sequence Synthetic polynucleotide
ucgacuccccccugccguacaggccagcgaauucuucccggccgucgaacuccuaucgggacucagccacgcugguccaaaguucaccucacuuccgaca

SEQ ID NO: 1128 - RNA, 100 nt, Artificial Sequence
  Description of Artificial Sequence Synthetic polynucleotide
uccuccaccccaagaaguacguggaagcguguuucaacaucacuucgaacuccuaucgggacucagccacgcugguccuuuaaacaccuguacgucgaca

SEQ ID NO: 1129 - RNA, 100 nt, Artificial Sequence
  Description of Artificial Sequence Synthetic polynucleotide
uccuccaccccaagaaguacguggaagcguguuucaacaucacuucgaacuccuaucgggacucagccacgcugguccuuuaaacaccugauagucgaca

SEQ ID NO: 1130 - RNA, 100 nt, Artificial Sequence
  Description of Artificial Sequence Synthetic polynucleotide
uccugucgggcaacccagccguccccuagugaucuuccaucacuucgaacuccuaucgggacucagccacgcugggcgaaaguugcacugauaaaggaca

SEQ ID NO: 1131 - RNA, 100 nt, Artificial Sequence
  Description of Artificial Sequence Synthetic polynucleotide
uccuguccgaaaacccgauggucccacauugaucuaagaucacuggcuuguccuggcgggggagagccacgcugguucaugcucgaccugacauucgaca

SEQ ID NO: 1132 - RNA, 100 nt, Artificial Sequence
  Description of Artificial Sequence Synthetic polynucleotide
uccuguccgaaaacccgauggucccacauugaucuaagaucacuggcuuguccuggcgggggagagccacgcugguucaugcucgaccugauagucgaca

SEQ ID NO: 1133 - RNA, 100 nt, Artificial Sequence
  Description of Artificial Sequence Synthetic polynucleotide
uccugucgggcaacccaaucguccccuagugaucccacaucaccgucuucuccuggcgggggagagccacgcugguucauacguaaccugaaccucgaca

SEQ ID NO: 1134 - RNA, 100 nt, Artificial Sequence
  Description of Artificial Sequence Synthetic polynucleotide
uccugucgggcaacccaaucguccccuagugaucccacaucaccgucuucuccuggcgggggagagccacgcugguucauacguaaccugauagucgaca

SEQ ID NO: 1135 - RNA, 100 nt, Artificial Sequence
  Description of Artificial Sequence Synthetic polynucleotide
uccugugaaccaaccgcaacguccaaucgugaugcaccaucagcagcuacuccuggcgggggagagccacgcugguucaccagaaaccugagcaucgaca

SEQ ID NO: 1136 - RNA, 100 nt, Artificial Sequence
  Description of Artificial Sequence Synthetic polynucleotide
uccugugaaccaaccgcaacguccaaucgugaugcaccaucagcagcuacuccuggcgggggagagccacgcugguucaccagaaaccugauagucgaca

SEQ ID NO: 1137 - RNA, 100 nt, Artificial Sequence
  Description of Artificial Sequence Synthetic polynucleotide
uccugcggcccaacggauacgucagcgcgugagacuccaucuggcaaaacuccuggcgggggagagccacgcugguucauucaaaaccugacauucgaca

SEQ ID NO: 1138 - RNA, 100 nt, Artificial Sequence
  Description of Artificial Sequence Synthetic polynucleotide
uccugcggcccaacggauacgucagcgcgugagacuccaucuggcaaaacuccuggcgggggagagccacgcugguucauucaaaaccugauagucgaca

SEQ ID NO: 1139 - RNA, 100 nt, Artificial Sequence
  Description of Artificial Sequence Synthetic polynucleotide
uccuaagccccaaaagguacguaagagcgugugguuccauaccaaggaacuccuggcgggggagagccacgcugguucacgucgaaccugaaccucgaca

SEQ ID NO: 1140 - RNA, 100 nt, Artificial Sequence
  Description of Artificial Sequence Synthetic polynucleotide
uccuaagccccaaaagguacguaagagcgugugguuccauaccaaggaacuccuggcgggggagagccacgcugguucacgucgaaccugauagucgaca

SEQ ID NO: 1141 - RNA, 100 nt, Artificial Sequence
  Description of Artificial Sequence Synthetic polynucleotide
uccauccccccauggcguacggagcagcguaaacuuccacgugagcgaacuccuggcgggggagagccacgcugguucaugucgaaccugacacucgaca

SEQ ID NO: 1142 - RNA, 100 nt, Artificial Sequence
  Description of Artificial Sequence Synthetic polynucleotide
uccuguccaggaacccggcagucccacucugaucuaggaucacugacauguccuggcgggggagagccacgcugguucaacgacguccugauuaccgaca

SEQ ID NO: 1143 - RNA, 100 nt, Artificial Sequence
  Description of Artificial Sequence Synthetic polynucleotide
uccuguccaggaacccggcagucccacucugaucuaggaucacugacauguccuggcgggggagagccacgcugguucaacgacguccugauagucgaca

SEQ ID NO: 1144 - RNA, 100 nt, Artificial Sequence
  Description of Artificial Sequence Synthetic polynucleotide
uccugucgggcaacccuaucgucccccggugauccggcaucaccccuuccuccuggcgggggagagccacgcugguucaauuaacuccugaucaccgaca

SEQ ID NO: 1145 - RNA, 100 nt, Artificial Sequence
  Description of Artificial Sequence Synthetic polynucleotide
uccugucgggcaacccuaucgucccccggugauccggcaucaccccuuccuccuggcgggggagagccacgcugguucaauuaacuccugauagucgaca

SEQ ID NO: 1146 - RNA, 100 nt, Artificial Sequence
  Description of Artificial Sequence Synthetic polynucleotide
uccugguacccaacgauuacgucggcgcgugaaaguccaucuccguuaacuccuggcgggggagagccacgcugguucaauccgauccugauuaacgaca

SEQ ID NO: 1147 - RNA, 100 nt, Artificial Sequence
  Description of Artificial Sequence Synthetic polynucleotide
uccugguacccaacgauuacgucggcgcgugaaaguccaucuccguuaacuccuggcgggggagagccacgcugguucaauccgauccugauagucgaca

SEQ ID NO: 1148 - RNA, 100 nt, Artificial Sequence
  Description of Artificial Sequence Synthetic polynucleotide
uccccacccccacgacguacgacacagcguccacuuccacugaagcgaacuccuggcgggggagagccacgcugguucaauuaaauccugauucgcgaca

SEQ ID NO: 1149 - RNA, 100 nt, Artificial Sequence
  Description of Artificial Sequence Synthetic polynucleotide
uccuguccggaaacccgauggucccaagaugaucucgaaucacucaaccguccuggcgggggagagccacgcugguucaaauacugacugauaacagaca

SEQ ID NO: 1150 - RNA, 100 nt, Artificial Sequence
  Description of Artificial Sequence Synthetic polynucleotide
uccuguccggaaacccgauggucccaagaugaucucgaaucacucaaccguccuggcgggggagagccacgcugguucaaauacugacugauagucgaca

SEQ ID NO: 1151 - RNA, 100 nt, Artificial Sequence
  Description of Artificial Sequence Synthetic polynucleotide
uccuguggaccaaccgugacguccguccgugauaaaccaucaagcgcuacuccuggcgggggagagccacgcugguucaaacauguacugauaugggaca

SEQ ID NO: 1152 - RNA, 100 nt, Artificial Sequence
  Description of Artificial Sequence Synthetic polynucleotide
uccugaagcccaacgguuacgucagcgcgugaaaauccauccagauuaacuccuggcgggggagagccacgcugguucaaacaauugcugauauuagaca

SEQ ID NO: 1153 - RNA, 100 nt, Artificial Sequence
  Description of Artificial Sequence Synthetic polynucleotide
uccugaagcccaacgguuacgucagcgcgugaaaauccauccagauuaacuccuggcgggggagagccacgcugguucaaacaauugcugauagucgaca

SEQ ID NO: 1154 - RNA, 100 nt, Artificial Sequence
  Description of Artificial Sequence Synthetic polynucleotide
uccuuggccccaagaaguacguacaagcgugcaauuccauguugaagaacuccuggcgggggagagccacgcugguucaaaccagugcugauacaagaca

SEQ ID NO: 1155 - RNA, 100 nt, Artificial Sequence
  Description of Artificial Sequence Synthetic polynucleotide
uccuuggccccaagaaguacguacaagcgugcaauuccauguugaagaacuccuggcgggggagagccacgcugguucaaaccagugcugauagucgaca

SEQ ID NO: 1156 - RNA, 100 nt, Artificial Sequence
  Description of Artificial Sequence Synthetic polynucleotide
uccuguccacaaacccgguggucccauauugaucuaagaucacugauguguccuggcgggggagagccacgcugguucaaagccgaggugauagggaaca

SEQ ID NO: 1157 - RNA, 100 nt, Artificial Sequence
  Description of Artificial Sequence Synthetic polynucleotide
uccuguccacaaacccgguggucccauauugaucuaagaucacugauguguccuggcgggggagagccacgcugguucaaagccgaggugauagucgaca

SEQ ID NO: 1158 - RNA, 100 nt, Artificial Sequence
  Description of Artificial Sequence Synthetic polynucleotide
uccugucagacaacccucccguccccuggugaucccgcaucacggaagccuccuggcgggggagagccacgcugguucaaagaauuggugauaggguaca

SEQ ID NO: 1159 - RNA, 100 nt, Artificial Sequence
  Description of Artificial Sequence Synthetic polynucleotide
uccugucagacaacccucccguccccuggugaucccgcaucacggaagccuccuggcgggggagagccacgcugguucaaagaauuggugauagucgaca

SEQ ID NO: 1160 - RNA, 100 nt, Artificial Sequence
  Description of Artificial Sequence Synthetic polynucleotide
uccuguggaccaaccaccacguccgcccgugaugcaccaucaaacaauacuccuggcgggggagagccacgcugguucaaagccccggugauaggguaca

SEQ ID NO: 1161 - RNA, 100 nt, Artificial Sequence
  Description of Artificial Sequence Synthetic polynucleotide
uccuguggaccaaccaccacguccgcccgugaugcaccaucaaacaauacuccuggcgggggagagccacgcugguucaaagccccggugauagucgaca

SEQ ID NO: 1162 - RNA, 100 nt, Artificial Sequence
  Description of Artificial Sequence Synthetic polynucleotide
uccugcggcccaacagauacgucggugcgugaggauccaucuggcaaaacuccuggcgggggagagccacgcugguucaaagcuguagugauaggguaca

SEQ ID NO: 1163 - RNA, 100 nt, Artificial Sequence
  Description of Artificial Sequence Synthetic polynucleotide
uccugcggcccaacagauacgucggugcgugaggauccaucuggcaaaacuccuggcgggggagagccacgcugguucaaagcuguagugauagucgaca

SEQ ID NO: 1164 - RNA, 100 nt, Artificial Sequence
  Description of Artificial Sequence Synthetic polynucleotide
uccucagccccaagaaguacguaggagcgugugauuccaugugggagaacuccuggcgggggagagccacgcugguucaaagauaugaugauagggaaca

SEQ ID NO: 1165 - RNA, 100 nt, Artificial Sequence
  Description of Artificial Sequence Synthetic polynucleotide
uccucagccccaagaaguacguaggagcgugugauuccaugugggagaacuccuggcgggggagagccacgcugguucaaagauaugaugauagucgaca

SEQ ID NO: 1166 - RNA, 100 nt, Artificial Sequence
  Description of Artificial Sequence Synthetic polynucleotide
uccuacaccccaagagguacguaaaagcgugccuaagcaucacuucgaacuccuggcgggggagagccacagacagucaaagcagaccugagaaucgaca

SEQ ID NO: 1167 - RNA, 100 nt, Artificial Sequence
  Description of Artificial Sequence Synthetic polynucleotide
uccuguacaccaaccgagacguccguacgugauggaccaucaaacacuacuccuggcgggggagagccacgcuuaucccaaguucuuaugauaggguaca

SEQ ID NO: 1168 - RNA, 100 nt, Artificial Sequence
  Description of Artificial Sequence Synthetic polynucleotide
uccuguacaccaaccgagacguccguacgugauggaccaucaaacacuacuccuggcgggggagagccacgcuuaucccaaguucaccugauagucgaca

SEQ ID NO: 1169 - RNA, 100 nt, Artificial Sequence
  Description of Artificial Sequence Synthetic polynucleotide
uccuggaacccaacgguuacgucggcgcgugaagauccauccauaaaaacuccuggcgggggagagccacgcuaugagcaaguucugaugauaggguaca

SEQ ID NO: 1170 - RNA, 100 nt, Artificial Sequence
  Description of Artificial Sequence Synthetic polynucleotide
uccuggaacccaacgguuacgucggcgcgugaagauccauccauaaaaacuccuggcgggggagagccacgcuaugagcaaguucaccugauagucgaca

SEQ ID NO: 1171 - RNA, 100 nt, Artificial Sequence
  Description of Artificial Sequence Synthetic polynucleotide
uccaaacccccauggcguacgaaacagcguaaacuuccagccgaacgaacuccuggcgggggagagccacgcucgacgcaaguucugaugauaggguaca

SEQ ID NO: 1172 - RNA, 100 nt, Artificial Sequence
  Description of Artificial Sequence Synthetic polynucleotide
uccaaacccccauggcguacgaaacagcguaaacuuccagccgaacgaacuccuggcgggggagagccacgcucgacgcaaguucaccugauagucgaca

SEQ ID NO: 1173 - RNA, 100 nt, Artificial Sequence
  Description of Artificial Sequence Synthetic polynucleotide
uccugucccggcacccgucuaucccagaacgaucuugauaaucuucgaacuccuggcgggggagagccacgcuacugacaaguucuagugauaggauaca

SEQ ID NO: 1174 - RNA, 100 nt, Artificial Sequence
  Description of Artificial Sequence Synthetic polynucleotide
uccuguccggaaacccgaaagucccaaauugaucuggggcgacuucgaacuccuggcgggggagagccacgcuagccacaaguucuuaugauaggguaca

SEQ ID NO: 1175 - RNA, 100 nt, Artificial Sequence
  Description of Artificial Sequence Synthetic polynucleotide
uccugucaggcaacccacucgucccuaagugauccgauuacacuucgaacuccuggcgggggagagccacgcuuacgauaaguuccgaugauaggguaca

SEQ ID NO: 1176 - RNA, 100 nt, Artificial Sequence
  Description of Artificial Sequence Synthetic polynucleotide
uccugucgaacaacccauccgucccucggugaucauacaucaccuaucucuccuggcgggggagagccacgcuguaaaccaguucaacggauagucgaca

SEQ ID NO: 1177 - RNA, 100 nt, Artificial Sequence
  Description of Artificial Sequence Synthetic polynucleotide
uccugcaacccaacaaauacgucaacgcgugaauauccaucugagaaaacuccuggcgggggagagccacgcugcaaacuaguucaggggauagucgaca

SEQ ID NO: 1178 - RNA, 100 nt, Artificial Sequence
  Description of Artificial Sequence Synthetic polynucleotide
uccuuagccccaaagaguacguagaagcguguaguuccauauaaaggaacuccuggcgggggagagccacgcugcaagcuaguucaacggauagucgaca

SEQ ID NO: 1179 - RNA, 100 nt, Artificial Sequence
  Description of Artificial Sequence Synthetic polynucleotide
ucccaacccccacaacguacgcggcagcguauacuuccacgugagcgaacuccuggcgggggagagccacgcugugcccuaguucaacggauagucgaca

SEQ ID NO: 1180 - RNA, 100 nt, Artificial Sequence
  Description of Artificial Sequence Synthetic polynucleotide
uccuguccacaaacccgaaagucccaaauugaucuggaugaacuucgaacuccuggcgggggagagccacgcugucuguuaguucagacgauagucgaca

SEQ ID NO: 1181 - RNA, 100 nt, Artificial Sequence
  Description of Artificial Sequence Synthetic polynucleotide
ucggauccccccugccguacuggccagcgguaauguccaucacuucgaacuccuggcgggggagagccacgcugacgacuaguucagaggauagucgaca

SEQ ID NO: 1182 - RNA, 100 nt, Artificial Sequence
  Description of Artificial Sequence Synthetic polynucleotide
uccuguccacaaacccgguagucccacacugaucugagaucacuggcacguccuggcgggggagagccacgcuggauagccguucacauaauagucgaca

SEQ ID NO: 1183 - RNA, 100 nt, Artificial Sequence
  Description of Artificial Sequence Synthetic polynucleotide
uccugucgggcaacccaaccgucccucggugaucccacaucacaugugucuccuggcgggggagagccacgcuggaaguuuguucacgaaauagucgaca

SEQ ID NO: 1184 - RNA, 100 nt, Artificial Sequence
  Description of Artificial Sequence Synthetic polynucleotide
uccuguaggccaaccgcaacguccguucgugauggaccaucaacaguuacuccuggcgggggagagccacgcugggugcucguucacggaauagucgaca

SEQ ID NO: 1185 - RNA, 100 nt, Artificial Sequence
  Description of Artificial Sequence Synthetic polynucleotide
uccugaagcccaacgaauacgucgaugcgugacaguccaucuuuaacaacuccuggcgggggagagccacgcugggagaccguucacacaauagucgaca

SEQ ID NO: 1186 - RNA, 100 nt, Artificial Sequence
  Description of Artificial Sequence Synthetic polynucleotide
uccuaggccccaagggguacgugagagcguguaguuccauagugaggaacuccuggcgggggagagccacgcuggcugccuguucacgauauagucgaca

SEQ ID NO: 1187 - RNA, 100 nt, Artificial Sequence
  Description of Artificial Sequence Synthetic polynucleotide
uccugucgaacaacccuuccgucccccggugauccuacaucacaacccucuccuggcgggggagagccacgcugguagcuuuuucacccauuagucgaca

SEQ ID NO: 1188 - RNA, 100 nt, Artificial Sequence
  Description of Artificial Sequence Synthetic polynucleotide
uccuguaggccaaccgccacguccguucgugauagaccaucaauauauacuccuggcgggggagagccacgcugguauuccuuucaccgucuagucgaca

SEQ ID NO: 1189 - RNA, 100 nt, Artificial Sequence
  Description of Artificial Sequence Synthetic polynucleotide
uccuggagcccaacgacuacgucagugcgugaggauccaucuuccuaaacuccuggcgggggagagccacgcugguguacgauucacccucuagucgaca

SEQ ID NO: 1190 - RNA, 100 nt, Artificial Sequence
  Description of Artificial Sequence Synthetic polynucleotide
uccuuagccccaaaggguacgugggagcgugcgguuccaugugacagaacuccuggcgggggagagccacgcuggugugguauucaccaccuagucgaca

SEQ ID NO: 1191 - RNA, 100 nt, Artificial Sequence
  Description of Artificial Sequence Synthetic polynucleotide
uccgggcccccacagcguacgagacagcguaugcuuccagauucacgaacuccuggcgggggagagccacgcuggugaugcuuucaccaccuagucgaca

SEQ ID NO: 1192 - RNA, 100 nt, Artificial Sequence
  Description of Artificial Sequence Synthetic polynucleotide
uccuguccaggaacccgcuggucccacacugaucucagaucacugguucauccuggcgggggagagccacgcugguuguucgcucaccucacagucgaca

SEQ ID NO: 1193 - RNA, 100 nt, Artificial Sequence
  Description of Artificial Sequence Synthetic polynucleotide
uccuguccaggaacccgcuggucccacacugaucucagaucacugguucauccuggcgggggagagccacgcugguuguucgcucaccugauagucgaca

SEQ ID NO: 1194 - RNA, 100 nt, Artificial Sequence
  Description of Artificial Sequence Synthetic polynucleotide
uccugucaagcaacccaaccguccccuggugaucagacaucacgcgcuucuccuggcgggggagagccacgcugguuaauuuaucaccuuaaagucgaca

SEQ ID NO: 1195 - RNA, 100 nt, Artificial Sequence
  Description of Artificial Sequence Synthetic polynucleotide
uccugucaagcaacccaaccguccccuggugaucagacaucacgcgcuucuccuggcgggggagagccacgcugguuaauuuaucaccugauagucgaca

SEQ ID NO: 1196 - RNA, 100 nt, Artificial Sequence
  Description of Artificial Sequence Synthetic polynucleotide
uccuguaggccaaccaccacguccguacgugaugcgccaucagcaguuacuccuggcgggggagagccacgcugguugcucuaucaccuucgagucgaca

SEQ ID NO: 1197 - RNA, 100 nt, Artificial Sequence
  Description of Artificial Sequence Synthetic polynucleotide
uccugacacccaacgaauacgucgacgcgugacgauccaucuaccaaaacuccuggcgggggagagccacgcugguuguuaacucaccucuaagucgaca

SEQ ID NO: 1198 - RNA, 100 nt, Artificial Sequence
  Description of Artificial Sequence Synthetic polynucleotide
uccucgaccccaagaaguacguggaagcgugcgauuccaugucaaggaacuccuggcgggggagagccacgcugguugugcacucaccuaccagucgaca

SEQ ID NO: 1199 - RNA, 100 nt, Artificial Sequence
  Description of Artificial Sequence Synthetic polynucleotide
ucggaucccccuugccguacccgccagcgaucucuuccuuugugucgaacuccuggcgggggagagccacgcugguuaacuugucaccucccagucgaca

SEQ ID NO: 1200 - RNA, 100 nt, Artificial Sequence
  Description of Artificial Sequence Synthetic polynucleotide
uccugucaagcaacccccucguccccuagugaucgaacaucacaugccucuccuggcgggggagagccacgcugguucucuuaccaccugcuugucgaca

SEQ ID NO: 1201 - RNA, 100 nt, Artificial Sequence
  Description of Artificial Sequence Synthetic polynucleotide
uccugucaagcaacccccucguccccuagugaucgaacaucacaugccucuccuggcgggggagagccacgcugguucucuuaccaccugauagucgaca

SEQ ID NO: 1202 - RNA, 100 nt, Artificial Sequence
  Description of Artificial Sequence Synthetic polynucleotide
uccugugagccaaccaucacguccaaccgugaugcgccaucagaggccacuccuggcgggggagagccacgcugguuccucagccaccugcccgucgaca

SEQ ID NO: 1203 - RNA, 100 nt, Artificial Sequence
  Description of Artificial Sequence Synthetic polynucleotide
uccugugagccaaccaucacguccaaccgugaugcgccaucagaggccacuccuggcgggggagagccacgcugguuccucagccaccugauagucgaca

SEQ ID NO: 1204 - RNA, 100 nt, Artificial Sequence
  Description of Artificial Sequence Synthetic polynucleotide
uccugauacccaacgaauacgucaaugcgugagacuccaucuggaauaacuccuggcgggggagagccacgcugguuccccacccaccugccugucgaca

SEQ ID NO: 1205 - RNA, 100 nt, Artificial Sequence
  Description of Artificial Sequence Synthetic polynucleotide
uccugauacccaacgaauacgucaaugcgugagacuccaucuggaauaacuccuggcgggggagagccacgcugguuccccacccaccugauagucgaca

SEQ ID NO: 1206 - RNA, 100 nt, Artificial Sequence
  Description of Artificial Sequence Synthetic polynucleotide
uccuuggccccaaaggguacgugagagcguguaauuccauaguauagaacuccuggcgggggagagccacgcugguucuccccacaccugcuugucgaca

SEQ ID NO: 1207 - RNA, 100 nt, Artificial Sequence
  Description of Artificial Sequence Synthetic polynucleotide
uccauccccccauggcguacgaggcagcguacgcuuccaaaaagccgaacuccuggcgggggagagccacgcugguucccccggcaccuguuugucgaca

SEQ ID NO: 1208 - RNA, 100 nt, Artificial Sequence
  Description of Artificial Sequence Synthetic polynucleotide
uccuguccacaaacccggcggucccauacugaucugggaucacucaaauauccuggcgggggagagccacgcuggcagaaaguuguacugauaaaggaca

SEQ ID NO: 1209 - RNA, 100 nt, Artificial Sequence
  Description of Artificial Sequence Synthetic polynucleotide
uccugucaaacaacccuuccgucccuuggugauccggcaucacguaccucuccuggcgggggagagccacgcuggcgaaaaguuguacugauacuagaca

SEQ ID NO: 1210 - RNA, 100 nt, Artificial Sequence
  Description of Artificial Sequence Synthetic polynucleotide
uccugaagcccaacaacuacgucgaugcgugacaguccaucucacaaaacuccuggcgggggagagccacgcugggagaaaguugcacugauacaggaca

SEQ ID NO: 1211 - RNA, 100 nt, Artificial Sequence
  Description of Artificial Sequence Synthetic polynucleotide
uccuuggccccaaaaaguacguaggagcgugccguuccauaagggggaacuccuggcgggggagagccacgcuggcaaaaaguuauacugauaagggaca

SEQ ID NO: 1212 - RNA, 100 nt, Artificial Sequence
  Description of Artificial Sequence Synthetic polynucleotide
uccauacccccauagcguacgaaacagcguuuacuuccacguagacgaacuccuggcggggcucagccaugcuauuaguaaguucaccugauagucgaca

SEQ ID NO: 1213 - RNA, 100 nt, Artificial Sequence
  Description of Artificial Sequence Synthetic polynucleotide
ucgccucccccucgccguaccagccagcggguucuucccugcggucgaacuccuggcggggcucagccauauacgaucaaaguucaccugauagucgaca

SEQ ID NO: 1214 - RNA, 100 nt, Artificial Sequence
  Description of Artificial Sequence Synthetic polynucleotide
ucgccucccccucgccguaccagccagcggguucuucccugcggucgaacuccuggcggggcucagccauauacgaucaaaggcaaccugagcaucgaca

SEQ ID NO: 1215 - RNA, 100 nt, Artificial Sequence
  Description of Artificial Sequence Synthetic polynucleotide
uccucggccccaagaaguacgugggagcgugcuaccgcaucacuucgaacuccuggcggggcucagccauaagcucucaaaguucaccugauagucgaca

SEQ ID NO: 1216 - DNA, 32 bp, Homo sapiens
taggctttctggctttttaccggaaagcccct

SEQ ID NO: 1217 - RNA, 32 nt, Homo sapiens
uaggcuuucuggcuuuuuaccggaaagccccu

SEQ ID NO: 1218 - DNA, 31 bp, Mus sp.
caggttttctgacttcggtcggaaaacccct

SEQ ID NO: 1219 - RNA, 31 nt, Mus sp.
cagguuuucugacuucggucggaaaaccccu

SEQ ID NO: 1220 - DNA, 11 bp, Artificial Sequence
  Description of Artificial Sequence Synthetic oligonucleotide
aatttttggag

SEQ ID NO: 1221 - RNA, 11 nt, Artificial Sequence
  Description of Artificial Sequence Synthetic oligonucleotide
aauuuuuggag

SEQ ID NO: 1222 - DNA, 249 bp, Artificial Sequence
  Description of Artificial Sequence Synthetic polynucleotide
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 1223 - DNA, 341 bp, Artificial Sequence
  Description of Artificial Sequence Synthetic polynucleotide
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 1224 - DNA, 263 bp, Artificial Sequence
  Description of Artificial Sequence Synthetic polynucleotide
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 1225 - DNA, 257 bp, Artificial Sequence
  Description of Artificial Sequence Synthetic polynucleotide
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 1226 - DNA, 393 bp, Artificial Sequence
  Description of Artificial Sequence Synthetic polynucleotide
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 1227 - DNA, 698 bp, Artificial Sequence
  Description of Artificial Sequence Synthetic polynucleotide
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacc

SEQ ID NO: 1228 - DNA, 105 bp, Unknown
  Description of Unknown target sequence
  modified_base (1)..(1): a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 1229 - DNA, 105 bp, Artificial Sequence
  Description of Artificial Sequence Synthetic polynucleotide
  modified_base (105)..(105): a, c, t, g, unknown or other
agcagccacaactccgggaacccctttgaaagtcctttcccgaatacatccacggctaaacttctcctttacaccacactgtcgtcgattaggccactcccagtn

(19) United States
(12) Patent Application Publication
(10) Pub. No.: US 2024/0279656 A1
(43) Pub. Date: Aug. 22, 2024

(54) Engineered Guide RNAs and Polynucleotides

(71) Applicant: Shape Therapeutics Inc., Seattle, WA (US)

(72) Inventors: Richard Thomas SULLIVAN, Seattle, WA (US); Brian John BOOTH, West Roxbury, MA (US); Adrian BRIGGS, Seattle, WA (US); Yiannis SAVVA, Seattle, WA (US)

(21) Appl. No.: 18/568,902

(22) PCT Filed: Jun. 28, 2022
(86) PCT No.: PCT/US2022/035304

§ 371 Date: Dec. 11, 2023

Related U.S. Application Data
(60) Provisional application No. 63/345,060, filed on May 24, 2022.
(60) Provisional application No. 63/339,615, filed on May 9, 2022.
(60) Provisional application No. 63/303,669, filed on Jan. 27, 2022.
(60) Provisional application No. 63/277,659, filed on Nov. 10, 2021.
(60) Provisional application No. 63/216,179, filed on Jun. 29, 2021.

(51) Int. Cl. C12N 15/113; C12N 15/10; C12N 15/86

(52) U.S. Cl. CPC C12N 15/113; C12N 15/102; C12N 15/86; C12N 2310/11; C12N 2750/14143

(57) ABSTRACT

Disclosed herein are engineered guide RNAs and compositions comprising the same for treatment of diseases or conditions in a subject. Also disclosed herein are methods of treating diseases or conditions in a subject by administering engineered guide RNAs or pharmaceutical compositions described herein.

### CROSS REFERENCE

This application claims priority under 35 U.S.C. § 119 from Provisional Application Ser. No. 63/216,179, filed Jun. 29, 2021, Provisional Application Ser. No. 63/277,659, filed Nov. 10, 2021, Provisional Application Ser. No. 63/303,669, filed Jan. 27, 2022, Provisional Application Ser. No. 63/339,615, filed May 9, 2022, and Provisional Application Ser. No. 63/345,060, filed May 24, 2022, the disclosures of which are incorporated herein by reference in their entirety.

### BACKGROUND

Compositions that mediate RNA editing can be viable therapies for genetic diseases. However, highly efficacious compositions that can maximize on-target RNA editing while minimizing off-target RNA editing are needed. Moreover, compositions that are capable of facilitating RNA editing are also needed.

### SUMMARY

Disclosed herein are engineered guide RNAs and composition comprising engineered guide RNAs, where: (a) the engineered guide RNA, upon hybridization to a c.1 translation initiation sequence (TIS) of a target MAPT RNA, forms a guide-target RNA scaffold with the sequence of the target MAPT RNA, wherein the c.1 TIS is an AUG starting at the 18th nucleotide in Exon 1 of the target MAPT RNA; (b) formation of the guide-target RNA scaffold substantially forms one or more structural features selected from the group consisting of: a bulge, an internal loop, and a hairpin; (c) the structural feature is not present within the engineered guide RNA prior to the hybridization of the engineered guide RNA to the MAPT target RNA; and (d) upon hybridization of the engineered guide RNA to the c.1 TIS of the target MAPT RNA, the engineered guide RNA facilitates RNA editing of one or more target adenosines in the c.1 TIS of the target MAPT RNA by an RNA editing entity. In some embodiments, the one or more structural features comprises: a first 6/6 symmetric internal loop at a position selected from the group consisting of: 34, 33, 32, 31, 30, 26, 25, 24, 23, and 22; relative to the target adenosine at position 0. In some embodiments, the first 6/6 symmetric internal loop is at position 34, relative to the target adenosine at position 0. In some embodiments, the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some embodiments, the engineered guide RNA comprises at least about: 80%, 85%, 90%, 92%, 95%, 97%, or 99% sequence identity to SEQ ID NO: 478. In some embodiments, the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some embodiments, the engineered guide RNA comprises at least about: 80%, 85%, 90%, 92%, 95%, 97%, or 99% sequence identity to SEQ ID NO: 479. In some embodiments, the first 6/6 symmetric internal loop is at position 33, relative to the target adenosine at position 0. In some embodiments, the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 592. In some embodiments, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 593. In some embodiments, the one or more structural features further comprises at least one structural feature selected from the group consisting of: a 3/3 symmetric bulge at position −21 relative to position 0, a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 639. In some embodiments, the one or more structural features further comprises at least one structural feature selected from the group consisting of: a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 640. In some embodiments, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, and any combination thereof. In some embodiments, the engineered guide RNA comprises at least about: 80%, 85%, 90%, 92%, 95%, 97%, or 99% sequence identity to SEQ ID NO: 686. In some embodiments, the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 687. In some embodiments, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 688. In some embodiments, the one or more structural features further comprises at least one structural feature selected from the group consisting of: a 3/3 symmetric bulge at position −21 relative to position 0, a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 695. In some embodiments, the one or more structural features further comprises at least one structural feature selected from the group consisting of: a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 696. In some embodiments, the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 714. In some embodiments, the one or more structural features further comprises at least one structural feature selected from the group consisting of: a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 715. In some embodiments, the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 722. In some embodiments, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 723. In some embodiments, the one or more structural features further comprises at least one structural feature selected from the group consisting of: a 3/3 symmetric bulge at position −22 relative to position 0, a 3/3 symmetric bulge at position −13 relative to position 0, a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 1059. In some embodiments, the one or more structural features further comprises at least one structural feature selected from the group consisting of: a 3/3 symmetric bulge at position −21 relative to position 0, a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 1116. In some embodiments, the one or more structural features further comprises at least one structural feature selected from the group consisting of: a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 1117. In some embodiments, the one or more structural features further comprises at least one structural feature selected from the group consisting of: a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 1215. In some embodiments, the first 6/6 symmetric internal loop is at position 32, relative to the target adenosine at position 0. In some embodiments, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 438. In some embodiments, the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 439. In some embodiments, the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 816. In some embodiments, the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 817. In some embodiments, the first 6/6 symmetric internal loop is at position 31, relative to the target adenosine at position 0. In some embodiments, the one or more structural features further comprises at least one structural feature selected from the group consisting of: a 3/3 symmetric bulge at position −19 relative to position 0, a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some embodiments, the engineered guide RNA comprises at least about: 80%, 85%, 90%, 92%, 95%, 97%, or 99% sequence identity to SEQ ID NO: 162. In some embodiments, the one or more structural features further comprises at least one structural feature selected from the group consisting of: a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some embodiments, the engineered guide RNA comprises at least about: 80%, 85%, 90%, 92%, 95%, 97%, or 99% sequence identity to SEQ ID NO: 163. In some embodiments, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some embodiments, the engineered guide RNA comprises at least about: 80%, 85%, 90%, 92%, 95%, 97%, or 99% sequence identity to SEQ ID NO: 929. In some embodiments, the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some embodiments, the engineered guide RNA comprises at least about: 80%, 85%, 90%, 92%, 95%, 97%, or 99% sequence identity to SEQ ID NO: 930. In some embodiments, the first 6/6 symmetric internal loop is at position 30, relative to the target adenosine at position 0. In some embodiments, the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 538. In some embodiments, the one or more structural features further comprises at least one structural feature selected from the group consisting of: a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 539. In some embodiments, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 604. In some embodiments, the one or more structural features further comprises at least one structural feature selected from the group consisting of: a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 605. In some embodiments, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 619. In some embodiments, the one or more structural features further comprises at least one structural feature selected from the group consisting of a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 620. In some embodiments, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 677. In some embodiments, the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 678. In some embodiments, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 684. In some embodiments, the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 685. In some embodiments, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 720. In some embodiments, the one or more structural features further comprises at least one structural feature selected from the group consisting of: a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 721. In some embodiments, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 733. In some embodiments, the one or more structural features further comprises at least one structural feature selected from the group consisting of: a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 734. In some embodiments, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 763. In some embodiments, the one or more structural features further comprises at least one structural feature selected from the group consisting of: a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 764. In some embodiments, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 775. In some embodiments, the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 776. In some embodiments, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 795. In some embodiments, the one or more structural features further comprises at least one structural feature selected from the group consisting of: a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some embodiments, the engineered guide RNA comprises at least about: 80%, 85%, 90%, 92%, 95%, 97%, or 99% sequence identity to SEQ ID NO: 796. In some embodiments, the first 6/6 symmetric internal loop is at position 26, relative to the target adenosine at position 0. In some embodiments, the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 464. In some embodiments, the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 465. In some embodiments, the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, and any combination thereof. In some embodiments, the engineered guide RNA comprises at least about: 80%, 85%, 90%, 92%, 95%, 97%, or 99% sequence identity to SEQ ID NO: 583. In some embodiments, the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 584. In some embodiments, the one or more structural features further comprises at least one structural feature selected from the group consisting of: a 6/6 symmetric internal loop at position −20 relative to position 0, a U/U mismatch at position −11 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 589. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, and any combination thereof. In some embodiments, the engineered guide RNA comprises at least about: 80%, 85%, 90%, 92%, 95%, 97%, or 99% sequence identity to SEQ ID NO: 610. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 611. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 618. In some embodiments, the one or more structural features further comprises at least one structural feature selected from the group consisting of: a 6/6 symmetric internal loop at position −20 relative to position 0, a A/G mismatch at position −7 relative to position 0, a C/U mismatch at position −4 relative to position 0, a 2/2 symmetric bulge at position −1 relative to position 0, a G/U Wobble at position 8 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 662. In some embodiments, the one or more structural features further comprises at least one structural feature selected from the group consisting of a 6/6 symmetric internal loop at position −20 relative to position 0, a C/A mismatch at position −5 relative to position 0, a 2/2 symmetric bulge at position −1 relative to position 0, a G/U Wobble at position 3 relative to position 0, a 2/2 symmetric bulge at position 7 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 664. In some embodiments, the one or more structural features further comprises at least one structural feature selected from the group consisting of: a 6/6 symmetric internal loop at position −20 relative to position 0, a A/C mismatch at position −7 relative to position 0, a C/U mismatch at position −4 relative to position 0, a 2/2 symmetric bulge at position −1 relative to position 0, a 2/2 symmetric bulge at position 10 relative to position 0, an A/C mismatch at position 19 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 672. In some embodiments, the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, and any combination thereof. In some embodiments, the engineered guide RNA comprises at least about: 80%, 85%, 90%, 92%, 95%, 97%, or 99% sequence identity to SEQ ID NO: 675. In some embodiments, the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 676. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, and any combination thereof. In some embodiments, the engineered guide RNA comprises at least about: 80%, 85%, 90%, 92%, 95%, 97%, or 99% sequence identity to SEQ ID NO: 699. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 700. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, and any combination thereof. In some embodiments, the engineered guide RNA comprises at least about: 80%, 85%, 90%, 92%, 95%, 97%, or 99% sequence identity to SEQ ID NO: 718. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 719. In some embodiments, the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a C/C mismatch at position 9 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 745. In some embodiments, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 750. In some embodiments, the one or more structural features further comprises at least one structural feature selected from the group consisting of a 6/6 symmetric internal loop at position −20 relative to position 0, a A/A mismatch at position −7 relative to position 0, a 2/2 symmetric bulge at position −1 relative to position 0, a C/U mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 762. In some embodiments, the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a U/U mismatch at position 5 relative to position 0, a G/U Wobble at position 8 relative to position 0, a C/U mismatch at position 14 relative to position 0, a G/G mismatch at position 17 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 1099. In some embodiments, the one or more structural features further comprises at least one structural feature selected from the group consisting of a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a C/U mismatch at position 9 relative to position 0, a C/U mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 1120. In some embodiments, the one or more structural features further comprises at least one structural feature selected from the group consisting of: a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 1213. In some embodiments, the one or more structural features further comprises at least one structural feature selected from the group consisting of: a 6/6 symmetric internal loop at position −22 relative to position 0, a 3/3 symmetric bulge mismatch at position −13 relative to position 0, a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some embodiments, the engineered guide RNA comprises at least about: 80%, 85%, 90%, 92%, 95%, 97%, or 99% sequence identity to SEQ ID NO: 1214. In some embodiments, the first 6/6 symmetric internal loop is at position 25, relative to the target adenosine at position 0. In some embodiments, the one or more structural features further comprises at least one structural feature selected from the group consisting of: a 3/3 symmetric bulge at position −25 relative to position 0, a 3/3 symmetric bulge at position −16 relative to position 0, a 6/6 symmetric internal loop at position −4 relative to position 0, a G/G mismatch at position −1 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some embodiments, the engineered guide RNA comprises at least about: 80%, 85%, 90%, 92%, 95%, 97%, or 99% sequence identity to SEQ ID NO: 88. In some embodiments, the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some embodiments, the engineered guide RNA comprises at least about: 80%, 85%, 90%, 92%, 95%, 97%, or 99% sequence identity to SEQ ID NO: 903. In some embodiments, the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some embodiments, the engineered guide RNA comprises at least about: 80%, 85%, 90%, 92%, 95%, 97%, or 99% sequence identity to SEQ ID NO: 1179. In some embodiments, the one or more structural features further comprises at least one structural feature selected from the group consisting of: a 6/6 symmetric internal loop at position −4 relative to position 0, a G/G mismatch at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some embodiments, the engineered guide RNA comprises at least about: 80%, 85%, 90%, 92%, 95%, 97%, or 99% sequence identity to SEQ ID NO: 1212. In some embodiments, the first 6/6 symmetric internal loop is at position 24, relative to the target adenosine at position 0. In some embodiments, the one or more structural features further comprises at least one structural feature selected from the group consisting of: a 3/3 symmetric bulge at position −18 relative to position 0, a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 118. In some embodiments, the one or more structural features further comprises at least one structural feature selected from the group consisting of: a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 119. In some embodiments, the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 270. In some embodiments, the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 271. In some embodiments, the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 403. In some embodiments, the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some embodiments, the engineered guide RNA comprises at least about: 80%, 85%, 90%, 92%, 95%, 97%, or 99% sequence identity to SEQ ID NO: 1081. In some embodiments, the first 6/6 symmetric internal loop is at position 23, relative to the target adenosine at position 0. In some embodiments, the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 401. In some embodiments, the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 402. In some embodiments, the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 458. In some embodiments, the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some embodiments, the engineered guide RNA comprises at least about: 80%, 85%, 90%, 92%, 95%, 97%, or 99% sequence identity to SEQ ID NO: 459. In some embodiments, the first 6/6 symmetric internal loop is at position 22, relative to the target adenosine at position 0. In some embodiments, the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some embodiments, the engineered guide RNA comprises at least about: 80%, 85%, 90%, 92%, 95%, 97%, or 99% sequence identity to SEQ ID NO: 1135. In some embodiments, the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some embodiments, the engineered guide RNA comprises at least about: 80%, 85%, 90%, 92%, 95%, 97%, or 99% sequence identity to SEQ ID NO: 1136. In some embodiments, the one or more structural features comprises at least a first 6/6 symmetric internal loop and at least a second 6/6 symmetric loop. In some embodiments, upon hybridization of the engineered guide RNA to the sequence of the target MAPT RNA, the engineered guide RNA facilitates RNA editing of one or more adenosines in the sequence of the target MAPT RNA by an RNA editing entity. In some embodiments, the RNA editing entity comprises ADAR1, ADAR2, ADAR3, or any combination thereof. In some embodiments, the engineered guide RNA is encoded by an engineered polynucleotide. In some embodiments, the engineered polynucleotide is comprised in or on a vector. In some embodiments, the vector is a viral vector, and wherein the engineered polynucleotide is encapsidated in the viral vector. In some embodiments, the viral vector is an adeno-associated viral (AAV) vector or a derivative thereof. In some embodiments, the viral vector is an adeno-associated viral (AAV) vector and wherein the AAV vector is AAV1, AAV2, AAV3, AAV4, AAV5, AAV6, AAV7, AAV8, AAV9, AAV10, AAV11, or a derivative, a chimera, or a variant of any of these. In some embodiments, the AAV vector is a recombinant AAV (rAAV) vector, a hybrid AAV vector, a chimeric AAV vector, a self-complementary AAV (scAAV) vector, or any combination thereof. In some embodiments, the engineered guide RNA has at least 80%, 85%, 90%, 92%, 95%, 97%, or 99% sequence identity to any one of SEQ ID NO: 2-SEQ ID NO: 1215. In some embodiments, the composition is in unit dose form. In some embodiments, a composition described herein is for use in treatment of a disease or condition. In some embodiments, the disease or condition comprises a Tauopathy. In some embodiments, the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome.

Also disclosed herein are pharmaceutical compositions comprising: (a) an engineered guide RNA described herein or a composition comprising an engineered guide RNA described herein; and (b) a pharmaceutically acceptable: excipient, carrier, or diluent. In some embodiments, the pharmaceutical composition is in unit dose form. In some embodiments, a pharmaceutical composition described herein is for use in treatment of a disease or condition. In some embodiments, the disease or condition comprises a Tauopathy. In some embodiments, the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome.

Also disclosed herein are methods of treating a disease or a condition in a subject in need thereof, the method comprising administering to the subject a therapeutically effective amount of an engineered guide RNA described herein, a composition comprising an engineered guide RNA described herein, or a pharmaceutical composition comprising an engineered guide RNA described herein. In some embodiments, the disease or condition comprises a Tauopathy. In some embodiments, the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome. In some embodiments, the subject is a human or a non-human animal. In some embodiments, the administering is sufficient to treat one or more symptoms of the disease of condition. In some embodiments, the one of more symptoms treated comprises a reduction in memory loss, a reduction in confusion, or any combination thereof, as compared to prior to the administering. In some embodiments, the administering is sufficient to reduce aggregation of tau protein, relative to: (a) a level of aggregation prior to the administering; (b) a level of accumulated aggregation in the subject in the absence of the administering; or (c) both. In some embodiments, the administering is sufficient to reduce an amount of: (a) neurofibrillary tangles; (b) neuronal cell death; (c) neuroinflammation; or (d) any combination thereof; relative to an amount prior to the administering. In some embodiments, the amount of phosphorylated tau protein is decreased in the subject after the administering, relative to an amount of phosphorylated tau protein prior to the administering. In some embodiments, a ratio of a 4R isoform of Tau to a 3R isoform of tau is increased upon administration, relative to a ratio prior to the administering.

Also disclosed herein are methods of editing a c.1 translation initiation sequence (TIS) of a MAPT RNA, the method comprising contacting the MAPT RNA with an engineered guide RNA described herein or a composition comprising an engineered guide RNA described herein, and an RNA editing entity, thereby editing the MAPT RNA. In some embodiments, at least 40%, at least 50%, at least 60%, at least 70%, at least 80%, at least 85%, at least 90%, at least 95%, or at least 99% of the pre-mRNA transcripts of MAPT have at least one edit. In some embodiments, the editing of MAPT RNA facilitates a protein knockdown. In some embodiments, the protein knockdown comprises a reduction of at least 10%, relative to an amount of protein present prior to the contacting. In some embodiments, the protein knockdown comprises a reduction of from about 10% to about 25%, relative to an amount of protein present prior to the contacting. In some embodiments, the protein knockdown comprises a reduction of at least 50%, relative to an amount of protein present prior to the contacting. In some embodiments, the protein knockdown comprises a knockdown of tau. In some embodiments, the knockdown is measured in an in vitro assay. In some embodiments, the knockdown is measured in an in vivo assay. In some embodiments, the knockdown is measured in a human subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the present disclosure are set forth with particularity in the appended claims. A better understanding of the features and advantages of the present disclosure will be obtained by reference to the following detailed description that sets forth illustrative embodiments, in which exemplary principles of the present disclosure are utilized, and the accompanying drawings of which:

FIG. 1 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side).

FIG. 2 is a plot showing, on the x-axis, the sequence similarity of the MAPT TIS-targeting engineered guide RNAs of the present disclosure to a canonical guide RNA design and, on the y-axis, the edited fraction by an ADAR2 enzyme. These data highlight the diverse sequence space represented by the MAPT TIS-targeting engineered guide RNAs of the present disclosure, which have a range of different structural features that drive sequence diversity and which exhibit high on-target editing efficiency.

FIG. 3 shows a schematic of the MAPT genomic landscape. Engineered guide RNAs of the present disclosure can target any TIS in MAPT, including at the sites indicated as “ntl”, “nt31”, “nt91”, or a combination thereof. “ntl” may also be referred to herein as “c.1” (coding nucleotide 1) and is the 18^{th} nt of Exon 1. “nt31” may also be referred to herein as “c.31” and is the 48^{th} nt of Exon 1. “nt9l” may also be referred to herein as “c.91” and is the 108^{th} nt of Exon 1.

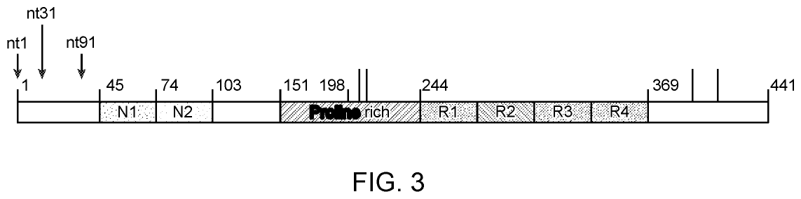

### DETAILED DESCRIPTION

### RNA Editing

RNA editing can refer to a process by which RNA is enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from: cytidine to give rise to uridine, or C-to-U editing; or from adenosine to inosine, or A-to-I editing). RNA editing can be used to correct mutations (e.g., correction of a missense mutation) in order to restore protein expression and to introduce mutations or edit coding or non-coding regions of RNA to inhibit RNA translation and effect protein knockdown.

Described herein are engineered guide RNAs that facilitate RNA editing by an RNA editing entity (e.g., an adenosine Deaminase Acting on RNA (ADAR)) or biologically active fragments thereof. For example, engineered guide RNAs of the present disclosure can facilitate editing of a transcription initiation site (e.g. the c.1 transcription initiation site) of a target MAPT mRNA (for example, an engineered guide RNA of any one of SEQ ID NO: 2-1215). In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADARs 1-3). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain.

The engineered guide RNAs (e.g. an engineered guide RNA of any one of SEQ ID NO: 2-1215 as recited in Table 1) of the present disclosure facilitate RNA editing (for example, of a MAPT c.1 transcription initiation site) by endogenous ADAR enzymes. In some embodiments, exogenous ADAR can be delivered alongside the engineered guide RNAs disclosed herein to facilitate RNA editing. In some embodiments, the ADAR is human ADAR1. In some embodiments, the ADAR is human ADAR2. In some embodiments, the ADAR is human ADAR3. In some embodiments, the ADAR is human ADAR1, human ADAR2, human ADAR2, or any combination thereof.

The present disclosure, in some embodiments, provides engineered guide RNAs that facilitate edits at particular regions in a target RNA (e.g., mRNA or pre-mRNA). For example, the engineered guide RNAs disclosed herein can target a coding sequence or a non-coding sequence of an RNA. For example, a target region in a coding sequence of an RNA can be a translation initiation site (TIS). In some embodiments, the target region in a non-coding sequence of an RNA can be a polyadenylation (polyA) signal sequence.

TIS. In some embodiments, the engineered guide RNAs of the present disclosure target the adenosine at a translation initiation site (TIS). In some embodiments, an engineered guide RNA of the present disclosure (e.g. an engineered guide RNA of any one of SEQ ID NO: 2-1215 as recited in Table 1) can target the c.1 TIS corresponding to the canonical TIS at nucleotide position 151 of the MAPT reference transcript of NM_001377265.1. The engineered guide RNAs facilitate ADAR-mediated RNA editing of the TIS (AUG) to GUG. This results in inhibition of RNA translation and, thereby, protein knockdown. Protein knockdown can also be referred to as reduced expression of wild-type protein. Engineered guide RNAs of the present disclosure can target one or any combination of the TISs of MAPT. For example, in some embodiments, engineered guide RNAs of the present disclosure target the TIS at the 18^{th} nt of Exon 1 (also referred to as the TIS at “c.1,” for coding nucleotide 1″), such as an engineered guide RNA of any one of SEQ ID Nos: 2-1215. In some embodiments, engineered guide RNAs of the present disclosure target the TIS at the 48^{th} nt of Exon 1 (also referred to as the TIS at “c.31”). In some embodiments, engineered guide RNAs of the present disclosure target the TIS at the 108^{th} nt of Exon 1 (also referred to as the TIS at “c.91”). In some embodiments, engineered guide RNAs of the present disclosure target the TIS at the 6^{th} nt of Exon 5 (also referred to as the TIS at “c.379”). In some embodiments, engineered guide RNAs of the present disclosure target at least one TIS selected from the group consisting of c.1, c.31, c.91, and c.379. In some embodiments, engineered guide RNAs of the present disclosure target at least two TISs selected from the group consisting of c.1, c.31, c.91, and c.379. In some embodiments, engineered guide RNAs of the present disclosure target at least three TISs selected from the group consisting of c.1, c.31, c.91, and c.379. In some embodiments, engineered guide RNAs of the present disclosure target the c.1 TIS, the c.31 TIS, the c.91 TIS, and the c.379 TIS. In some embodiments, an engineered guide RNA of the present disclosure targets the c.1 TIS. In some embodiments, an engineered guide RNA targeting the c.1 TIS is an engineered guide RNA having at least 80%, 85%, 90%, 92%, 95%, 97%, or 99% sequence identity to any one of SEQ ID NO: 2-SEQ ID NO: 1215.

3′UTR. In some embodiments, the engineered guide RNAs of the present disclosure target one or more adenosines in the 3′ untranslated region (3′UTR). In some embodiments, an engineered guide RNA facilitates ADAR-mediated RNA editing of the one or more adenosines in the 3′UTR, thereby reducing mRNA export from the nucleus and inhibiting translation, thereby resulting in protein knockdown.

5′UTR. In some embodiments, the engineered guide RNAs of the present disclosure target one or more adenosines in the 5′ untranslated region (5′ UTR). In some embodiments, an engineered guide RNA of the present disclosure can target a Kozak sequence of the 5′ UTR. In some embodiments, an engineered guide RNA of the present disclosure can target an internal ribosomal entry site (IRES) of the 5′ UTR. In some embodiments, an engineered guide RNA of the present disclosure can target an iron response element (IRE) of the 5′ UTR. In some embodiments, an engineered guide RNA facilitates ADAR-mediated RNA editing of one or more adenosines the 5′UTR (including one or more adenosines present in one or more structures of the 5′ UTR). In some instances, extensive or hyper editing of a plurality of adenosines can be facilitated via an engineered guide RNA of the present disclosure, which can result in ribosomal stalling of the mRNA transcript, thereby resulting in protein knockdown.

PolyA Signal Sequence. In some embodiments, the engineered guide RNAs of the present disclosure target one or more adenosines in the polyA signal sequence. In some embodiments, an engineered guide RNA facilitates ADAR-mediated RNA editing of the one or more adenosines in the polyA signal sequence, thereby resulting in disruption of RNA processing and degradation of the target mRNA and, thereby, protein knockdown. In some embodiments, a target can have one or more polyA signal sequences. In these instances, one or more engineered guide RNAs, varying in their respective sequences, of the present disclosure can be multiplexed to target adenosines in the one or more polyA signal sequences. In some embodiments, engineered guide RNAs of the present disclosure targeting the canonical TIS (the c.1 TIS) of MAPT can be multiplexed with one or more additional engineered guide RNAs targeting a different TIS of MAPT, such as the c.11 TIS, the c.31 TIS, or the c.91 TIS. Alternatively, or in addition, one or more engineered guide RNAs of the present disclosure targeting the canonical (c.1) TIS of MAPT may be multiplexed with or more engineered guide RNAs targeting a different sequence of MAPT, such as the 5′UTR region of MAPT (e.g., a Kozak sequence, an internal ribosomal entry site (IRES), or an iron response element (TRE) of the 5′ UTR). In each of these cases, the multiplexed engineered guide RNAs can be delivered together in the same viral vector or the each of the distinct engineered guide RNAs can be delivered together but in separate vectors. In any of these cases, the engineered guide RNAs of the present disclosure facilitated ADAR-mediated RNA editing of adenosines to inosines (read as guanosines by cellular machinery) in the polyA signal sequence, resulting in protein knockdown.

### Engineered Guide RNAs

Disclosed herein are engineered guide RNAs (e.g. an engineered guide RNA of any one of SEQ ID NO: 2-1215 as recited in Table 1) and engineered polynucleotides encoding the same for site-specific, selective editing of a target RNA (for example, the c.1 TIS corresponding to the canonical TIS at nucleotide position 151 of the MAPT reference transcript of NM_001377265.1) via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA as described herein comprises a targeting domain with complementarity to a target RNA described herein. As such, a guide RNA can be engineered to site-specifically/selectively target and hybridize to a particular target RNA, thus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### A. Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule (e.g. the c.1 TIS corresponding to the canonical TIS at nucleotide position 151 of the MAPT reference transcript of NM_001377265.1). A targeting sequence can also be referred to as a “targeting domain” or a “targeting region”.

In some cases, a targeting domain of an engineered guide allows the engineered guide to target an RNA sequence through base pairing, such as Watson Crick base pairing. In some examples, the targeting sequence can be located at either the N-terminus or C-terminus of the engineered guide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

The targeting sequence can have sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can facilitate editing of a base of a nucleotide of in a target sequence of a target RNA that results in modulating the expression of a polypeptide encoded by the target RNA. In some instances, modulation can be decrease expression of the polypeptide. In some cases, an engineered guide can be configured to facilitate an editing of a base of a nucleotide or polynucleotide of a region of an RNA by an RNA editing entity (e.g., ADAR). In order to facilitate editing, an engineered polynucleotide of the disclosure can recruit an RNA editing entity (e.g., ADAR). Various RNA editing entity recruiting domains can be utilized. In some examples, a recruiting domain comprises: Glutamate ionotropic receptor AMPA type subunit 2 (GluR2), an Alu sequence, or, in the case of recruiting APOBEC, an APOBEC recruiting domain.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence hybridizes to a target sequence of a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In an embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain can comprise at least about 80% sequence homology to at least about 10, 15, 20, 25, or 30 nucleotides of SEQ ID NO: 1. In some examples, a recruiting domain can comprise at least about 90%, 95%, 96%, 97%, 98%, or 99% sequence homology and/or length to SEQ ID NO: 1.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of engineered guide RNAs. In some cases, a recruiting domain can be on an N-terminus, middle, or C-terminus of an engineered guide RNA. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribo- and deoxyribonucleotides can in some cases not be excluded.

C. Engineered Guide RNAs with Latent Structure

In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double stranded RNA (dsRNA) substrate is formed upon hybridization of an engineered guide RNA of the present disclosure to a target RNA (for example, a MAPT c.1 TIS). The resulting dsRNA substrate is also referred to herein as a “guide-target RNA scaffold.”

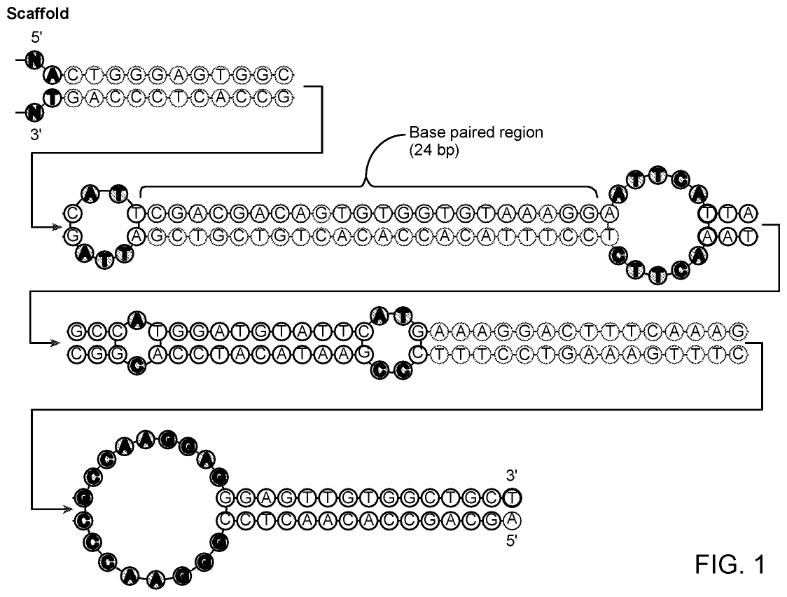

FIG. 1 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence can be absent, an engineered guide RNA can be still capable of associating with a subject RNA editing entity (e.g., ADAR) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A guide-target RNA scaffold is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A guide-target RNA scaffold is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A guide-target RNA scaffold is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A guide-target RNA scaffold is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some embodiments, an asymmetric bulge can be a 1/0 asymmetric bulge. In some embodiments, a 1/0 asymmetric bulge can be a U deletion. A “U deletion” refers to a 1/0 asymmetric bulge in which a U nucleotide of an engineered guide RNA that would be situated opposite a non-target A of a target RNA in the guide-target RNA scaffold is deleted from the engineered guide RNA. In some instances, a 1/0 asymmetric bulge comprising a U deletion can reduce editing of the non-target A, relative to a comparable guide RNA lacking the U deletion.

In some cases, a structural feature can be an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

The present disclosure provides engineered guide RNAs (for example, an engineered guide RNA of any one of SEQ ID NO: 2-1215 as recited in Table 1) that target a sequence of a MAPT target RNA (for example, the c.1 TIS corresponding to the canonical TIS at nucleotide position 151 of the MAPT reference transcript of NM_001377265.1).

In some cases, an engineered guide RNA comprises one or more structural features that manifest as latent structures which result in editing of a target adenosine (defined as position 0) in a c.1 TIS target sequence of a MAPT RNA. In some embodiments, the one or more structural features comprises a first 6/6 symmetric internal loop and a second symmetric 6/6 internal loop. In some embodiments, the one or more structural features comprises: a first 6/6 symmetric internal loop at a position selected from the group consisting of: 34, 33, 32, 31, 30, 26, 25, 24, 23, and 22, relative to the target adenosine at position 0.

In some cases, the one or more structural features comprises the first 6/6 symmetric internal loop is at position 34, relative to the target adenosine at position 0. In some cases, the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 478. In some cases, an engineered guide RNA of the present disclosure that targets a c.1 TIS target sequence of a MAPT RNA has a sequence of SEQ ID NO: 478.

In some cases, the one or more structural features comprises the first 6/6 symmetric internal loop is at position 34, relative to the target adenosine at position 0. In some cases, the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 479. In some cases, an engineered guide RNA of the present disclosure that targets a c.1 TIS target sequence of a MAPT RNA has a sequence of SEQ ID NO: 479.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 592. In some cases, an engineered guide RNA of the present disclosure that targets a c.1 TIS target sequence of a MAPT RNA has a sequence of SEQ ID NO: 592.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 593. In some cases, an engineered guide RNA of the present disclosure that targets a c.1 TIS target sequence of a MAPT RNA has a sequence of SEQ ID NO: 593.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 639. In some cases, an engineered guide RNA of the present disclosure that targets a c.1 TIS target sequence of a MAPT RNA has a sequence of SEQ ID NO: 639.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 640. In some cases, an engineered guide RNA of the present disclosure that targets a c.1 TIS target sequence of a MAPT RNA has a sequence of SEQ ID NO: 640.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 686. In some cases, an engineered guide RNA of the present disclosure that targets a c.1 TIS target sequence of a MAPT RNA has a sequence of SEQ ID NO: 686.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 687. In some cases, an engineered guide RNA of the present disclosure that targets a c.1 TIS target sequence of a MAPT RNA has a sequence of SEQ ID NO: 687.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 688. In some cases, an engineered guide RNA of the present disclosure that targets a c.1 TIS target sequence of a MAPT RNA has a sequence of SEQ ID NO: 688.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 695. In some cases, an engineered guide RNA of the present disclosure that targets a c.1 TIS target sequence of a MAPT RNA has a sequence of SEQ ID NO: 695.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 696. In some cases, an engineered guide RNA of the present disclosure that targets a c.1 TIS target sequence of a MAPT RNA has a sequence of SEQ ID NO: 696.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 714. In some cases, an engineered guide RNA of the present disclosure that targets a c.1 TIS target sequence of a MAPT RNA has a sequence of SEQ ID NO: 714.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 715. In some cases, an engineered guide RNA of the present disclosure that targets a c.1 TIS target sequence of a MAPT RNA has a sequence of SEQ ID NO: 715.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 722. In some cases, an engineered guide RNA of the present disclosure that targets a c.1 TIS target sequence of a MAPT RNA has a sequence of SEQ ID NO: 722.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 723. In some cases, an engineered guide RNA of the present disclosure that targets a c.1 TIS target sequence of a MAPT RNA has a sequence of SEQ ID NO: 723.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −22 relative to position 0, a 3/3 symmetric bulge at position −13 relative to position 0, a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1059. In some cases, an engineered guide RNA of the present disclosure that targets a c.1 TIS target sequence of a MAPT RNA has a sequence of SEQ ID NO: 1059.

In some cases, the one or more structural features comprises the first 6/6 symmetric internal loop is at position 33, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1116. In some cases, an engineered guide RNA of the present disclosure that targets a c.1 TIS target sequence of a MAPT RNA has a sequence of SEQ ID NO: 1116.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1117. In some cases, an engineered guide RNA of the present disclosure that targets a c.1 TIS target sequence of a MAPT RNA has a sequence of SEQ ID NO: 1117.

In some cases, the one or more structural features comprises the first 6/6 symmetric internal loop is at position 33, relative to the target adenosine at position 0. In some cases, the one or more structural features further comprises at least one structural feature selected from the group consisting of: a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1117. In some cases, an engineered guide RNA of the present disclosure that targets a c.1 TIS target sequence of a MAPT RNA has a sequence of SEQ ID NO: 1215.

In some cases, the one or more structural features comprises the first 6/6 symmetric internal loop is at position 32, relative to the target adenosine at position 0. In some cases, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 438. In some cases, an engineered guide RNA of the present disclosure that targets a c.1 TIS target sequence of a MAPT RNA has a sequence of SEQ ID NO: 438.

In some cases, the one or more structural features comprises the first 6/6 symmetric internal loop is at position 32, relative to the target adenosine at position 0. In some cases, the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 439. In some cases, an engineered guide RNA of the present disclosure that targets a c.1 TIS target sequence of a MAPT RNA has a sequence of SEQ ID NO: 439.

In some cases, the one or more structural features comprises the first 6/6 symmetric internal loop is at position 32, relative to the target adenosine at position 0. In some cases, the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 816. In some cases, an engineered guide RNA of the present disclosure that targets a c.1 TIS target sequence of a MAPT RNA has a sequence of SEQ ID NO: 816.

In some cases, the one or more structural features comprises the first 6/6 symmetric internal loop is at position 32, relative to the target adenosine at position 0. In some cases, the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 817. In some cases, an engineered guide RNA of the present disclosure that targets a c.1 TIS target sequence of a MAPT RNA has a sequence of SEQ ID NO: 817.

In some cases, the one or more structural features comprises the first 6/6 symmetric internal loop is at position 31, relative to the target adenosine at position 0. In some cases, the one or more structural features further comprises at least one structural feature selected from the group consisting of: a 3/3 symmetric bulge at position −19 relative to position 0, a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 162. In some cases, an engineered guide RNA of the present disclosure that targets a c.1 TIS target sequence of a MAPT RNA has a sequence of SEQ ID NO: 162.

In some cases, the one or more structural features comprises the first 6/6 symmetric internal loop is at position 31, relative to the target adenosine at position 0. In some cases, the one or more structural features further comprises at least one structural feature selected from the group consisting of: a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 163. In some cases, an engineered guide RNA of the present disclosure that targets a c.1 TIS target sequence of a MAPT RNA has a sequence of SEQ ID NO: 163.

In some cases, the one or more structural features comprises the first 6/6 symmetric internal loop is at position 31, relative to the target adenosine at position 0. In some cases, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 929. In some cases, an engineered guide RNA of the present disclosure that targets a c.1 TIS target sequence of a MAPT RNA has a sequence of SEQ ID NO: 929.

In some cases, the one or more structural features comprises the first 6/6 symmetric internal loop is at position 31, relative to the target adenosine at position 0. In some cases, the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 930. In some cases, an engineered guide RNA of the present disclosure that targets a c.1 TIS target sequence of a MAPT RNA has a sequence of SEQ ID NO: 930.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 538. In some cases, an engineered guide RNA of the present disclosure that targets a c.1 TIS target sequence of a MAPT RNA has a sequence of SEQ ID NO: 538.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 539. In some cases, an engineered guide RNA of the present disclosure that targets a c.1 TIS target sequence of a MAPT RNA has a sequence of SEQ ID NO: 539.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 604. In some cases, an engineered guide RNA of the present disclosure that targets a c.1 TIS target sequence of a MAPT RNA has a sequence of SEQ ID NO: 604.

In some cases, the one or more structural features comprises the first 6/6 symmetric internal loop is at position 30, relative to the target adenosine at position 0. In some cases, the one or more structural features further comprises at least one structural feature selected from the group consisting of: a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 605. In some cases, an engineered guide RNA of the present disclosure that targets a c.1 TIS target sequence of a MAPT RNA has a sequence of SEQ ID NO: 605.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 619. In some cases, an engineered guide RNA of the present disclosure that targets a c.1 TIS target sequence of a MAPT RNA has a sequence of SEQ ID NO: 619.

In some cases, the one or more structural features comprises the first 6/6 symmetric internal loop is at position 30, relative to the target adenosine at position 0. In some cases, the one or more structural features further comprises at least one structural feature selected from the group consisting of: a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 620. In some cases, an engineered guide RNA of the present disclosure that targets a c.1 TIS target sequence of a MAPT RNA has a sequence of SEQ ID NO: 620.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 677. In some cases, an engineered guide RNA of the present disclosure that targets a c.1 TIS target sequence of a MAPT RNA has a sequence of SEQ ID NO: 677.

In some cases, the one or more structural features comprises the first 6/6 symmetric internal loop is at position 30, relative to the target adenosine at position 0. In some cases, the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 678. In some cases, an engineered guide RNA of the present disclosure that targets a c.1 TIS target sequence of a MAPT RNA has a sequence of SEQ ID NO: 678.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 684. In some cases, an engineered guide RNA of the present disclosure that targets a c.1 TIS target sequence of a MAPT RNA has a sequence of SEQ ID NO: 684.

In some cases, the one or more structural features comprises the first 6/6 symmetric internal loop is at position 30, relative to the target adenosine at position 0. In some cases, the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 685. In some cases, an engineered guide RNA of the present disclosure that targets a c.1 TIS target sequence of a MAPT RNA has a sequence of SEQ ID NO: 685.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 720. In some cases, an engineered guide RNA of the present disclosure that targets a c.1 TIS target sequence of a MAPT RNA has a sequence of SEQ ID NO: 720.

In some cases, the one or more structural features comprises the first 6/6 symmetric internal loop is at position 30, relative to the target adenosine at position 0. In some cases, the one or more structural features further comprises at least one structural feature selected from the group consisting of: a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 721. In some cases, an engineered guide RNA of the present disclosure that targets a c.1 TIS target sequence of a MAPT RNA has a sequence of SEQ ID NO: 721.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 733. In some cases, an engineered guide RNA of the present disclosure that targets a c.1 TIS target sequence of a MAPT RNA has a sequence of SEQ ID NO: 733.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 734. In some cases, an engineered guide RNA of the present disclosure that targets a c.1 TIS target sequence of a MAPT RNA has a sequence of SEQ ID NO: 734.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 763. In some cases, an engineered guide RNA of the present disclosure that targets a c.1 TIS target sequence of a MAPT RNA has a sequence of SEQ ID NO: 763.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 764. In some cases, an engineered guide RNA of the present disclosure that targets a c.1 TIS target sequence of a MAPT RNA has a sequence of SEQ ID NO: 764.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 775. In some cases, an engineered guide RNA of the present disclosure that targets a c.1 TIS target sequence of a MAPT RNA has a sequence of SEQ ID NO: 775.

In some cases, the one or more structural features comprises the first 6/6 symmetric internal loop is at position 30, relative to the target adenosine at position 0. In some cases, the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 776. In some cases, an engineered guide RNA of the present disclosure that targets a c.1 TIS target sequence of a MAPT RNA has a sequence of SEQ ID NO: 776.

In some cases, the one or more structural features comprises the first 6/6 symmetric internal loop is at position 30, relative to the target adenosine at position 0. In some cases, the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 795. In some cases, an engineered guide RNA of the present disclosure that targets a c.1 TIS target sequence of a MAPT RNA has a sequence of SEQ ID NO: 795.

In some cases, the one or more structural features comprises the first 6/6 symmetric internal loop is at position 30, relative to the target adenosine at position 0. In some cases, the one or more structural features further comprises at least one structural feature selected from the group consisting of: a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 796. In some cases, an engineered guide RNA of the present disclosure that targets a c.1 TIS target sequence of a MAPT RNA has a sequence of SEQ ID NO: 796.

In some cases, the one or more structural features comprises the first 6/6 symmetric internal loop is at position 26, relative to the target adenosine at position 0. In some cases, the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 464. In some cases, an engineered guide RNA of the present disclosure that targets a c.1 TIS target sequence of a MAPT RNA has a sequence of SEQ ID NO: 464.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 465. In some cases, an engineered guide RNA of the present disclosure that targets a c.1 TIS target sequence of a MAPT RNA has a sequence of SEQ ID NO: 465.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 583. In some cases, an engineered guide RNA of the present disclosure that targets a c.1 TIS target sequence of a MAPT RNA has a sequence of SEQ ID NO: 583.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 584. In some cases, an engineered guide RNA of the present disclosure that targets a c.1 TIS target sequence of a MAPT RNA has a sequence of SEQ ID NO: 584.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a U/U mismatch at position −11 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 589. In some cases, an engineered guide RNA of the present disclosure that targets a c.1 TIS target sequence of a MAPT RNA has a sequence of SEQ ID NO: 589.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 610. In some cases, an engineered guide RNA of the present disclosure that targets a c.1 TIS target sequence of a MAPT RNA has a sequence of SEQ ID NO: 610.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 611. In some cases, an engineered guide RNA of the present disclosure that targets a c.1 TIS target sequence of a MAPT RNA has a sequence of SEQ ID NO: 611.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 618. In some cases, an engineered guide RNA of the present disclosure that targets a c.1 TIS target sequence of a MAPT RNA has a sequence of SEQ ID NO: 618.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a A/G mismatch at position −7 relative to position 0, a C/U mismatch at position −4 relative to position 0, a 2/2 symmetric bulge at position −1 relative to position 0, a G/U Wobble at position 8 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 662. In some cases, an engineered guide RNA of the present disclosure that targets a c.1 TIS target sequence of a MAPT RNA has a sequence of SEQ ID NO: 662.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 3 relative to position 0, a 2/2 symmetric bulge at position 7 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 664. In some cases, an engineered guide RNA of the present disclosure that targets a c.1 TIS target sequence of a MAPT RNA has a sequence of SEQ ID NO: 664.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a A/C mismatch at position −7 relative to position 0, a C/U mismatch at position −4 relative to position 0, a 2/2 symmetric bulge at position −1 relative to position 0, a 2/2 symmetric bulge at position 10 relative to position 0, an A/C mismatch at position 19 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 672. In some cases, an engineered guide RNA of the present disclosure that targets a c.1 TIS target sequence of a MAPT RNA has a sequence of SEQ ID NO: 672.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 675. In some cases, an engineered guide RNA of the present disclosure that targets a c.1 TIS target sequence of a MAPT RNA has a sequence of SEQ ID NO: 675.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 676. In some cases, an engineered guide RNA of the present disclosure that targets a c.1 TIS target sequence of a MAPT RNA has a sequence of SEQ ID NO: 676.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 699. In some cases, an engineered guide RNA of the present disclosure that targets a c.1 TIS target sequence of a MAPT RNA has a sequence of SEQ ID NO: 699.

sequence of a MAPT RNA has a sequence of SEQ ID NO: 676.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 700. In some cases, an engineered guide RNA of the present disclosure that targets a c.1 TIS target sequence of a MAPT RNA has a sequence of SEQ ID NO: 700.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 718. In some cases, an engineered guide RNA of the present disclosure that targets a c.1 TIS target sequence of a MAPT RNA has a sequence of SEQ ID NO: 718.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 719. In some cases, an engineered guide RNA of the present disclosure that targets a c.1 TIS target sequence of a MAPT RNA has a sequence of SEQ ID NO: 719.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a C/C mismatch at position 9 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 745. In some cases, an engineered guide RNA of the present disclosure that targets a c.1 TIS target sequence of a MAPT RNA has a sequence of SEQ ID NO: 745.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 750. In some cases, an engineered guide RNA of the present disclosure that targets a c.1 TIS target sequence of a MAPT RNA has a sequence of SEQ ID NO: 750.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a A/A mismatch at position −7 relative to position 0, a 2/2 symmetric bulge at position −1 relative to position 0, a C/U mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 762. In some cases, an engineered guide RNA of the present disclosure that targets a c.1 TIS target sequence of a MAPT RNA has a sequence of SEQ ID NO: 762.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a U/U mismatch at position 5 relative to position 0, a G/U Wobble at position 8 relative to position 0, a C/U mismatch at position 14 relative to position 0, a G/G mismatch at position 17 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1099. In some cases, an engineered guide RNA of the present disclosure that targets a c.1 TIS target sequence of a MAPT RNA has a sequence of SEQ ID NO: 1099.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a C/U mismatch at position 9 relative to position 0, a C/U mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1120. In some cases, an engineered guide RNA of the present disclosure that targets a c.1 TIS target sequence of a MAPT RNA has a sequence of SEQ ID NO: 1120.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1213. In some cases, an engineered guide RNA of the present disclosure that targets a c.1 TIS target sequence of a MAPT RNA has a sequence of SEQ ID NO: 1213.

In some cases, the one or more structural features comprises the first 6/6 symmetric internal loop is at position 26, relative to the target adenosine at position 0. In some cases, the one or more structural features further comprises at least one structural feature selected from the group consisting of: a 6/6 symmetric internal loop at position −22 relative to position 0, a 3/3 symmetric bulge mismatch at position −13 relative to position 0, a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1214. In some cases, an engineered guide RNA of the present disclosure that targets a c.1 TIS target sequence of a MAPT RNA has a sequence of SEQ ID NO: 1214.

In some cases, the one or more structural features comprises the first 6/6 symmetric internal loop is at position 25, relative to the target adenosine at position 0. In some cases, the one or more structural features further comprises at least one structural feature selected from the group consisting of: a 3/3 symmetric bulge at position −25 relative to position 0, a 3/3 symmetric bulge at position −16 relative to position 0, a 6/6 symmetric internal loop at position −4 relative to position 0, a G/G mismatch at position −1 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 88. In some cases, an engineered guide RNA of the present disclosure that targets a c.1 TIS target sequence of a MAPT RNA has a sequence of SEQ ID NO: 88.

In some cases, the one or more structural features comprises the first 6/6 symmetric internal loop is at position 25, relative to the target adenosine at position 0. In some cases, the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 903. In some cases, an engineered guide RNA of the present disclosure that targets a c.1 TIS target sequence of a MAPT RNA has a sequence of SEQ ID NO: 903.

In some cases, the one or more structural features comprises the first 6/6 symmetric internal loop is at position 25, relative to the target adenosine at position 0. In some cases, the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1179. In some cases, an engineered guide RNA of the present disclosure that targets a c.1 TIS target sequence of a MAPT RNA has a sequence of SEQ ID NO: 1179.

In some cases, the one or more structural features comprises the first 6/6 symmetric internal loop is at position 25, relative to the target adenosine at position 0. In some cases, the one or more structural features further comprises at least one structural feature selected from the group consisting of: a 6/6 symmetric internal loop at position −4 relative to position 0, a G/G mismatch at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1212. In some cases, an engineered guide RNA of the present disclosure that targets a c.1 TIS target sequence of a MAPT RNA has a sequence of SEQ ID NO: 1212.

In some cases, the one or more structural features comprises the first 6/6 symmetric internal loop is at position 24, relative to the target adenosine at position 0. In some cases, the one or more structural features further comprises at least one structural feature selected from the group consisting of: a 3/3 symmetric bulge at position −18 relative to position 0, a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 118. In some cases, an engineered guide RNA of the present disclosure that targets a c.1 TIS target sequence of a MAPT RNA has a sequence of SEQ ID NO: 118.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 119. In some cases, an engineered guide RNA of the present disclosure that targets a c.1 TIS target sequence of a MAPT RNA has a sequence of SEQ ID NO: 119.

In some cases, the one or more structural features comprises the first 6/6 symmetric internal loop is at position 24, relative to the target adenosine at position 0. In some cases, the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 270. In some cases, an engineered guide RNA of the present disclosure that targets a c.1 TIS target sequence of a MAPT RNA has a sequence of SEQ ID NO: 270.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 271. In some cases, an engineered guide RNA of the present disclosure that targets a c.1 TIS target sequence of a MAPT RNA has a sequence of SEQ ID NO: 271.

In some cases, the one or more structural features comprises the first 6/6 symmetric internal loop is at position 24, relative to the target adenosine at position 0. In some cases, the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 403. In some cases, an engineered guide RNA of the present disclosure that targets a c.1 TIS target sequence of a MAPT RNA has a sequence of SEQ ID NO: 403.

In some cases, the one or more structural features comprises the first 6/6 symmetric internal loop is at position 24, relative to the target adenosine at position 0. In some cases, the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1081. In some cases, an engineered guide RNA of the present disclosure that targets a c.1 TIS target sequence of a MAPT RNA has a sequence of SEQ ID NO: 1081.

In some cases, the one or more structural features comprises the first 6/6 symmetric internal loop is at position 23, relative to the target adenosine at position 0. In some cases, the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 401. In some cases, an engineered guide RNA of the present disclosure that targets a c.1 TIS target sequence of a MAPT RNA has a sequence of SEQ ID NO: 401.

In some cases, the one or more structural features comprises the first 6/6 symmetric internal loop is at position 23, relative to the target adenosine at position 0. In some cases, the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 402. In some cases, an engineered guide RNA of the present disclosure that targets a c.1 TIS target sequence of a MAPT RNA has a sequence of SEQ ID NO: 402.

In some cases, the one or more structural features comprises the first 6/6 symmetric internal loop is at position 23, relative to the target adenosine at position 0. In some cases, the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 458. In some cases, an engineered guide RNA of the present disclosure that targets a c.1 TIS target sequence of a MAPT RNA has a sequence of SEQ ID NO: 458.

In some cases, the one or more structural features comprises the first 6/6 symmetric internal loop is at position 23, relative to the target adenosine at position 0. In some cases, the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 459. In some cases, an engineered guide RNA of the present disclosure that targets a c.1 TIS target sequence of a MAPT RNA has a sequence of SEQ ID NO: 459.

In some cases, the one or more structural features comprises the first 6/6 symmetric internal loop is at position 22, relative to the target adenosine at position 0. In some cases, the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1135. In some cases, an engineered guide RNA of the present disclosure that targets a c.1 TIS target sequence of a MAPT RNA has a sequence of SEQ ID NO: 1135.

In some cases, the one or more structural features comprises the first 6/6 symmetric internal loop is at position 22, relative to the target adenosine at position 0. In some cases, the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. In some cases, an engineered guide RNA of the present disclosure that targets a c.1 TIS target sequence of a MAPT RNA has at least 80%, 85%, 90%, 92%, 95%, 97%, or 99% sequence identity to a guide RNA comprising SEQ ID NO: 1136. In some cases, an engineered guide RNA of the present disclosure that targets a c.1 TIS target sequence of a MAPT RNA has a sequence of SEQ ID NO: 1136.

### Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both sequences.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl. In some embodiments, a circular engineered guide RNA can comprise a guide RNA from any one of SEQ ID NOs: 2-1215 as recited in Table 1 that target MAPT c.1 TIS.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. Circular guide RNAs are described in PCT/US2021/034301, which is incorporated by reference in its entirety.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g. increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing the first 18 nt of the U7 snRNA (which naturally hybridizes to the spacer element of histone pre-mRNA) with a short targeting (or antisense) sequence of a disease gene, redirects the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated Ut, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild-type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g. an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g. a pre-mRNA or a mature RNA) at a greater efficiency than a comparable polynucleotide lacking such features. Promoters and snRNA components are described in PCT/US2021/028618, which is incorporated by reference in its entirety.

Disclosed herein are engineered RNAs comprising (a) an engineered guide RNA as described herein, and (b) a U7 snRNA hairpin sequence, a SmOPT sequence, or a combination thereof. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 1216 or RNA: UAGGCUUUCUGGCUUUUUACCGGAAAGCCCCU (SEQ ID NO: 1217). In some cases, a mouse U7 hairpin sequence comprises CAGGTTTTCTGACTTCGGTCGGAAAACCCCT (SEQ ID NO: 1218 or RNA: CAGGUUUUCUGACUUCGGUCGGAAAACCCCU SEQ ID NO: 1219). In some embodiments, the SmOPT sequence has a sequence of AATTTTTGGAG (SEQ ID NO: 1220 or RNA: AAUUUUUGGAG SEQ ID NO: 1221). In some embodiments, a guide RNA from any one of SEQ ID NOs: 2-1215 as recited in Table 1 that target MAPT c.1 TIS can comprise a guide RNA comprising a U7 hairpin sequence (e.g., a human or a mouse U7 hairpin sequence), an SmOPT sequence, or a combination thereof. In some cases, a combination of a U7 hairpin sequence and a SmOPT sequence can comprise a SmOPT U7 hairpin sequence, wherein the SmOPT sequence is linked to the U7 sequence. In some cases, a U7 hairpin sequence, an SmOPT sequence, or a combination thereof is downstream (e.g., 3′) of the engineered guide RNA disclosed herein.

Also disclosed herein are promoters for driving the expression of a guide RNA disclosed herein. In some cases, the promoters for driving expression can be 5′ to the guide RNA sequence disclosed herein. In some cases, a promoter can comprise a U1 promoter, a U7 promoter, a U6 promoter or any combination thereof. In some cases, a promoter can comprise a CMV promoter. In some cases, a U7 promoter, or a U6 promoter can be a mouse U7 promoter, or a mouse U6 promoter. In some cases, a U1 promoter, a U7 promoter, or a U6 promoter can be a human U1 promoter, a human U7 promoter, or a human U6 promoter. In some cases, a human U6 promoter can comprise a sequence with at least about: 70%, 75%, 80%, 85%, 90%, 95%, or 99% sequence identity to: GAGGGCCTATTTCCCATGATTCCTTCATATTTGCATATACGATACAAGGCTGTTAGA GAGATAATTAGAATTAATTTGACTGTAAACACAAAGATATTAGTACAAAATACGTG ACGTAGAAAGTAATAATTTCTTGGGTAGTTTGCAGTTTTAAAATTATGTTTTAAAAT GGACTATCATATGCTTACCGTAACTTGAAAGTATTTCGATTTCTTGGCTTTATATATC TTGTGGAAAGGACGAAACACC (SEQ ID NO: 1222). In some cases, a mouse U6 promoter can comprise a sequence with at least about: 70%, 75%, 80%, 85%, 90%, 95%, or 99% sequence identity to: GTACTGAGTCGCCCAGTCTCAGATAGATCCGACGCCGCCATCTCTAGGCCCGCGCCG GCCCCCTCGCACAGACTTGTGGGAGAAGCTCGGCTACTCCCCTGCCCCGGTTAATTT GCATATAATATTTCCTAGTAACTATAGAGGCTTAATGTGCGATAAAAGACAGATAAT CTGTTCTTTTTAATACTAGCTACATTTTACATGATAGGCTTGGATTTCTATAAGAGAT ACAAATACTAAATTATTATTTTAAAAAACAGCACAAAAGGAAACTCACCCTAACTG TAAAGTAATTGTGTGTTTTGAGACTATAAATATCCCTTGGAGAAAAGCCTTGTTTG (SEQ ID NO: 1223). In some cases, a human U7 promoter can comprise a sequence with at least about: 70%, 75%, 80%, 85%, 90%, 95%, or 99% sequence identity to: TTAACAACAACGAAGGGGCTGTGACTGGCTGCTTTCTCAACCAATCAGCACCGAACT CATTTGCATGGGCTGAGAACAAATGTTCGCGAACTCTAGAAATGAATGACTTAAGT AAGTTCCTTAGAATATTATTTTTCCTACTGAAAGTTACCACATGCGTCGTTGTTTATA CAGTAATAGGAACAAGAAAAAAGTCACCTAAGCTCACCCTCATCAATTGTGGAGTT CCTTTATATCCCATCTTCTCTCCAAACACATACGCA (SEQ ID NO: 1224). In some cases, a mouse U7 promoter can comprise a sequence with at least about: 70%, 75%, 80%, 85%, 90%, 95%, or 99% sequence identity to: TTAACAACATAGGAGCTGTGATTGGCTGTTTTCAGCCAATCAGCACTGACTCATTTG CATAGCCTTTACAAGCGGTCACAAACTCAAGAAACGAGCGGTTTTAATAGTCTTTTA GAATATTGTTTATCGAACCGAATAAGGAACTGTGCTTTGTGATTCACATATCAGTGG AGGGGTGTGGAAATGGCACCTTGATCTCACCCTCATCGAAAGTGGAGTTGATGTCCT TCCCTGGCTCGCTACAGACGCACTTCCGC (SEQ ID NO: 1225). In some cases, a human U1 promoter can comprise a sequence with at least about: 70%, 75%, 80%, 85%, 90%, 95%, or 99% sequence identity to: TAAGGACCAGCTTCTTTGGGAGAGAACAGACGCAGGGGCGGGAGGGAAAAAGGGA GAGGCAGACGTCACTTCCTCTTGGCGACTCTGGCAGCAGATTGGTCGGTTGAGTGGC AGAAAGGCAGACGGGGACTGGGCAAGGCACTGTCGGTGACATCACGGACAGGGCG ACTTCTATGTAGATGAGGCAGCGCAGAGGCTGCTGCTTCGCCACTTGCTGCTTCGCC ACGAAGGGAGTTCCCGTGCCCTGGGAGCGGGTTCAGGACCGCTGATCGGAAGTGAG AATCCCAGCTGTGTGTCAGGGCTGGAAAGGGCTCGGGAGTGCGCGGGGCAAGTGAC CGTGTGTGTAAAGAGTGAGGCGTATGAGGCTGTGTCGGGGCAGAGCCCGAAGATCT C (SEQ ID NO: 1226). In some cases, a CMV promoter can comprise a sequence with at least about: 70%, 75%, 80%, 85%, 90%, 95%, or 99% sequence identity to:

(SEQ ID NO: 1227)
ATACGCGTTGACATTGATTATTGACTAGTTATTAATAGTAATCAATTAC
GGGGTCATTAGTTCATAGCCCATATATGGAGTTCCGCGTTACATAACTT
ACGGTAAATGGCCCGCCTGGCTGACCGCCCAACGACCCCCGCCCATTGA
CGTCAATAATGACGTATGTTCCCATAGTAACGCCAATAGGGACTTTCCA
TTGACGTCAATGGGTGGAGTATTTACGGTAAACTGCCCACTTGGCAGTA
CATCAAGTGTATCATATGCCAAGTACGCCCCCTATTGACGTCAATGACG
GTAAATGGCCCGCCTGGCATTATGCCCAGTACATGACCTTATGGGACTT
TCCTACTTGGCAGTACATCTACGTATTAGTCATCGCTATTACCATGGTG
ATGCGGTTTTGGCAGTACATCAATGGGCGTGGATAGCGGTTTGACTCAC
GGGGATTTCCAAGTCTCCACCCCATTGACGTCAATGGGAGTTTGTTTTG
GCACCAAAATCAACGGGACTTTCCAAAATGTCGTAACAACTCCGCCCCA
TTGACGCAAATGGGCGGTAGGCGTGTACGGTGGGAGGTCTATATAAGCA
GAGCTCGTTTAGTGAACCGTCAGATCGCCTGGAGACGCCATCCACGCTG
TTTTGACCTCCATAGAAGACACCGGGACCGATCCAGCCTCCGGACTCTA
GAGGATCGAACC.

### Targets and Methods of Treatment

The present disclosure provides for compositions of engineered guide RNAs or engineered polynucleotides encoding guide RNAs and methods of use thereof, such as methods of treatment. In some embodiments, the engineered polynucleotides of the present disclosure encode guide RNAs targeting a coding sequence of an RNA (e.g., a TIS such as the c.1 TIS, the c.31 TIS, the c.91 TIS, or the c.379 TIS of MAPT). In some embodiments, the engineered polynucleotides of the present disclosure encode guide RNAs target the c.1 TIS and have at least 80%, 85%, 90%, 92%, 95%, 97%, or 99% sequence identity to any one of SEQ ID NO: 2-SEQ ID NO: 1215. In some embodiments, the engineered polynucleotides of the present disclosure encode guide RNAs targeting a non-coding sequence of an RNA (e.g., a polyA sequence). In some embodiments, the present disclosure provides compositions of one or more than one engineered polynucleotides encoding more than one engineered guide RNAs targeting the TIS, the polyA sequence, or any other part of a coding sequence or non-coding sequence. The engineered guide RNAs disclosed herein facilitate ADAR-mediated RNA editing of adenosines in the TIS, the polyA sequence, any part of a coding sequence of an RNA, any part of a non-coding sequence of an RNA, or any combination thereof.

The present disclosure provides for engineered guide RNAs that facilitate MAPT RNA editing when contacted with MAPT RNA to knockdown expression of Tau protein. Knockdown via an engineered guide RNA of the present disclosure result in a reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of Tau protein, relative to an amount prior to contacting the engineered guide RNA with the MAPT RNA. Tau pathology can be a key driver of a broad spectrum of neurodegenerative diseases, collectively known as Tauopathies. For example, diseases where Tau can play a primary role include, but are not limited to, Alzheimer's disease (AD), frontotemporal dementia (FTD), Parkinson's disease, progressive supranuclear palsy (PSP), corticobasal degeneration (CBD), and chronic traumatic encephalopathy. Tauopathies are characterized by the intracellular accumulation of neurofibrillary tangles (NFTs) composed of aggregated, misfolded Tau (MAPT gene). Thus, engineered guide RNAs of the present disclosure targeting MAPT RNA for ADAR-mediated editing to knockdown Tau protein can be capable of preventing or ameliorating disease progression in a number of diseases, including, but not limited to, AD, FTD, autism, traumatic brain injury, Parkinson's disease, and Dravet syndrome.

Thus, the engineered guide RNAs of the present disclosure can target MAPT for RNA editing, thereby, driving a reduction in Tau protein expression. In some embodiments, Tau protein expression is reduced in human neurons. In some embodiments, the present disclosure provides compositions of engineered guide RNAs that target MAPT and facilitated ADAR-mediated RNA editing of MAPT to reduce pathogenic levels of Tau by targeting key adenosines for deamination that are present in the translational initiation sites (TISs). In some embodiments, the engineered guide RNAs of the present disclosure target a coding sequence in MAPT. For example, the coding sequence can be a translation initiation site (TIS) (AUG) of MAPT and the engineered guide RNA can facilitate ADAR-mediated RNA editing of AUG to GUG. Engineered guide RNAs of the present disclosure can target one or more of the TISs in MAPT to reduce or completely inhibit Tau protein expression. For example, in some embodiments, an engineered guide RNA targets the AUG at the 18^{th} nucleotide in Exon 1 (c.1, Nm_005910.5; GRCh37/Hg19; also referred to as “c.1” for coding nucleotide 1), which is referred to as the conventional or canonical TIS and is also referred to as the c.1 TIS or the AUG at the 18^{th} nucleotide in Exon 1 corresponding to the canonical TIS at nucleotide position 151 of the MAPT reference transcript of NM_001377265.1. In some embodiments, the engineered polynucleotides of the present disclosure encode guide RNAs target the c.1 TIS and have at least 80%, 85%, 90%, 92%, 95%, 97%, or 99% sequence identity to any one of SEQ ID NO: 2-SEQ ID NO: 1215. In some embodiments, an engineered guide RNA targets the AUG at the 48th nucleotide in Exon 1 (c.31). In some embodiments, an engineered guide RNA targets the AUG at the 6th nucleotide in Exon 5 (c.379). With reference to the 2N4R Tau isoform containing 441 amino acids (Np_005901; GRCh37/Hg19), these three TISs correspond to methionines (Met) 1, 11 and 127, respectively. In some embodiments, an engineered guide RNA targets the AUG at the 108th nucleotide in Exon 1 (c.91). In some embodiments, one or more than one engineered guide RNAs of the present disclosure target any one or any combination of said four TISs. For example, a single engineered guide RNA of the present disclosure can be designed to target more than one of the above four TISs. In some embodiments, more than one engineered guide RNAs are designed to each independently target more than one of the above four TISs. In some embodiments, engineered guide RNAs of the present disclosure can target any one or any combination of the TISs in Exon 1 (c.1, c.31, and c.91). Targeting these sites in MAPT facilitate edits that result in inhibition of translation and a reduction in expression of the Tau protein. In some embodiments, the ratio of 3R to 4R isoforms of Tau can be measured by protein analysis (e.g., using an ELISA or flow cytometry) to evaluate the effect of RNA editing, with a 1 to 1 ratio representing the ratio in healthy adult brain. In some embodiments, any of the engineered guide RNAs disclosed herein are packaged in an AAV vector and are virally delivered.

As disclosed herein, editing of a target sequence of a MAPT RNA by an engineered guide RNA via ADAR can be used to reduce expression of tau protein. A reduction in tau can be utilized to treat a disease of condition associated with tau aggregation. In some embodiments, the disease or condition is a tauopathy. Editing of a target MAPT RNA as described herein, with concomitant reduction in tau protein levels, can be utilized to reduce or prevent aggregation of tau protein. Thus, one or more symptoms associated with aggregation of tau (e.g. tauopathies) can be treated by administration of an engineered guide RNA described herein.

As disclosed herein, administration of an engineered guide RNA described herein that targets MAPT RNA to a subject can be used to treat a disease or condition associated with tau pathophysiology, including treatment one of one or more symptoms associated with the disease or condition. In some embodiments, the disease or condition can be Alzheimer's disease (AD). In some embodiments, the disease or condition can be frontotemporal dementia (FTD). In some embodiments, the disease or condition can be Parkinson's disease. In some embodiments, the disease or condition can be progressive supranuclear palsy (PSP). In some embodiments, the disease or condition can be corticobasal degeneration (CBD). In some embodiments, the disease or condition can be chronic traumatic encephalopathy. In some embodiments, the disease or condition can be autism. In some embodiments, the disease or condition can be traumatic brain injury. In some embodiments, the disease or condition can be Dravet syndrome. In some embodiments, one or more symptoms of a disease or condition described herein can be treated by administration of an engineered guide RNA targeting MAPT RNA as described herein. For example, administration of an engineered guide RNA can be sufficient to reduce resting tremors, muscle stiffness, difficulty standing, difficulty walking, difficulty with bodily movements, involuntary movements, muscle rigidity, problems with coordination, rhythmic muscle contractions, slow bodily movement, bradykinesia, slow shuffling gait, or any combination thereof. In some embodiments, treatment of a disease or condition described herein can include improvement in cognitive manifestations associated with the disease or condition. For example, treatment can include a reduction in memory loss, a reduction in confusion, an increase in learning, an increase in communication ability, an increased attention span, an improved quality of life, increased recognition of people or objects, decrease in hallucinations, decrease in delusions, decrease in paranoia, decrease in impulsive behavior, decrease in restlessness, decrease in anxiety, decrease in repetitive statements or movement, or any combination thereof. In some embodiments, behavioral or cognitive tests can be administered to monitor the progression of treatment as a function of time.

As disclosed herein, administration of an engineered guide RNA of the present disclosure can be used to reduce tau protein levels through knockdown in order to treat a disease or condition associated with aberrant levels of tau (including disease associated with tau aggregation). While a reduction is obtained through administration, residual tau protein can still be present after the administering. In some cases, the presence of reduced tau protein levels treats the disease or condition, without reducing the level of tau protein levels to zero. Such levels can be determined in an in vitro assay using a sample obtained from a subject. In some cases, reduction in tau levels by administration of an engineered guide RNA of the current disclosure can manifest with improved pathophysiology associated with functional tau protein. For example, administration of an engineered guide RNA of the present disclosure can reduce neurofibrillary tangles, neuronal cell death, neuroinflammation, or other manifestations, relative to an amount prior to the administering.

In some cases, levels of certain biomarkers can be measured as a function of time to monitor the progression of treatment. In some embodiments, the biomarkers can be tau isoforms. In some aspects, various isoforms of tau protein (e.g. 1R, 2R, 3R, 4R 5R or 6R) can be modulated upon administration of an engineered guide RNA of the present disclosure. For example, in some instances the 4R isoform of tau can be increase or maintained upon administration. In some embodiments, the 3R isoform of tau can be decreased or maintained upon administration. In some embodiments, the ratio of the 4R isoform of tau to the 3R isoform of tau can be increase or maintained upon administration. In some cases, a biomarker can be post-translationally modified tau. In some aspects, levels of post-translationally modified tau can be modulated upon administration of an engineered guide RNA of the present disclosure. For example, the level of phosphorylated tau can be decreased or maintained upon administration. In some embodiments, the ratio of phosphorylated tau to non-phosphorylated tau can be decreased or maintained upon administration. In some instances, biomarker levels can be determined in an in vitro assay. In some instances, biomarker levels can be determined in vivo using, for example, using an imaging technique such as MRI.

In some embodiments, the engineered guide RNAs target a non-coding sequence in MAPT. The non-coding sequence can be a polyA signal sequence and the engineered guide RNA can facilitate ADAR-mediated RNA editing of one or more adenosines in the polyA signal sequence of MAPT. In some embodiments, engineered guide RNAs of the present disclosure can be multiplexed to target more than one polyA signal sequences in MAPT. In some embodiments, engineered guide RNAs of the present disclosure can be multiplexed to target the TIS and one or more polyA signal sequences in MAPT. In some embodiments, engineered guide RNAs of the present disclosure targeting the canonical TIS (the c.1 TIS) of MAPT can be multiplexed with one or more additional engineered guide RNAs targeting a different TIS of MAPT, such as the c.11 TIS, the c.31 TIS, or the c.91 TIS. In some embodiments, engineered guide RNAs of the present disclosure targeting the MAPT TIS (e.g., the canonical c.1 TIS) can be multiplexed with one or more engineered guide RNAs of a different sequence targeting a different region of MAPT, such as the 5′UTR region of MAPT (e.g., a Kozak sequence, an internal ribosomal entry site (IRES), or an iron response element (IRE) of the 5′ UTR). In some embodiments, engineered guide RNAs can be multiplexed to target a non-coding sequence and a coding sequence in MAPT. In each of these cases, the multiplexed engineered guide RNAs can be delivered together in the same viral vector or the each of the distinct engineered guide RNAs can be delivered together but in separate vectors. The engineered guide RNAs of the present disclosure facilitated ADAR-mediated RNA editing of MAPT, thereby, effecting protein knockdown.

In some embodiments, the engineered guide RNAs target multiple translation initiation sequences (TIS). In some instances, an engineered guide RNA of the present disclosure can comprise multiple targeting domains that hybridize to multiple sequences within a MAPT RNA. For example, an engineered guide RNA can have a first targeting domain that hybridizes to a sequence comprising the AUG in the 18th nucleotide in Exon 1 (c.1; canonical TIS of MAPT), and a second targeting domain that hybridizes to a second sequence of the target MAPT RNA selected from the group consisting of: (a) an AUG in the 48th nucleotide in Exon 1 (c.31), (b) an AUG in the 108th nt of Exon 1 (c.91), (c) an AUG in the 6th nucleotide in Exon 5 (c.379), and any combination thereof.

The present disclosure, in some embodiments, provides engineered guide RNAs that facilitate edits at multiple adenosines. Hydrolytic deamination of multiple adenosines in an RNA can be referred to as hyper-editing. In some cases, hyper-editing can occur in cis (e.g. in an Alu element) or in trans (e.g. in a target RNA by an engineered guide RNA). In some cases, hyper-editing can comprise editing in the polyA signal sequence of the MAPT target RNA. In some cases, hyper-editing can introduce edits in at least 2 or more nucleotides of a subject target RNA. In some cases, hyper-editing can introduce at least or at most about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or at least or at most about 100 edits in a region of a target RNA. In an embodiment, hyper-editing can occur in an untranslated region, translated region, 3′UTR, 5′UTR, or any combinations thereof.

In some embodiments, the engineered guide RNAs of the present disclosure facilitated ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5% editing of a target adenosine. In some embodiments, the engineered guide RNAs of the present disclosure can facilitate at least 10% editing of a target adenosine. 15% editing of a target adenosine. In some embodiments, the engineered guide RNAs of the present disclosure can facilitate at least 20% editing of a target adenosine. In some embodiments, the engineered guide RNAs of the present disclosure can facilitate at least 25% editing of a target adenosine. In some embodiments, the engineered guide RNAs of the present disclosure can facilitate at least 30% editing of a target adenosine. In some embodiments, the engineered guide RNAs of the present disclosure can facilitate at least 35% editing of a target adenosine. In some embodiments, the engineered guide RNAs of the present disclosure can facilitate at least 40% editing of a target adenosine. In some embodiments, the engineered guide RNAs of the present disclosure can facilitate at least 45% editing of a target adenosine. In some embodiments, the engineered guide RNAs of the present disclosure can facilitate at least 50% editing of a target adenosine. In some embodiments, the engineered guide RNAs of the present disclosure can facilitate at least 55% editing of a target adenosine. In some embodiments, the engineered guide RNAs of the present disclosure can facilitate at least 60% editing of a target adenosine. In some embodiments, the engineered guide RNAs of the present disclosure can facilitate at least 65% editing of a target adenosine. In some embodiments, the engineered guide RNAs of the present disclosure can facilitate at least 70% editing of a target adenosine. In some embodiments, the engineered guide RNAs of the present disclosure can facilitate at least 75% editing of a target adenosine. In some embodiments, the engineered guide RNAs of the present disclosure can facilitate at least 80% editing of a target adenosine. In some embodiments, the engineered guide RNAs of the present disclosure can facilitate at least 85% editing of a target adenosine. In some embodiments, the engineered guide RNAs of the present disclosure can facilitate at least 90% editing of a target adenosine. In some embodiments, the engineered guide RNAs of the present disclosure can facilitate at least 95% editing of a target adenosine. In some embodiments, the engineered guide RNAs of the present disclosure can facilitate 100% editing of a target adenosine. In some embodiments, the engineered guide RNAs of the present disclosure can facilitate from 5 to 20% editing of a target adenosine. In some embodiments, the engineered guide RNAs of the present disclosure can facilitate from 20 to 40% editing of a target adenosine. In some embodiments, the engineered guide RNAs of the present disclosure can facilitate from 40 to 60% editing of a target adenosine. In some embodiments, the engineered guide RNAs of the present disclosure can facilitate from 60 to 80% editing of a target adenosine. In some embodiments, the engineered guide RNAs of the present disclosure can facilitate from 80 to 100% editing of a target adenosine. In some embodiments, the engineered guide RNAs of the present disclosure can facilitate from 60 to 80% editing of a target adenosine. In some embodiments, the engineered guide RNAs of the present disclosure can facilitate from 70 to 90% editing of a target adenosine. In some embodiments, the engineered guide RNAs of the present disclosure can facilitate at least 70% or more editing of a target adenosine. In some embodiments, the engineered guide RNAs of the present disclosure can facilitate at least 80% or more editing of a target adenosine. In some embodiments, the engineered guide RNAs of the present disclosure can facilitate up to 90% or more editing of a target adenosine. Optionally, additionally, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 30% editing of an off-target adenosine. In some embodiments, the engineered guide RNAs of the present disclosure can facilitate these levels of on-target RNA editing while maintaining less than 25% editing of an off-target adenosine. In some embodiments, the engineered guide RNAs of the present disclosure can facilitate these levels of on-target RNA editing while maintaining less than 20% editing of an off-target adenosine. In some embodiments, the engineered guide RNAs of the present disclosure can facilitate these levels of on-target RNA editing while maintaining less than 15% editing of an off-target adenosine. In some embodiments, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 9% editing of an off-target adenosine. In some embodiments, the engineered guide RNAs of the present disclosure can facilitate these levels of on-target RNA editing while maintaining less than 8% editing of an off-target adenosine. In some embodiments, the engineered guide RNAs of the present disclosure can facilitate these levels of on-target RNA editing while maintaining less than 7% editing of an off-target adenosine. In some embodiments, the engineered guide RNAs of the present disclosure can facilitate these levels of on-target RNA editing while maintaining less than 6% editing of an off-target adenosine. In some embodiments, the engineered guide RNAs of the present disclosure can facilitate these levels of on-target RNA editing while maintaining less than 5% editing of an off-target adenosine. In some embodiments, the engineered guide RNAs of the present disclosure can facilitate these levels of on-target RNA editing while maintaining less than 4% editing of an off-target adenosine. In some embodiments, the engineered guide RNAs of the present disclosure can facilitate these levels of on-target RNA editing while maintaining less than 3% editing of an off-target adenosine. In some embodiments, the engineered guide RNAs of the present disclosure can facilitate these levels of on-target RNA editing while maintaining less than 2% editing of an off-target adenosine. In some embodiments, the engineered guide RNAs of the present disclosure can facilitate these levels of on-target RNA editing while maintaining less than 1% editing of an off-target adenosine. In some embodiments, the engineered guide RNAs of the present disclosure can facilitate these levels of on-target RNA editing while maintaining 0% editing of an off-target adenosine. In some embodiments, the engineered guide RNAs of the present disclosure can facilitate at least 70% editing of a target adenosine while maintaining less than 30% editing of an off-target adenosine. In some embodiments, the engineered guide RNAs of the present disclosure can facilitate at least 70% editing of a target adenosine while maintaining less than 29% editing of an off-target adenosine. In some embodiments, the engineered guide RNAs of the present disclosure can facilitate at least 70% editing of a target adenosine while maintaining less than 28% editing of an off-target adenosine. In some embodiments, the engineered guide RNAs of the present disclosure can facilitate at least 70% editing of a target adenosine while maintaining less than 27% editing of an off-target adenosine. In some embodiments, the engineered guide RNAs of the present disclosure can facilitate at least 70% editing of a target adenosine while maintaining less than 26% editing of an off-target adenosine. In some embodiments, the engineered guide RNAs of the present disclosure can facilitate at least 70% editing of a target adenosine while maintaining less than 25% editing of an off-target adenosine. In some embodiments, the engineered guide RNAs of the present disclosure can facilitate at least 70% editing of a target adenosine while maintaining less than 24% editing of an off-target adenosine. In some embodiments, the engineered guide RNAs of the present disclosure can facilitate at least 70% editing of a target adenosine while maintaining less than 23% editing of an off-target adenosine. In some embodiments, the engineered guide RNAs of the present disclosure can facilitate at least 70% editing of a target adenosine while maintaining less than 22% editing of an off-target adenosine. In some embodiments, the engineered guide RNAs of the present disclosure can facilitate at least 70% editing of a target adenosine while maintaining less than 21% editing of an off-target adenosine. In some embodiments, the engineered guide RNAs of the present disclosure can facilitate at least 70% editing of a target adenosine while maintaining less than 20% editing of an off-target adenosine. In some embodiments, the engineered guide RNAs of the present disclosure can facilitate at least 70% editing of a target adenosine while maintaining less than 19% editing of an off-target adenosine. In some embodiments, the engineered guide RNAs of the present disclosure can facilitate at least 70% editing of a target adenosine while maintaining less than 18% editing of an off-target adenosine. In some embodiments, the engineered guide RNAs of the present disclosure can facilitate at least 70% editing of a target adenosine while maintaining less than 17% editing of an off-target adenosine. In some embodiments, the engineered guide RNAs of the present disclosure can facilitate at least 70% editing of a target adenosine while maintaining less than 16% editing of an off-target adenosine. In some embodiments, the engineered guide RNAs of the present disclosure can facilitate at least 70% editing of a target adenosine while maintaining less than 15% editing of an off-target adenosine. In some embodiments, the engineered guide RNAs of the present disclosure can facilitate at least 70% editing of a target adenosine while maintaining less than 14% editing of an off-target adenosine. In some embodiments, the engineered guide RNAs of the present disclosure can facilitate at least 70% editing of a target adenosine while maintaining less than 13% editing of an off-target adenosine. In some embodiments, the engineered guide RNAs of the present disclosure can facilitate at least 70% editing of a target adenosine while maintaining less than 12% editing of an off-target adenosine. In some embodiments, the engineered guide RNAs of the present disclosure can facilitate at least 70% editing of a target adenosine while maintaining less than 11% editing of an off-target adenosine. In some embodiments, the engineered guide RNAs of the present disclosure can facilitate at least 70% editing of a target adenosine while maintaining less than 10% editing of an off-target adenosine. In some embodiments, the engineered guide RNAs of the present disclosure can facilitate at least 70% editing of a target adenosine while maintaining less than 9% editing of an off-target adenosine. In some embodiments, the engineered guide RNAs of the present disclosure can facilitate at least 70% editing of a target adenosine while maintaining less than 8% editing of an off-target adenosine. In some embodiments, the engineered guide RNAs of the present disclosure can facilitate at least 70% editing of a target adenosine while maintaining less than 7% editing of an off-target adenosine. In some embodiments, the engineered guide RNAs of the present disclosure can facilitate at least 70% editing of a target adenosine while maintaining less than 6% editing of an off-target adenosine. In some embodiments, the engineered guide RNAs of the present disclosure can facilitate at least 70% editing of a target adenosine while maintaining less than 5% editing of an off-target adenosine. In some embodiments, the engineered guide RNAs of the present disclosure can facilitate at least 70% editing of a target adenosine while maintaining less than 4% editing of an off-target adenosine. In some embodiments, the engineered guide RNAs of the present disclosure can facilitate at least 70% editing of a target adenosine while maintaining less than 3% editing of an off-target adenosine. In some embodiments, the engineered guide RNAs of the present disclosure can facilitate at least 70% editing of a target adenosine while maintaining less than 2% editing of an off-target adenosine. In some embodiments, the engineered guide RNAs of the present disclosure can facilitate at least 70% editing of a target adenosine while maintaining less than 1% editing of an off-target adenosine. In some embodiments, the engineered guide RNAs of the present disclosure can facilitate at least 70% editing of a target adenosine while maintaining 0% editing of an off-target adenosine.

In some embodiments, the engineered guide RNAs of the present disclosure facilitate ADAR-mediated RNA editing of MAPT, which results in knockdown of protein levels. The knockdown in protein levels is quantitated as a reduction in expression of the Tau protein. The engineered guide RNAs of the present disclosure can facilitate from 1% to 100% Tau protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% Tau protein knockdown. In some embodiments, the engineered guide RNAs of the present disclosure facilitate from 30% to 60% Tau protein knockdown. Tau protein knockdown can be measured by an assay comparing a sample or subject treated with the engineered guide RNA to a control sample or subject not treated with the engineered guide RNA.

An engineered guide RNA of the present disclosure can be used in a method of treating a disorder in a subject in need thereof. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding an engineered guide RNA to a cell of a subject in need thereof and expressing the engineered guide RNA in the cell. In some embodiments, an engineered guide RNA of the present disclosure can be used to treat a genetic disorder (e.g., a Tauopathy such as AD, FTD, Parkinson's disease). In some embodiments, an engineered guide RNA of the present disclosure can be used to treat a condition associated with one or more mutations.

### Pharmaceutical Compositions

The compositions described herein (e.g., compositions comprising an engineered guide RNA or an engineered polynucleotide) can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like, or any combination thereof. In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a triglyceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

### Delivery

An engineered guide RNA of the present disclosure (such as an engineered guide RNA with a polynucleotide sequence of any one of SEQ ID NO: 2-1215 as recited in Table 1 that target the MAPT c.1 TIS) or an engineered polynucleotide of the present disclosure (e.g., an engineered polynucleotide encoding an engineered guide RNA) can be delivered via a delivery vehicle. In some embodiments, the delivery vehicle is a vector. A vector can facilitate delivery of the engineered polynucleotide into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the viral vector can be of a specific serotype. In some embodiments, the viral vector can be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV.rh8 serotype, AAV.rh10 serotype, AAV.rh20 serotype, AAV.rh39 serotype, AAV.Rh74 serotype, AAV.RHM4-1 serotype, AAV.hu37 serotype, AAV.Anc80 serotype, AAV.Anc80L65 serotype, AAV.7m8 serotype, AAV.PHP.B serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, and AAVhu68 serotype, a derivative of any of these serotypes, or any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered polynucleotide of the present disclosure (e.g., an engineered polynucleotide encoding an engineered guide RNA) in an AAV vector. In some examples, methods of producing the delivery vectors described herein comprise, (a) introducing into a cell: (i) a polynucleotide comprising a promoter and an engineered guide RNA disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof, (c) assembling an AAV particle; and (d) packaging the engineered guide RNA disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes can be not the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/6, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered guide RNA or the engineered polynucleotide is a non-viral vector system. In some embodiments, the non-viral vector system comprises cationic lipids, or polymers. For example, the non-viral vector system comprises can be a liposome or polymeric nanoparticle. In some embodiments, the engineered guide RNA or a non-viral vector comprising the engineered guide RNA is delivered to a cell by hydrodynamic injection or ultrasound.

### Administration

Administration can refer to methods that can be used to enable the delivery of a composition described herein (e.g. comprising an engineered guide RNA or an engineered polynucleotide encoding the same) to the desired site of biological action. For example, an engineered guide RNA (such as an engineered guide RNA with a polynucleotide sequence of any one of SEQ ID NO: 2-1215 as recited in Table 1 that targets the MAPT c.1 TIS) can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat (including prevent) a disease in the subject.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The appropriate dosage and treatment regimen for the methods of treatment described herein vary with respect to the particular disease being treated, the gRNA and/or ADAR (or a vector encoding the gRNA and/or ADAR) being delivered, and the specific condition of the subject. In some examples, the administration can be over a period of time until the desired effect (e.g., reduction in symptoms can be achieved). In some examples, administration can be 1, 2, 3, 4, 5, 6, or 7 times per week. In some examples, administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be over a period of 1, 2, 3, 4, 5, 6, 7, 8, 9, or 10 weeks. In some examples, administration can be over a period of 2, 3, 4, 5, 6 or more months. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more. In some examples, treatment can be resumed following a period of remission.

### Definitions

Unless defined otherwise, all terms of art, notations and other technical and scientific terms or terminology used herein is intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that can be not designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode a polypeptide during translation, whereas DNA can encode an mRNA molecule during transcription.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” can refer to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together—optionally to preserve the conformation of the fused protein domains, prevent unfavorable interactions between the fused protein domains which can compromise their respective functions, or both.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject may not be necessarily diagnosed or suspected of being at high risk for the disease

The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g. physiological pH, physiological temperature, physiological salt concentration, etc.).

As used herein, the terms “treatment” or “treating” can be used in reference to a pharmaceutical or other intervention regimen for obtaining beneficial or desired results in the recipient. Beneficial or desired results include but can be not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. A composition comprising an engineered guide RNA, wherein: a) the engineered guide RNA, upon hybridization to a sequence of a target MAPT RNA, forms a guide-target RNA scaffold with the sequence of the target MAPT RNA; b) formation of the guide-target RNA scaffold substantially forms one or more structural features selected from the group consisting of: a bulge, an internal loop, and a hairpin; and c) the structural feature is not present within the engineered guide RNA prior to the hybridization of the engineered guide RNA to the MAPT target RNA; and d) upon hybridization of the engineered guide RNA to the sequence of the target MAPT RNA, the engineered guide RNA facilitates RNA editing of one or more target adenosines in the sequence of the target MAPT RNA by an RNA editing entity. Embodiment 2. The composition of Embodiment 1, wherein the sequence of the target MAPT RNA is within the 3′ untranslated region (UTR). Embodiment 3. The composition of Embodiment 1, wherein the sequence of the target MAPT RNA is within the 5′ untranslated region (UTR). Embodiment 4. The composition of Embodiment 3, wherein the sequence of the target MAPT RNA in the 5′ UTR is a Kozak sequence. Embodiment 5. The composition of Embodiment 3, wherein the sequence of the target MAPT RNA in the 5′ UTR is an internal ribosomal entry site (IRES). Embodiment 6. The composition of Embodiment 3, wherein the sequence of the target MAPT RNA in the 5′ UTR is an iron response element (IRE). Embodiment 7. The composition of Embodiment 1, wherein the sequence of the target MAPT RNA comprises a translation initiation site (TIS). Embodiment 8. The composition of Embodiment 7, wherein the translation initiation site is an AUG starting at the 18th nucleotide in Exon 1 (c.1). Embodiment 9. The composition of Embodiment 7, wherein the translation initiation site is the c.1 TIS corresponding to the canonical TIS at nucleotide position 151 of the MAPT reference transcript of NM_001377265.1. Embodiment 10. The composition of any one of Embodiments 7-9, wherein the one or more structural features comprises: a first 6/6 symmetric internal loop at a position selected from the group consisting of: 34, 33, 32, 31, 30, 26, 25, 24, 23, and 22; relative to the target adenosine at position 0. Embodiment 11. The composition of Embodiment 10, wherein the first 6/6 symmetric internal loop is at position 34, relative to the target adenosine at position 0. Embodiment 12. The composition of Embodiment 11, wherein the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. Embodiment 13. The composition of Embodiment 12, wherein the engineered guide RNA comprises at least about: 80%, 85%, 90%, 92%, 95%, 97%, or 99% sequence identity to SEQ ID NO: 478. Embodiment 14. The composition of Embodiment 13, wherein the engineered guide RNA comprises SEQ ID NO: 478. Embodiment 15. The composition of Embodiment 11, wherein the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. Embodiment 16. The composition of Embodiment 15, wherein the engineered guide RNA comprises at least about: 80%, 85%, 90%, 92%, 95%, 97%, or 99% sequence identity to SEQ ID NO: 479. Embodiment 17. The composition of Embodiment 16, wherein the engineered guide RNA comprises SEQ ID NO: 479. Embodiment 18. The composition of Embodiment 10, wherein the first 6/6 symmetric internal loop is at position 33, relative to the target adenosine at position 0. Embodiment 19. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 20. The composition of Embodiment 19, wherein the engineered guide RNA comprises at least about: 80%, 85%, 90%, 92%, 95%, 97%, or 99% sequence identity to SEQ ID NO: 592. Embodiment 21. The composition of Embodiment 20, wherein the engineered guide RNA comprises SEQ ID NO: 592. Embodiment 22. The composition of Embodiment 18, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 23. The composition of Embodiment 22, wherein the engineered guide RNA comprises at least about: 80%, 85%, 90%, 92%, 95%, 97%, or 99% sequence identity to SEQ ID NO: 593. Embodiment 24. The composition of Embodiment 23, wherein the engineered guide RNA comprises SEQ ID NO: 593. Embodiment 25. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 26. The composition of Embodiment 25, wherein the engineered guide RNA comprises at least about: 80%, 85%, 90%, 92%, 95%, 97%, or 99% sequence identity to SEQ ID NO: 639. Embodiment 27. The composition of Embodiment 26, wherein the engineered guide RNA comprises SEQ ID NO: 639. Embodiment 28. The composition of Embodiment 18, wherein the one or more structural features further comprises at least one structural feature selected from the group consisting of: a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 29. The composition of Embodiment 28, wherein the engineered guide RNA comprises at least about: 80%, 85%, 90%, 92%, 95%, 97%, or 99% sequence identity to SEQ ID NO: 640. Embodiment 30. The composition of Embodiment 29, wherein the engineered guide RNA comprises SEQ ID NO: 640. Embodiment 31. The composition of Embodiment 18, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, and any combination thereof. Embodiment 32. The composition of Embodiment 31, wherein the engineered guide RNA comprises at least about: 80%, 85%, 90%, 92%, 95%, 97%, or 99% sequence identity to SEQ ID NO: 686. Embodiment 33. The composition of Embodiment 32, wherein the engineered guide RNA comprises SEQ ID NO: 686. Embodiment 34. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 35. The composition of Embodiment 34, wherein the engineered guide RNA comprises at least about: 80%, 85%, 90%, 92%, 95%, 97%, or 99% sequence identity to SEQ ID NO: 687. Embodiment 36. The composition of Embodiment 35, wherein the engineered guide RNA comprises SEQ ID NO: 687. Embodiment 37. The composition of Embodiment 18, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 38. The composition of Embodiment 37, wherein the engineered guide RNA comprises at least about: 80%, 85%, 90%, 92%, 95%, 97%, or 99% sequence identity to SEQ ID NO: 688. Embodiment 39. The composition of Embodiment 38, wherein the engineered guide RNA comprises SEQ ID NO: 688. Embodiment 40. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 41. The composition of Embodiment 40, wherein the engineered guide RNA comprises at least about: 80%, 85%, 90%, 92%, 95%, 97%, or 99% sequence identity to SEQ ID NO: 695. Embodiment 42. The composition of Embodiment 41, wherein the engineered guide RNA comprises SEQ ID NO: 695. Embodiment 43. The composition of Embodiment 18, wherein the one or more structural features further comprises at least one structural feature selected from the group consisting of: a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 44. The composition of Embodiment 43, wherein the engineered guide RNA comprises at least about: 80%, 85%, 90%, 92%, 95%, 97%, or 99% sequence identity to SEQ ID NO: 696. Embodiment 45. The composition of Embodiment 44, wherein the engineered guide RNA comprises SEQ ID NO: 696. Embodiment 46. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 47. The composition of Embodiment 46, wherein the engineered guide RNA comprises at least about: 80%, 85%, 90%, 92%, 95%, 97%, or 99% sequence identity to SEQ ID NO: 714. Embodiment 48. The composition of Embodiment 47, wherein the engineered guide RNA comprises SEQ ID NO: 714. Embodiment 49. The composition of Embodiment 18, wherein the one or more structural features further comprises at least one structural feature selected from the group consisting of: a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 50. The composition of Embodiment 49, wherein the engineered guide RNA comprises at least about: 80%, 85%, 90%, 92%, 95%, 97%, or 99% sequence identity to SEQ ID NO: 715. Embodiment 51. The composition of Embodiment 50, wherein the engineered guide RNA comprises SEQ ID NO: 715. Embodiment 52. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 53. The composition of Embodiment 52, wherein the engineered guide RNA comprises at least about: 80%, 85%, 90%, 92%, 95%, 97%, or 99% sequence identity to SEQ ID NO: 722. Embodiment 54. The composition of Embodiment 53, wherein the engineered guide RNA comprises SEQ ID NO: 722. Embodiment 55. The composition of Embodiment 18, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 56. The composition of Embodiment 55, wherein the engineered guide RNA comprises at least about: 80%, 85%, 90%, 92%, 95%, 97%, or 99% sequence identity to SEQ ID NO: 723. Embodiment 57. The composition of Embodiment 56, wherein the engineered guide RNA comprises SEQ ID NO: 723. Embodiment 58. The composition of Embodiment 18, wherein the one or more structural features further comprises at least one structural feature selected from the group consisting of: a 3/3 symmetric bulge at position −22 relative to position 0, a 3/3 symmetric bulge at position −13 relative to position 0, a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 59. The composition of Embodiment 58, wherein the engineered guide RNA comprises at least about: 80%, 85%, 90%, 92%, 95%, 97%, or 99% sequence identity to SEQ ID NO: 1059. Embodiment 60. The composition of Embodiment 59, wherein the engineered guide RNA comprises SEQ ID NO: 1059. Embodiment 61. The composition of Embodiment 18, wherein the one or more structural features further comprises at least one structural feature selected from the group consisting of: a 3/3 symmetric bulge at position −21 relative to position 0, a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 62. The composition of Embodiment 61, wherein the engineered guide RNA comprises at least about: 80%, 85%, 90%, 92%, 95%, 97%, or 99% sequence identity to SEQ ID NO: 1116. Embodiment 63. The composition of Embodiment 62, wherein the engineered guide RNA comprises SEQ ID NO: 1116. Embodiment 64. The composition of Embodiment 18, wherein the one or more structural features further comprises at least one structural feature selected from the group consisting of: a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 65. The composition of Embodiment 64, wherein the engineered guide RNA comprises at least about: 80%, 85%, 90%, 92%, 95%, 97%, or 99% sequence identity to SEQ ID NO: 1117. Embodiment 66. The composition of Embodiment 65, wherein the engineered guide RNA comprises SEQ ID NO: 1117. Embodiment 67. The composition of Embodiment 18, wherein the one or more structural features further comprises at least one structural feature selected from the group consisting of: a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 68. The composition of Embodiment 67, wherein the engineered guide RNA comprises at least about: 80%, 85%, 90%, 92%, 95%, 97%, or 99% sequence identity to SEQ ID NO: 1215. Embodiment 69. The composition of Embodiment 68, wherein the engineered guide RNA comprises SEQ ID NO: 1215. Embodiment 70. The composition of Embodiment 10, wherein the first 6/6 symmetric internal loop is at position 32, relative to the target adenosine at position 0. Embodiment 71. The composition of Embodiment 70, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 72. The composition of Embodiment 71, wherein the engineered guide RNA comprises at least about: 80%, 85%, 90%, 92%, 95%, 97%, or 99% sequence identity to SEQ ID NO: 438. Embodiment 73. The composition of Embodiment 72, wherein the engineered guide RNA comprises SEQ ID NO: 438. Embodiment 74. The composition of Embodiment 70, wherein the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 75. The composition of Embodiment 74, wherein the engineered guide RNA comprises at least about: 80%, 85%, 90%, 92%, 95%, 97%, or 99% sequence identity to SEQ ID NO: 439. Embodiment 76. The composition of Embodiment 75, wherein the engineered guide RNA comprises SEQ ID NO: 439. Embodiment 77. The composition of Embodiment 70, wherein the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 78. The composition of Embodiment 77, wherein the engineered guide RNA comprises at least about: 80%, 85%, 90%, 92%, 95%, 97%, or 99% sequence identity to SEQ ID NO: 816. Embodiment 79. The composition of Embodiment 78, wherein the engineered guide RNA comprises SEQ ID NO: 816. Embodiment 80. The composition of Embodiment 70, wherein the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 81. The composition of Embodiment 80, wherein the engineered guide RNA comprises at least about: 80%, 85%, 90%, 92%, 95%, 97%, or 99% sequence identity to SEQ ID NO: 817. Embodiment 82. The composition of Embodiment 81, wherein the engineered guide RNA comprises SEQ ID NO: 817. Embodiment 83. The composition of Embodiment 10, wherein the first 6/6 symmetric internal loop is at position 31, relative to the target adenosine at position 0. Embodiment 84. The composition of Embodiment 83, wherein the one or more structural features further comprises at least one structural feature selected from the group consisting of: a 3/3 symmetric bulge at position −19 relative to position 0, a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. Embodiment 85. The composition of Embodiment 84, wherein the engineered guide RNA comprises at least about: 80%, 85%, 90%, 92%, 95%, 97%, or 99% sequence identity to SEQ ID NO: 162. Embodiment 86. The composition of Embodiment 85, wherein the engineered guide RNA comprises SEQ ID NO: 162. Embodiment 87. The composition of Embodiment 83, wherein the one or more structural features further comprises at least one structural feature selected from the group consisting of: a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. Embodiment 88. The composition of Embodiment 87, wherein the engineered guide RNA comprises at least about: 80%, 85%, 90%, 92%, 95%, 97%, or 99% sequence identity to SEQ ID NO: 163. Embodiment 89. The composition of Embodiment 88, wherein the engineered guide RNA comprises SEQ ID NO: 163. Embodiment 90. The composition of Embodiment 83, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. Embodiment 91. The composition of Embodiment 90, wherein the engineered guide RNA comprises at least about: 80%, 85%, 90%, 92%, 95%, 97%, or 99% sequence identity to SEQ ID NO: 929. Embodiment 92. The composition of Embodiment 91, wherein the engineered guide RNA comprises SEQ ID NO: 929. Embodiment 93. The composition of Embodiment 83, wherein the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. Embodiment 94. The composition of Embodiment 93, wherein the engineered guide RNA comprises at least about: 80%, 85%, 90%, 92%, 95%, 97%, or 99% sequence identity to SEQ ID NO: 930. Embodiment 95. The composition of Embodiment 94, wherein the engineered guide RNA comprises SEQ ID NO: 930. Embodiment 96. The composition of Embodiment 10, wherein the first 6/6 symmetric internal loop is at position 30, relative to the target adenosine at position 0. Embodiment 97. The composition of Embodiment 96, wherein the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 98. The composition of Embodiment 97, wherein the engineered guide RNA comprises at least about: 80%, 85%, 90%, 92%, 95%, 97%, or 99% sequence identity to SEQ ID NO: 538. Embodiment 99. The composition of Embodiment 98, wherein the engineered guide RNA comprises SEQ ID NO: 538. Embodiment 100. The composition of Embodiment 96, wherein the one or more structural features further comprises at least one structural feature selected from the group consisting of: a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 101. The composition of Embodiment 100, wherein the engineered guide RNA comprises at least about: 80%, 85%, 90%, 92%, 95%, 97%, or 99% sequence identity to SEQ ID NO: 539. Embodiment 102. The composition of Embodiment 101, wherein the engineered guide RNA comprises SEQ ID NO: 539. Embodiment 103. The composition of Embodiment 96, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 104. The composition of Embodiment 103, wherein the engineered guide RNA comprises at least about: 80%, 85%, 90%, 92%, 95%, 97%, or 99% sequence identity to SEQ ID NO: 604. Embodiment 105. The composition of Embodiment 104, wherein the engineered guide RNA comprises SEQ ID NO: 604. Embodiment 106. The composition of Embodiment 96, wherein the one or more structural features further comprises at least one structural feature selected from the group consisting of: a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 107. The composition of Embodiment 106, wherein the engineered guide RNA comprises at least about: 80%, 85%, 90%, 92%, 95%, 97%, or 99% sequence identity to SEQ ID NO: 605. Embodiment 108. The composition of Embodiment 107, wherein the engineered guide RNA comprises SEQ ID NO: 605. Embodiment 109. The composition of Embodiment 96, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 110. The composition of Embodiment 109, wherein the engineered guide RNA comprises at least about: 80%, 85%, 90%, 92%, 95%, 97%, or 99% sequence identity to SEQ ID NO: 619. Embodiment 111. The composition of Embodiment 110, wherein the engineered guide RNA comprises SEQ ID NO: 619. Embodiment 112. The composition of Embodiment 96, wherein the one or more structural features further comprises at least one structural feature selected from the group consisting of: a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 113. The composition of Embodiment 112, wherein the engineered guide RNA comprises at least about: 80%, 85%, 90%, 92%, 95%, 97%, or 99% sequence identity to SEQ ID NO: 620. Embodiment 114. The composition of Embodiment 113, wherein the engineered guide RNA comprises SEQ ID NO: 620. Embodiment 115. The composition of Embodiment 96, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 116. The composition of Embodiment 115, wherein the engineered guide RNA comprises at least about: 80%, 85%, 90%, 92%, 95%, 97%, or 99% sequence identity to SEQ ID NO: 677. Embodiment 117. The composition of Embodiment 116, wherein the engineered guide RNA comprises SEQ ID NO: 677. Embodiment 118. The composition of Embodiment 96, wherein the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 119. The composition of Embodiment 118, wherein the engineered guide RNA comprises at least about: 80%, 85%, 90%, 92%, 95%, 97%, or 99% sequence identity to SEQ ID NO: 678. Embodiment 120. The composition of Embodiment 119, wherein the engineered guide RNA comprises SEQ ID NO: 678. Embodiment 121. The composition of Embodiment 96, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 122. The composition of Embodiment 121, wherein the engineered guide RNA comprises at least about: 80%, 85%, 90%, 92%, 95%, 97%, or 99% sequence identity to SEQ ID NO: 684. Embodiment 123. The composition of Embodiment 122, wherein the engineered guide RNA comprises SEQ ID NO: 684. Embodiment 124. The composition of Embodiment 96, wherein the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 125. The composition of Embodiment 124, wherein the engineered guide RNA comprises at least about: 80%, 85%, 90%, 92%, 95%, 97%, or 99% sequence identity to SEQ ID NO: 685. Embodiment 126. The composition of Embodiment 125, wherein the engineered guide RNA comprises SEQ ID NO: 685. Embodiment 127. The composition of Embodiment 96, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 128. The composition of Embodiment 127, wherein the engineered guide RNA comprises at least about: 80%, 85%, 90%, 92%, 95%, 97%, or 99% sequence identity to SEQ ID NO: 720. Embodiment 129. The composition of Embodiment 128, wherein the engineered guide RNA comprises SEQ ID NO: 720. Embodiment 130. The composition of Embodiment 96, wherein the one or more structural features further comprises at least one structural feature selected from the group consisting of: a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 131. The composition of Embodiment 130, wherein the engineered guide RNA comprises at least about: 80%, 85%, 90%, 92%, 95%, 97%, or 99% sequence identity to SEQ ID NO: 721. Embodiment 132. The composition of Embodiment 131, wherein the engineered guide RNA comprises SEQ ID NO: 721. Embodiment 133. The composition of Embodiment 96, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 134. The composition of Embodiment 133, wherein the engineered guide RNA comprises at least about: 80%, 85%, 90%, 92%, 95%, 97%, or 99% sequence identity to SEQ ID NO: 733. Embodiment 135. The composition of Embodiment 134, wherein the engineered guide RNA comprises SEQ ID NO: 733. Embodiment 136. The composition of Embodiment 96, wherein the one or more structural features further comprises at least one structural feature selected from the group consisting of: a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 137. The composition of Embodiment 136, wherein the engineered guide RNA comprises at least about: 80%, 85%, 90%, 92%, 95%, 97%, or 99% sequence identity to SEQ ID NO: 734. Embodiment 138. The composition of Embodiment 137, wherein the engineered guide RNA comprises SEQ ID NO: 734. Embodiment 139. The composition of Embodiment 96, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 140. The composition of Embodiment 139, wherein the engineered guide RNA comprises at least about: 80%, 85%, 90%, 92%, 95%, 97%, or 99% sequence identity to SEQ ID NO: 763. Embodiment 141. The composition of Embodiment 140, wherein the engineered guide RNA comprises SEQ ID NO: 763. Embodiment 142. The composition of Embodiment 96, wherein the one or more structural features further comprises at least one structural feature selected from the group consisting of: a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 143. The composition of Embodiment 142, wherein the engineered guide RNA comprises at least about: 80%, 85%, 90%, 92%, 95%, 97%, or 99% sequence identity to SEQ ID NO: 764. Embodiment 144. The composition of Embodiment 143, wherein the engineered guide RNA comprises SEQ ID NO: 764. Embodiment 145. The composition of Embodiment 96, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 146. The composition of Embodiment 145, wherein the engineered guide RNA comprises at least about: 80%, 85%, 90%, 92%, 95%, 97%, or 99% sequence identity to SEQ ID NO: 775. Embodiment 147. The composition of Embodiment 146, wherein the engineered guide RNA comprises SEQ ID NO: 775. Embodiment 148. The composition of Embodiment 96, wherein the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 149. The composition of Embodiment 148, wherein the engineered guide RNA comprises at least about: 80%, 85%, 90%, 92%, 95%, 97%, or 99% sequence identity to SEQ ID NO: 776. Embodiment 150. The composition of Embodiment 149, wherein the engineered guide RNA comprises SEQ ID NO: 776. Embodiment 151. The composition of Embodiment 96, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 152. The composition of Embodiment 151, wherein the engineered guide RNA comprises at least about: 80%, 85%, 90%, 92%, 95%, 97%, or 99% sequence identity to SEQ ID NO: 795. Embodiment 153. The composition of Embodiment 152, wherein the engineered guide RNA comprises SEQ ID NO: 795. Embodiment 154. The composition of Embodiment 96, wherein the one or more structural features further comprises at least one structural feature selected from the group consisting of: a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof. Embodiment 155. The composition of Embodiment 154, wherein the engineered guide RNA comprises at least about: 80%, 85%, 90%, 92%, 95%, 97%, or 99% sequence identity to SEQ ID NO: 796. Embodiment 156. The composition of Embodiment 155, wherein the engineered guide RNA comprises SEQ ID NO: 796. Embodiment 157. The composition of Embodiment 10, wherein the first 6/6 symmetric internal loop is at position 26, relative to the target adenosine at position 0. Embodiment 158. The composition of Embodiment 157, wherein the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 159. The composition of Embodiment 158, wherein the engineered guide RNA comprises at least about: 80%, 85%, 90%, 92%, 95%, 97%, or 99% sequence identity to SEQ ID NO: 464. Embodiment 160. The composition of Embodiment 159, wherein the engineered guide RNA comprises SEQ ID NO: 464. Embodiment 161. The composition of Embodiment 157, wherein the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 162. The composition of Embodiment 161, wherein the engineered guide RNA comprises at least about: 80%, 85%, 90%, 92%, 95%, 97%, or 99% sequence identity to SEQ ID NO: 465. Embodiment 163. The composition of Embodiment 162, wherein the engineered guide RNA comprises SEQ ID NO: 465. Embodiment 164. The composition of Embodiment 157, wherein the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, and any combination thereof. Embodiment 165. The composition of Embodiment 164, wherein the engineered guide RNA comprises at least about: 80%, 85%, 90%, 92%, 95%, 97%, or 99% sequence identity to SEQ ID NO: 583. Embodiment 166. The composition of Embodiment 165, wherein the engineered guide RNA comprises SEQ ID NO: 583. Embodiment 167. The composition of Embodiment 157, wherein the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 168. The composition of Embodiment 167, wherein the engineered guide RNA comprises at least about: 80%, 85%, 90%, 92%, 95%, 97%, or 99% sequence identity to SEQ ID NO: 584. Embodiment 169. The composition of Embodiment 167, wherein the engineered guide RNA comprises SEQ ID NO: 584. Embodiment 170. The composition of Embodiment 157, wherein the one or more structural features further comprises at least one structural feature selected from the group consisting of: a 6/6 symmetric internal loop at position −20 relative to position 0, a U/U mismatch at position −11 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 171. The composition of Embodiment 170, wherein the engineered guide RNA comprises at least about: 80%, 85%, 90%, 92%, 95%, 97%, or 99% sequence identity to SEQ ID NO: 589. Embodiment 172. The composition of Embodiment 171, wherein the engineered guide RNA comprises SEQ ID NO: 589. Embodiment 173. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, and any combination thereof. Embodiment 174. The composition of Embodiment 173, wherein the engineered guide RNA comprises at least about: 80%, 85%, 90%, 92%, 95%, 97%, or 99% sequence identity to SEQ ID NO: 610. Embodiment 175. The composition of Embodiment 174, wherein the engineered guide RNA comprises SEQ ID NO: 610. Embodiment 176. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 177. The composition of Embodiment 176, wherein the engineered guide RNA comprises at least about: 80%, 85%, 90%, 92%, 95%, 97%, or 99% sequence identity to SEQ ID NO: 611. Embodiment 178. The composition of Embodiment 177, wherein the engineered guide RNA comprises SEQ ID NO: 611. Embodiment 179. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 180. The composition of Embodiment 179, wherein the engineered guide RNA comprises at least about: 80%, 85%, 90%, 92%, 95%, 97%, or 99% sequence identity to SEQ ID NO: 618. Embodiment 181. The composition of Embodiment 180, wherein the engineered guide RNA comprises SEQ ID NO: 618. Embodiment 182. The composition of Embodiment 157, wherein the one or more structural features further comprises at least one structural feature selected from the group consisting of: a 6/6 symmetric internal loop at position −20 relative to position 0, a A/G mismatch at position −7 relative to position 0, a C/U mismatch at position −4 relative to position 0, a 2/2 symmetric bulge at position −1 relative to position 0, a G/U Wobble at position 8 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 183. The composition of Embodiment 182, wherein the engineered guide RNA comprises at least about: 80%, 85%, 90%, 92%, 95%, 97%, or 99% sequence identity to SEQ ID NO: 662. Embodiment 184. The composition of Embodiment 183, wherein the engineered guide RNA comprises SEQ ID NO: 662. Embodiment 185. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 3 relative to position 0, a 2/2 symmetric bulge at position 7 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 186. The composition of Embodiment 185, wherein the engineered guide RNA comprises at least about: 80%, 85%, 90%, 92%, 95%, 97%, or 99% sequence identity to SEQ ID NO: 664. Embodiment 187. The composition of Embodiment 186, wherein the engineered guide RNA comprises SEQ ID NO: 664. Embodiment 188. The composition of Embodiment 157, wherein the one or more structural features further comprises at least one structural feature selected from the group consisting of: a 6/6 symmetric internal loop at position −20 relative to position 0, a A/C mismatch at position −7 relative to position 0, a C/U mismatch at position −4 relative to position 0, a 2/2 symmetric bulge at position −1 relative to position 0, a 2/2 symmetric bulge at position 10 relative to position 0, an A/C mismatch at position 19 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 189. The composition of Embodiment 188, wherein the engineered guide RNA comprises at least about: 80%, 85%, 90%, 92%, 95%, 97%, or 99% sequence identity to SEQ ID NO: 672. Embodiment 190. The composition of Embodiment 189, wherein the engineered guide RNA comprises SEQ ID NO: 672. Embodiment 191. The composition of Embodiment 157, wherein the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, and any combination thereof. Embodiment 192. The composition of Embodiment 191, wherein the engineered guide RNA comprises at least about: 80%, 85%, 90%, 92%, 95%, 97%, or 99% sequence identity to SEQ ID NO: 675. Embodiment 193. The composition of Embodiment 192, wherein the engineered guide RNA comprises SEQ ID NO: 675. Embodiment 194. The composition of Embodiment 157, wherein the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 195. The composition of Embodiment 194, wherein the engineered guide RNA comprises at least about: 80%, 85%, 90%, 92%, 95%, 97%, or 99% sequence identity to SEQ ID NO: 676. Embodiment 196. The composition of Embodiment 195, wherein the engineered guide RNA comprises SEQ ID NO: 676. Embodiment 197. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, and any combination thereof. Embodiment 198. The composition of Embodiment 197, wherein the engineered guide RNA comprises at least about: 80%, 85%, 90%, 92%, 95%, 97%, or 99% sequence identity to SEQ ID NO: 699. Embodiment 199. The composition of Embodiment 198, wherein the engineered guide RNA comprises SEQ ID NO: 699. Embodiment 200. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 201. The composition of Embodiment 200, wherein the engineered guide RNA comprises at least about: 80%, 85%, 90%, 92%, 95%, 97%, or 99% sequence identity to SEQ ID NO: 700. Embodiment 202. The composition of Embodiment 201, wherein the engineered guide RNA comprises SEQ ID NO: 700. Embodiment 203. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, and any combination thereof. Embodiment 204. The composition of Embodiment 203, wherein the engineered guide RNA comprises at least about: 80%, 85%, 90%, 92%, 95%, 97%, or 99% sequence identity to SEQ ID NO: 718. Embodiment 205. The composition of Embodiment 204, wherein the engineered guide RNA comprises SEQ ID NO: 718. Embodiment 206. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 207. The composition of Embodiment 206, wherein the engineered guide RNA comprises at least about: 80%, 85%, 90%, 92%, 95%, 97%, or 99% sequence identity to SEQ ID NO: 719. Embodiment 208. The composition of Embodiment 207, wherein the engineered guide RNA comprises SEQ ID NO: 719. Embodiment 209. The composition of Embodiment 157, wherein the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a C/C mismatch at position 9 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 210. The composition of Embodiment 209, wherein the engineered guide RNA comprises at least about: 80%, 85%, 90%, 92%, 95%, 97%, or 99% sequence identity to SEQ ID NO: 745. Embodiment 211. The composition of Embodiment 210, wherein the engineered guide RNA comprises SEQ ID NO: 745. Embodiment 212. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 213. The composition of Embodiment 212, wherein the engineered guide RNA comprises at least about: 80%, 85%, 90%, 92%, 95%, 97%, or 99% sequence identity to SEQ ID NO: 750. Embodiment 214. The composition of Embodiment 213, wherein the engineered guide RNA comprises SEQ ID NO: 750. Embodiment 215. The composition of Embodiment 157, wherein the one or more structural features further comprises at least one structural feature selected from the group consisting of: a 6/6 symmetric internal loop at position −20 relative to position 0, a A/A mismatch at position −7 relative to position 0, a 2/2 symmetric bulge at position −1 relative to position 0, a C/U mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 216. The composition of Embodiment 215, wherein the engineered guide RNA comprises at least about: 80%, 85%, 90%, 92%, 95%, 97%, or 99% sequence identity to SEQ ID NO: 762. Embodiment 217. The composition of Embodiment 216, wherein the engineered guide RNA comprises SEQ ID NO: 762. Embodiment 218. The composition of Embodiment 157, wherein the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a U/U mismatch at position 5 relative to position 0, a G/U Wobble at position 8 relative to position 0, a C/U mismatch at position 14 relative to position 0, a G/G mismatch at position 17 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 219. The composition of Embodiment 218, wherein the engineered guide RNA comprises at least about: 80%, 85%, 90%, 92%, 95%, 97%, or 99% sequence identity to SEQ ID NO: 1099. Embodiment 220. The composition of Embodiment 219, wherein the engineered guide RNA comprises SEQ ID NO: 1099. Embodiment 221. The composition of Embodiment 157, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a C/U mismatch at position 9 relative to position 0, a C/U mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 222. The composition of Embodiment 221, wherein the engineered guide RNA comprises at least about: 80%, 85%, 90%, 92%, 95%, 97%, or 99% sequence identity to SEQ ID NO: 1120. Embodiment 223. The composition of Embodiment 222, wherein the engineered guide RNA comprises SEQ ID NO: 1120. Embodiment 224. The composition of Embodiment 157, wherein the one or more structural features further comprises at least one structural feature selected from the group consisting of: a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 225. The composition of Embodiment 224, wherein the engineered guide RNA comprises at least about: 80%, 85%, 90%, 92%, 95%, 97%, or 99% sequence identity to SEQ ID NO: 1213. Embodiment 226. The composition of Embodiment 225, wherein the engineered guide RNA comprises SEQ ID NO: 1213. Embodiment 227. The composition of Embodiment 157, wherein the one or more structural features further comprises at least one structural feature selected from the group consisting of: a 6/6 symmetric internal loop at position −22 relative to position 0, a 3/3 symmetric bulge mismatch at position −13 relative to position 0, a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof. Embodiment 228. The composition of Embodiment 227, wherein the engineered guide RNA comprises at least about: 80%, 85%, 90%, 92%, 95%, 97%, or 99% sequence identity to SEQ ID NO: 1214. Embodiment 229. The composition of Embodiment 228, wherein the engineered guide RNA comprises SEQ ID NO: 1214. Embodiment 230. The composition of Embodiment 10, wherein the first 6/6 symmetric internal loop is at position 25, relative to the target adenosine at position 0. Embodiment 231. The composition of Embodiment 230, wherein the one or more structural features further comprises at least one structural feature selected from the group consisting of: a 3/3 symmetric bulge at position −25 relative to position 0, a 3/3 symmetric bulge at position −16 relative to position 0, a 6/6 symmetric internal loop at position −4 relative to position 0, a G/G mismatch at position −1 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. Embodiment 232. The composition of Embodiment 231, wherein the engineered guide RNA comprises at least about: 80%, 85%, 90%, 92%, 95%, 97%, or 99% sequence identity to SEQ ID NO: 88. Embodiment 233. The composition of Embodiment 232, wherein the engineered guide RNA comprises SEQ ID NO: 88. Embodiment 234. The composition of Embodiment 230, wherein the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. Embodiment 235. The composition of Embodiment 234, wherein the engineered guide RNA comprises at least about: 80%, 85%, 90%, 92%, 95%, 97%, or 99% sequence identity to SEQ ID NO: 903. Embodiment 236. The composition of Embodiment 235, wherein the engineered guide RNA comprises SEQ ID NO: 903. Embodiment 237. The composition of Embodiment 230, wherein the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. Embodiment 238. The composition of Embodiment 237, wherein the engineered guide RNA comprises at least about: 80%, 85%, 90%, 92%, 95%, 97%, or 99% sequence identity to SEQ ID NO: 1179. Embodiment 239. The composition of Embodiment 238, wherein the engineered guide RNA comprises SEQ ID NO: 1179. Embodiment 240. The composition of Embodiment 230, wherein the one or more structural features further comprises at least one structural feature selected from the group consisting of: a 6/6 symmetric internal loop at position −4 relative to position 0, a G/G mismatch at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof. Embodiment 241. The composition of Embodiment 240, wherein the engineered guide RNA comprises at least about: 80%, 85%, 90%, 92%, 95%, 97%, or 99% sequence identity to SEQ ID NO: 1212. Embodiment 242. The composition of Embodiment 241, wherein the engineered guide RNA comprises SEQ ID NO: 1212. Embodiment 243. The composition of Embodiment 10, wherein the first 6/6 symmetric internal loop is at position 24, relative to the target adenosine at position 0. Embodiment 244. The composition of Embodiment 243, wherein the one or more structural features further comprises at least one structural feature selected from the group consisting of: a 3/3 symmetric bulge at position −18 relative to position 0, a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 245. The composition of Embodiment 244, wherein the engineered guide RNA comprises at least about: 80%, 85%, 90%, 92%, 95%, 97%, or 99% sequence identity to SEQ ID NO: 118. Embodiment 246. The composition of Embodiment 245, wherein the engineered guide RNA comprises SEQ ID NO: 118. Embodiment 247. The composition of Embodiment 243, wherein the one or more structural features further comprises at least one structural feature selected from the group consisting of: a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 248. The composition of Embodiment 247, wherein the engineered guide RNA comprises at least about: 80%, 85%, 90%, 92%, 95%, 97%, or 99% sequence identity to SEQ ID NO: 119. Embodiment 249. The composition of Embodiment 248, wherein the engineered guide RNA comprises SEQ ID NO: 119. Embodiment 250. The composition of Embodiment 243, wherein the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 251. The composition of Embodiment 250, wherein the engineered guide RNA comprises at least about: 80%, 85%, 90%, 92%, 95%, 97%, or 99% sequence identity to SEQ ID NO: 270. Embodiment 252. The composition of Embodiment 251, wherein the engineered guide RNA comprises SEQ ID NO: 270. Embodiment 253. The composition of Embodiment 243, wherein the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 254. The composition of Embodiment 253, wherein the engineered guide RNA comprises at least about: 80%, 85%, 90%, 92%, 95%, 97%, or 99% sequence identity to SEQ ID NO: 271. Embodiment 255. The composition of Embodiment 254, wherein the engineered guide RNA comprises SEQ ID NO: 271. Embodiment 256. The composition of Embodiment 243, wherein the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 257. The composition of Embodiment 256, wherein the engineered guide RNA comprises at least about: 80%, 85%, 90%, 92%, 95%, 97%, or 99% sequence identity to SEQ ID NO: 403. Embodiment 258. The composition of Embodiment 257, wherein the engineered guide RNA comprises SEQ ID NO: 403. Embodiment 259. The composition of Embodiment 243, wherein the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof. Embodiment 260. The composition of Embodiment 259, wherein the engineered guide RNA comprises at least about: 80%, 85%, 90%, 92%, 95%, 97%, or 99% sequence identity to SEQ ID NO: 1081. Embodiment 261. The composition of Embodiment 260, wherein the engineered guide RNA comprises SEQ ID NO: 1081. Embodiment 262. The composition of Embodiment 10, wherein the first 6/6 symmetric internal loop is at position 23, relative to the target adenosine at position 0. Embodiment 263. The composition of Embodiment 262, wherein the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 264. The composition of Embodiment 263, wherein the engineered guide RNA comprises at least about: 80%, 85%, 90%, 92%, 95%, 97%, or 99% sequence identity to SEQ ID NO: 401. Embodiment 265. The composition of Embodiment 264, wherein the engineered guide RNA comprises SEQ ID NO: 401. Embodiment 266. The composition of Embodiment 262, wherein the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 267. The composition of Embodiment 266, wherein the engineered guide RNA comprises at least about: 80%, 85%, 90%, 92%, 95%, 97%, or 99% sequence identity to SEQ ID NO: 402. Embodiment 268. The composition of Embodiment 267, wherein the engineered guide RNA comprises SEQ ID NO: 402. Embodiment 269. The composition of Embodiment 262, wherein the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 270. The composition of Embodiment 269, wherein the engineered guide RNA comprises at least about: 80%, 85%, 90%, 92%, 95%, 97%, or 99% sequence identity to SEQ ID NO: 458. Embodiment 271. The composition of Embodiment 270, wherein the engineered guide RNA comprises SEQ ID NO: 458. Embodiment 272. The composition of Embodiment 262, wherein the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof. Embodiment 273. The composition of Embodiment 272, wherein the engineered guide RNA comprises at least about: 80%, 85%, 90%, 92%, 95%, 97%, or 99% sequence identity to SEQ ID NO: 459. Embodiment 274. The composition of Embodiment 273, wherein the engineered guide RNA comprises SEQ ID NO: 459. Embodiment 275. The composition of Embodiment 10, wherein the first 6/6 symmetric internal loop is at position 22, relative to the target adenosine at position 0. Embodiment 276. The composition of Embodiment 275, wherein the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. Embodiment 277. The composition of Embodiment 276, wherein the engineered guide RNA comprises at least about: 80%, 85%, 90%, 92%, 95%, 97%, or 99% sequence identity to SEQ ID NO: 1135. Embodiment 278. The composition of Embodiment 277, wherein the engineered guide RNA comprises SEQ ID NO: 1135. Embodiment 279. The composition of Embodiment 275, wherein the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof. Embodiment 280. The composition of Embodiment 279, wherein the engineered guide RNA comprises at least about: 80%, 85%, 90%, 92%, 95%, 97%, or 99% sequence identity to SEQ ID NO: 1136. Embodiment 281. The composition of Embodiment 280, wherein the engineered guide RNA comprises SEQ ID NO: 1136. Embodiment 282. The composition of any one of Embodiments 1-281, wherein the one or more structural features comprises at least a first 6/6 symmetric internal loop and at least a second 6/6 symmetric loop Embodiment 283. The composition of any one of Embodiments 1-281, wherein the one or more structural features comprises the bulge, wherein the bulge is a symmetric bulge. Embodiment 284. The composition of any one of Embodiments 1-281, wherein the one or more structural features comprises the bulge, wherein the bulge is an asymmetric bulge. Embodiment 285. The composition of any one of Embodiments 1-284, wherein the one or more structural features comprises the internal loop, wherein the internal loop is a symmetric internal loop. Embodiment 286. The composition of any one of Embodiments 1-284, wherein the one or more structural features comprises the internal loop, wherein the internal loop is an asymmetric internal loop. Embodiment 287. The composition of any one of Embodiments 1-286, wherein the guide-target RNA scaffold comprises a Wobble base pair. Embodiment 288. The composition of any one of Embodiments 1-287, wherein the one or more structural features comprises the hairpin, wherein the hairpin is a recruitment hairpin or a non-recruitment hairpin. Embodiment 289. The composition of any one of Embodiments 1-288, wherein upon hybridization of the engineered guide RNA to the sequence of the target MAPT RNA, the engineered guide RNA facilitates RNA editing of one or more adenosines in the sequence of the target MAPT RNA by an RNA editing entity. Embodiment 290. The composition of Embodiment 289, wherein the RNA editing entity comprises ADAR1, ADAR2, ADAR3, or any combination thereof. Embodiment 291. The composition of any one of Embodiments 1-290, wherein the engineered guide RNA hybridizes to the sequence of the target MAPT RNA via a first targeting domain; and wherein the engineered guide RNA hybridizes to a second sequence of the target MAPT RNA via a second targeting domain. Embodiment 292. The composition of Embodiment 291, wherein the sequence of the MAPT RNA is the AUG in the 18th nucleotide in Exon 1 (c.1), and wherein the second sequence of the target MAPT RNA is selected from the group consisting of: an AUG in the 48th nucleotide in Exon 1 (c.31), an AUG in the 108th nt of Exon 1 (c.91), an AUG in the 6th nucleotide in Exon 5 (c.379), and any combination thereof. Embodiment 293. The composition of any one of Embodiments 1-292, wherein the engineered guide RNA is encoded by an engineered polynucleotide. Embodiment 294. The composition of Embodiment 293, wherein the engineered polynucleotide is comprised in or on a vector. Embodiment 295. The composition of Embodiment 294, wherein the vector is a viral vector, and wherein the engineered polynucleotide is encapsidated in the viral vector. Embodiment 296. The composition of Embodiment 295, wherein the viral vector is an adeno-associated viral (AAV) vector or a derivative thereof. Embodiment 297. The composition of Embodiment 295, wherein the viral vector is an adeno-associated viral (AAV) vector and wherein the AAV vector is AAV1, AAV2, AAV3, AAV4, AAV5, AAV6, AAV7, AAV8, AAV9, AAV10, AAV11, or a derivative, a chimera, or a variant of any of these. Embodiment 298. The composition of any one of Embodiments 296-297, wherein the AAV vector is a recombinant AAV (rAAV) vector, a hybrid AAV vector, a chimeric AAV vector, a self-complementary AAV (scAAV) vector, or any combination thereof. Embodiment 299. The composition of any one of Embodiments 1-298, wherein the engineered guide RNA has at least 80%, 85%, 90%, 92%, 95%, 97%, or 99% sequence identity to any one of SEQ ID NO: 2-SEQ ID NO: 1215. Embodiment 300. The composition of any one of Embodiments 1-299, wherein the engineered guide RNA has a sequence of any one of SEQ ID NO: 2-SEQ ID NO: 1215. Embodiment 301. The composition of any one of Embodiments 1-300, in unit dose form. Embodiment 302. A pharmaceutical composition comprising: the composition of any one of Embodiments 1-301; and a pharmaceutically acceptable: excipient, carrier, or diluent. Embodiment 303. The pharmaceutical composition of Embodiment 302, in unit dose form. Embodiment 304. A method of treating a disease or a condition in a subject in need thereof, the method comprising administering to the subject a therapeutically effective amount of the composition of any one of Embodiments 1-301 or the pharmaceutical composition of Embodiment 302 or 303. Embodiment 305. The method of Embodiment 304, wherein the disease or condition comprises a Tauopathy. Embodiment 306. The method of Embodiment 304, wherein the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome. Embodiment 307. The method of any one of Embodiments 304-306, wherein the subject is a human or a non-human animal. Embodiment 308. The method of any one of Embodiments 304-307, wherein the administering is sufficient to treat one or more symptoms of the disease of condition. Embodiment 309. The method of Embodiment 308, wherein the one of more symptoms treated comprises a reduction in memory loss, a reduction in confusion, or any combination thereof, as compared to prior to the administering. Embodiment 310. The method of Embodiment 308-309, wherein the administering is sufficient to reduce aggregation of tau protein, relative to: a level of aggregation prior to the administering; a level of accumulated aggregation in the subject in the absence of the administering; or both. Embodiment 311. The method of Embodiment 308-309, wherein the administering is sufficient to reduce an amount of: neurofibrillary tangles; neuronal cell death; neuroinflammation; or any combination thereof, relative to an amount prior to the administering. Embodiment 312. The composition of Embodiment 308, wherein the amount of phosphorylated tau protein is decreased in the subject after the administering, relative to an amount of phosphorylated tau protein prior to the administering. Embodiment 313. The composition of Embodiment 308, wherein a ratio of a 4R isoform of Tau to a 3R isoform of tau is increased upon administration, relative to a ratio prior to the administering. Embodiment 314. The composition of any one of Embodiments 1-301 or the pharmaceutical composition of Embodiment 302 or 303 for use in treatment of a disease or condition. Embodiment 315. The composition or the pharmaceutical composition for use of Embodiment 314, wherein the disease or condition comprises a Tauopathy. Embodiment 316. The composition or the pharmaceutical composition for use of Embodiment 314, wherein the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome. Embodiment 317. A method of editing a MAPT RNA, the method comprising contacting the MAPT RNA with the composition of any one of Embodiments 1-301 and an RNA editing entity, thereby editing the MAPT RNA. Embodiment 318. The method of Embodiment 317, wherein the editing comprises editing one or more adenosines within the 3′ untranslated region (UTR) of the MAPT RNA. Embodiment 319. The method of Embodiment 317, wherein the editing comprises editing one or more adenosines within the 5′ untranslated region (UTR) of the MAPT RNA. Embodiment 320. The method of Embodiment 317, wherein the editing comprises editing one or more adenosines of a transcription initiation site (TIS) of the MAPT RNA. Embodiment 321. The method of Embodiment 320, wherein the translation initiation site is the c.1 TIS corresponding to the canonical TIS at nucleotide position 151 of the MAPT reference transcript of NM_001377265.1. Embodiment 322. The method of any one of Embodiments 320-321, wherein the MAPT RNA comprises a pre-mRNA transcript of MAPT. Embodiment 323. The method of Embodiment 322, wherein at least 40%, at least 50%, at least 60%, at least 70%, at least 80%, at least 85%, at least 90%, at least 95%, or at least 99% of the pre-mRNA transcripts of MAPT have at least one edit. Embodiment 324. The method of Embodiment 317, wherein the editing of MAPT RNA facilitates a protein knockdown. Embodiment 325. The method of Embodiment 324, wherein the protein knockdown comprises a reduction of at least 10%, relative to an amount of protein present prior to the contacting. Embodiment 326. The method of Embodiment 324, wherein the protein knockdown comprises a reduction of from about 10% to about 25%, relative to an amount of protein present prior to the contacting. Embodiment 327. The method of Embodiment 324, wherein the protein knockdown comprises a reduction of at least 50%, relative to an amount of protein present prior to the contacting. Embodiment 328. The method of Embodiment 324, wherein the protein knockdown comprises a knockdown of tau. Embodiment 329. The method of any one of Embodiments 324-328, wherein the knockdown is measured in an in vitro assay. Embodiment 330. The method of any one of Embodiments 324-328, wherein the knockdown is measured in an in vivo assay. Embodiment 331. The method of any one of Embodiments 324-328, wherein the knockdown is measured in a human subject.

### EXAMPLES

The following illustrative examples are representative of embodiments of the stimulation, systems, and methods described herein and are not meant to be limiting in any way.

### Example 1

### Engineered Guide RNAs for Editing MAPT TIS

This example describes engineered guide RNAs for editing MAPT RNA to knockdown expression of the Tau protein. Engineered guide RNAs of the present disclosure are designed to target a translation initiation site (TIS) of MAPT (e.g., an AUG in the 18th nt in Exon 1 (c.1), an AUG in the 48^{th} nt in Exon 1 (c.31), an AUG in the 108^{th} nt of Exon 1 (c.91), an AUG in the 6^{th} nt in Exon 5 (c.379), or any combination thereof) RNA and facilitate ADAR-mediated RNA editing of AUG (the TIS) to GUG, thus, inhibiting MAPT translation. Editing results in knockdown of the Tau protein. Engineered guide RNAs are packaged and delivered in AAV virus and are administered to a subject in need thereof. Upon administration to of the engineered guide RNAs, in vitro or in vivo, the engineered guide RNAs edit the MAPT TIS, thereby reducing Tau protein expression. Upon administration to a subject having a Tauopathy (e.g., AD, FTD, Parkinson's disease), the engineered guide RNAs are therapeutically effective and reduce symptoms and/or cure the Tauopathy.

### Example 2

### Engineered Guide RNA Compositions Targeting the MAPT c.1 TIS

This example describes sequences of engineered guide RNAs that target the c.1 TIS corresponding to the canonical TIS at nucleotide position 151 of the MAPT reference transcript of NM_001377265.1. Self-annealing RNA structures, which comprised (i) the engineered guide RNAs shown in TABLE 1 and (ii) the RNA sequences of the MAPT region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing by next generation sequencing (NGS). Engineered guide RNAs that displayed greater than 50% on-target editing of the MAPT c.1 TIS for ADAR1 and/or ADAR2, as quantified at a read depth of >200, are shown in TABLE 1. All polynucleotide sequences encoding for the engineered guide RNAs of TABLE 1, are also encompassed herein, which are represented by each of the sequences shown in TABLE 1, with a T substituted for each U. For each sequence, the structural features formed in the double stranded RNA substrate upon hybridization of the guide RNA to the target MAPT RNA, are shown in the second column of TABLE 1. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, in SEQ ID NO: 2, “−22_3-3 bulge-symmetric CUA-GAU” is read as a structural feature formed in a guide-target RNA scaffold (target MAPT c.1 TIS RNA sequence hybridized to an engineered guide RNA of SEQ ID NO: 2), where
a. the structural feature starts 22 nucleotides upstream (5′) (the −22 position) from the target A (0 position) of the target RNA sequence
b. three contiguous bases from the target RNA sequence and three contiguous bases from the engineered guide RNA form the structural feature
c. the structural feature is a symmetric bulge
d. a sequence of CUA from the target RNA side and a sequence of GAU from the engineered guide RNA side participate in forming the symmetric bulge.

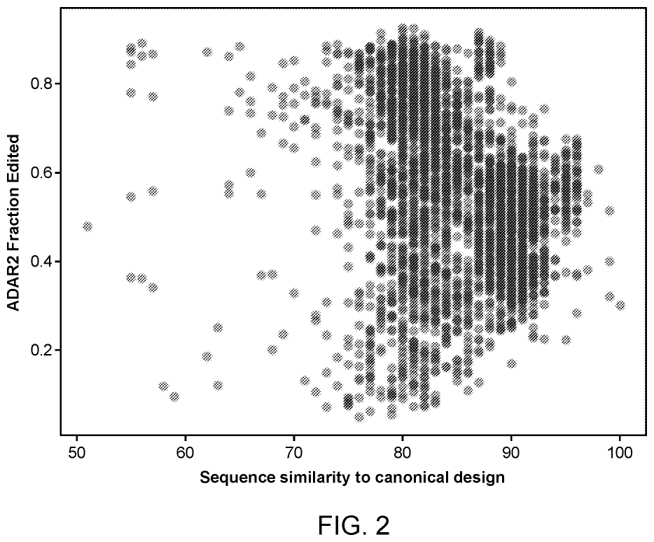

For reference, FIG. 1 can be used as an aid to visualize the structural features and the nomenclature disclosed herein. FIG. 2 is a plot showing, on the x-axis, the sequence similarity of the MAPT c.1 TIS-targeting engineered guide RNAs of the present disclosure to a canonical guide RNA design and, on the y-axis, the edited fraction by an ADAR2 enzyme. Table 1 further includes the amount of on target editing achieved via ADAR1 or ADAR2 seperately, as well as ADAR1 and ADAR2. The specificity of each guide was also calculated for each engineered guide via ADAR1, ADAR2, and ADAR1+ADAR2. Specificity as provided in Table 1 was calculated using the formula: Specificity=(fraction on-target editing+1)/(sum(non-synonymous off-target editing)). These data highlight the diverse sequence space represented by the MAPT c.1 TIS-targeting engineered guide RNAs of the present disclosure, which have a range of different structural features that drive sequence diversity and which exhibit high on-target editing efficiency.

Lengthy table referenced here
US20240279656A1-20240822-T00001
Please refer to the end of the specification for access instructions.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

LENGTHY TABLES
The patent application contains a lengthy table section. A copy of the table is available in electronic form from the USPTO web site (https://seqdata.uspto.gov/?pageRequest=docDetail&DocID=US20240279656A1). An electronic copy of the table will also be available from the USPTO upon request and payment of the fee set forth in 37 CFR 1.19(b)(3).

What is claimed is:

1. A composition comprising an engineered guide RNA, wherein:
a) the engineered guide RNA, upon hybridization to a c.1 translation initiation sequence (TIS) of a target MAPT RNA, forms a guide-target RNA scaffold with the sequence of the target MAPT RNA, wherein the c.1 TIS is an AUG starting at the 18th nucleotide in Exon 1 of the target MAPT RNA;
b) formation of the guide-target RNA scaffold substantially forms one or more structural features selected from the group consisting of: a bulge, an internal loop, and a hairpin;
c) the structural feature is not present within the engineered guide RNA prior to the hybridization of the engineered guide RNA to the MAPT target RNA; and
d) upon hybridization of the engineered guide RNA to the c.1 TIS of the target MAPT RNA, the engineered guide RNA facilitates RNA editing of one or more target adenosines in the c.1 TIS of the target MAPT RNA by an RNA editing entity.

2. The composition of claim 1, wherein the one or more structural features comprises: a first 6/6 symmetric internal loop at a position selected from the group consisting of: 34, 33, 32, 31, 30, 26, 25, 24, 23, and 22; relative to the target adenosine at position 0.

3. The composition of claim 2, wherein the first 6/6 symmetric internal loop is at position 34, relative to the target adenosine at position 0.

4. The composition of claim 3, wherein the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof.

5. The composition of claim 4, wherein the engineered guide RNA comprises at least about: 80%, 85%, 90%, 92%, 95%, 97%, or 99% sequence identity to SEQ ID NO: 478.

6. The composition of claim 3, wherein the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof.

7. The composition of claim 6, wherein the engineered guide RNA comprises at least about: 80%, 85%, 90%, 92%, 95%, 97%, or 99% sequence identity to SEQ ID NO: 479.

8. The composition of claim 2, wherein the first 6/6 symmetric internal loop is at position 33, relative to the target adenosine at position 0.

9. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

10. The composition of claim 9, wherein the engineered guide RNA comprises at least about: 80%, 85%, 90%, 92%, 95%, 97%, or 99% sequence identity to SEQ ID NO: 592.

11. The composition of claim 8, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

12. The composition of claim 11, wherein the engineered guide RNA comprises at least about: 80%, 85%, 90%, 92%, 95%, 97%, or 99% sequence identity to SEQ ID NO: 593.

13. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

14. The composition of claim 13, wherein the engineered guide RNA comprises at least about: 80%, 85%, 90%, 92%, 95%, 97%, or 99% sequence identity to SEQ ID NO: 639.

15. The composition of claim 8, wherein the one or more structural features further comprises at least one structural feature selected from the group consisting of: a 6/6 symmetric internal loop at position −9 relative to position 0, a C/U mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

16. The composition of claim 15, wherein the engineered guide RNA comprises at least about: 80%, 85%, 90%, 92%, 95%, 97%, or 99% sequence identity to SEQ ID NO: 640.

17. The composition of claim 8, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, and any combination thereof.

18. The composition of claim 17, wherein the engineered guide RNA comprises at least about: 80%, 85%, 90%, 92%, 95%, 97%, or 99% sequence identity to SEQ ID NO: 686.

19. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

20. The composition of claim 19, wherein the engineered guide RNA comprises at least about: 80%, 85%, 90%, 92%, 95%, 97%, or 99% sequence identity to SEQ ID NO: 687.

21. The composition of claim 8, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

22. The composition of claim 21, wherein the engineered guide RNA comprises at least about: 80%, 85%, 90%, 92%, 95%, 97%, or 99% sequence identity to SEQ ID NO: 688.

23. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a G/G mismatch at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

24. The composition of claim 23, wherein the engineered guide RNA comprises at least about: 80%, 85%, 90%, 92%, 95%, 97%, or 99% sequence identity to SEQ ID NO: 695.

25. The composition of claim 8, wherein the one or more structural features further comprises at least one structural feature selected from the group consisting of: a 6/6 symmetric internal loop at position −9 relative to position 0, a C/C mismatch at position −5 relative to position 0, a 2/2 symmetric bulge at position −1 relative to position 0, a G/U Wobble at position 2 relative to position 0, a G/U Wobble at position 6 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

26. The composition of claim 25, wherein the engineered guide RNA comprises at least about: 80%, 85%, 90%, 92%, 95%, 97%, or 99% sequence identity to SEQ ID NO: 696.

27. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

28. The composition of claim 27, wherein the engineered guide RNA comprises at least about: 80%, 85%, 90%, 92%, 95%, 97%, or 99% sequence identity to SEQ ID NO: 714.

29. The composition of claim 8, wherein the one or more structural features further comprises at least one structural feature selected from the group consisting of: a 6/6 symmetric internal loop at position −9 relative to position 0, a 2/2 symmetric bulge at position −1 relative to position 0, a G/U Wobble at position 6 relative to position 0, a G/G mismatch at position 8 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

30. The composition of claim 29, wherein the engineered guide RNA comprises at least about: 80%, 85%, 90%, 92%, 95%, 97%, or 99% sequence identity to SEQ ID NO: 715.

31. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

32. The composition of claim 31, wherein the engineered guide RNA comprises at least about: 80%, 85%, 90%, 92%, 95%, 97%, or 99% sequence identity to SEQ ID NO: 722.

33. The composition of claim 8, wherein the one or more structural features further comprises at least one structural feature selected from the group consisting of: a 6/6 symmetric internal loop at position −9 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

34. The composition of claim 33, wherein the engineered guide RNA comprises at least about: 80%, 85%, 90%, 92%, 95%, 97%, or 99% sequence identity to SEQ ID NO: 723.

35. The composition of claim 8, wherein the one or more structural features further comprises at least one structural feature selected from the group consisting of: a 3/3 symmetric bulge at position −22 relative to position 0, a 3/3 symmetric bulge at position −13 relative to position 0, a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

36. The composition of claim 35, wherein the engineered guide RNA comprises at least about: 80%, 85%, 90%, 92%, 95%, 97%, or 99% sequence identity to SEQ ID NO: 1059.

37. The composition of claim 8, wherein the one or more structural features further comprises at least one structural feature selected from the group consisting of: a 3/3 symmetric bulge at position −21 relative to position 0, a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

38. The composition of claim 37, wherein the engineered guide RNA comprises at least about: 80%, 85%, 90%, 92%, 95%, 97%, or 99% sequence identity to SEQ ID NO: 1116.

39. The composition of claim 8, wherein the one or more structural features further comprises at least one structural feature selected from the group consisting of: a 6/6 symmetric internal loop at position −9 relative to position 0, an A/C mismatch at position −7 relative to position 0, a 2/2 symmetric bulge at position −1 relative to position 0, a C/C mismatch at position 9 relative to position 0, a C/U mismatch at position 14 relative to position 0, a G/U Wobble at position 18 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

40. The composition of claim 39, wherein the engineered guide RNA comprises at least about: 80%, 85%, 90%, 92%, 95%, 97%, or 99% sequence identity to SEQ ID NO: 1117.

41. The composition of claim 8, wherein the one or more structural features further comprises at least one structural feature selected from the group consisting of: a 6/6 symmetric internal loop at position −1 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

42. The composition of claim 41, wherein the engineered guide RNA comprises at least about: 80%, 85%, 90%, 92%, 95%, 97%, or 99% sequence identity to SEQ ID NO: 1215.

43. The composition of claim 2, wherein the first 6/6 symmetric internal loop is at position 32, relative to the target adenosine at position 0.

44. The composition of claim 43, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

45. The composition of claim 44, wherein the engineered guide RNA comprises at least about: 80%, 85%, 90%, 92%, 95%, 97%, or 99% sequence identity to SEQ ID NO: 438.

46. The composition of claim 43, wherein the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

47. The composition of claim 46, wherein the engineered guide RNA comprises at least about: 80%, 85%, 90%, 92%, 95%, 97%, or 99% sequence identity to SEQ ID NO: 439.

48. The composition of claim 43, wherein the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

49. The composition of claim 48, wherein the engineered guide RNA comprises at least about: 80%, 85%, 90%, 92%, 95%, 97%, or 99% sequence identity to SEQ ID NO: 816.

50. The composition of claim 43, wherein the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

51. The composition of claim 50, wherein the engineered guide RNA comprises at least about: 80%, 85%, 90%, 92%, 95%, 97%, or 99% sequence identity to SEQ ID NO: 817.

52. The composition of claim 2, wherein the first 6/6 symmetric internal loop is at position 31, relative to the target adenosine at position 0.

53. The composition of claim 52, wherein the one or more structural features further comprises at least one structural feature selected from the group consisting of: a 3/3 symmetric bulge at position −19 relative to position 0, a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof.

54. The composition of claim 53, wherein the engineered guide RNA comprises at least about: 80%, 85%, 90%, 92%, 95%, 97%, or 99% sequence identity to SEQ ID NO: 162.

55. The composition of claim 52, wherein the one or more structural features further comprises at least one structural feature selected from the group consisting of: a 6/6 symmetric internal loop at position −7 relative to position 0, a G/G mismatch at position −1 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof.

56. The composition of claim 55, wherein the engineered guide RNA comprises at least about: 80%, 85%, 90%, 92%, 95%, 97%, or 99% sequence identity to SEQ ID NO: 163.

57. The composition of claim 52, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof.

58. The composition of claim 57, wherein the engineered guide RNA comprises at least about: 80%, 85%, 90%, 92%, 95%, 97%, or 99% sequence identity to SEQ ID NO: 929.

59. The composition of claim 52, wherein the one or more structural features further comprises at least one structural feature selected from the group consisting of: a 6/6 symmetric internal loop at position −6 relative to position 0, a 2/2 symmetric bulge at position −1 relative to position 0, a 3/3 symmetric bulge at position 6 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof.

60. The composition of claim 59, wherein the engineered guide RNA comprises at least about: 80%, 85%, 90%, 92%, 95%, 97%, or 99% sequence identity to SEQ ID NO: 930.

61. The composition of claim 2, wherein the first 6/6 symmetric internal loop is at position 30, relative to the target adenosine at position 0.

62. The composition of claim 61, wherein the one or more structural features further comprises at least one structural feature selected from the group consisting of: a 3/3 symmetric bulge at position −21 relative to position 0, a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

63. The composition of claim 62, wherein the engineered guide RNA comprises at least about: 80%, 85%, 90%, 92%, 95%, 97%, or 99% sequence identity to SEQ ID NO: 538.

64. The composition of claim 61, wherein the one or more structural features further comprises at least one structural feature selected from the group consisting of: a 6/6 symmetric internal loop at position −9 relative to position 0, a 2/2 symmetric bulge at position −1 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

65. The composition of claim 64, wherein the engineered guide RNA comprises at least about: 80%, 85%, 90%, 92%, 95%, 97%, or 99% sequence identity to SEQ ID NO: 539.

66. The composition of claim 61, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

67. The composition of claim 66, wherein the engineered guide RNA comprises at least about: 80%, 85%, 90%, 92%, 95%, 97%, or 99% sequence identity to SEQ ID NO: 604.

68. The composition of claim 61, wherein the one or more structural features further comprises at least one structural feature selected from the group consisting of: a 7/7 symmetric internal loop at position −5 relative to position 0, a 2/2 symmetric bulge at position −1 relative to position 0, a C/U mismatch at position 4 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

69. The composition of claim 68, wherein the engineered guide RNA comprises at least about: 80%, 85%, 90%, 92%, 95%, 97%, or 99% sequence identity to SEQ ID NO: 605.

70. The composition of claim 61, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

71. The composition of claim 70, wherein the engineered guide RNA comprises at least about: 80%, 85%, 90%, 92%, 95%, 97%, or 99% sequence identity to SEQ ID NO: 619.

72. The composition of claim 61, wherein the one or more structural features further comprises at least one structural feature selected from the group consisting of: a 7/7 symmetric internal loop at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

73. The composition of claim 72, wherein the engineered guide RNA comprises at least about: 80%, 85%, 90%, 92%, 95%, 97%, or 99% sequence identity to SEQ ID NO: 620.

74. The composition of claim 61, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

75. The composition of claim 74, wherein the engineered guide RNA comprises at least about: 80%, 85%, 90%, 92%, 95%, 97%, or 99% sequence identity to SEQ ID NO: 677.

76. The composition of claim 61, wherein the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

77. The composition of claim 76, wherein the engineered guide RNA comprises at least about: 80%, 85%, 90%, 92%, 95%, 97%, or 99% sequence identity to SEQ ID NO: 678.

78. The composition of claim 61, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

79. The composition of claim 78, wherein the engineered guide RNA comprises at least about: 80%, 85%, 90%, 92%, 95%, 97%, or 99% sequence identity to SEQ ID NO: 684.

80. The composition of claim 61, wherein the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a C/U mismatch at position 11 relative to position 0, a G/G mismatch at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

81. The composition of claim 80, wherein the engineered guide RNA comprises at least about: 80%, 85%, 90%, 92%, 95%, 97%, or 99% sequence identity to SEQ ID NO: 685.

82. The composition of claim 61, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

83. The composition of claim 82, wherein the engineered guide RNA comprises at least about: 80%, 85%, 90%, 92%, 95%, 97%, or 99% sequence identity to SEQ ID NO: 720.

84. The composition of claim 61, wherein the one or more structural features further comprises at least one structural feature selected from the group consisting of: a 7/7 symmetric internal loop at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

85. The composition of claim 84, wherein the engineered guide RNA comprises at least about: 80%, 85%, 90%, 92%, 95%, 97%, or 99% sequence identity to SEQ ID NO: 721.

86. The composition of claim 61, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

87. The composition of claim 86, wherein the engineered guide RNA comprises at least about: 80%, 85%, 90%, 92%, 95%, 97%, or 99% sequence identity to SEQ ID NO: 733.

88. The composition of claim 61, wherein the one or more structural features further comprises at least one structural feature selected from the group consisting of: a 6/6 symmetric internal loop at position −6 relative to position 0, a 2/2 symmetric bulge at position −1 relative to position 0, a G/A mismatch at position 6 relative to position 0, a C/U mismatch at position 9 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

89. The composition of claim 88, wherein the engineered guide RNA comprises at least about: 80%, 85%, 90%, 92%, 95%, 97%, or 99% sequence identity to SEQ ID NO: 734.

90. The composition of claim 61, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

91. The composition of claim 90, wherein the engineered guide RNA comprises at least about: 80%, 85%, 90%, 92%, 95%, 97%, or 99% sequence identity to SEQ ID NO: 763.

92. The composition of claim 61, wherein the one or more structural features further comprises at least one structural feature selected from the group consisting of: a 6/6 symmetric internal loop at position −6 relative to position 0, a 2/2 symmetric bulge at position −1 relative to position 0, a C/U mismatch at position 10 relative to position 0, a G/U Wobble at position 20 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

93. The composition of claim 92, wherein the engineered guide RNA comprises at least about: 80%, 85%, 90%, 92%, 95%, 97%, or 99% sequence identity to SEQ ID NO: 764.

94. The composition of claim 61, wherein the one or more structural features further comprises at least one structural feature selected from the group consisting of: a 3/3 symmetric bulge at position −18 relative to position 0, a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

95. The composition of claim 94, wherein the engineered guide RNA comprises at least about: 80%, 85%, 90%, 92%, 95%, 97%, or 99% sequence identity to SEQ ID NO: 775.

96. The composition of claim 61, wherein the one or more structural features further comprises at least one structural feature selected from the group consisting of: a 7/7 symmetric internal loop at position −5 relative to position 0, a 2/2 symmetric bulge at position −1 relative to position 0, a G/U Wobble at position 2 relative to position 0, a G/G mismatch at position 8 relative to position 0, a G/U Wobble at position 18 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

97. The composition of claim 96, wherein the engineered guide RNA comprises at least about: 80%, 85%, 90%, 92%, 95%, 97%, or 99% sequence identity to SEQ ID NO: 776.

98. The composition of claim 61, wherein the one or more structural features further comprises at least one structural feature selected from the group consisting of: a 3/3 symmetric bulge at position −18 relative to position 0, a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

99. The composition of claim 98, wherein the engineered guide RNA comprises at least about: 80%, 85%, 90%, 92%, 95%, 97%, or 99% sequence identity to SEQ ID NO: 795.

100. The composition of claim 61, wherein the one or more structural features further comprises at least one structural feature selected from the group consisting of: a 6/6 symmetric internal loop at position −6 relative to position 0, a 2/2 symmetric bulge at position −1 relative to position 0, a C/A mismatch at position 10 relative to position 0, a C/A mismatch at position 14 relative to position 0, a 3/3 symmetric bulge at position 42 relative to position 0, a 3/3 symmetric bulge at position 51 relative to position 0, a 3/3 symmetric bulge at position 60 relative to position 0, and any combination thereof.

101. The composition of claim 100, wherein the engineered guide RNA comprises at least about: 80%, 85%, 90%, 92%, 95%, 97%, or 99% sequence identity to SEQ ID NO: 796.

102. The composition of claim 2, wherein the first 6/6 symmetric internal loop is at position 26, relative to the target adenosine at position 0.

103. The composition of claim 102, wherein the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

104. The composition of claim 103, wherein the engineered guide RNA comprises at least about: 80%, 85%, 90%, 92%, 95%, 97%, or 99% sequence identity to SEQ ID NO: 464.

105. The composition of claim 102, wherein the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

106. The composition of claim 105, wherein the engineered guide RNA comprises at least about: 80%, 85%, 90%, 92%, 95%, 97%, or 99% sequence identity to SEQ ID NO: 465.

107. The composition of claim 102, wherein the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, and any combination thereof.

108. The composition of claim 107, wherein the engineered guide RNA comprises at least about: 80%, 85%, 90%, 92%, 95%, 97%, or 99% sequence identity to SEQ ID NO: 583.

109. The composition of claim 102, wherein the one or more structural features further comprises at least one structural feature selected from the group consisting of: a 6/6 symmetric internal loop at position −20 relative to position 0, a C/U mismatch at position −4 relative to position 0, a 2/2 symmetric bulge at position −1 relative to position 0, a G/U Wobble at position 8 relative to position 0, a C/A mismatch at position 11 relative to position 0, a G/U Wobble at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

110. The composition of claim 109, wherein the engineered guide RNA comprises at least about: 80%, 85%, 90%, 92%, 95%, 97%, or 99% sequence identity to SEQ ID NO: 584.

111. The composition of claim 102, wherein the one or more structural features further comprises at least one structural feature selected from the group consisting of: a 6/6 symmetric internal loop at position −20 relative to position 0, a U/U mismatch at position −11 relative to position 0, a C/A mismatch at position −5 relative to position 0, a 2/2 symmetric bulge at position −1 relative to position 0, a G/U Wobble at position 6 relative to position 0, a C/U mismatch at position 10 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

112. The composition of claim 111, wherein the engineered guide RNA comprises at least about: 80%, 85%, 90%, 92%, 95%, 97%, or 99% sequence identity to SEQ ID NO: 589.

113. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, and any combination thereof.

114. The composition of claim 113, wherein the engineered guide RNA comprises at least about: 80%, 85%, 90%, 92%, 95%, 97%, or 99% sequence identity to SEQ ID NO: 610.

115. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/C mismatch at position 9 relative to position 0, a G/U Wobble at position 18 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

116. The composition of claim 115, wherein the engineered guide RNA comprises at least about: 80%, 85%, 90%, 92%, 95%, 97%, or 99% sequence identity to SEQ ID NO: 611.

117. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/G mismatch at position 18 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

118. The composition of claim 117, wherein the engineered guide RNA comprises at least about: 80%, 85%, 90%, 92%, 95%, 97%, or 99% sequence identity to SEQ ID NO: 618.

119. The composition of claim 102, wherein the one or more structural features further comprises at least one structural feature selected from the group consisting of: a 6/6 symmetric internal loop at position −20 relative to position 0, a A/G mismatch at position −7 relative to position 0, a C/U mismatch at position −4 relative to position 0, a 2/2 symmetric bulge at position −1 relative to position 0, a G/U Wobble at position 8 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

120. The composition of claim 119, wherein the engineered guide RNA comprises at least about: 80%, 85%, 90%, 92%, 95%, 97%, or 99% sequence identity to SEQ ID NO: 662.

121. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 3 relative to position 0, a 2/2 symmetric bulge at position 7 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

122. The composition of claim 121, wherein the engineered guide RNA comprises at least about: 80%, 85%, 90%, 92%, 95%, 97%, or 99% sequence identity to SEQ ID NO: 664.

123. The composition of claim 102, wherein the one or more structural features further comprises at least one structural feature selected from the group consisting of: a 6/6 symmetric internal loop at position −20 relative to position 0, a A/C mismatch at position −7 relative to position 0, a C/U mismatch at position −4 relative to position 0, a 2/2 symmetric bulge at position −1 relative to position 0, a 2/2 symmetric bulge at position 10 relative to position 0, an A/C mismatch at position 19 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

124. The composition of claim 123, wherein the engineered guide RNA comprises at least about: 80%, 85%, 90%, 92%, 95%, 97%, or 99% sequence identity to SEQ ID NO: 672.

125. The composition of claim 102, wherein the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, and any combination thereof.

126. The composition of claim 125, wherein the engineered guide RNA comprises at least about: 80%, 85%, 90%, 92%, 95%, 97%, or 99% sequence identity to SEQ ID NO: 675.

127. The composition of claim 102, wherein the one or more structural features further comprises at least one structural feature selected from the group consisting of: a 6/6 symmetric internal loop at position −20 relative to position 0, a C/C mismatch at position −5 relative to position 0, a 2/2 symmetric bulge at position −1 relative to position 0, a G/U Wobble at position 8 relative to position 0, a C/C mismatch at position 12 relative to position 0, a G/G mismatch at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

128. The composition of claim 127, wherein the engineered guide RNA comprises at least about: 80%, 85%, 90%, 92%, 95%, 97%, or 99% sequence identity to SEQ ID NO: 676.

129. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, and any combination thereof.

130. The composition of claim 129, wherein the engineered guide RNA comprises at least about: 80%, 85%, 90%, 92%, 95%, 97%, or 99% sequence identity to SEQ ID NO: 699.

131. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a C/A mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

132. The composition of claim 131, wherein the engineered guide RNA comprises at least about: 80%, 85%, 90%, 92%, 95%, 97%, or 99% sequence identity to SEQ ID NO: 700.

133. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, and any combination thereof.

134. The composition of claim 133, wherein the engineered guide RNA comprises at least about: 80%, 85%, 90%, 92%, 95%, 97%, or 99% sequence identity to SEQ ID NO: 718.

135. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a U/C mismatch at position 5 relative to position 0, a C/A mismatch at position 9 relative to position 0, a C/A mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

136. The composition of claim 135, wherein the engineered guide RNA comprises at least about: 80%, 85%, 90%, 92%, 95%, 97%, or 99% sequence identity to SEQ ID NO: 719.

137. The composition of claim 102, wherein the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a C/C mismatch at position 9 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

138. The composition of claim 137, wherein the engineered guide RNA comprises at least about: 80%, 85%, 90%, 92%, 95%, 97%, or 99% sequence identity to SEQ ID NO: 745.

139. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

140. The composition of claim 139, wherein the engineered guide RNA comprises at least about: 80%, 85%, 90%, 92%, 95%, 97%, or 99% sequence identity to SEQ ID NO: 750.

141. The composition of claim 102, wherein the one or more structural features further comprises at least one structural feature selected from the group consisting of: a 6/6 symmetric internal loop at position −20 relative to position 0, a A/A mismatch at position −7 relative to position 0, a 2/2 symmetric bulge at position −1 relative to position 0, a C/U mismatch at position 10 relative to position 0, a G/U Wobble at position 20 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

142. The composition of claim 141, wherein the engineered guide RNA comprises at least about: 80%, 85%, 90%, 92%, 95%, 97%, or 99% sequence identity to SEQ ID NO: 762.

143. The composition of claim 102, wherein the one or more structural features further comprises at least one structural feature selected from the group consisting of: a 6/6 symmetric internal loop at position −20 relative to position 0, a 3/3 symmetric bulge at position −5 relative to position 0, a 2/2 symmetric bulge at position −1 relative to position 0, a U/U mismatch at position 5 relative to position 0, a G/U Wobble at position 8 relative to position 0, a C/U mismatch at position 14 relative to position 0, a G/G mismatch at position 17 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

144. The composition of claim 143, wherein the engineered guide RNA comprises at least about: 80%, 85%, 90%, 92%, 95%, 97%, or 99% sequence identity to SEQ ID NO: 1099.

145. The composition of claim 102, wherein the one or more structural features further comprises at least one structural feature selected from the group consisting of: a 6/6 symmetric internal loop at position −20 relative to position 0, a C/A mismatch at position −5 relative to position 0, a 2/2 symmetric bulge at position −1 relative to position 0, a G/U Wobble at position 6 relative to position 0, a C/U mismatch at position 9 relative to position 0, a C/U mismatch at position 14 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

146. The composition of claim 145, wherein the engineered guide RNA comprises at least about: 80%, 85%, 90%, 92%, 95%, 97%, or 99% sequence identity to SEQ ID NO: 1120.

147. The composition of claim 102, wherein the one or more structural features further comprises at least one structural feature selected from the group consisting of: a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

148. The composition of claim 147, wherein the engineered guide RNA comprises at least about: 80%, 85%, 90%, 92%, 95%, 97%, or 99% sequence identity to SEQ ID NO: 1213.

149. The composition of claim 102, wherein the one or more structural features further comprises at least one structural feature selected from the group consisting of: a 6/6 symmetric internal loop at position −22 relative to position 0, a 3/3 symmetric bulge mismatch at position −13 relative to position 0, a 6/6 symmetric internal loop at position −1 relative to position 0, a 3/3 symmetric bulge at position 38 relative to position 0, a 3/3 symmetric bulge at position 47 relative to position 0, a 3/3 symmetric bulge at position 56 relative to position 0, a 3/3 symmetric bulge at position 65 relative to position 0, and any combination thereof.

150. The composition of claim 149, wherein the engineered guide RNA comprises at least about: 80%, 85%, 90%, 92%, 95%, 97%, or 99% sequence identity to SEQ ID NO: 1214.

151. The composition of claim 2, wherein the first 6/6 symmetric internal loop is at position 25, relative to the target adenosine at position 0.

152. The composition of claim 151, wherein the one or more structural features further comprises at least one structural feature selected from the group consisting of: a 3/3 symmetric bulge at position −25 relative to position 0, a 3/3 symmetric bulge at position −16 relative to position 0, a 6/6 symmetric internal loop at position −4 relative to position 0, a G/G mismatch at position −1 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof.

153. The composition of claim 152, wherein the engineered guide RNA comprises at least about: 80%, 85%, 90%, 92%, 95%, 97%, or 99% sequence identity to SEQ ID NO: 88.

154. The composition of claim 151, wherein the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof.

155. The composition of claim 154, wherein the engineered guide RNA comprises at least about: 80%, 85%, 90%, 92%, 95%, 97%, or 99% sequence identity to SEQ ID NO: 903.

156. The composition of claim 151, wherein the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof.

157. The composition of claim 156, wherein the engineered guide RNA comprises at least about: 80%, 85%, 90%, 92%, 95%, 97%, or 99% sequence identity to SEQ ID NO: 1179.

158. The composition of claim 151, wherein the one or more structural features further comprises at least one structural feature selected from the group consisting of: a 6/6 symmetric internal loop at position −4 relative to position 0, a G/G mismatch at position −1 relative to position 0, a 3/3 symmetric bulge at position 6 relative to position 0, a 3/3 symmetric bulge at position 37 relative to position 0, a 3/3 symmetric bulge at position 46 relative to position 0, a 3/3 symmetric bulge at position 55 relative to position 0, a 3/3 symmetric bulge at position 64 relative to position 0, and any combination thereof.

159. The composition of claim 158, wherein the engineered guide RNA comprises at least about: 80%, 85%, 90%, 92%, 95%, 97%, or 99% sequence identity to SEQ ID NO: 1212.

160. The composition of claim 2, wherein the first 6/6 symmetric internal loop is at position 24, relative to the target adenosine at position 0.

161. The composition of claim 160, wherein the one or more structural features further comprises at least one structural feature selected from the group consisting of: a 3/3 symmetric bulge at position −18 relative to position 0, a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

162. The composition of claim 161, wherein the engineered guide RNA comprises at least about: 80%, 85%, 90%, 92%, 95%, 97%, or 99% sequence identity to SEQ ID NO: 118.

163. The composition of claim 160, wherein the one or more structural features further comprises at least one structural feature selected from the group consisting of: a 6/6 symmetric internal loop at position −6 relative to position 0, a G/G mismatch at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

164. The composition of claim 163, wherein the engineered guide RNA comprises at least about: 80%, 85%, 90%, 92%, 95%, 97%, or 99% sequence identity to SEQ ID NO: 119.

165. The composition of claim 160, wherein the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

166. The composition of claim 165, wherein the engineered guide RNA comprises at least about: 80%, 85%, 90%, 92%, 95%, 97%, or 99% sequence identity to SEQ ID NO: 270.

167. The composition of claim 160, wherein the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

168. The composition of claim 167, wherein the engineered guide RNA comprises at least about: 80%, 85%, 90%, 92%, 95%, 97%, or 99% sequence identity to SEQ ID NO: 271.

169. The composition of claim 160, wherein the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

170. The composition of claim 169, wherein the engineered guide RNA comprises at least about: 80%, 85%, 90%, 92%, 95%, 97%, or 99% sequence identity to SEQ ID NO: 403.

171. The composition of claim 160, wherein the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 36 relative to position 0, a 3/3 symmetric bulge at position 45 relative to position 0, a 3/3 symmetric bulge at position 54 relative to position 0, a 3/3 symmetric bulge at position 63 relative to position 0, and any combination thereof.

172. The composition of claim 171, wherein the engineered guide RNA comprises at least about: 80%, 85%, 90%, 92%, 95%, 97%, or 99% sequence identity to SEQ ID NO: 1081.

173. The composition of claim 2, wherein the first 6/6 symmetric internal loop is at position 23, relative to the target adenosine at position 0.

174. The composition of claim 173, wherein the one or more structural features further comprises at least one structural feature selected from the group consisting of: a 3/3 symmetric bulge at position −17 relative to position 0, a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

175. The composition of claim 174, wherein the engineered guide RNA comprises at least about: 80%, 85%, 90%, 92%, 95%, 97%, or 99% sequence identity to SEQ ID NO: 401.

176. The composition of claim 173, wherein the one or more structural features further comprises at least one structural feature selected from the group consisting of: a 6/6 symmetric internal loop at position −5 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

177. The composition of claim 176, wherein the engineered guide RNA comprises at least about: 80%, 85%, 90%, 92%, 95%, 97%, or 99% sequence identity to SEQ ID NO: 402.

178. The composition of claim 173, wherein the one or more structural features further comprises at least one structural feature selected from the group consisting of: a 3/3 symmetric bulge at position −19 relative to position 0, a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

179. The composition of claim 178, wherein the engineered guide RNA comprises at least about: 80%, 85%, 90%, 92%, 95%, 97%, or 99% sequence identity to SEQ ID NO: 458.

180. The composition of claim 173, wherein the one or more structural features further comprises at least one structural feature selected from the group consisting of: a 6/6 symmetric internal loop at position −7 relative to position 0, a 2/2 symmetric bulge at position −1 relative to position 0, a 3/3 symmetric bulge at position 35 relative to position 0, a 3/3 symmetric bulge at position 44 relative to position 0, a 3/3 symmetric bulge at position 53 relative to position 0, a 3/3 symmetric bulge at position 62 relative to position 0, and any combination thereof.

181. The composition of claim 180, wherein the engineered guide RNA comprises at least about: 80%, 85%, 90%, 92%, 95%, 97%, or 99% sequence identity to SEQ ID NO: 459.

182. The composition of claim 2, wherein the first 6/6 symmetric internal loop is at position 22, relative to the target adenosine at position 0.

183. The composition of claim 182, wherein the one or more structural features further comprises at least one structural feature selected from the group consisting of: a 3/3 symmetric bulge at position −22 relative to position 0, a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof.

184. The composition of claim 183, wherein the engineered guide RNA comprises at least about: 80%, 85%, 90%, 92%, 95%, 97%, or 99% sequence identity to SEQ ID NO: 1135.

185. The composition of claim 182, wherein the one or more structural features further comprises at least one structural feature selected from the group consisting of: a 6/6 symmetric internal loop at position −10 relative to position 0, a 2/2 symmetric bulge at position −1 relative to position 0, a 3/3 symmetric bulge at position 6 relative to position 0, a 3/3 symmetric bulge at position 34 relative to position 0, a 3/3 symmetric bulge at position 43 relative to position 0, a 3/3 symmetric bulge at position 52 relative to position 0, a 3/3 symmetric bulge at position 61 relative to position 0, and any combination thereof.

186. The composition of claim 185, wherein the engineered guide RNA comprises at least about: 80%, 85%, 90%, 92%, 95%, 97%, or 99% sequence identity to SEQ ID NO: 1136.

187. The composition of any one of claims 1-186, wherein the one or more structural features comprises at least a first 6/6 symmetric internal loop and at least a second 6/6 symmetric loop

188. The composition of any one of claims 1-187, wherein upon hybridization of the engineered guide RNA to the sequence of the target MAPT RNA, the engineered guide RNA facilitates RNA editing of one or more adenosines in the sequence of the target MAPT RNA by an RNA editing entity.

189. The composition of claim 188, wherein the RNA editing entity comprises ADAR1, ADAR2, ADAR3, or any combination thereof.

190. The composition of any one of claims 1-189, wherein the engineered guide RNA is encoded by an engineered polynucleotide.

191. The composition of claim 190, wherein the engineered polynucleotide is comprised in or on a vector.

192. The composition of claim 191, wherein the vector is a viral vector, and wherein the engineered polynucleotide is encapsidated in the viral vector.

193. The composition of claim 192, wherein the viral vector is an adeno-associated viral (AAV) vector or a derivative thereof.

194. The composition of claim 192, wherein the viral vector is an adeno-associated viral (AAV) vector and wherein the AAV vector is AAV1, AAV2, AAV3, AAV4, AAV5, AAV6, AAV7, AAV8, AAV9, AAV10, AAV11, or a derivative, a chimera, or a variant of any of these.

195. The composition of any one of claims 193-194, wherein the AAV vector is a recombinant AAV (rAAV) vector, a hybrid AAV vector, a chimeric AAV vector, a self-complementary AAV (scAAV) vector, or any combination thereof.

196. The composition of any one of claims 1-195, wherein the engineered guide RNA has at least 80%, 85%, 90%, 92%, 95%, 97%, or 99% sequence identity to any one of SEQ ID NO: 2-SEQ ID NO: 1215.

197. The composition of any one of claims 1-196, in unit dose form.

198. A pharmaceutical composition comprising:
a) the composition of any one of claims 1-197; and
b) a pharmaceutically acceptable: excipient, carrier, or diluent.

199. The pharmaceutical composition of claim 198, in unit dose form.

200. A method of treating a disease or a condition in a subject in need thereof, the method comprising administering to the subject a therapeutically effective amount of the composition of any one of claims 1-197 or the pharmaceutical composition of claim 198 or 199.

201. The method of claim 200, wherein the disease or condition comprises a Tauopathy.

202. The method of claim 200, wherein the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome.

203. The method of any one of claims 200-202, wherein the subject is a human or a non-human animal.

204. The method of any one of claims 200-203, wherein the administering is sufficient to treat one or more symptoms of the disease of condition.

205. The method of claim 204, wherein the one of more symptoms treated comprises a reduction in memory loss, a reduction in confusion, or any combination thereof, as compared to prior to the administering.

206. The method of claim 204-205, wherein the administering is sufficient to reduce aggregation of tau protein, relative to:
(a) a level of aggregation prior to the administering;
(b) a level of accumulated aggregation in the subject in the absence of the administering; or
(c) both.

207. The method of claim 204-205, wherein the administering is sufficient to reduce an amount of:
(a) neurofibrillary tangles;
(b) neuronal cell death;
(c) neuroinflammation; or
(d) any combination thereof;
relative to an amount prior to the administering.

208. The composition of claim 204, wherein the amount of phosphorylated tau protein is decreased in the subject after the administering, relative to an amount of phosphorylated tau protein prior to the administering.

209. The composition of claim 204, wherein a ratio of a 4R isoform of Tau to a 3R isoform of tau is increased upon administration, relative to a ratio prior to the administering.

210. The composition of any one of claims 1-197 or the pharmaceutical composition of claim 198 or 199 for use in treatment of a disease or condition.

211. The composition or the pharmaceutical composition for use of claim 210, wherein the disease or condition comprises a Tauopathy.

212. The composition or the pharmaceutical composition for use of claim 210, wherein the disease or condition comprises Alzheimer's disease (AD), frontotemporal dementia (FTD), Parkinson's disease, progressive supranuclear palsy (PSP), corticobasal degeneration (CBD), chronic traumatic encephalopathy, autism, traumatic brain injury, or Dravet syndrome.

213. A method of editing a c.1 translation initiation sequence (TIS) of a MAPT RNA, the method comprising contacting the MAPT RNA with the composition of any one of claims 1-197 and an RNA editing entity, thereby editing the MAPT RNA.

214. The method of claim 213, wherein at least 40%, at least 50%, at least 60%, at least 70%, at least 80%, at least 85%, at least 90%, at least 95%, or at least 99% of the pre-mRNA transcripts of MAPT have at least one edit.

215. The method of claim 213, wherein the editing of MAPT RNA facilitates a protein knockdown.

216. The method of claim 215, wherein the protein knockdown comprises a reduction of at least 10%, relative to an amount of protein present prior to the contacting.

217. The method of claim 215, wherein the protein knockdown comprises a reduction of from about 10% to about 25%, relative to an amount of protein present prior to the contacting.

218. The method of claim 215, wherein the protein knockdown comprises a reduction of at least 50%, relative to an amount of protein present prior to the contacting.

219. The method of claim 215, wherein the protein knockdown comprises a knockdown of tau.

220. The method of any one of claims 215-219, wherein the knockdown is measured in an in vitro assay.

221. The method of any one of claims 215-219, wherein the knockdown is measured in an in vivo assay.

222. The method of any one of claims 215-219, wherein the knockdown is measured in a human subject.
